SEQUENCE LISTING (129 sequences)

SEQ ID NO: 1 - protein, 439 aa, Aedes aegypti
MWFFKVGALLFLAALVSANNATTGPKVLCYYDGQMSLREGLGKITVTDIELALPFCTHLLYGFAGVNPETYRLKALDESLELDSGKGQYRLATTLKRRYPNLKVLLSVGGYKDLTEEKPFEKYLTLLESAGSRTAFVNSVYSTLKTYDFDGLDLAWQFPQTKPKRIRGWTGKVWHGFKKLFTGDSVLDPKADEHREEFTALVRDLKNALVADNFILGLTVLPHVNESIFMDVPLLKDNLDYVNLASFDQQTPERNPKEGDYTAPIYEPSERVEGNNVDAEASYWQGTPAGKIVIGIPTYGRGWKLVEKSGITGVPPIPADGPSIPGPHSGINGFYSWAEVCAKLPNPGNANLQGADQPLRKIGDPTRRFGAYAFRIPDENEEHGIWLSYEDPDTAGNKAAYVKAKGLGGISIFDLGNDDVRGACAGDKFPILRAAKYRL

SEQ ID NO: 2 - protein, 312 aa, Aedes aegypti
MSPSKKILVLLFFPILLVSSHPIPAEDPAKQCNLSEDDLTKLKAAISSASSAKAANEDILPSTTLAACPMLKNFTEMLKTVATDMEVLKTQGVSNMEVQLLRESFEEKLNDLAKNKDIFERQANQDTSKAEGEMVEKINKLQLEMAKLQEEIEEQTKQMYVDMIEYIFERLKMNDTEAIDSYAQIVMKTKMHELIMKLKTDRLVLWEMVKYVEGKKNKWVGRKVLNTILDQVNKLKLYKPEEVEIGKNSLVVVWCWKFNSETVYGTTDEDQKSFHLAKLFFPKEKGCKECADVKSRTMCNNDYPKVMVKAFG

SEQ ID NO: 3 - protein, 316 aa, Aedes aegypti
METSLPITVVFLIVLITGAQTKPTQGSCTLTDEDISDIKSAVQKASKAAVNDIVLDPTLIDKCPMLEKITASLKSVATEIVQMRDSAISTDQVDQLKQNFEDQVNQIVKSRDIFEKQSGTQATKEHGEMLERMTALQVKVTELEQQIAKQTASMYEDMAELIFQRLQMNSTESVRSYTKHMMEEKLEELMNKLETNYRIYLGALRFLNHMNDQELIGKVFDGILKRLGDMKADSDDVKENGRNLLVNLLCWTVNNDFLGKKYKERQVDLYRMALKFYPKTYEKAANEADVRSRQFCEENFPANLITWFAVSWNDRG

SEQ ID NO: 4 - protein, 332 aa, Aedes aegypti
MFPPRKFLLSSFILAALHVTAAPLWDAKDPEQLRFITSRCMEDWYPKAKNPKAALQNWLGWKLEPSDDQATQCYTKCVLEKIGFYEPGEKRFKGVRVMQQWETFHKYLNADREKVHDLTSTFDFIPPLKSSSCSEVFEAFKKVNGKHSETIRAILFGKGESSKKYYQEKGVKIKQKEQSLFMHCEALNYPKGSPQRKDLCGIRKYQMGSGIVFERHMECIFKGLRYMTSKNELDVDEIARDFIVVKKKPDAMKAMMKTCKANLKEKNPGKIAVHYYKCLMNDSKVTNDFKEAFDYREVRSKDYFAALTGKLKPYSRSDVRKQVDDIDKIQCS

SEQ ID NO: 5 - protein, 215 aa, Aedes aegypti
MKYLLTFLMALSLVNLMLTRPTPEDDGGTSEEPQTQETTGSDEKNGASEEPNADDASKPDDVEEKGDDDTAKKEDDGESKDGEGSEKSDKEKGEPKNDPRETYNKVIEQLDQIKVDNVEDGHERSELAADIQRYLRNPIVDVIGSAGDFSKIAKCFKSMVGDAKKAIEEDVKGFKECTAKKDSNAYQCSQDRSTVQDKIAKMSSKIASCVASNRS

SEQ ID NO: 6 - protein, 11 aa, Artificial sequence
  Aedes aegypti
NLKVLLSVGGY

SEQ ID NO: 7 - protein, 15 aa, Artificial sequence
  Aedes aegypti
FKVGALLFLAALVSA

SEQ ID NO: 8 - protein, 29 aa, Artificial sequence
  Aedes aegypti
TALVRDLKNALVADNFILGLTVLPHVNES

SEQ ID NO: 9 - protein, 24 aa, Artificial sequence
  Aedes aegypti
KITVTDIELALPFCTHLLYGFAGV

SEQ ID NO: 10 - protein, 14 aa, Artificial sequence
  Aedes aegypti
TAFVNSVYSTLKTY

SEQ ID NO: 11 - protein, 19 aa, Artificial sequence
  Aedes aegypti
FMDVPLLKDNLDYVNLASF

SEQ ID NO: 12 - protein, 12 aa, Artificial sequence
  Aedes aegypti
ITGVPPIPADGP

SEQ ID NO: 13 - protein, 16 aa, Artificial sequence
  Aedes aegypti
RGACAGDKFPILRAAK

SEQ ID NO: 14 - protein, 10 aa, Artificial sequence
  Aedes aegypti
NSLVVVWCWK

SEQ ID NO: 15 - protein, 22 aa, Artificial sequence
  Aedes aegypti
KKILVLLFFPILLVSSHPIPAE

SEQ ID NO: 16 - protein, 12 aa, Artificial sequence
  Aedes aegypti
LPSTTLAACPML

SEQ ID NO: 17 - protein, 14 aa, Artificial sequence
  Aedes aegypti
TDRLVLWEMVKYVE

SEQ ID NO: 18 - protein, 10 aa, Artificial sequence
  Aedes aegypti
SFHLAKLFFP

SEQ ID NO: 19 - protein, 19 aa, Artificial sequence
  Aedes aegypti
LNTILDQVNKLKLYKPEEV

SEQ ID NO: 20 - protein, 13 aa, Artificial sequence
  Aedes aegypti
TKLKAAISSASSA

SEQ ID NO: 21 - protein, 12 aa, Artificial sequence
  Aedes aegypti
QMYVDMIEYIFE

SEQ ID NO: 22 - protein, 15 aa, Artificial sequence
  Aedes aegypti
SLPITVVFLIVLITG

SEQ ID NO: 23 - protein, 11 aa, Artificial sequence
  Aedes aegypti
NLLVNLLCWTV

SEQ ID NO: 24 - protein, 16 aa, Artificial sequence
  Aedes aegypti
ALQVKVTELEQQIAKQ

SEQ ID NO: 25 - protein, 29 aa, Artificial sequence
  Aedes aegypti
IKSAVQKASKAAVNDIVLDPTLIDKCPML

SEQ ID NO: 26 - protein, 13 aa, Artificial sequence
  Aedes aegypti
ITASLKSVATEIV

SEQ ID NO: 27 - protein, 10 aa, Artificial sequence
  Aedes aegypti
ANLITWFAVS

SEQ ID NO: 28 - protein, 11 aa, Artificial sequence
  Aedes aegypti
IGKVFDGILKR

SEQ ID NO: 29 - protein, 17 aa, Artificial sequence
  Aedes aegypti
ERQVDLYRMALKFYPKT

SEQ ID NO: 30 - protein, 13 aa, Artificial sequence
  Aedes aegypti
ATQCYTKCVLEKI

SEQ ID NO: 31 - protein, 13 aa, Artificial sequence
  Aedes aegypti
KIAVHYYKCLMND

SEQ ID NO: 32 - protein, 21 aa, Artificial sequence
  Aedes aegypti
RKFLLSSFILAALHVTAAPLW

SEQ ID NO: 33 - protein, 9 aa, Artificial sequence
  Aedes aegypti
RDFIVVKKK

SEQ ID NO: 34 - protein, 22 aa, Artificial sequence
  Aedes aegypti
TFDFIPPLKSSSCSEVFEAFKK

SEQ ID NO: 35 - protein, 11 aa, Artificial sequence
  Aedes aegypti
LFMHCEALNYP

SEQ ID NO: 36 - protein, 12 aa, Artificial sequence
  Aedes aegypti
YFAALTGKLKPY

SEQ ID NO: 37 - protein, 12 aa, Artificial sequence
  Aedes aegypti
KMSSKIASCVAS

SEQ ID NO: 38 - protein, 16 aa, Artificial sequence
  Aedes aegypti
LLTFLMALSLVNLMLT

SEQ ID NO: 39 - protein, 22 aa, Artificial sequence
  Aedes aegypti
SELAADIQRYLRNPIVDVIGSA

SEQ ID NO: 40 - protein, 15 aa, Artificial sequence
  Aedes aegypti
NKVIEQLDQIKVDNV

SEQ ID NO: 41 - protein, 13 aa, Artificial sequence
  Aedes aegypti
FSKIAKCFKSMVG

SEQ ID NO: 42 - protein, 14 aa, Artificial sequence
  Aedes aegypti
AYQCSQDRSTVQDK

SEQ ID NO: 43 - protein, 291 aa, Aedes aegypti
MNRQLWIIIFAILCVAQAEEDNPTTEKMEELGIATINNFTREFYSYVEAVSQVLADLELTTTASITQIKHRIKHLLQEKCNLCSAKAEGPALDQGYVTTSNGSVIPVSYEQTRFGGGWIVLMQRYDGTVRFNRSWAEYRDGFGMVGHEFWLGLERIHQMTKDAEYELMIEMQDFEGNYKYAGYDAFAVGPEEERYPLAKVGKFNKTAYVDSFGKHRGYGFSTYDNDDNGCSNQYGRGGWWYYRKSCFGASLTGIWQNKQDWKSISWVWFSTEKKQVPLKFARMMMRLKTAE

SEQ ID NO: 44 - protein, 321 aa, Aedes aegypti
MKLPLLLAIVTTFSVVASTGPFDPEEMLFTFTRCMEDNLEDGPNRLPMLAKWKEWINEPVDSPATQCFGKCVLVRTGLYDPVAQKFDASVIQEQFKAYPSLGEKSKVEAYANAVQQLPSTNNDCAAVFKAYDPVHKAHKDTSKNLFHGNKELTKGLYEKLGKDIRQKKQSYFEFCENKYYPAGSDKRQQLCKIRQYTVLDDALFKEHTDCVMKGIRYITKNNELDAEEVKRDFMQVNKDTKALEKVLNDCKSKEPSNAGEKSWHYYKCLVESSVKDDFKEAFDYREVRSQIYAFNLPKKQVYSKPAVQSQVMEIDGKQCPQ

SEQ ID NO: 45 - protein, 339 aa, Aedes aegypti
MVQFPVLLITLSLAFEVHSSYAENRRLQLVRDIDGTQQLVNPNPYRVLNAHLERSFNAQSDIIFRLYTRKNPEKHQILKPNDTSSILNSNFNADLPTRFLIHGWNQNGESDILIELRRSYLSVEDFNVIGVDWGEGALTINYVMARKRVESVGLVTSQLIDTLVDASGVILDSIYVIGHSLGAHVAGIVGKHQRGQLNTIVGLDPAGPLFSLNSSDILNQNHAQYVEMVSTGARLLGTYEPLGDANFYPNGGLEQAGCGLDLFGICAHARSWIYFAETVTNGKGFRGIKCAMIEDLEGETCNLSGLPNVWMGGEPSNHERGVKGIFMVHTNSEAPFAKD

SEQ ID NO: 46 - protein, 290 aa, Aedes aegypti
MILQFWVVTFSVLFAARADENHSILIKLNDLDHRFTQMFSQQFYRHTQQVTDRVSALKISIDTNLLELDQQIQQALDGIQSNESSSSASATKPPGLTTIPIGSEPRVPALYERERYGGDWLVVMHRYDGSVKFDRTWAEYRDGFGMVGQEFWYGLERLHQLTKEKSYELMVEMEDFNGSLKYAWYDKFVVGPEEQRYALVELGTFNGTTDGDSLKPHKGSGFSTYDNDDFGCSNKYAKGGWWYYSGKCYGSSLTGIWKNELAYSSIVWMKFSDVSNTPLKLVRMMIRPKN

SEQ ID NO: 47 - protein, 14 aa, Artificial sequence
  Aedes aegypti
LWIIIFAILCVAQA

SEQ ID NO: 48 - protein, 16 aa, Artificial sequence
  Aedes aegypti
FYSYVEAVSQVLADLE

SEQ ID NO: 49 - protein, 9 aa, Artificial sequence
  Aedes aegypti
GSVIPVSYE

SEQ ID NO: 50 - protein, 22 aa, Artificial sequence
  Aedes aegypti
ITQIKHRIKHLLQEKCNLCSAK

SEQ ID NO: 51 - protein, 8 aa, Artificial sequence
  Aedes aegypti
KKQVPLKF

SEQ ID NO: 52 - protein, 7 aa, Artificial sequence
  Aedes aegypti
YPLAKVG

SEQ ID NO: 53 - protein, 11 aa, Artificial sequence
  Aedes aegypti
RKSCFGASLTG

SEQ ID NO: 54 - protein, 7 aa, Artificial sequence
  Aedes aegypti
GWIVLMQ

SEQ ID NO: 55 - protein, 8 aa, Artificial sequence
  Aedes aegypti
LDQGYVTT

SEQ ID NO: 56 - protein, 10 aa, Artificial sequence
  Aedes aegypti
YAGYDAFAVG

SEQ ID NO: 57 - protein, 7 aa, Artificial sequence
  Aedes aegypti
TAYVDSF

SEQ ID NO: 58 - protein, 7 aa, Artificial sequence
  Aedes aegypti
ISWVWFS

SEQ ID NO: 59 - protein, 7 aa, Artificial sequence
  Aedes aegypti
WLGLERI

SEQ ID NO: 60 - protein, 16 aa, Artificial sequence
  Aedes aegypti
PLLLAIVTTFSVVAST

SEQ ID NO: 61 - protein, 11 aa, Artificial sequence
  Aedes aegypti
WHYYKCLVESS

SEQ ID NO: 62 - protein, 19 aa, Artificial sequence
  Aedes aegypti
PVDSPATQCFGKCVLVRTG

SEQ ID NO: 63 - protein, 14 aa, Artificial sequence
  Aedes aegypti
CAAVFKAYDPVHKA

SEQ ID NO: 64 - protein, 29 aa, Artificial sequence
  Aedes aegypti
YREVRSQIYAFNLPKKQVYSKPAVQSQVM

SEQ ID NO: 65 - protein, 13 aa, Artificial sequence
  Aedes aegypti
KVEAYANAVQQLP

SEQ ID NO: 66 - protein, 23 aa, Artificial sequence
  Aedes aegypti
YDPVAQKFDASVIQEQFKAYPSL

SEQ ID NO: 67 - protein, 15 aa, Artificial sequence
  Aedes aegypti
QQLCKIRQYTVLDDA

SEQ ID NO: 68 - protein, 8 aa, Artificial sequence
  Aedes aegypti
LEKVLNDC

SEQ ID NO: 69 - protein, 12 aa, Artificial sequence
  Aedes aegypti
YFEFCENKYYPA

SEQ ID NO: 70 - protein, 11 aa, Artificial sequence
  Aedes aegypti
FKEHTDCVMKG

SEQ ID NO: 71 - protein, 17 aa, Artificial sequence
  Aedes aegypti
FPVLLITLSLAFEVHSS

SEQ ID NO: 72 - protein, 44 aa, Artificial sequence
  Aedes aegypti
VESVGLVTSQLIDTLVDASGVILDSIYVIGHSLGAHVAGIVGKH

SEQ ID NO: 73 - protein, 23 aa, Artificial sequence
  Aedes aegypti
QAGCGLDLFGICAHARSWIYFAE

SEQ ID NO: 74 - protein, 6 aa, Artificial sequence
  Aedes aegypti
LQLVRD

SEQ ID NO: 75 - protein, 16 aa, Artificial sequence
  Aedes aegypti
TQQLVNPNPYRVLNAH

SEQ ID NO: 76 - protein, 8 aa, Artificial sequence
  Aedes aegypti
TINYVMAR

SEQ ID NO: 77 - protein, 7 aa, Artificial sequence
  Aedes aegypti
AQYVEMV

SEQ ID NO: 78 - protein, 18 aa, Artificial sequence
  Aedes aegypti
NTIVGLDPAGPLFSLNSS

SEQ ID NO: 79 - protein, 7 aa, Artificial sequence
  Aedes aegypti
KGIFMVH

SEQ ID NO: 80 - protein, 9 aa, Artificial sequence
  Aedes aegypti
CNLSGLPNV

SEQ ID NO: 81 - protein, 16 aa, Artificial sequence
  Aedes aegypti
DILIELRRSYLSVEDF

SEQ ID NO: 82 - protein, 8 aa, Artificial sequence
  Aedes aegypti
PTRFLIHG

SEQ ID NO: 83 - protein, 8 aa, Artificial sequence
  Aedes aegypti
SDIIFRLY

SEQ ID NO: 84 - protein, 7 aa, Artificial sequence
  Aedes aegypti
GIKCAMI

SEQ ID NO: 85 - protein, 8 aa, Artificial sequence
  Aedes aegypti
GARLLGTY

SEQ ID NO: 86 - protein, 13 aa, Artificial sequence
  Aedes aegypti
QFWVVTFSVLFAA

SEQ ID NO: 87 - protein, 24 aa, Artificial sequence
  Aedes aegypti
LAYSSIVWMKFSDVSNTPLKLVRM

SEQ ID NO: 88 - protein, 8 aa, Artificial sequence
  Aedes aegypti
DWLVVMHR

SEQ ID NO: 89 - protein, 8 aa, Artificial sequence
  Aedes aegypti
EPRVPALY

SEQ ID NO: 90 - protein, 9 aa, Artificial sequence
  Aedes aegypti
HSILIKLND

SEQ ID NO: 91 - protein, 8 aa, Artificial sequence
  Aedes aegypti
RYALVELG

SEQ ID NO: 92 - protein, 10 aa, Artificial sequence
  Aedes aegypti
TDRVSALKIS

SEQ ID NO: 93 - protein, 11 aa, Artificial sequence
  Aedes aegypti
SGKCYGSSLTG

SEQ ID NO: 94 - protein, 14 aa, Artificial sequence
  Aedes aegypti
SLKYAWYDKFVVGP

SEQ ID NO: 95 - protein, 7 aa, Artificial sequence
  Aedes aegypti
LERLHQL

SEQ ID NO: 96 - protein, 17 aa, Artificial sequence
  Aedes aegypti
DTNLLELDQQIQQALDG

SEQ ID NO: 97 - protein, 10 aa, Artificial sequence
  Aedes aegypti
SQQFYRHTQQ

SEQ ID NO: 98 - protein, 6 aa, Artificial sequence
  Aedes aegypti
DGSVKF

SEQ ID NO: 99 - protein, 10 aa, Artificial sequence
  Aedes aegypti
PPGLTTIPIG

SEQ ID NO: 100 - protein, 651 aa, Aedes aegypti
MAKAPAVGIDLGTTYSCVGVFQHGKVEIIANDQGNRTTPSYVAFTDTERLIGDAAKNQVAMNPTNTIFDAKRLIGRKFDDPAIQADMKHWPFDVISVEGKPKIQVEYKGETKNFFPEEISSMVLTKMKETAEAYLGKTVSNAVVTVPAYFNDSQRQATKDAGTISGLNVLRIINEPTAAAIAYGLDKKTAGERNVLIFDLGGGTFDVSILSIDDGIFEVKSTAGDTHLGGEDFDNRLVNHFAQEFKRKHKKDLSTNKRALRRLRTACERAKRTLSSSTQASIEIDSLFEGTDFYTSITRARFEELNADLFRSTMEPVEKAIRDAKMDKASIHDIVLVGGSTRIPKVQKLLQDFFNGKELNKSINPDEAVAYGAAVQAAILHGDKSEEVQDLLLLDVTPLSLGIETAGGVMSVLIKRNTTIPTKQTQTFTTYSDNQPGVLIQVFEGERAMTKDNNLLGKFELSGIPPAPRGVPQIEVTFDIDANGILNVTALEKSTNKENKITITNDKGRLSKEDIERMVNEAEKYRSEDEKQKETISAKNALESYCFNMKATMEDDKLKDKITDSDKTLIMDKCNDTIKWLDANQLAEKEEYEHRQKELESVCNPIITKLYQSAGGAPGGMPGFPGGAPGAGAGAAPGAGSGSGPTIEEVD

SEQ ID NO: 101 - protein, 496 aa, Aedes aegypti
MSVNRTISAHQAAKEHVLAVSRDFISQPRLTYKTVSGVNGPLVILDEVKFPKFAEIVQLRLNDGTVRSGQVLEVSGSKAVVQVFEGTSGIDAKNTVCEFTGDILRTPVSEDMLGRVFNGSGKPIDKGPPILAEDFLDIQGQPINPWSRIYPEEMIQTGISAIDVMNSIARGQKIPIFSAAGLPHNEIAAQICRQAGLVKHTGKSVLDEHEDNFAIVFAAMGVNMETARFFKQDFEENGSMENVCLFLNLANDPTIERIITPRLALTAAEFLAYQCEKHVLVILTDMSSYAEALREVSAAREEVPGRRGFPGYMYTDLATIYERAGRVEGRNGSITQIPILTMPNDDITHPIPDLTGYITEGQIYVDRQLHNRQIYPPVNVLPSLSRLMKSAIGEGMTRKDHSDVSNQLYACYAIGKDVQAMKAVVGEEALTPDDLLYLEFLTKFEKNFISQGNYENRTVFESLDIGWQLLRIFPKEMLKRIPASILAEFYPRDSRH

SEQ ID NO: 102 - protein, 530 aa, Aedes aegypti
MPANIIMKILITSILILKLAIHVVPQHLISSGASAVESKPVSARPTYEDYKRQRENFLQAEEYHFLGANVTLNENEQLVNKFLMRLKLEEMVKGFNDSYNFIPARHIFEVLDRFGQSKVFKVIQRLPKGGVLHAHDMALGSTDLIVNATYRENLWQKGNFGVSHGPQFKFSKEKPGKEWSLVSEIRQWMTDKVYDAKVGEIFSLYNADPLNAYKSLDDVWSKFQNLFGSLAPLITFAPVWRQYYHDSLKQFYDDHVQYLEFRGVLPDVYDLDGKIYSAEEIVQMYYEETEEFKSSHPEFIGAKFIYAPGRFATDDEFLKIIDTAKRLHKKFPTFLAGFDLVGQEDPGRSLLEFAPALLKLPASINFFFHAGETNWYGMKTDQNLIDAVLLGSKRIGHGFAVLKHPKVLKEIKRRQICIEINPISNQVLKLVQDQRNHPAALLFSDNYPVVVSSDDPSFWRSTPLSHDFYVAFTGIASAKQDLRLLKQLALNSIEYSAMNSEEKTSAKEKWSQAWHDQISALATDIVAGSV

SEQ ID NO: 103 - protein, 562 aa, Aedes aegypti
MAGRPGYSEVIFLYVVSVAVIARATDNMPVNKDVSKLFPLTLIHINDLHARFEETNMKSNVCTQKDQCIAGIARVYQKIKDLLKEYESKNPIYLNAGDNFQGTLWYNLLRWNVTADFIKKLKPAAMTLGNHEFDHTPKGLAPYLAELNKEGIPTIVANLVMNNDPDLKSSKIPKSIKLTVGKRKIGIIGVLYDKTHEIAQTGKVTLSNAVEAVRREAAALKKDNIDIIVVLSHCSYEEDKKIAAEAGDDIDVIVGAHSHSFLYSPDSKQPHDPKDKVEGPYPTLVESKNKRKIPIVQAKSFGKYVGRLTLYFDEEGEVKNWEGYPVFIDHKVQQDPQILKDLVPWRAKVEAIGSTVVGETMIELDRDSCRDQECTLGVLYADGFADQYTNDTFRPFAIIQAGNFRNPIKVGKITNGDIIEAAPFGSTADLIRLKGADIWDVAEHSFALDDEGRTNCLQVSGLRIVIDISKPVRSRVKKIEVMDYTNPKSDKLKPLDKEAEYYIVVPSYLADGKDGFSAMKRATARRTGPLDSDVFKNYVEKIKKVDNLKLGRVIVCKGSKCT

SEQ ID NO: 104 - protein, 572 aa, Aedes aegypti
MIDQCACSHQLSAALSTEDMLRTSSIVFLTCCLTFLIEGSSFKLKIIHFNDIHARFDEVTNSSSPCSGNGETCVAGIARLVTTIEKLRKQNENHLVLNAGDVFQGTIWYTLLKWNVSQQFMNMVKADAMTLGNHEFDDSFPVLIPFLENTKNVTPVVVSNLVFPKQLSRDVTKFRSLIKEDPLVLTVGGQSIGIIGVIFDETDKIGNSDPLKFKSSIETVRIAAKQLKSKGVNIIIVLSHCGVFDDKKIAEQAGEDIDIIVGGHTHTLLYNGDPPSKHAALDKYPIVVETGNNHKVLIVQAFCHGHYVGNIDLTFDDEGEITAFEGQPIYQENRIEKNALVEARVRELRKDVEVKSLVKVGESKLELSNDCRLKDCTFGSVLADAYVWHFRSRSNAPMIAMIHPGNFRISLAAGAITRGQILTALPFNSNANRVTVLGSTIKKAIEFGTSINPRRCSFNALQTAGIKIDVDYGKPVGNRTVILLKTGGKYKRLVESKKYDILVNSYVFKGGDGFDMFKHLAVKGRAPFDAELLEQYIVARKGIQKSGLLQSRMNVSHVEKALSEVKSCKQSR

SEQ ID NO: 105 - protein, 418 aa, Aedes aegypti
MCSTGFCLVFFLAQVVFQMNYSEQQTTVVMENGAISESEINVDIVMEQYILKFYTKRFVEGQNLVVAPLLTFRVFMSLYKAMDASAKFDLHSLLGIQQDTSVEKMSEIEAFANKHTLPVDEKQISVETRLYYDKSIGNARSVLTAKSLKPIGTSFSDKRAFCEKVNSWIRNAPIKGTDNLVRDYDLNNETQAFVAGALSIYWNTQLKSSTDQKGFQGENVKFLEGSISAGYAKLDNLKVEVVELISDKVDGVKLWLIMPDRASSIKDFNDQLSVESIRQIENGLTAQKVDVSLALPMVTIEYNSQEDAYVTEVFEVFSSLFTKPSVKLVDGKDDLYVIKNFLMKCILRFVESDASADSKAQSTGMLVKFDRPFVMMMLSKEGNVPILLANYFSPTDKLRALEAKERRLKAEANEHLDL

SEQ ID NO: 106 - protein, 417 aa, Aedes aegypti
MNLWIIGFCSIYFACSVRSQFTSVPVSYDAQNDHNEFSWNAFKKVFTDYKENFVMSPYSLRRLFSCFQGVKLLTSASGTNLQQELSNVLKIVPNQQPSGQDHRPYVEQWVRYSSAKYLNRTAMAVAIGSEKVSTVYESIINNCVIYTGHLQPSNAQRMGQVINDALKNITNNAVQSYLTDTDINPNWKFFAIDSWQFEGLWKFKFQEEFSATCYFYASREKKGLTKFLYLEEMLKYGNFPEWNVQAVELPYHDQSPLSCLLMMPLDGNYESLIHSMNQSRFKEVLSKLNEIKTTVRIPQFGLQTTVPGRQLLESMGMKVPFNQGVFKVFEQGQDVALGEIVQKMEMSIAADGEKQAQSFVDKRQDKQFTAHQPFLFVVYDRNELVPILVGFYLKTPPEAAMGLEDKQKCDDPPVGYQ

SEQ ID NO: 107 - protein, 291 aa, Aedes aegypti; identical to SEQ ID NO: 43

SEQ ID NO: 108 - protein, 290 aa, Aedes aegypti
MILQFWFVTFSVLFAARADENHSILIKLNDLDHRFTQMFSQQFYRHTREVTDRVSALKASIDTNLLELDQQIQQALDGIQSNESSSSTSATKSSGLTTIPIGSEPRVPALYERERYGGDWLVVMHRYDGSVKFDRTWAEYRDGFGMVGQEFWYGLERLHQLTKEKSYELMVEMEDFNGNLKYAWYDKFVVGPEEQRYALVELGTFNGTTDGDSLKPHKGSGFSTYDNDDFGCSNKYAKGGWWYYSGKCYGSSLTGIWKNELAYSSIVWVKFSDVSNTPLKLVRMMIRPKN

SEQ ID NO: 109 - protein, 312 aa, Aedes aegypti
MSPSNKILVLLLFPILLVSSHPIPAEDPAKQCNLSEDDLTKLKAAISGASSAKAANEDILPNTTLAACPMLKNFTEMLKTVATDMEVLKTQGVSNMEVQLLRESFEEKLNDLAKNKDIFERQANQDTSKAEGEMVEKINKLQLEMAKLQEEIEEQTKQMYVDMIEYIFERLKMNDTEAIDSYAQIVMKTKMHELIMKLKTDRLVLWEMVKYVEGKKNKWVGRKVLNTILDQVNKLKLYKPEEVEIGKNSLVVVWCWKFNSETVYGTTDEDQKSFHLAKLFFPKEKGCKECANVKSRTMCNNDYPKVMVKAFG

SEQ ID NO: 110 - protein, 321 aa, Aedes aegypti; identical to SEQ ID NO: 44

SEQ ID NO: 111 - protein, 332 aa, Aedes aegypti
MHSPKSFLLLAVVFVALRVTAAPLWNAKNPEQLQYIAARCMEEWSPKAKDPKAALKNWMEWKLQPSNEEATQCYTKCMLENIGYYEPGEKRLKGVRVMQQWETFNRYQSADRNKVHDLTDTFDFIKPLKSSSCSDVFNAYKDVHAKHLETIKAILFCDGKSAEKYYKDKGKNVKQKGESIFVHCEEIHYPVGSPQRNELCKVRKYELGTGKPFENLMECIFKGVRYFNDKNELNIDEIARDFTQVGKKPDAVKAAMENCKSKTKETDPGKKAVEYYKCLLADSKVKKDFMEAFDYREIRSKDYYAQITGKLKPYSASDVRKEVNDIDSNKCV

SEQ ID NO: 112 - protein, 568 aa, Aedes aegypti
MKLKVYICQVIFSFLAVSVFCEENCNIPESELSKIDHVLRHMEKPIYSEEQFASDNEECTNLLNGIHAQLRRLTQRYKLMNKGYVKVEEYQRMADDYEKQLKTLNDELVELQQHTSEKASATIAKLKEDIKKLDEEVGTLHEKLKGIKQDFEKVKRDLCVTYLNSNQMSKAKAKLKEMASTYLIEIVQQQLNKSNANIMPMLEFSAAIPDLDDMGEAYKEIYKFLEEQKRLEGEDSVLLEATVLKMNASLKEGSNITDERRTQIEGLLKDLATKSTIVFSTWTKELKKINDAVVIKNALDHMFVSQMKVFGALVGDTSDFGSIRNFVKLTVVCNNYYKVAAYKELIDRKIGNALGTIMFDLLTLEVNEMKFDPHVPDEIPKLFEATLSSLPNSLTELRTCLGKVQIYNKKTNKCVVATGNDFDVHKDKLGDFYRVVVADYGCTSFRLEASGDKASVRIVTPSGNPMSNVNLHLEGNSLHNYVATPKSNKPDRTPSSSDEWILDANYNNDTIKIESQFSDYKTKKTEVDHLLVRDINHLPHVLVARYGFMGLKNSDAKDTIEWNLKCGS

SEQ ID NO: 113 - protein, 316 aa, Aedes aegypti; identical to SEQ ID NO: 3

SEQ ID NO: 114 - protein, 215 aa, Aedes aegypti; identical to SEQ ID NO: 5

SEQ ID NO: 115 - protein, 183 aa, Aedes aegypti
MHSLKSSPLLAAVFLALHVTGAPFWNAKNPDELQSIAARCMDEWSPKAKDPKAALKNWKEWRLQPSNDEATKCYTKCMLENIGFYEPAEKRLKGVRIMQQWETFSRYQSADREKVHDLTDTFNFIRPLKSSSCTDVFNAYKDVHARHLETIKAILFCDGKSAEKYYKDKGKTSKQKKVLCTGS

SEQ ID NO: 116 - protein, 322 aa, Aedes aegypti
MKTSLPIVVLLTAVISGVHPNPTPKSCTVSEEDLTTIRNAIQKASRASLDDVNLDEDLIAKCPLLKTITASLKSVASEIATLKDTGISEEQVDELKQSYEQQVNEIVKSRDIFEKQSGGDVMKEQGAMINRMTELQVQVAQLQQQIGEQTSRMYDDMAELIFQRLAMNSTDSIRNYTAHMMEQKLHTLMTKLETNYRIFLGALRYLDHLGDQPLIDKVFDGILKRLDEMSLETNKERENGKYVLVNLLCWTVNNRFLTEKYRKKQLELFRIALKFYPKTGNKEANEADIRGRQFCDANFPVNVITWFAVSRAAEGWGLRGTL

SEQ ID NO: 117 - protein, 614 aa, Aedes aegypti
MSTLKKISDEDRESKFGYVFAVSGPVVTAERMSGSAMYELVRVGYYELVGEIIRLEGDMATIQVYEETSGVTVGDPVLRTGKPLSVELGPGIMGSIFDGIQRPLKDINELTSSIYIPKGVNIPCLSRTQSWGFNPLNVKVGSHITGGDLYGLVHENTLVKHKLLVPPRAKGTVRYIAPPGNYTVDDIILETEFDGEINKWSMLQVWPVRQPRPVTEKLPANHPLLTGQRVLDSLFPCVQGGTTAIPGAFGCGKTVISQALSKYSNSDVIIYVGCGERGNEMSEVLRDFPELSVEIDGVTESIMKRTALVANTSNMPVAAREASIYTGITLSEYFRDMGYNVSMMADSTSRWAEALREISGRLAEMPADSGYPAYLGARLASFYERAGRVKCLGNPEREGSVSIVGAVSPPGGDFSDPVTSATLGIVQVFWGLDKKLAQRKHFPSINWLISYSKYMRALDDFYDKNFQEFVPLRTKVKEILQEEEDLSEIVQLVGKASLAETDKITLEVAKLLKDDFLQQNSYSAYDRFCPFYKTVGMLRNMIGFYDMARHAVETTAQSENKITWNVIRDSMGNILYQLSSMKFKDPVKDGEAKIKADFDQLYEDLQQAFRNLED

SEQ ID NO: 118 - protein, 564 aa, Aedes aegypti
MAGKPGIQLFVIFILLSSFAAVVWTTDNMPADKDVSKLFPLTLIHINDLHARFDETNMKSNACTAKDQCIAGIARVYQKIQDLLKEYKSKNAIYLNAGDNFQGTLWYNLLRWQVTADFITKLKPTAMTLGNHEFDHTPKGLAPYLAELDKAGIPTLVANLVMNDDPDLKSSKIQKSIKVTVGGKTIGIIGVLYDKTHEIAQTGKVTLSNAVETVKREAAALKKDKVDIIVVLSHCSYDEDKKIAKEAGQDIDVIVGAHSHSFLYSKESNKPYDQKDKIEGPYPTIVESNNKRKIPIVQAKSFGKYVGRLTLYFDNEGEVKHWEGYPEFIDNKVKQDPKILEALIPWRKKVQEIGSTKVGETTIELDRDSCRDKECTLGVLYADAFADHYTNSSFRPFAIIQAGNFRNPIKVGKITNGDIIEAAPFGSTADLIRLKGDSLWAVAEHSFALDDENRTNCLQVSGLRIVIDPSKKIGSRVVKIDVMDNRNPKSEDLKPLDKNAEYFIALPSYLADGKDGFSAMKKATARWTGPLDSDVFKSYVEKIKKVDKLKWGRVIVCKAGSPCT

SEQ ID NO: 119 - protein, 567 aa, Aedes aegypti
MKWSVYIALLVFAFLTSPVFSEENCNIPESELSKIDDVLRHMEKPIYSEDHYTSNNEECTNLLNGIHAQLRRLTQRYKLMNKGYVKVEEYKRMAEDYENQLKTLNAELLELQEHTSDKANAAIAKLKEDIKKLDEDVDTLHNKLKGIKQDFEKVKRDLCLTYLNSNQMSNAKAKVKEMASTYLIEIIQQRLNTKYANIIPMLDFSTAIPDLDDRGEAYKEIYKFIETHERLDGEDAVLLEASLLKMNATLKEGSNITDERRTEIEKMLKELAEKSAVVFKTWSTELKGIEDTIIKYALDHLFVNQMKVFGGIVGDTFEFAPIRHLLKLLVVCNNYYKVAAYKELIDRKIGNVLGTIMFDLTTLEANEMSFDLHVPDEIPKLFNATLGSLPNSLTQLLPCLNKVHVYNAKTNMCIVAPEDRFDVQQEKLTDFHRVVLAKYGCTAFRLESSPNKASVKFVKPSGNALSSINLQLENDQWHSHVGTPTANKPDRKPSSSDEWILDANYVNDTVKIQSEFNEYKASQAEVDHLLVMDVKYLPHVVVGRYGVRGLKRSSAKDTIEWYLKCAS

SEQ ID NO: 120 - protein, 256 aa, Aedes aegypti
MAFNGIALLITATIFIGSCYANYCDSSLCRQGPHVACNAPQQFGPACGNNRKFVPMDSKLKTIILNTHNKLRAEIANGMHGFPQAARMPTLVWDDELAHIASFNARKCIFAHDKCRNTRQFKFSGQNLAITTFYGFNFQAGDRAENFTQEWFNEHKDCPKSYVDAYPSSHRGPQIGHFTQLVNDRTWKVGCSMMHYITNGKMINYYLVCNYTMTNMIGEPIYTKGRTGSKCETGQNPQFKGLCSPREKVKSESYNG

SEQ ID NO: 121 - protein, 422 aa, Aedes aegypti
MCSTGLCLVFFIAQAVFLMNYSEQQTTVVMENGAISEKETNVDEVMTQFIMKFYTKRFVEGQNLVVAPLLIFRVFMSMYGEMDASAKFDLHSLVGIPQEASAEKMSEFEAFANKYALPVGVQRNLVETRLYYDKSIGKIRSSLEAKSLKPFPTNFADKQTFCNEVNTWIRNTPINGTDDLVHDYYLNNETAAFVAGALSIDWNMQLKTSSDVKAFEGENVKFLEGSISTRYAKLDNLKVEVVEMVTDNLSGVKLWLIMPDEASSIKKFNDQLSIASIRQIEKGLTALQKEDVALTVPMVTIEYNSQEDAYVTEVFEVFSSLFSKPAVKPWFRVSVKDDLYAVKNFLMKCILRFVGSDAPADSKGQSTEKAVSFNRPFVMMILSKESNVPILLANYFSPKDKLRALEAKERHLRMKAKEHLDL

SEQ ID NO: 122 - protein, 533 aa, Aedes aegypti
MKILLAVVFVLNLTNLAVPQHLITSSPSLPESKPVGRRPTYEEYKQQRESFLQTEDHHLLGANVTLTENEQLVNKFIMQMKLDEMEKGFNDSYNFIPARHIFEVLDRFGQSKVFNVIRRLPKGGVLHAHDMALGSTDLIVNATYLENLWQKGNFGLNHGPEFKFSRERPGKEWSLVSEIRQWMTNEVYDAKVAEVFSLYNADPLNAYKSLDNVWSKFQNLFACLAPLITFAPVWRQYYHDSLKQFYDDHVQYLEFRGVLPEVYDLDGKVYSAEEIVQLYYEETEQFKAKYPDFIGVKFIYAPGRYASDEEFQKLLDTTNRLHKKFPNFLAGFDLVGQEDPGRSLFEFAPALLKLPASINFFFHAGETNWYGMKTDQNLVDAVLLGTKRIGHGFAVLKHPKVLKEIKRRQICIEINPISNQVLKLVQDQRNHPAALLFSDNYPVVVSSDDPSFGRSTPLSHDFYVAFTGIASAKQDWRWLKQLALNSIEYSAMNSEEKTVAKEKWNQAWDHQFSRLAVDFVAGKILENWIMKIV

SEQ ID NO: 123 - protein, 389 aa, Aedes aegypti
MQPRILHLTVLATIIGVALTANVPSTPGRKLNIPAFSNAGKTKGIEIWRIENFQPVAVPKAEYGKFYTGDSYLVLNTNEDKNKKKSYDVHFWLGLKTTQDEAGSAAILTVQLDDLLGGGPVQHREVEGSESDLFLSYFKGGIRYLEGGVASGFKHVQTNAAHPKRLFHVKGAKNIRLRQVELAVSAMNKGDCFILDSDRDVFVWVGPKANRVEKLKAINVANDIRDRDHNGRATVHIVDEFSTLSDQESFFKSLGSGSPSTVPDQSTAKEDAAFEKADAARVELYKVTDSKAGKLAVEPITQKPLKQEMLKPDDAFILDTGSGLYVWIGKSATQQEKTQSLVKAQEFIKNKKYPAWTPVERIVQNAETAPFKHFFQTWRDAGSTGSRLV

SEQ ID NO: 124 - protein, 259 aa, Aedes aegypti
MKSIVSITITVLAIICEGQATNYCDPSLCARGTPHIACNGLSTLSRTCGAGSFEVALNRADQQLIVDLHNKLRSKVAMGQQKNSAGQRFQQACRMATLQWDPELAHIAATNARRCVYGHDTCRNTASMKFAGQNIAIKYYYGMTFTNEQLLTGFINSWFSEFKDATPQQIARYPANYRGPAIGHFTQIVSDRTSRIGCSMVSYNKNGFINKLFVCNYGLTNIINQPVYVAGNVCSGCTTGCNKVFPGLCNTAERVSNNP

SEQ ID NO: 125 - protein, 566 aa, Aedes aegypti
MKVYICQVIFSFLAVSVFCEENCNIPESELSKIDHVLRHMEKPIYSEEQFASDNEECTNLLNGIHAQLRRLTQRYKLMNKGYVKVEEYQRMADNYEKQLKTLNDELVELQQHTSEKASATIAKLKEDIKKLDEEVGTLHEKLKGIKQDFEKVKRDLCVTYLNSNQMSKAKAKLKEMASTYLIEIVQQQLNKSNANIMPMLEFSAAIPDLDDMGEAYKEIYKFLEEQKRLEGEDSVLLEATVLKMNASLKEGSNITDERRTQIEGLLKDLATKSTIVFSTWTKELKKINDAVVIKNALDHMFVSQMKVFGALVGDTSDFGSIRNFVKLTIVCNNYYKVAAYKELIDRKIGNALGTIMFDLLTLEVNEMKFDPHVPDEIPKLFEATLSSLPNSLTELRTCLGKVQIYNKKTNKCVVATGNDFDVHKDKLGDFYRVVVADYGCTSFRLEASGDKASVRIVTPSGNPMSNVNLHLEGNSLHNYVATPKSNKPDRTPSSSDEWILDANYNNDTIKIESQFSDYKTKKTEVDHLLVRDINHLPHVLVARYGFMGLKNSDAKDTIEWNLKCGS

SEQ ID NO: 126 - DNA, 42 bp, Artificial sequence
  PCR primer
taatacgactcactataggggatggacagatgtctcttcgtg

SEQ ID NO: 127 - DNA, 40 bp, Artificial sequence
  PCR primer
taatacgactcactatagggccaaatccaatccatcgaaa

SEQ ID NO: 128 - DNA, 38 bp, Artificial sequence
  PCR primer
taatacgactcactataggggtgagcaagggcgaggag

SEQ ID NO: 129 - DNA, 44 bp, Artificial sequence
  PCR primer
taatacgactcactatagggcatgatatagacgttgtggctgtt

(19) United States
(12) Patent Application Publication
(10) Pub. No.: US 2021/0338776 A1
(43) Pub. Date: Nov. 4, 2021

(54) TREATMENTS AGAINST MOSQUITO-BORNE VIRUSES BASED ON MOSQUITO SALIVARY GLAND PROTEINS

(71) Applicant: YALE UNIVERSITY, New Haven, CT (US)

(72) Inventors: Erol FIKRIG, Guilford, CT (US); Ryuta URAKI, New Haven, CT (US); Andrew HASTINGS, Hamden, CT (US)

(73) Assignee: YALE UNIVERSITY, New Haven, CT (US)

(21) Appl. No.: 17/274,086

(22) PCT Filed: Sep. 5, 2019
(86) PCT No.: PCT/US19/49713

§ 371 Date: Mar. 5, 2021

Related U.S. Application Data
(60) Provisional application No. 62/735,597, filed on Sep. 24, 2018.
(60) Provisional application No. 62/727,906, filed on Sep. 6, 2018.

(51) Int. Cl. A61K 38/17

(52) U.S. Cl. CPC A61K 38/1767

(57) ABSTRACT

The invention is directed to compositions comprising mosquito (e.g., Aedes aegypti) salivary polypeptides and related methods for preventing and/or treating mosquito-borne viral infections such as infections caused by flaviviruses and alphaviruses. The flavivirus that is prevented or treated includes Zika virus, West Nile virus, Dengue virus, tick-borne encephalitis virus, and yellow fever virus.

### CROSS-REFERENCE TO RELATED APPLICATIONS

This application claims priority to U.S. Provisional Application No. 62/727,906, filed Sep. 6, 2018, which application is herein incorporated by reference in its entirety, and U.S. Provisional Application No. 62/735,597, filed Sep. 24, 2018, which application is herein incorporated by reference in its entirety.

### STATEMENT REGARDING FEDERALLY SPONSORED RESEARCH

This invention was made with government support under AI089992, AI127865, and AI145779 awarded by National Institutes of Health. The government has certain rights in the invention.

### TECHNICAL FIELD OF THE INVENTION

The present invention is directed to compositions comprising mosquito (e.g., Aedes aegypti) salivary polypeptides and related methods for preventing and/or treating mosquito-borne viral infections such as infections caused by flaviviruses and alphaviruses.

### BACKGROUND

Zika virus (ZIKV) is a member of the flavivirus family along with West Nile virus and Dengue virus. ZIKV was first isolated in 1947 from a sentinel monkey in the Zika forest of Uganda (1, 2). The first case of ZIKV infection in humans was reported in Nigeria in 1954 (3). For half a century, serologic evidence suggests that the virus circulated in Africa and Southeast Asia (4-11). Most recently, a large epidemic in the Americas, affecting well over a million people, has caused significant concern over this virus's potential to spread worldwide (12-14). Historically, Zika virus has manifested as a relatively mild self-limiting illness like dengue virus, with fever, rash and headache, and up to 80% of infected individuals remaining asymptomatic (9, 15, 16). However, these new epidemics have come with significantly more severe symptoms including Guillain-Barre syndrome (GBS) and birth defects (17-19). Zika virus can infect testes and is detected in semen, leading to sexual transmission (20-23). This has led to warnings to pregnant women to be mindful of insect repellant use and to limit travel to epidemic areas. Overall, this seeming increase in disease severity and rapid spread has led to increasing alarm across the globe.

The major mosquito vector for Zika virus is the Aedes species (Ae. aegypti and Ae. albopictus), which most likely originated in Africa and is now endemic in tropical and subtropical locations (31). This presents a significant public health risk that should be addressed. Although there are many efforts to develop Zika virus specific vaccines, there is currently no available commercial vaccine.

West Nile virus (WNV) is a single-stranded positive-sense RNA virus in the genus Flavivirus, which normally circulates in a bird-mosquito transmission cycle and is a common human mosquito-borne flaviviral infection in North America and other regions in the world (53-55). WNV can also infect horses, and other non-avian vertebrate hosts (56). Despite substantial efforts, effective FDA-approved preventive or therapeutic measures are not yet available (53,56,57).

Culex mosquito spp., now endemic in tropical and subtropical regions as well as more temperate areas, are the major vectors for WNV worldwide (57). However, the virus also has been isolated from Aedes aegypti mosquitoes, which is present in tropical and subtropical locations as well (58,59) and are a potential threat for transmission of WNV to humans (60). Although the vector competence of WNV in Ae. aegypti is lower than that of Culex spp., multiple factors can affect vector distribution, including climate change, and this may influence the vectorial capacity of Aedes mosquitoes for WNV in the future (61,62). Moreover, in laboratory studies, Ae. aegypti readily feed on mice and a well-annotated whole genome sequence is available (63).

When mosquitoes take a blood meal, they inoculate saliva into the skin (64). Mosquito saliva contains molecules which modulate various host responses, including coagulation, platelet aggregation, thrombin activation, vasodilation, and other mammalian host pathways (65,66).

Mosquitoes inject numerous salivary proteins into the skin of a host during blood feeding, and these molecules are capable of modulating various host responses (65,66). Indeed, mosquito saliva enhances transmission and pathogenicity of specific arboviruses (31,75). Although mosquito saliva can increase arboviral infectivity, only a limited number of specific salivary proteins have been characterized that influence these processes. The biogenic amine-binding D7 protein partially inhibits dengue infection, while saliva serine protease CLIPA3 enhances dissemination of dengue virus into the mammalian host (25,67). In addition, salivary factor LTRIN from A. aegypti facilitates the transmission of Zika virus by inhibiting NFκB signaling during infection (68). Despite these efforts, much remains to be discovered about how specific salivary factors facilitate mosquito-borne virus infection, and whether targeting these proteins can prevent or delay infection.

Components of saliva enhance the pathogenicity and transmission of arboviruses including WNV, dengue, Zika, and Semliki Forest viruses, suggesting that certain salivary proteins are important for influencing flavivirus infectivity during transmission from vector to host (25,31,32,67,68). The expression of AgBR1 in the salivary glands is up-regulated after blood feeding and AgBR1 belongs to a family of proteins that have lost chitinolytic activity (41,70), however the function of this protein in the vertebrate host remains unclear. To determine whether this effect extends beyond Zika virus to another flavivirus, the influence of AgBR1 antibodies against Ae. aegypti-borne WNV infection in mice was examined.

Saliva from arthropod vectors, such as ticks, sand flies and mosquitoes, is capable of enhancing transmission and pathogenicity of important human pathogens such as arboviruses and Leishmania (24-31). A variety of salivary proteins have been discovered. See, e.g., Int. Pat. Appl. Pub. WO 2011/1104684 and (24-26), incorporated by reference herein in its entirety.

### SUMMARY OF THE INVENTION

As specified in the Background Section, there is a great need in the art to develop effective vaccines and treatments for mosquito-borne viruses such as, e.g., flaviviruses and alphaviruses. The present invention addresses this and other needs by providing compositions and methods disclosed herein.

In one aspect, the invention provides a composition comprising one or more polypeptides, wherein said one or more polypeptides are selected from the group consisting of LOC5573204 bacteria-responsive protein 1 (AgBR1), LOC5578630, LOC5578631, LOC5567956, LOC5580038, LOC5566287, LOC5567958, LOC5568702, LOC110675548, and fragments, derivatives or variants thereof.

In another aspect, the invention provides a composition comprising a nucleic acid molecule encoding one or more polypeptides, wherein said one or more polypeptides are selected from the group consisting of LOC5573204 (AgBR1), LOC5578630, LOC5578631, LOC5567956, LOC5580038, LOC5566287, LOC5567958, LOC5568702, LOC110675548, and fragments, derivatives or variants thereof.

In a further aspect, the invention provides a composition comprising an antibody which recognizes a polypeptide, wherein said polypeptide is selected from the group consisting of LOC5573204 (AgBR1), LOC5578630, LOC5578631, LOC5567956, LOC5580038, LOC5566287, LOC5567958, LOC5568702, LOC110675548, and fragments, derivatives or variants thereof.

In yet another aspect, the invention provides a composition comprising a molecule which inhibits a function of a mosquito salivary polypeptide or inhibits the interaction between a mosquito salivary polypeptide and a host cell or inhibits the interaction between a mosquito salivary polypeptide and a mosquito-borne infectious agent, wherein said mosquito salivary polypeptide is selected from the group consisting of LOC5573204 (AgBR1), LOC5578630, LOC5578631, LOC5567956, LOC5580038, LOC5566287, LOC5567958, LOC5568702, LOC110675548, and fragments, derivatives or variants thereof. In one embodiment, the mosquito-borne infectious agent is a mosquito-borne virus (e.g., a flavivirus [such as, e.g., Zika virus, West Nile virus, Dengue virus, tick-borne encephalitis virus, or yellow fever virus], or an alphavirus [such as, e.g., Chikungunya virus (CHIKV), Ross River virus, O'nyong'nyong virus, Semliki Forest virus]).

In one embodiment of any of the above compositions of the invention, the polypeptide is LOC5573204 bacteria-responsive protein 1 (AgBR1) or a fragment, derivative or variant thereof. In one specific embodiment, the polypeptide comprises the sequence SEQ ID NO: 1. In one specific embodiment, the polypeptide consists of the sequence SEQ ID NO: 1. In one specific embodiment, the polypeptide is a fragment of LOC5573204 (e.g., the polypeptide fragment of LOC5573204 comprising the sequence selected from SEQ ID NOS: 6-13).

In one embodiment of any of the above compositions of the invention, the polypeptide is LOC5578630 or a fragment, derivative or variant thereof. In one specific embodiment, the polypeptide LOC5578630 comprises the sequence SEQ ID NO: 2. In one specific embodiment, the polypeptide LOC5578630 consists of the sequence SEQ ID NO: 2. In one specific embodiment, the polypeptide is a fragment of LOC5578630 (e.g., the polypeptide fragment of LOC5578630 comprising the sequence selected from SEQ ID NOS: 14-21).

In one embodiment of any of the above compositions of the invention, the polypeptide is LOC5578631 or a fragment, derivative or variant thereof. In one specific embodiment, the polypeptide LOC5578631 comprises the sequence SEQ ID NO: 3. In one specific embodiment, the polypeptide LOC5578631 consists of the sequence SEQ ID NO: 3. In one specific embodiment, the polypeptide is a fragment of LOC5578631 (e.g., the polypeptide fragment of LOC5578631 comprising the sequence selected from SEQ ID NOS: 22-29).

In one embodiment of any of the above compositions of the invention, the polypeptide is LOC5567956 or a fragment, derivative or variant thereof. In one specific embodiment, the polypeptide LOC5567956 comprises the sequence SEQ ID NO: 4. In one specific embodiment, the polypeptide LOC5567956 consists of the sequence SEQ ID NO: 4. In one specific embodiment, the polypeptide is a fragment of LOC5567956 (e.g., the polypeptide fragment of LOC5567956 comprising the sequence selected from SEQ ID NOS: 30-36).

In one embodiment of any of the above compositions of the invention, the polypeptide is LOC5580038 or a fragment, derivative or variant thereof. In one specific embodiment, the polypeptide LOC5580038 comprises the sequence SEQ ID NO: 5. In one specific embodiment, the polypeptide LOC5580038 consists of the sequence SEQ ID NO: 5. In one specific embodiment, the polypeptide is a fragment of LOC5580038 (e.g., the polypeptide fragment of LOC5580038 comprising the sequence selected from SEQ ID NOS: 37-42).

In one embodiment of any of the above compositions of the invention, the polypeptide is LOC5566287 or a fragment, derivative or variant thereof. In one specific embodiment, the polypeptide LOC5566287 comprises the sequence SEQ ID NO: 43. In one specific embodiment, the polypeptide LOC5566287 consists of the sequence SEQ ID NO: 43. In one specific embodiment, the polypeptide is a fragment of LOC5566287 (e.g., the polypeptide fragment of LOC5566287 comprising the sequence selected from SEQ ID NOS: 47-59).

In one embodiment of any of the above compositions of the invention, the polypeptide is LOC5567958 or a fragment, derivative or variant thereof. In one specific embodiment, the polypeptide LOC5567958 comprises the sequence SEQ ID NO: 44. In one specific embodiment, the polypeptide LOC5567958 consists of the sequence SEQ ID NO: 44. In one specific embodiment, the polypeptide is a fragment of LOC5567958 (e.g., the polypeptide fragment of LOC5567958 comprising the sequence selected from SEQ ID NOS: 60-70).

In one embodiment of any of the above compositions of the invention, the polypeptide is LOC5568702 or a fragment, derivatives or variant thereof. In one specific embodiment, the polypeptide LOC5568702 comprises the sequence SEQ ID NO: 45. In one specific embodiment, the polypeptide LOC5568702 consists of the sequence SEQ ID NO: 45. In one specific embodiment, the polypeptide is a fragment of LOC5568702 (e.g., the polypeptide fragment of LOC5568702 comprising the sequence selected from SEQ ID NOS: 71-85).

In one embodiment of any of the above compositions of the invention, the polypeptide is LOC110675548 or a fragment, derivative or variant thereof. In one specific embodiment, the polypeptide LOC110675548 comprises the sequence SEQ ID NO: 46. In one specific embodiment, the polypeptide LOC110675548 consists of the sequence SEQ ID NO: 44. In one specific embodiment, the polypeptide is a fragment of LOC110675548 (e.g., the polypeptide fragment of LOC110675548 comprising the sequence selected from SEQ ID NOS: 86-99).

In one embodiment of any of the above compositions of the invention, the composition further comprises a carrier or excipient.

In one embodiment of any of the above compositions of the invention, the composition further comprises an adjuvant.

In one embodiment of any of the above compositions of the invention, the composition comprises at least two different polypeptides.

In another aspect, the invention provides a method of preventing or treating a disease in a subject in need thereof, wherein the disease is associated with a mosquito-borne infectious agent, said method comprising administering to said subject an effective amount of any of the compositions of the invention. In one embodiment, the mosquito is Aedes aegypti. In one embodiment, the mosquito-borne infectious agent is a mosquito-borne virus (e.g., a flavivirus [such as, e.g., Zika virus, West Nile virus, Dengue virus, tick-borne encephalitis virus, or yellow fever virus], or an alphavirus [such as, e.g., Chikungunya virus (CHIKV), Ross River virus, O'nyong'nyong virus, Semliki Forest virus]). In one embodiment, the subject is human.

These and other aspects of the present invention will be apparent to those of ordinary skill in the art in the following description, drawings and claims.

### BRIEF DESCRIPTION OF THE DRAWINGS

FIGS. 1A-1B show the reactivity if serum of mice bitten by A. aegypti mosquitoes against mosquito salivary gland extract (SGE). FIG. 1A and FIG. 1B show that ELISA (A) and Western blot (B) analysis of SGE were performed by probing with naïve mouse serum and serum from mice bitten by mosquitoes.

FIGS. 2A-2B show the reactivity if serum of human bitten by A. aegypti mosquitoes against mosquito salivary gland extract (SGE). FIGS. 2A and 2B show that ELISA (A) and Western blot (B) analysis of SGE were performed by probing with naïve human serum and serum from human bitten by mosquitoes.

FIGS. 3A-3C show a yeast surface display (YSD) approach to identify mosquito antigenic proteins in mice bitten by Aedes aegypti. EBY-100 yeast cells, transformed with an Ae. aegypti salivary gland cDNA library, were induced overnight before magnetic sorting. In FIG. 3A, after each magnetic sort, binding of transformed yeast cells using IgG from mice bitten by mosquitoes (red) and IgG derived from normal mouse serum (blue) was analyzed using flow cytometry (FACS). In FIG. 3B, the percentages of IgG-binding yeast cells were determined by FACS analysis as shown. In FIG. 3C, antigenic A. aegypti salivary proteins were identified by a YSD library using serum from mice bitten by A. aegypti.

FIGS. 4A-4C show a yeast surface display (YSD) approach to identify mosquito antigenic proteins in humans bitten by Aedes aegypti. EBY-100 yeast cells, transformed with an Ae. aegypti salivary gland cDNA library, were induced overnight before magnetic sorting. In FIG. 4A, after each magnetic sort, binding of transformed yeast cells using IgG from humans bitten by mosquitoes (red) and IgG derived from normal human serum (blue) was analyzed using flow cytometry (FACS). In FIG. 4B, the percentages of IgG-binding yeast cells were determined by FACS analysis as shown. In FIG. 4C, antigenic A. aegypti salivary proteins were identified by a YSD library using serum from human bitten by A. aegypti.

FIGS. 5A-5D show purification of nine identified proteins and specificity of rabbit serum against them. Identified proteins produced in Drosophila S2 cells and purified using the TALON resin. FIG. 5A is an SDS-PAGE gel stained with Coomassie Brilliant Blue, of recombinant proteins SP (LOC5578630), NeSt1 (LOC5578631), D7Bclu (LOC5567956), AILP (LOC5580038), AgBR1 (LOC5573204), FibRP (LOC5566287), AnLP (LOC110675548), Aada2 (LOC5567958) and Lipase (LOC5568702) (0.25 μg protein). FIG. 5B is an immunoblot showing that anti-His antibody is able to recognize proteins. In FIG. 5C, recombinant AgBR1 (0.25 μg protein) was run on SDS-PAGE and stained with Coomassie Brilliant Blue. FIG. 5D shows detection of AgBR1 protein using an anti-His antibody. Data are representative of two independent experiments.

FIGS. 6A-6I show titration curves for the sera of mice exposed to each of the antigenic proteins listed in FIGS. 2 and 3, i.e. SP, SSP, D7Bclu, AILP, AgBR1, FibRP, Aada2, Lipase, and AnLP by using ELISA. As a control, a titration curve for naïve serum is also shown in FIG. 6J.

FIGS. 7A-7E show that a mixture of antiserum against each antigenic protein protects mice from mosquito-borne Zika virus infection. FIG. 7A shows an experimental murine model of Zika virus transmission. A mouse is treated with an antibody (e.g., to a mosquito salivary protein) the day before exposure to the Zika virus by a bite from an infected Ae. aegypti mosquito. Viremia and survival are checked at days 1, 3, 5, 7, and 9. FIG. 7B shows that the Zika virus level in salivary glands was the same in the CTL and Ab-mix groups by using real-time PCR (RT-PCR). Control group of mice (CTL) were treated with naïve rabbit serum, and the Ab-mix groups of mice (Ab-mix) were treated with the mixture of antiserum against SP, SSP, D7Bclu, AILP, AgBR1, FibRP, Aada2, Lipase, and AnLP. Error bars represent mean±SEM. Each data point represents one aliquot of tested salivary gland extract. FIGS. 7C-7E show that antibody treatment suppressed Zika virus replication in mouse blood. Viremia and survival were checked on days 1, 3, 5, 7, and 9. Viremia was lower, and survival was greater, in mice treated with the antibody mixture. Antibody treatment suppressed Zika virus replication in the blood.

FIGS. 8A-8B show no effect of the mixture of antisera against needle-injected Zika virus infection. Mice were administrated with the mixture of antisera one day before subcutaneous Zika virus injection. In FIG. 8A, blood from mice was collected every other day for 9 days, and analyzed for Zika virus infection by qRT-PCR. Zika virus RNA levels were normalized to mouse β actin RNA levels. Error bars represent mean±SEM. Each data point represents one mouse. Normalized viral RNA levels were analyzed using Wilcoxon-Mann-Whitney test. In FIG. 8B, mice were monitored daily for survival after Zika virus infection. Survival was assessed using a Gehan-Wilcoxon test.

FIG. 9 shows the effect of AgBR1 in vitro, and that recombinant AgBR1 stimulates IL-6 but not TNF-α or IL-1β expression levels in vitro. Splenocytes were isolated from mice and treated with AgBR1 (5 μg/ml), D7Bclu (5 μg/ml) or BSA (5 μg/ml). Cells were harvested 6 hours or 24 hours after treatment, and the expression levels of 116, Tnfa and Il1b were examined by qRT-PCR. Data were derived from two independent experiments and were analyzed by two-way ANOVA. n=5 or 6 biologically independent samples pooled from two separate experiments. Data are presented as mean±s.e.m.

FIGS. 10A-10C show the effect of AgBR1 in vivo, with AgBR1 enhancing Zika virus replication and diseases. In FIG. 10A, AgBR1 protein (5.1 μM, 10 μg in 40 μl) was co-inoculated with Zika virus, and blood was collected every other day for 9 days from mice. Error bars represent mean±SEM. Each data point represents one mouse. Normalized viral RNA levels were analyzed using the Wilcoxon-Mann-Whitney test. (Zika virus: n=12, Zika virus+AgBR1: n=11 pooled from two separate experiments.) In FIG. 10B, injected mice were monitored for survival after infection. Survival and median survival time (MST) was assessed using the Gehan-Wilcoxon test. Survival data shown are pooled from two independent experiments (Zika virus: n=12, Zika virus+AgBR1: n=11). FIG. 10C is shows that the concentration of AgBR1 in mosquito saliva can be estimated to be between 1.6-8.2 μM.

FIGS. 11A-11D show that AgBR1 antiserum protects mice from mosquito-borne Zika virus infection. FIG. 11A shows workflow of passive immunization and mosquito-borne Zika virus infection. FIG. 11B shows Zika virus RNA levels in the salivary glands at 10 days after intrathoracic injection. n=34 (Control) or 38 (AgBR1 antiserum) biologically independent samples pooled from five separate experiments. Data represent mean±s.e.m. FIG. 11C shows Zika virus RNA levels in blood in mice. Data represent mean±s.e.m. Each data point represents one mouse. Normalized viral RNA levels were analyzed using two-sided Wilcoxon-Mann-Whitney test. n=17 (Control) or 19 (AgBR1 antiserum) biologically independent samples pooled from five separate experiments. In FIG. 11D, survival and median survival time (MST) were assessed using the Gehan-Wilcoxon test. n=17 (Control) or 19 (AgBR1 antiserum) biologically independent samples pooled from five separate experiments. FIG. 11E shows that the partial protective effect of AgBR1 antibodies was specific for mosquito-borne, and not needle-injected, Zika virus infection in mice.

FIGS. 12A-12B show no effect of AgBR1 antiserum against needle-injected Zika virus infection. Mice were administrated with AgBR1 antiserum one day before subcutaneous Zika virus injection. In FIG. 12A, blood from mice was collected every other day for 9 days and analyzed for Zika virus infection by qRT-PCR. Zika virus RNA levels were normalized to mouse β actin RNA levels. Error bars represent mean±SEM. Each data point represents one mouse. Normalized viral RNA levels were analyzed using Wilcoxon-Mann-Whitney test. In FIG. 12B, mice were monitored daily for survival after Zika virus infection. Survival was assessed using a Gehan-Wilcoxon test.

FIGS. 13A-13D show the protective effects of different antibodies on mice infected with Zika virus. Mice were administrated with antiserum against SP or D7Bclu one day before Zika virus-infected mosquito feeding. Immunized mice were monitored for survival for 35 days after infected mosquito-feeding. Blood was collected every other day for 9 days from mice fed on by Zika virus-infected mosquitoes and analyzed for Zika virus infection by qRT-PCR. Zika virus RNA levels were normalized to mouse β-actin RNA levels. Mice immunized with naïve serum served as controls. Error bars represent mean±SEM. Each data point represents one mouse. Normalized viral RNA levels were analyzed using Wilcoxon-Mann-Whitney test. In FIGS. 13B and 13D, mice were monitored for survival for 35 days after infected mosquito-feeding. Data shown are pooled from at least two independent experiments. Survival was assessed by a Gehan-Wilcoxon test. The results show that antisera against abundant proteins recognized in the yeast display assay were not protective against mosquito-borne Zika virus infection.

FIGS. 14A-14H show the suppression of neutrophil recruitment at the mosquito bite site in mice administered AgBR1 antiserum. Mice were inoculated with AgBR1 antiserum or control serum prior to Zika virus-infected mosquito feeding. Twenty-four hours post feeding, the bitten ears together with control contralateral non-bitten ears were harvested for tissue sectioning. FIG. 14A shows hematoxylin and eosin staining of the ears of mice 24 hours post-feeding. Scale bar, 200 μm (left panels) and 50 μm (right panels). Data are representative of two independent experiments with similar results. FIG. 14B shows the histological findings were scored in terms of inflammation, neutrophil infiltration, mononuclear cell infiltration and edema. In FIG. 14C, the total histology scores of the bite sites were compared between the AgBR1 antiserum and control group. Data are presented as means±SEM. Statistical analysis was performed using two-sided Wilcoxon-Mann-Whitney test. n=5 (Control) or 6 (AgBR1 antiserum) biologically independent samples pooled from two separate experiments. FIG. 14D shows Imaging Mass Cytometry (IMC) labeling of ears of mice 24 hours post Zika virus-infected mosquito feeding. Sections of the ear skins were labeled with antibodies against CD3 (170Er), CD11b (149Sm), MHCII (174Yb), Ly6G (141Pr) or DNA (193Ir). Scale bar, 100 μm. In FIGS. 14E and 14F, both bitten and resting ears were harvested and enzymatically digested to obtain a single-cell suspension. The population of CD45⁺CD11b⁺Ly6G⁺ (neutrophils) was analyzed using flow cytometry. Data are representative of two independent experiments with similar results. FIG. 14F shows the percent of CD45⁺CD11b⁺Ly6G⁺ (neutrophils) cells in CD45⁺ leukocyte cells at 24 h after Zika virus-infected mosquito feeding. Each dot represents one mouse. Significance was calculated using a two-way ANOVA test for multiple comparisons. Data are presented as mean±s.e.m. Each dot represents one mouse (n=7; resting skin of mice treated with control serum, n=9; bitten skin of mice treated with control serum n=11; resting skin of mice treated with AgBR1 antiserum, n=11 biologically independent samples pooled from two separate experiments; bitten skin of mice treated with AgBR1 antiserum). The data in FIGS. 14G-14I show that neither the D7Bclu nor SP antisera altered the viremia or protected mice from lethal mosquito-borne Zika virus infection.

FIGS. 15A-15D show the host responses in mice treated with AgBR1 antiserum at the mosquito bite site. At 24 hours post feeding, the sites bitten by Zika virus-infected mosquitoes were collected, and total RNA was extracted for RNA-seq. In FIG. 15A, the top panel shows 536 genes (54.4%) within 986 differentially expressed genes (P<0.05) were upregulated at the bitten sites of mice administered control serum. The bottom left panel shows that among these 536 genes, 78 genes were significantly upregulated at the bitten site of mice administered control serum compared with mice injected with AgBR1 antiserum. The bottom right panel shows that among the 536 genes, 272 genes were differentially upregulated in bitten sites of mice administered AgBR1 antiserum compared with the resting sites of mice inoculated with control serum. In FIG. 15B, GSEA of inflammatory responses (Hallmark) and cytokine-cytokine receptor interaction (KEGG) pathway enriched at bite sites of mice compared with resting sites in control mice. In FIG. 15C, the top panel is a Venn diagram depicting the overlap of genes differentially expressed across the conditions. The bottom panel is a heat map of hierarchical clustering performed on 18 upregulated genes across the conditions (Fold change >1.5, P<0.05). In FIGS. 15A-15C, the parameters are as follows: Control-resting skin: n=2, Control-bitten skin: n=2, AgBR1 antiserum-bitten skin: n=2 biologically independent samples. Normalized read counts were statistically modeled using Partek Flow's Gene Specific Analysis (GSA) approach. FIG. 15D shows a QRT-PCR based analysis of Il1b and Il6 expression, which is normalized to mouse β actin RNA levels. Each dot represents one bitten or control site. Data are presented as mean±s.e.m. Significance was determined by two-way ANOVA test. (Control-resting skin: n=13, Control-bitten skin: n=13, AgBR1 antiserum-resting skin: n=13, AgBR1 antiserum-bitten skin: n=13 biologically independent samples pooled from two separate experiments.)

FIG. 15E shows that the direct inoculation of AgBR1 into the skin significantly induces Il1b and Il6 expression.

FIGS. 16A-16C show the effect of active immunization with AgBR1 on mosquito-borne Zika infection in mice. Active immunization with AgBR1 reduces mosquito-borne Zika infection in mice. In FIG. 16A, AG129 mice were immunized with AgBR1 or OVA in Freund's adjuvant. Two weeks after final boost, the sera from immunized mice were examined for specific antibodies with ELISA. Sera from AgBR1-immunized mice recognized AgBR1 (right panel), but not ovalbumin, OVA (left panel). In FIG. 16B, blood was collected every other day for 9 days from immunized mice fed on by Zika virus-infected mosquitoes and analyzed for Zika virus infection by qRT-PCR. Zika virus RNA levels in mice were normalized to mouse β actin RNA levels. Error bars represent mean±SEM. Each data point represents one mouse. Normalized viral RNA levels were compared using Wilcoxon-Mann-Whitney test. In FIG. 16C, mice were monitored for survival for 30 days after infected mosquito feeding. (Left panel) Survival and median survival time (MST) were assessed by a Gehan-Wilcoxon test (n=18/group).

FIGS. 17A-17D show the protective effects of antibodies against mosquito salivary proteins on mice infected with West Nile virus. Anti-AgBR1 antiserum protected mice against West Nile virus transmission. Mice were administrated with anti-AgBR1 serum one day before WNV-infected mosquito feeding. Immunized mice were monitored for survival.

FIG. 17A shows a schematic of the experiment, whose results are shown in FIGS. 17B-17D. In these experiments, mice were administrated AgBR1 antiserum one day before WNV-infected mosquito feeding, and immunized mice were monitored for survival for 10 days after infected mosquito-feeding. FIG. 17B shows the results of an experiment in which the virus levels in blood of mice fed by an infected mosquito was assayed. In this experiment, blood was collected every other day for 7 days from mice fed on by WNV-infected mosquitoes and analyzed by qRT-PCR. WNV RNA levels were normalized to mouse β actin RNA levels. Mice immunized with naïve serum served as controls. In FIG. 17B, the error bars represent mean±SEM. Each data point represents one mouse. Normalized viral RNA levels were analyzed using one-tailed Wilcoxon-Mann-Whitney test (n=13/each group biologically independent samples pooled from three separate experiments). FIG. 17C shows the weight of mice fed by an infected mosquito in an experiment in which mice were monitored daily after WNV infection. Error bars represent mean±SEM. Weight at each time point were compared using one-tailed Wilcoxon-Mann-Whitney test (n=13/each group biologically independent samples pooled from three separate experiments). FIG. 17D shows the results of an experiment in which survival was assessed by a Gehan-Wilcoxon test (n=13/each group biologically independent samples pooled from three separate experiments).

FIGS. 17E and 17F show that AgBR1 antiserum modulates host responses at the WNV-infected mosquito bite site. The expression levels of several cytokines were analyzed by qRT-PCR at (a) 6 hours (FIG. 17E) or 24 hours (FIG. 17F) after bites of infected mosquitoes, which is normalized to mouse β actin RNA levels. Error bars represent mean±SEM. Each dot represents one bitten or control site. Significance was determined by two-way ANOVA test (6 hours; n=19/control group, n=15/AgBR1 antiserum group, 24 hours; n=15/control group, n=17/AgBR1 antiserum group biologically independent samples pooled from two separate experiments). FIGS. 17G-17I show the results of identical passive immunization experiments using SP antiserum. SP antiserum did not alter viremia, weight loss or survival time after lethal mosquito-borne WNV infection. FIG. 17J is an immunoblot of Ae. aegypti and Culex pipiens salivary glands probed with rabbit AgBR1 antiserum, the immunoblot showing that AgBR1 antiserum also specifically recognizes a protein in Culex pipiens salivary glands.

FIGS. 18A-18C show the effects of double-stranded RNAs (dsRNA) against AgBR1 on the levels of AgBR1 and Zika virus in mice. Mosquitoes treated with 200 ng AgBR1- or GFP-dsRNA were used to isolate total RNA at day 13 post dsRNA injection. In FIG. 18A, mRNA levels were determined by qRT-PCR and normalized to mosquito Rp49 RNA levels. In FIG. 18B, AgBR1-dsRNA- or GFP-dsRNA-treated mosquitoes were collected 13 days after gene silencing. The SGE was run by SDS-PAGE and probed with rabbit AgBR1 antisera. In FIG. 18C, silencing AgBR1 does not alter ZIKV infection at day 10 after virus injection. Viral burden was examined at day 10 after infection by qRT-PCR and normalized to mosquito Rp49 RNA. The results shown are pooled from three independent experiments. Significance was calculated using the non-parametric Mann-Whitney test.

The primer sequences of dsRNA against AgBR1 are:

(SEQ ID NO: 126)
F-taatacgactcactatagggGATGGACAGATGTCTCTTCGTG;
(SEQ ID NO: 127)
R-taatacgactcactatagggCCAAATCCAATCCATCGAAA.

The primer sequences of dsRNA against GFP are:

(SEQ ID NO: 128)
F-TAATACGACTCACTATAGGGGTGAGCAAGGGCGAGGAG;
(SEQ ID NO: 129)
R-TAATACGACTCACTATAGGGCATGATATAGACGTTGTGGCTGTT.

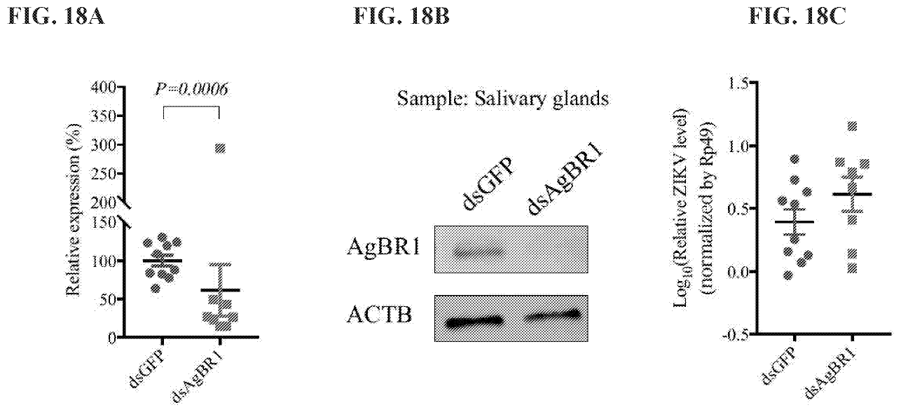

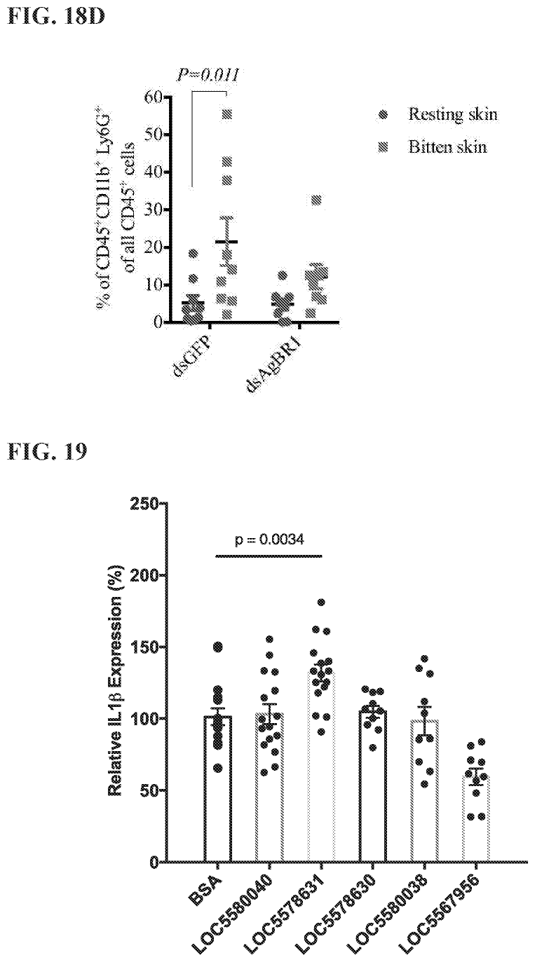

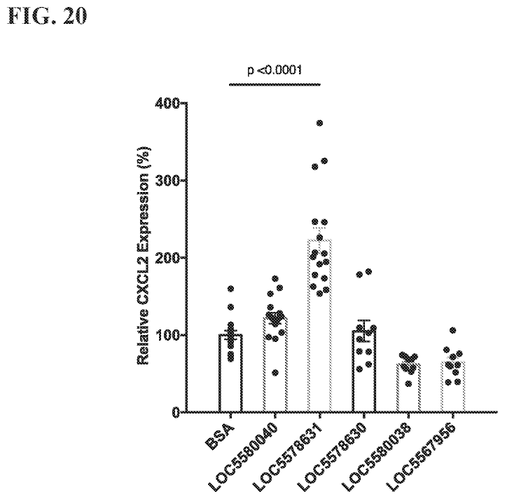

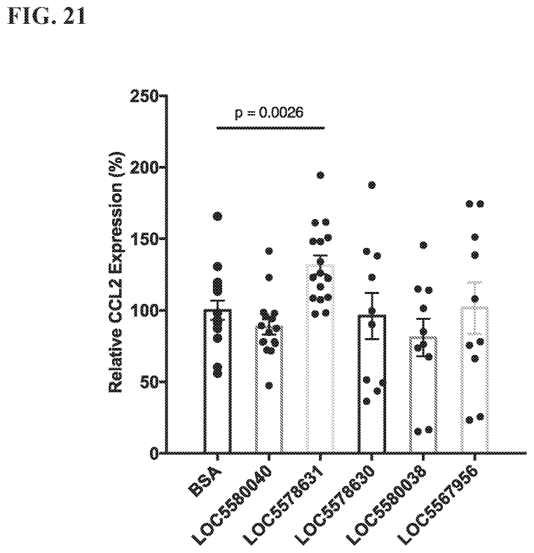

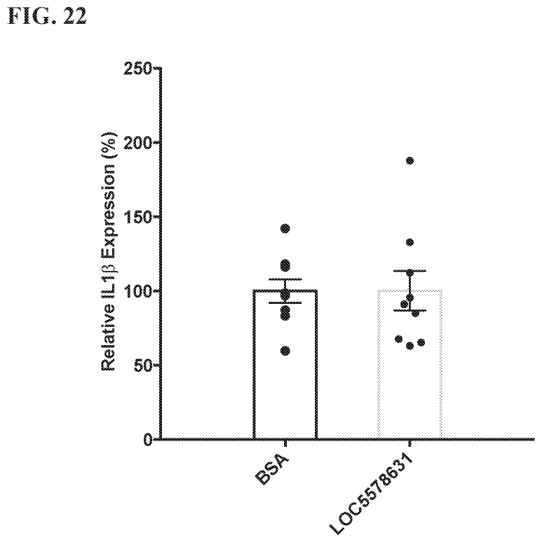

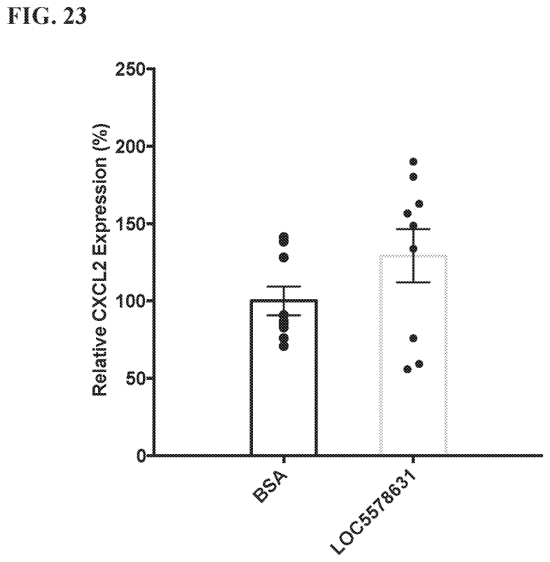

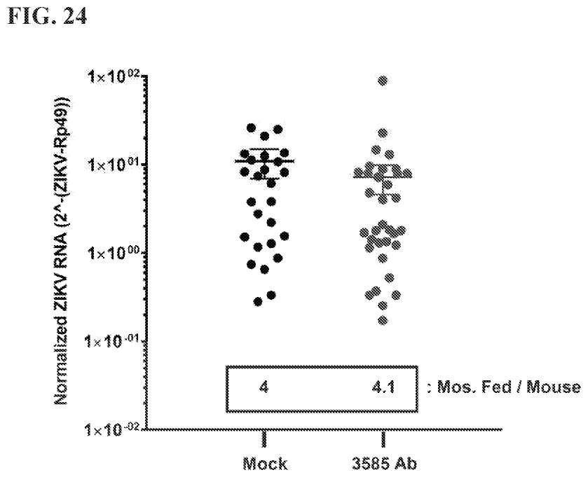

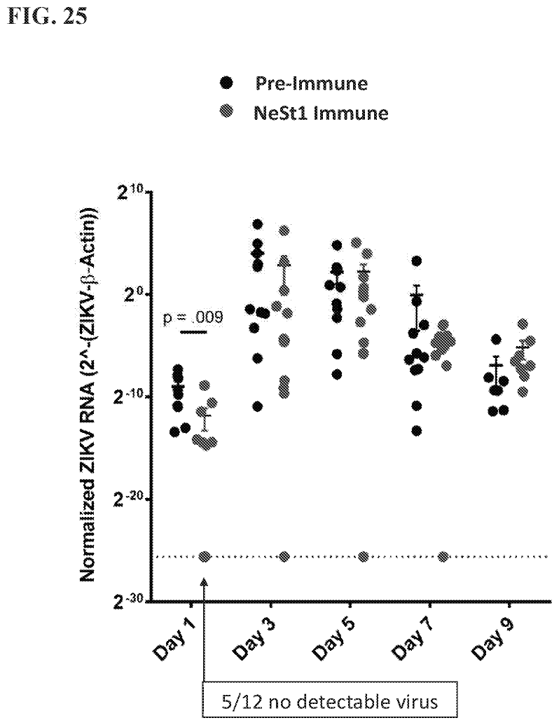

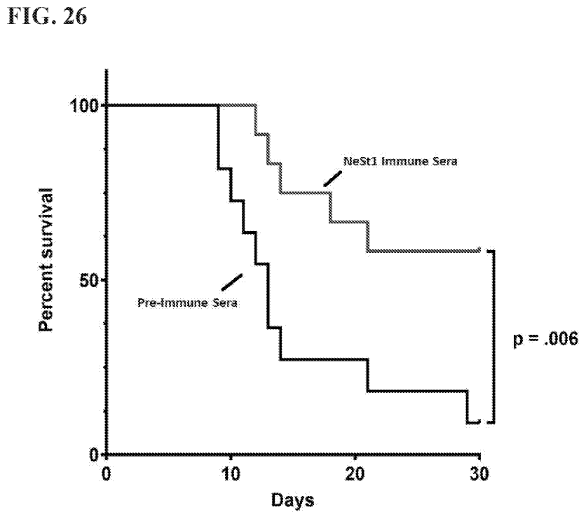

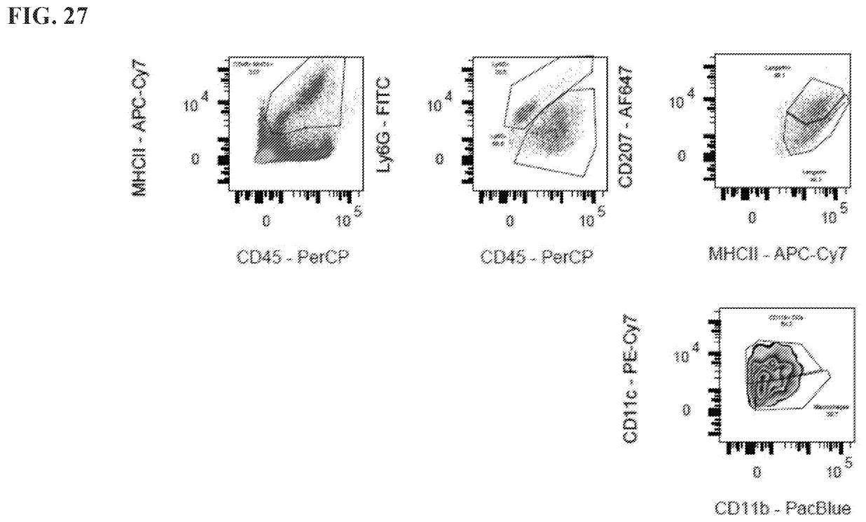

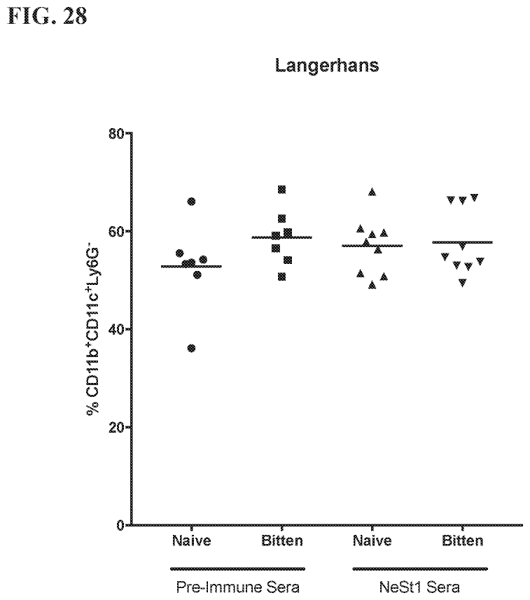

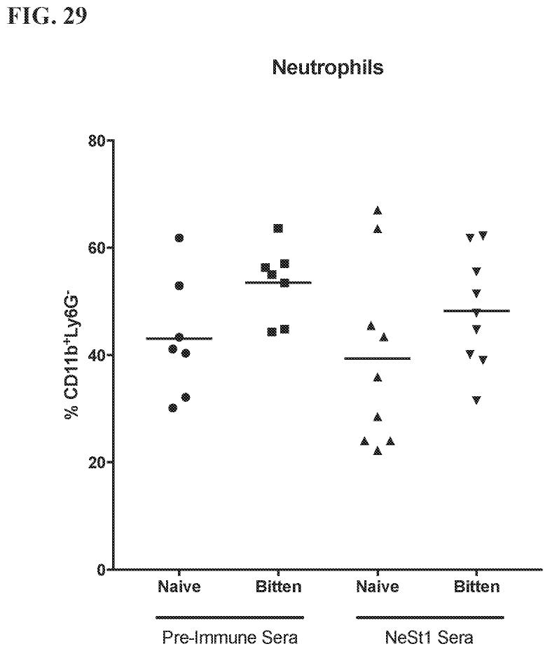

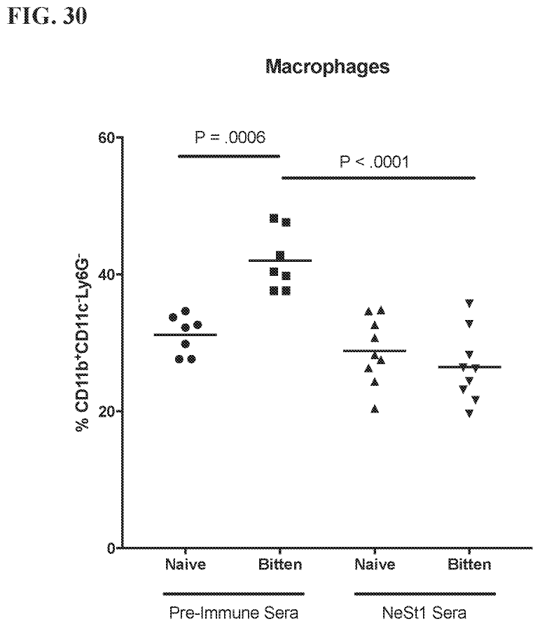

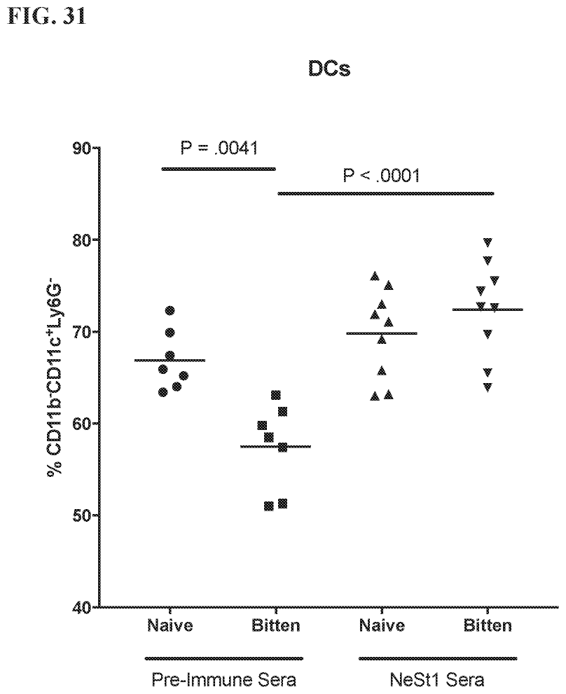

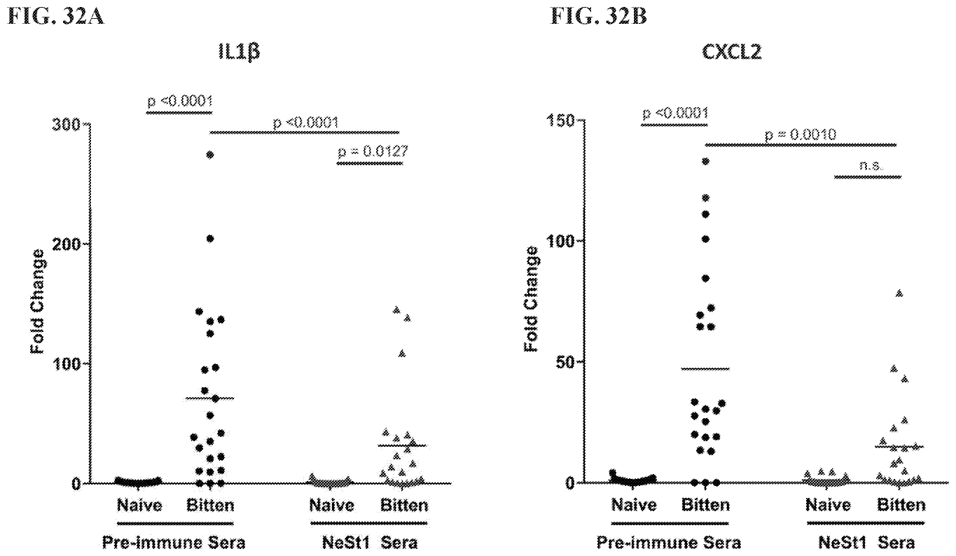

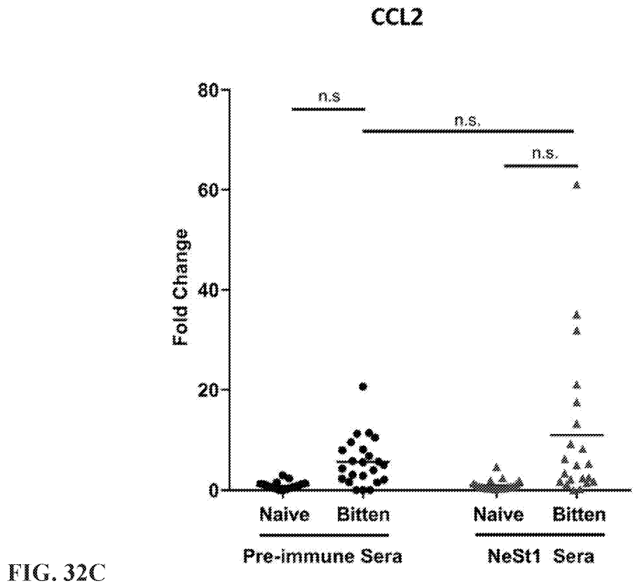

In FIG. 18D, a partial knock-down of AgBR1 impacts neutrophil recruitment in the skin. The percent of CD45⁺CD11b⁺Ly6G⁺ (neutrophils) cells in CD45⁺ leukocyte cells at 24 h after dsRNA-treated and Zika virus-infected mosquito feeding. Each dot represents one mouse. Significance was calculated using a two-way ANOVA test for multiple comparisons.

FIG. 19 shows IL1β ex vivo expression in purified neutrophils from bone marrow of naïve mice after treatment with antigenic proteins. NeSt1 protein activates neutrophils to express IL1β ex vivo. N=8-16 technical replicates from 4 mice for each protein.

FIG. 20 shows CXCL2 ex vivo expression in purified neutrophils from bone marrow of naïve mice after treatment with antigenic proteins. NeSt1 protein activates neutrophils to express CXCL2 ex vivo. N=8-16 technical replicates from 4 mice for each protein.

FIG. 21 shows CCL2 ex vivo expression in purified neutrophils from bone marrow of naïve mice after treatment with antigenic proteins. NeSt1 protein activates neutrophils to express CCL2 ex vivo. N=8-16 technical replicates from 4 mice for each protein.

FIG. 22 shows in vitro expression of IL1β in RAW macrophage cells after treatment with antigenic proteins. NeSt1 protein does not activate RAW macrophage cells to express IL1β. N=8 technical replicates and 2 biological replicates.

FIG. 23 shows in vitro expression of CXCL2 in RAW macrophage cells after treatment with antigenic proteins. NeSt1 protein does not activate RAW macrophage cells to express CXCL2. N=8 technical replicates and 2 biological replicates.

FIG. 24 shows similar Zika virus levels by qRT-PCR in mosquitoes fed on naïve AG129 mice and similar numbers (˜4) of infected mosquitoes fed on each mouse. Mosquito ZIKV burden is similar in insects fed on mice passively immunized with pre-immune sera and NeSt1 antisera.

FIG. 25 shows lower Zika virus level by qRT-PCR in AG129 treated with rabbit sera against mosquito protein NeSt1 (LOC5578631) at day 1 after feeding by ZIKV infected mosquitoes. Zika virus infected mosquitoes were allowed to feed on naïve AG129 mice and mice were bled every other day for 9 days. Viremia was measured by isolating RNA from serum and analyzing by qRT-PCR. N=12 mice/group in 3 independent experiments. Significance calculated by one-way ANOVA with post hoc Tukey test for multiple comparisons. Passive immunization against NeSt1 protein protects against early replication of ZIKV.

FIG. 26 shows serum against NeSt1 (LOC5578631) protects mice from severe Zika virus pathogenesis. Mice treated with NeSt1 antiserum and naïve rabbit serum were fed on by Zika virus infected mosquito and tracked for severe morbidity daily. N=12 mice/group in 3 independent experiments. Significance was calculated using log-rank test. Passive immunization against NeSt1 protein protects against pathogenesis of ZIKV.

FIG. 27 shows flow gating for experiments in FIGS. 28-31 in which mice treated with naïve rabbit serum or NeSt1 antiserum before feeding uninfected mosquitoes on ears and then isolating cells and analyzing by flow cytometry. MHCII⁺CD45⁺ cells were gated, Ly6G⁺ (neutrophils) were gated from this population. From MHCII⁺CD45⁺Ly6G⁻ cells, CD207⁺ (langerhan cells) were gated. From MHCII⁺CD45⁺Ly6G⁻ CD207⁻ cells, CD11c⁺ (dendritic cells) and CD11c⁻ (macrophages) were gated.

FIG. 28 shows similar numbers of MHCII⁺CD45⁺Ly6G⁻ CD207⁺ Langerhan cells in naïve and bitten ears in mice treated with naïve or NeSt1 antiserum. No significant differences between groups. N=8/group in two independent experiments. Passive immunization against NeSt1 does not affect the percentage of Langerhan cells at the bite site.

FIG. 29 shows more MHCII⁺CD45⁺Ly6G⁺ neutrophils in bitten compared to naïve ears in mice treated with naïve or NeSt1 antiserum. No significant differences between groups. N=8/group in two independent experiments. Passive immunization against NeSt1 does not affect the infiltration or expansion of neutrophils at the local bite site.

FIG. 30 shows higher percentage of MHCII⁺CD45⁺Ly6G⁻CD207⁻CD11c⁻ macrophages in bitten compared to naïve ears in mice treated with naïve but not NeSt1 antiserum. Significance calculated with one-way ANOVA with post hoc Tukey test for multiple comparisons. N=8/group in two independent experiments. Passive immunization against NeSt1 prevents the infiltration or expansion of macrophages at the local bite site.

FIG. 31 shows lower percentage of MHCII⁺CD45⁺Ly6G⁻ CD207⁻CD11c⁺ dendritic cells in bitten compared to naïve ears in mice treated with naïve but not NeSt1 antiserum. Significance calculated with one-way ANOVA with post hoc Tukey test for multiple comparisons. N=8/group in two independent experiments. Passive immunization against NeSt1 decreases the percentage of dendritic cells at the local bite site.

FIGS. 32A-32C show that blocking NeSt1 reduces induction of IL-1β and CXCL2 expression in vivo. Five-week-old C57BL/6 mice were passively immunized with preimmune rabbit sera or rabbit sera against NeSt1 protein. After 24 h, mosquitoes were allowed to bite the right ear (bitten), and the left ear was left alone (naive). cDNA was generated, and qPCR was used to measure IL-1β (FIG. 32A), CXCL2 (FIG. 32B), or CCL2 (FIG. 32C) expression. The data were normalized to mouse β-actin with the ΔΔC_{T} method and are presented as percentages of the average ΔΔC_{T} value of naive ear tissue (n=8 to 16 technical replicates from four mice in at least two independent biological replicates for each protein). Error bars represent the SEM. Significance was calculated by two-way ANOVA with a post hoc Tukey test for multiple comparisons. FIG. 32A shows serum against NeSt1 (LOC5578631) significantly reduces IL1β expression at site of blood feeding by Aedes aegypti mosquito. FIG. 32B shows serum against NeSt1 (LOC5578631) significantly reduces CXCL2 expression at site of blood feeding by Aedes aegypti mosquito. FIG. 32C shows serum against NeSt1 (LOC5578631) does not significantly reduce CCL2 expression at site of blood feeding by Aedes aegypti mosquito.

### DETAILED DESCRIPTION OF THE INVENTION

The present invention is based on an unexpected discovery by the present inventors that passive or active immunization against certain Aedes aegypti mosquito salivary gland proteins prevents and/or treats infection by flaviviruses such as Zika virus and West Nile virus.

As the global disease burden attributable to Zika virus continues to increase, new and creative strategies for vaccine design against this and other mosquito-borne viruses are needed. Although there are many efforts to develop Zika virus specific vaccines, there is currently no available commercial vaccine. The compositions and methods described herein utilize a new paradigm to develop a next-generation vaccine against Zika virus and other mosquito-borne viruses so as to address this important public health need. Human vaccines against infectious diseases are currently based upon components of specific pathogens, but the approach described herein targets arthropod vector proteins that affect pathogen transmission. The benefits of this novel approach include the possibility of developing vaccines that are effective for multiple viruses carried by the same arthropod vector and the shifting of selective evolutionary pressure away from the virus.

Without wishing to be bound by theory, it is hypothesized that salivary gland proteins from the mosquito vector, Ae. aegypti, are capable of enhancing virus infection of the mammalian host, and that viral transmission can be interrupted by mounting a robust immune response toward one or more of these proteins. The salivary gland is the last organ in the mosquito vector that viruses are in contact with before being inoculated into a host. Mosquito saliva is important for successful blood feeding.

The passive and active immunization approaches based on mosquito salivary gland proteins disclosed herein can be used alone or in conjunction with more conventional vaccines targeting viral components to increase their efficacy.

As disclosed in the Examples section, below, a yeast display screening conducted by the present inventors identified several Ae. aegypti antigenic salivary proteins, i.e., LOC5573204, LOC5578630, LOC5578631, LOC5567956, LOC5580038, LOC5566287, LOC5567958, LOC5568702, and LOC110675548 (numbering according to the LOC nomenclature used in vectorbase and the NCBI database for the Aedes aegypti Liverpool strain reference genome sequence from the Aedes aegypti Genome Working Group, see https://www.vectorbase.org/). One of these antigenic proteins, LOC5573204 bacteria-responsive protein 1 (AgBR1), shows particular promise as a candidate for a vaccine development. Described herein is a study of the protective effects of blocking the mosquito AgBR1 protein or the mosquito NeSt1 protein in preventing severe mosquito-borne Zika virus infection in mice. Mammals such as mice and humans may be actively immunized against the AgBR1 and/or NeSt1 protein.

### Definitions

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals as well as experimental animal models. In a preferred embodiment, the subject is human.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The terms “derivative” and “analog” are used interchangeably and refer to a related modified form of a polypeptide, wherein at least one amino acid substitution, deletion, addition, or chemical modification has been made. The terms “functional derivative” and “functional analog” mean that such derivative/analog/variant retains substantially the same biological activity as the unmodified form, in vivo and/or in vitro. A term “variant” refers to polypeptide derivatives/analogs, wherein (i) one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The term “antibody,” as used herein, refers to an immunoglobulin molecule which specifically binds with an antigen. An antibody can be an intact immunoglobulin derived from a natural source or from a recombinant source. Such antibody can comprise an immunoreactive portion of an intact immunoglobulin. The antibody may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. Any macromolecule, including virtually all proteins or peptides, can serve as an antigen. Furthermore, antigens can be derived from recombinant or genomic DNA. Any DNA which comprises a nucleotide sequences or a partial nucleotide sequence encoding a protein that elicits an immune response therefore encodes an “antigen” as that term is used herein. Furthermore, an antigen need not be encoded solely by a full-length nucleotide sequence of a gene. Partial nucleotide sequences of more than one gene may be used, for example these nucleotide sequences may be arranged in various combinations to elicit a desired immune response. Moreover, an antigen need not be encoded by a “gene” at all. An antigen can be generated synthesized or can be derived from a biological sample.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences.

As used herein, the term “immune response” includes T-cell mediated and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Throughout this disclosure, various aspects of the invention can be presented in a range format. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

The invention provides an immunological composition comprising a polypeptide or combination of polypeptides derived from at least one mosquito salivary protein associated with a mosquito-borne virus such as, e.g., a flavivirus (e.g., Zika virus, West Nile virus, Dengue virus, tick-borne encephalitis virus, yellow fever virus) or an alphavirus (e.g., Chikungunya virus (CHIKV), Ross River virus, O'nyong'nyong virus, Semliki Forest virus), useful in eliciting an immune response. The compositions comprising one or more polypeptide of the invention not only are useful as agents for immunoprotection but are also useful as agents for treatment of an ongoing disease or disorder associated with infection by mosquito-borne viruses such as, e.g., flaviviruses (e.g., Zika virus, West Nile virus, Dengue virus, tick-borne encephalitis virus, yellow fever virus) and alphaviruses (e.g., Chikungunya virus (CHIKV), Ross River virus, O'nyong'nyong virus, Semliki Forest virus) in a subject.

### Compositions of the Invention

In one aspect, the present invention provides a polypeptide or a combination of polypeptides, or a polynucleotide or a combination of polynucleotides encoding such polypeptides, wherein such polypeptides comprise or are derived from a mosquito salivary protein and are useful in inducing an immune response which prevents and/or treats an infection by a mosquito-borne virus such as, e.g., a flavivirus (e.g., Zika virus, West Nile virus, Dengue virus, tick-borne encephalitis virus, yellow fever virus) or an alphavirus (e.g., Chikungunya virus (CHIKV), Ross River virus, O'nyong'nyong virus, Semliki Forest virus).

In one embodiment, the invention provides a vaccine composition comprising at least one polypeptide selected from the group consisting of LOC5573204 (AgBR1), LOC5578630, LOC5578631, LOC5567956, LOC5580038, LOC5566287, LOC5567958, LOC5568702, LOC110675548, and fragments, derivatives or variants thereof. In some embodiments, such fragments, derivatives or variants are capable of inducing an immune response which prevents and/or treats an infection by a mosquito-borne virus such as, e.g., a flavivirus (e.g., Zika virus, West Nile virus, Dengue virus, tick-borne encephalitis virus, yellow fever virus) or an alphavirus (e.g., Chikungunya virus (CHIKV), Ross River virus, O'nyong'nyong virus, Semliki Forest virus). In some embodiments, polypeptide comprises a sequence of any one of SEQ ID NOS: 100-125, or a sequence of a contiguous 10 amino acid portion of any one of SEQ ID NOS: 100-125. In some embodiments, the polypeptide LOC5573204 comprises the sequence SEQ ID NO: 1. In some embodiments, the polypeptide LOC5578630 comprises the sequence SEQ ID NO: 2. In some embodiments, the polypeptide LOC5578631 comprises the sequence SEQ ID NO: 3. In some embodiments, the polypeptide LOC5567956 comprises the sequence SEQ ID NO: 4. In some embodiments, the polypeptide LOC5580038 comprises the sequence SEQ ID NO: 5. In some embodiments, the polypeptide LOC5566287 comprises the sequence SEQ ID NO: 43. In some embodiments, the polypeptide LOC5567958 comprises the sequence SEQ ID NO: 44. In some embodiments, the polypeptide LOC5568702 comprises the sequence SEQ ID NO: 45. In some embodiments, the polypeptide LOC110675548 comprises the sequence SEQ ID NO: 46. In some embodiments, the polypeptide LOC5573204 consists of the sequence SEQ ID NO: 1. In some embodiments, the polypeptide LOC5578630 consists of the sequence SEQ ID NO: 2. In some embodiments, the polypeptide LOC5578631 consists of the sequence SEQ ID NO: 3. In some embodiments, the polypeptide LOC5567956 consists of the sequence SEQ ID NO: 4. In some embodiments, the polypeptide LOC5580038 consists of the sequence SEQ ID NO: 5. In some embodiments, the polypeptide LOC5566287 consists of the sequence SEQ ID NO: 43. In some embodiments, the polypeptide LOC5567958 consists of the sequence SEQ ID NO: 44. In some embodiments, the polypeptide LOC5568702 consists of the sequence SEQ ID NO: 45. In some embodiments, the polypeptide LOC110675548 consists of the sequence SEQ ID NO: 46. In some embodiments, the fragments of LOC5573204 comprise the sequence of any one of SEQ ID NOS: 6-13. In some embodiments, the fragments of LOC5578630 comprise the sequence of any one of SEQ ID NOS: 14-21. In some embodiments, the fragments of LOC5578631 comprise the sequence of any one of SEQ ID NOS: 22-29. In some embodiments, the fragments of LOC5567956 comprise the sequence of any one of SEQ ID NOS: 30-36. In some embodiments, the fragments of LOC5580038 comprise the sequence of any one of SEQ ID NOS: 37-42. In some embodiments, the fragments of LOC5566287 comprise the sequence of any one of SEQ ID NOS: 47-59. In some embodiments, the fragments of LOC5567958 comprise the sequence of any one of SEQ ID NOS: 60-70. In some embodiments, the fragments of LOC5568702 comprise the sequence of any one of SEQ ID NOS: 71-85. In some embodiments, the fragments of LOC110675548 comprise the sequence of any one of SEQ ID NOS: 86-99. In some embodiments, the polypeptides of the invention have at least 90% amino acid sequence identity to any of the above sequences, e.g., at least 91%, 92%, 93%, 94%, 95%, 96%, 97%, 98%, or 99% amino acid sequence identity.

In one embodiment, the vaccine compositions of the invention also comprise one or more carriers and/or excipients. In one embodiment, the vaccine compositions of the invention also comprise one or more adjuvant. In one embodiment, the vaccine compositions of the invention also comprise one or more additional immunogenic polypeptides (e.g., an immunogenic polypeptide derived from the target virus such as, e.g., antigens from viral envelope or capsid).

Polypeptides of the present invention can be prepared using well known techniques. For example, the polypeptides can be prepared synthetically, using either recombinant DNA technology or chemical synthesis. Polypeptides of the present invention may be synthesized individually, or as longer polypeptides composed of two or more polypeptides. The polypeptides of the present invention can be isolated, i.e., substantially free of other naturally occurring host cell proteins and fragments thereof.

The polypeptides of the present invention may contain modifications, such as glycosylation, aglycosylation, side chain oxidation, or phosphorylation; so long as the modifications do not destroy the immunologic activity of the polypeptides. Other modifications include incorporation of D-amino acids or other amino acid mimetics that can be used, for example, to increase the serum half-life of the polypeptides.

The polypeptides of the invention can be modified whereby the amino acid is substituted for a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note that the parenthetic letters indicate the one-letter codes of amino acids. As used herein, X stands for any amino acid.

The polypeptides of the invention can be prepared as a combination, which includes two or more of polypeptides of the invention, for use as a vaccine for the reduction, prevention, or treatment of a mosquito-borne virus infection (e.g., an infection by a flavivirus [such as, e.g., Zika virus, West Nile virus, Dengue virus, tick-borne encephalitis virus, yellow fever virus] or an infection by an alphavirus [e.g., Chikungunya virus (CHIKV), Ross River virus, O'nyong'nyong virus, Semliki Forest virus]). The polypeptides may be in a cocktail or may be conjugated to each other using standard techniques. For example, the polypeptides can be expressed as a single polypeptide sequence. The polypeptides in the combination may be the same or different.

The present invention should also be construed to encompass “analogs,” “mutants,” “derivatives,” and “variants” of the polypeptides of the invention (or of the DNA encoding the same) which analogs, mutants, derivatives and variants are polypeptides which are altered in one or more amino acids (or, when referring to the nucleotide sequence encoding the same, are altered in one or more base pairs) such that the resulting polypeptide (or DNA) is not identical to the sequences recited herein, but has the same biological property as the polypeptides disclosed herein.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art several alternative polynucleotides are possible due to redundant codons, while retaining the biological activity of the translated polypeptides.

It is to be understood explicitly that the scope of the present invention encompasses homologs, analogs, variants, fragments, derivatives and salts, including shorter and longer polypeptides and polynucleotides, as well as polypeptide and polynucleotide analogs with one or more amino acid or nucleic acid substitutions, as well as amino acid or nucleic acid derivatives, non-natural amino or nucleic acids and synthetic amino or nucleic acids as are known in the art, with the stipulation that these modifications must preserve the immunologic activity of the original molecule. Specifically, any active fragments of the active polypeptides as well as extensions, conjugates and mixtures are included and are disclosed herein according to the principles of the present invention.

In one embodiment, the compositions of the invention comprise a nucleic acid sequence encoding one or more of the above polypeptides, derivatives, variants or fragments. Such nucleic acid sequence can be included in a vector, e.g., an expression vector.

The nucleic acids of the invention may encompass an RNA or a DNA sequence encoding a polypeptide of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

Further, any number of procedures may be used for the generation of mutant, derivative or variant forms of a protein of the invention using recombinant DNA methodology well known in the art such as, for example, that described in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). Procedures for the introduction of amino acid changes in a polypeptide or polypeptide by altering the DNA sequence encoding the polypeptide are well known in the art and are also described in these, and other, treatises.

The nucleic acids encoding the polypeptide or combinations of polypeptides of the invention of the invention can be incorporated into suitable vectors, including but not limited to, plasmids and recombinant viral vectors (e.g., retroviral vectors, lentiviral vectors, adenoviral vectors, adeno-associated virus (AAV) vectors, herpes virus vectors). Such vectors are well known in the art and are therefore not described in detail herein.

In one embodiment, the invention includes a nucleic acid sequence encoding one or more polypeptides of the invention operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). The incorporation of a desired polynucleotide into a vector and the choice of vectors is well-known in the art as described in, for example, Sambrook et al. (2012), and in Ausubel et al. (1997).

Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.)

For expression of the desired nucleotide sequences of the invention, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

A promoter and/or enhancer may be employed that effectively directs the expression of the DNA segment in the cell type, organelle, and organism chosen for expression. The promoters employed may be constitutive, tissue-specific, inducible, and/or useful under the appropriate conditions to direct high-level expression of the introduced DNA segment, such as is advantageous in the large-scale production of recombinant proteins and/or polypeptides. The promoter may be heterologous or endogenous.

One example of a constitutive promoter sequence is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter, where the promoter is active only in a desired tissue. Tissue-specific promoters are well known in the art and include, but are not limited to, the HER-2 promoter and the PSA associated promoter sequences.

The expression vector to be introduced into a cell can also contain either a selectable marker gene or a reporter gene or both to facilitate identification and selection of expressing cells from the population of cells sought to be transfected or infected through viral vectors. Such introduction of the expression vector may facilitate assessing the expression of the nucleotide sequences encoding the polypeptide or combinations of polypeptides of the invention. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the expression vector is modified to increase the expression of the desired polypeptide. For example, the vector can undergo codon optimization to improve expression in a given mammal. For example, the vector can be codon-optimized for human expression. In another embodiment, the expression vector comprises an effective secretory leader. An exemplary leader is an IgE leader sequence. In another embodiment, the expression vector comprises a Kozak element to initiate translation. In another embodiment, the nucleic acid is removed of cis-acting sequence motifs/RNA secondary structures that would impede translation. Such modifications, and others, are known in the art for use in DNA vaccines (Kutzler et al, 2008, Nat. Rev. Gen. 9: 776-788; PCT App. No. PCT/US2007/000886; PCT App. No.; PCT/US2004/018962).

In various embodiments, the vaccine compositions of the invention are effective to induce an immune response to the antigen in a subject (e.g., a human). In some embodiments, said immune response is effective to prevent and/or treat mosquito-borne viral infections such as infections caused by flaviviruses (e.g., Zika virus, West Nile virus, Dengue virus, tick-borne encephalitis virus, and yellow fever virus) and alphaviruses (e.g., Chikungunya virus (CHIKV), Ross River virus, O'nyong'nyong virus, and Semliki Forest virus).

In some embodiments, the vaccine composition comprises an additional immunostimulatory agent or nucleic acids encoding such an agent. Immunostimulatory agents include but are not limited to an additional antigen, an immunomodulator, an antigen presenting cell or an adjuvant. Non-limiting examples of suitable adjuvants include alum and such, cholera toxin, salmonella toxin, but are not limited thereto. In other embodiments, one or more of the additional agent(s) is covalently bonded to the antigen or an immunostimulatory agent, in any combination. In certain embodiments, the vaccine composition is conjugated to or comprises HLA anchor motif amino acids.

In one embodiment, the vaccine composition is administered in combination with an adjuvant. Non-limiting examples of suitable adjuvants include cholera toxin, salmonella toxin, alum and such, but are not limited thereto. In another embodiment, the vaccine is administered in the absence of an adjuvant.

In a non-limiting example, a nucleic encoding an antigen can also be formulated with an adjuvant. The various compositions described herein may further comprise additional components. For example, one or more vaccine components may be comprised in a lipid or liposome or nanoparticle.

Vaccine compositions of the present invention, and its various components, may be prepared and/or administered by any method disclosed herein or as would be known to one of ordinary skill in the art, in light of the present disclosure.

In one embodiment, the polypeptide vaccine of the invention includes, but is not limited to at least one polypeptide, or a fragment thereof, optionally mixed with adjuvant substances. In some embodiments, the polypeptide is introduced together with an antigen presenting cell (APC). The most common cells used for the latter type of vaccine are bone marrow and peripheral blood derived dendritic cells, as these cells express costimulatory molecules that help activation of T cells. WO 00/06723 discloses a cellular vaccine composition which includes an APC presenting tumor associated antigen polypeptides. Presenting the polypeptide can be effected by loading the APC with a polynucleotide (e.g., DNA, RNA) encoding the polypeptide or loading the APC with the polypeptide itself.

Vaccine compositions may further comprise a pharmaceutically acceptable carrier, diluent or excipient to form a pharmaceutical formulation, or unit dosage form. The total active ingredients (e.g., polypeptides and inhibitors) in such formulations include from 0.1 to 99.9% by weight of the formulation. The active ingredients (e.g., polypeptides and inhibitors) for administration may be present as a powder or as granules; as a solution, a suspension or an emulsion.

Pharmaceutical formulations containing the compositions of the invention can be prepared by procedures known in the art using well known and readily available ingredients. The compositions of the invention can also be formulated as solutions appropriate for parenteral administration, for instance by intramuscular, subcutaneous or intravenous routes.

Thus, the composition may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Passive Immunization Compositions of the Invention

The present invention also encompasses various compositions comprising antibodies which interact with the polypeptides of the invention (preferably specifically and selectively). Such antibody-containing compositions can be used for passive immunization against mosquito-borne viral infections such as infections caused by flaviviruses (e.g., Zika virus, West Nile virus, Dengue virus, tick-borne encephalitis virus, yellow fever virus) and alphaviruses (e.g., Chikungunya virus (CHIKV), Ross River virus, O'nyong'nyong virus, Semliki Forest virus). An antibody can be an intact immunoglobulin derived from a natural source or from a recombinant source. Such antibody can comprise an immunoreactive portion of an intact immunoglobulin. The antibody may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

Inhibitor Compositions of the Invention

The invention also includes inhibitor compositions and methods for inhibiting a function of one or more of the mosquito salivary polypeptides disclosed herein or an interaction between the mosquito salivary polypeptides disclosed herein and host cells or proteins or between the mosquito salivary polypeptides disclosed herein and a mosquito-borne virus. The inhibitor compositions of the invention include, but are not limited to, a chemical compound, a protein, a peptide, a peptidomimetic, an antibody, a ribozyme, a small molecule chemical compound, a glycan, an antisense nucleic acid molecule (e.g., siRNA, miRNA, etc.), or combinations thereof. In some embodiments, the inhibitor compositions bind to the mosquito salivary polypeptide. In other embodiments, the inhibitor compositions bind to the host cell or virus.

Genetically Modified Mosquitos of the Invention

The invention also includes mosquitoes that have been genetically modified (e.g., using CRISPR-Cas9 technology) to alter or eliminate one or more of the mosquito salivary polypeptides disclosed herein.

Methods of the Invention

The compositions of the invention comprising mosquito salivary polypeptides of the invention or nucleotides encoding such polypeptides can be used as immunostimulatory agents to prevent and/or treat a mosquito-borne viral infection such as, e.g., an infection by a flavivirus (e.g., Zika virus, West Nile virus, Dengue virus, tick-borne encephalitis virus, yellow fever virus) or an alphavirus (e.g., Chikungunya virus (CHIKV), Ross River virus, O'nyong'nyong virus, Semliki Forest virus) in a subject (e.g., human or veterinary animal) and associated disease or disorder. In one embodiment, the composition of the invention comprises a AgBR1 polypeptide, or a variant thereof.

The present invention also encompasses a method of inducing anti-mosquito-borne virus (e.g., anti-flavivirus or anti-alphavirus) immunity using the compositions described herein.

In another embodiment, the methods of the invention comprise administering to the subject a bacterium or virus comprising a nucleic acid sequence encoding at least one mosquito salivary protein. In another embodiment, the methods of the invention comprise administering to the subject a bacterium or virus expressing at least a portion of at least one mosquito salivary protein. In another embodiment, the methods of the invention comprise administering to the subject a bacterium or virus comprising at least a portion of at least one mosquito salivary protein.

Effectiveness of the resulting immune response can be determined for detecting the induction of cytotoxic T lymphocytes (CTL) which are well known in the art. A method for evaluating the inducing action of CTL using dendritic cells (DCs) as APC is well known in the art. DC is a representative APC having the strongest CTL inducing action among APCs. In this method, the polypeptide or combination of polypeptides is initially contacted with DC and then this DC is contacted with T cells. Detection of T cells having cytotoxic effects against the cells of interest after the contact with DC shows that the polypeptide or combination of polypeptides has an activity of inducing the cytotoxic T cells. Furthermore, the induced immune response can be also examined by measuring IFN-gamma produced and released by CTL in the presence of antigen-presenting cells that carry immobilized polypeptide or combination of polypeptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

Generally, when using a polypeptide for cellular immunotherapy, efficiency of the CTL-induction can be increased by combining a plurality of polypeptides having different structures and contacting them with DC. Therefore, when stimulating DC with protein fragments, it is advantageous in certain embodiments to use a mixture of multiple types of fragments.

The induction of immunity by the compositions of the invention can be further confirmed by observing the induction of antibody production.

Thus, the invention provides a method for treating or preventing an infection by a mosquito-borne virus such as, e.g., a flavivirus (such as but not limited to, Zika virus, West Nile virus, Dengue virus, tick-borne encephalitis virus, and yellow fever virus) or an alphavirus (such as but not limited to, Chikungunya virus (CHIKV), Ross River virus, O'nyong'nyong virus, and Semliki Forest virus). The therapeutic compounds or compositions of the invention may be administered prophylactically or therapeutically to subjects suffering from, at risk of developing, or susceptible to developing an infection by a mosquito-borne virus such as, e.g., a flavivirus or an alphavirus. Such subjects may be identified using standard clinical methods. In the context of the present invention, prophylactic administration occurs prior to the manifestation of overt clinical symptoms of a disease, such that a disease or disorder is prevented or alternatively delayed in its progression.

The polypeptide or combination of polypeptides of the invention having immunological activity, or a polynucleotide or vector encoding such a polypeptide or combination of polypeptides, may optionally be combined with an adjuvant. An adjuvant refers to a compound that enhances the immune response against the polypeptide or combination of polypeptides when administered together (or successively) with the polypeptide having immunological activity. Non-limiting examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

Administration of the compositions of the invention can comprise, for example, intramuscular, intravenous, peritoneal, subcutaneous, intradermal, as well as topical administration.

The actual dose and schedule can vary depending on whether the compositions are administered in combination with other pharmaceutical compositions, or depending on inter-individual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the compositions can be further approximated through analogy to compounds known to exert the desired effect.

Administration of the therapeutic composition in accordance with the present invention may be continuous or intermittent, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

Compositions of the invention can be administered singly or in any combination. Further, infection inhibitors (e.g., immunogenic compositions comprising viral antigens or nucleic acids encoding such viral antigens) and active compounds can be administered singly or in any combination in a temporal sense, in that they may be administered concurrently, or before, and/or after each other.

The present disclosure is not limited to treatment of a disease or disorder that is caused by a flavivirus or an alphavirus but can be useful for treating other mosquito-borne diseases.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Materials and Methods for Examples 1-7

All experiments were performed in accordance with guidelines from the Guide for the Care and Use of Laboratory Animals of the NIH. The animal experimental protocols were approved by the Institutional Animal Care and Use Committee (IACUC) at the Yale University School of Medicine (Assurance number A3230-01). All infection experiments were performed in a biosafety level 2 animal facility, according to Yale University regulations. Every effort was made to minimize murine pain and distress. Mice were anesthetized with ketamine/xylazine for mosquito infection experiments and euthanized as suggested by the Yale IACUC.

### Viruses and Cell Lines

Vero cells (ATCC) were maintained in DMEM containing 10% FBS and antibiotics at 37° C. with 5% CO₂. Aedes albopictus C6/36 cells were grown in DMEM supplemented with 10% FBS, 1% tryptose phosphate, and antibiotics at 30° C. with 5% CO₂. Drosophila S2 cells (ATCC) were passaged in Schneider's Drosophila media with 10% FBS at 28° C. A Mexican strain of Zika virus (Accession number KX446950), MEX2-81, was propagated in C6/36 insect cells.

### Mosquitoes and Animals

A. aegypti (Ho Chi Minh strain, obtained from the J. Powell laboratory at Yale) mosquitoes were maintained on 10% sucrose feeders inside a 12″×12″×12″ metal mesh cage (BioQuip #1450B) at 28° C. and ˜80% humidity. Egg masses were generated via blood meal feeding on naïve mice. All mosquitoes were housed in a warm chamber in a space approved for BSL2 and ACL3 research. Four to six-week old gender mixed Ifnαr 1^{-/-}Ifnγr^{-/-} mice (AG129-SV129 background) were used in the Zika virus infection studies (30). Mice were randomly chosen for experimental groups. For isolating splenocytes, 5-week old male C57BL/6 mice were purchased from the Jackson Laboratory. All mice were kept in a pathogen-free facility at Yale University.

### Yeast Display Screening

To prepare RNA for library construction, salivary glands from about 300 A. aegypti mosquitoes, which had previously fed on mice once, were harvested. RNA was purified with the RNeasy Mini Kit (QIAGEN) and the purity of collected RNA was validated by gel electrophoresis confirm the presence of 18S and 28S rRNA. The cDNAs were synthesized by a modified SMART™ cDNA synthesis kit according to protocols by Bio S&T Inc. (Quebec, Canada). After generating double strand cDNAs by primer extension and cDNA normalization, cDNAs were directionally cloned into the yeast expression vector pYD1 (Invitrogen, CA) to generate a salivary gland expression library (Invitrogen, CA). Digestion of plasmids purified from 10 clones of the pYD1-salivary gland library showed an average insert size of 1.2 kb and 100% of the clones contained inserts. The total number of primary clones was over 1 million. Plasmid DNA was purified from the library using the QIAGEN Plasmid Midi Kit (QIAGEN, CA, USA). Growth of transformed yeast cells and induction of recombinant protein production was done as previously described (12,50). Briefly, fresh Saccharomyces cerevisiae EBY100 cells (Invitrogen, CA) with 2 μg of plasmid DNA were electroporated and subsequently grown in SDCAA medium (2% dextrose, 0.67% yeast nitrogen base, 0.5% bacto amino acids, 30 mM NaHPO₄, 62 mM NaH₂PO₄) overnight at 30° C. with shaking at 200 rpm.

The induction of surface protein expression was performed as described previously (12,50). In brief, transformed yeast cells were grown for 2.4 hours at 30° C. in SGCAA medium (2% galactose, 0.67% yeast nitrogen base, 0.5% bacto amino acids, 30 mM NaHPO₄, 62 mM NaH₂PO₄). After induction with galactose, selection was performed by MACS separation (Miltenyi Biotec, Auburn, Calif.). Induced yeast cells were incubated with purified IgG derived from mice repeatedly bitten by A. aegypti. For MACS separation, an LS column (Miltenyi Biotec Cat #130-042-401) was placed onto the magnet and stand assembly. After washing the column, induced yeast cells were applied to the column. After passing through the column, bound yeast cells were eluted by removing the column from the magnet and adding SDCAA medium to the column. Then, eluted yeast cells were propagated for additional rounds of sorting. After 4 rounds of magnetic sorting, plasmids were recovered using a Zymoprep™ II Yeast Plasmid Miniprep kit (Zymo Research), transformed into E. coli DH5α-competent cells (Invitrogen, CA), and sequenced.

### Purification of Recombinant Proteins and Antiserum Preparation

AgBR1 (LOC5573204), SP (LOC5578630) and D7Bclu (LOC5567956) were cloned in-frame into the pMT-Bip-V5-His tag vector (Invitrogen, CA) and recombinant proteins expressed and purified using the Drosophila Expression System (Invitrogen, CA) as described earlier (50). AgBR1, SP and D7Bclu were purified from the supernatant by TALON Metal Affinity Resin (Clontech, CA) and eluted with 150 mM imidazole. The eluted samples were filtered through a 0.22-μm filter and concentrated with a 10-kDa concentrator (Sigma-Aldrich, MO) by centrifugation at 4° C., washed and dialyzed against PBS. Recombinant protein purities were assessed by SDS-PAGE and quantified using the BCA protein estimation kit (Thermo Scientific. IL). The PCR primer sequences for cloning are listed in Table 3.

To generate rabbit sera against recombinant proteins, rabbits were immunized subcutaneously with 80-150 μg of recombinant proteins in complete Freund's adjuvant and boosted twice at every two weeks with 80-150 μg of recombinant proteins in incomplete Freund's adjuvant. Rabbits were euthanized and sera were obtained by cardiac puncture two weeks after the final boost. Reactivity to recombinant proteins was examined by immunoblot and ELISA.

### Enzyme-Linked Immunosorbent Assay (ELISA)

Recombinant AgBR1, SP, D7Bclu, OVA, or salivary gland extracts in PBS (0.1 μg/50 μl/well) were coated on 96 well plates overnight at 4° C. After being blocked with 2% non-fat milk for one hour at room temperature, the plates were then incubated with serum samples serially diluted in PBS for one hour at room temperature. After being washed with PBS plus 0.05% Tween-20 (PBS-T) (Sigma) three times, the plates were incubated with HRP-conjugated secondary antibodies. Enzyme activity was detected by incubation with 100 μl of 3,3′,5,5′-Tetramethylbenzidine solution (KPL, USA) for 15 minutes at room temperature in the dark. The reaction was stopped by the addition of 1M H₂SO₄. The optical density (OD) at 450 nm was measured with a microplate reader.

### Immunoblot

Recombinant proteins, BSA or salivary gland extracts were separated by SDS-PAGE using 4-20% Mini-Protean TGX gels (Bio-Rad) at 200 V for 25 min. Proteins were transferred onto a PVDF membrane for 60 min at 4 V. Blots were blocked in 1% non-fat milk in water for 60 min. Primary antibodies were diluted in 0.05% PBS-T and incubated with the blots for 1 h at room temperature or 4° C. overnight. HRP-conjugated secondary antibodies were diluted in PBS-T and incubated for 1 h at room temperature. After washing by PBS-T, the immunoblots were imaged with a LI-COR Odyssey imaging system.

Splenocyte Stimulation with Recombinant AgBR1

Splenocytes were isolated from C57BL/6 mice. Briefly, the spleens were minced in RPMI 1640 (Sigma-Aldrich) and forced gently through a 70 μm cell-strainer nylon mesh using a sterile syringe plunger and centrifuged at 400 g for 5 min. After washing once using cold PBS, spleen cells were incubated in 2 ml 0.83% NH₄Cl for 5 min, then placed in 20 ml PBS, centrifuged at 400 g for five minutes and then resuspended in RPMI 1640. The total number of cells was calculated using a hemocytometer. Isolated splenocytes were stimulated with 5 μg/ml recombinant AgBR1 or BSA and cultured with serum-free RPMI medium for 6 hours and 24 hours. Total RNA was extracted by the RNeasy Mini Kit (QIAGEN) according to the instructions. The cDNA was generated with iScript cDNA Synthesis Kit (Bio-rad) according to manufacturer's protocol. Gene expression was examined by quantitative RT-PCR (qRT-PCR) using IQ™ SYBR Green Supermix. Target gene mRNA levels were normalized to mouse β actin RNA levels according to the 2^{-ΔΔCt} calculations. The qRT-PCR primer sequences are listed in Table 3.

Co-Inoculation of Zika Virus with AgBR1

AG129 mice were inoculated via subcutaneous (footpad) with 3 PFU of Zika virus along with 10 μg AgBR1 (total volume; 40 μl). Survivals were monitored everyday post-infection. Mice exhibiting neurologic disease such as paralysis or weight loss of over 20% of body weight were euthanized. The weight loss is very rapid and usually begins three days before death, coinciding with neurological symptoms. Total RNA from murine blood was extracted in TRIzol Reagent and qRT-PCR was performed to examine Zika virus levels as previously described (23).

### Passive or Active Immunization Studies

Zika virus injection was performed as described in Uraki et al. (33). Briefly, Zika virus-filled needles were carefully inserted into the thorax of each mosquito and 69 nl of virus (100 PFU) was injected using a Nanoject II auto-nanoliter injector (Drummond). Infected mosquitoes were placed back in paper cups with mesh lids and maintained in triple containment for 10 days in a warm chamber. Mosquitoes were knocked-down on ice and salivary glands were dissected to examine the virus levels after mosquito feeding. RNA from salivary glands was purified with the RNeasy Mini Kit (QIAGEN), and cDNA was generated with iScript cDNA Synthesis Kit (Bio-rad) according to manufacturer's protocol. Gene expression was examined by quantitative RT-PCR (qRT-PCR) using IQ™ SYBR Green Supermix. Viral RNA levels were normalized to mosquito Rp49 RNA levels according to the 2^{-ΔΔCt} calculations.

For passive rabbit antiserum transfer experiments, mice were injected intraperitoneally with 150 μl per animal of antiserum against specific mosquito proteins or naive rabbit serum one day before challenge. On the same day, two infected mosquitoes were randomly aliquoted into individual cups with mesh covers. On the following day, mice were anesthetized with ketamine-xylazine and fed on by two Zika virus-infected mosquitoes. For active immunization, mice were immunized subcutaneously with 10 μg of AgBR1 or ovalbumin in complete Freund's adjuvant and boosted twice every 2 weeks with the same amount of AgBR1 or ovalbumin in incomplete Freund's adjuvant. Two weeks after the final immunization, mice were anesthetized with ketamine-xylazine and fed on by two Zika virus infected mosquitoes. The blood of fed mice was collected at 1, 3, 5, 7 and 9 days post infection. Survivals were monitored every day. Mice exhibiting weight loss of >20% of initial body weight or neurologic disease were euthanized. The weight loss is very rapid and usually begins three days before death, coinciding with neurological symptoms. Viremia levels were examined at 1, 3, 5, 7, 9 days post infection (dpi) as described above.

Needle Inoculation of Zika Virus after Passive Immunization

Mice were injected intraperitoneally with control serum or AgBR1 antiserum one day before challenge. On the following day, mice were inoculated via subcutaneous footpad injection with 0.3 PFU of Zika virus. Survivals were monitored everyday post-infection. Mice exhibiting weight loss of >20% of initial body weight or neurologic disease were euthanized. The weight loss is very rapid and usually begins three days before death, coinciding with neurological symptoms. Viremia levels were examined at 1, 3, 5, 7, 9 dpi as described above.

### Gene Silencing

RNA interference of genes expressed in the mosquito SGs was performed. Double stranded (ds) RNA targeting either a 400 bp region of the AgBR1 gene or an irrelevant green fluorescent protein (GFP) gene were transcribed using gene-specific primers designed with a T7 promoter and the MEGAScript RNAi kit (Thermo Fisher Scientific, Ambion). The primers for generating dsRNA are listed in Table 3. For silencing the AgBR1 gene, adult female A. aegypti mosquitoes were kept on ice and then transferred to a cold tray to receive a dsRNA injection. Two hundred ng of dsRNA in PBS were microinjected into the thorax of each mosquito using a Nanoject II Auto-Nanoliter Injector (Drummond). At day 3 post dsRNA injection, mosquitoes were knocked-down on ice and injected with Zika virus described above. At day 10 post virus injection, salivary glands were dissected to examine the AgBR1 expression levels by qRT-PCR and AgBR1 protein production by immunoblot.

Analysis of Local Immune Responses after Bites of Zika Virus Infected Mosquitoes or Intradermal Injection

For the analysis of local immune responses after bites of Zika virus infected mosquitoes, AG129 mice were allowed to be fed on the left ear by Zika virus-infected A. aegypti mosquitoes.

For the analysis of local immune responses after intradermal injection, AgBR1 was intradermally injected into the left ear. Briefly, the ear of an individual mouse was gently immobilized over a 14 ml Falcon tube covered with double stick tape. Five hundred nanoliters containing 0.5 μg/μl were injected intradermally into the dorsal ear using glass micropipettes with a 80 μm diameter beveled opening made as described elsewhere (33) and a Nanoject II Auto-Nanoliter Injector (Drummond).

One day later, mice were euthanized, and the locations bitten by mosquitoes or intradermally-injected locations and naïve skins were punched using a Disposable Biopsy Punch. Total RNA was extracted by the RNeasy Fibrous Tissue Mini Kit (QIAGEN) according to the manufacturer's instructions.

For qRT-PCR, the cDNA generation and analysis of gene expression was conducted as described above. Gene expression was queried using IQ™ SYBR Green Supermix. Target gene mRNA levels were normalized to mouse β actin RNA levels according to the 2^{-ΔΔCt} calculations. The qRT-PCR primer sequences are listed in Table 3.

For RNA-seq library preparation and sequencing, barcoded libraries were generated by standard Truseq mRNA library protocol (Illumina) and sequenced with a 2×75 bp paired-end protocol on the HiSeq 4000 Sequencing System (Illumina).

All the analysis of RNA-seq data was performed using Partek flow (v7.0). RNA-seq data were trimmed and mapped to a mm10 genome reference using STAR (2.5.0e). The aligned reads were quantified to ENSEMBL transcripts release 91 using the Partek’ E/M algorithm and the subsequent steps were performed on gene-level annotation followed by total count normalization. The gene-level data were normalized by dividing the gene counts by total number of reads followed by addition of a small offset (0.001). Differential expression was assessed by fitting the Partek's log-normal model with shrinkage (comparable in performance to limma-trend). Genes having geometric mean below value of 1.0 were filtered out from the analysis. Hierarchal clustering was performed on the genes, which were differentially expressed across the conditions (P value <0.05, fold change >1.5 for each comparison). Gene set enrichment analysis (GSEA) was performed on normalized gene expression counts of RNA-seq data as described previously (34). Gene sets with an estimated false discovery rate (FDR) of <0.05 were considered significant according to the GSEA guidelines.

### Histopathology

Ear skins of the bite site and non-bite site on the contralateral ear were harvested by punch biopsy, fixed in 4% paraformaldehyde/PBS, paraffin embedded, and processed for hematoxylin and eosin staining. The histological findings were scored for the severity and character of the inflammatory response using a blinded grading scale that was previously described (51), with minor modifications. Responses were graded as follows: 0, no response; 1, minimal response; 2, mild response; 3, moderate response; and 4, marked response. The responses were evaluated and graded on the histological sites with the most prominent responses in each specimen. The total histology score was calculated as the sum of scores, including inflammation, neutrophils, mononuclear cells and edema. The slides were blinded, randomized, and reread to determine the histology score by the same dermatopathologist throughout all studies.

### Imaging Mass Cytometry (IMC)

IMC was performed on slides dewaxed in xylene for 20 minutes according to previously described studies (52). After hydration in sequential concentrations of ethanol (100%, 95%, 80%, 70%) for 5 min, slides were incubated with antigen retrieval solution at 90-95° C. for 20 min. Slides were then cooled to room temperature and washed with ddH₂O and PBS (lacking Ca⁺⁺ or Mg⁺⁺) for 5 min. After blocking with 3% BSA in PBS for 45 min, slides were labeled with metal-conjugated antibodies against CD3 (170Er-Polyclonal, C-Terminal), CD11b (149Sm-EPR1344), MHCII (174Yb-M5/114.15.2) and Ly6G (141Pr-1A8) diluted in PBS with 0.5% BSA at 4° C. overnight. After being washed with 0.1% Triton-X in PBS and then PBS, slides were labeled with intercalator-Ir (1:2,000 dilution) in PBS (lacking Ca⁺⁺ or Mg⁺⁺) for 30 minutes at room temperature. After being washed again with ddH₂O for 5 min, the slides were dried. Tissues were laser ablated using a 200 Hz Hyperion™ Imaging System (Fluidigm Corp., South San Francisco, Calif. and the aerosol containing the ion cloud was directly transported to a Helios Mass Cytometer (Fluidigm). Images of labeled slides were obtained using the MCD viewer 1.0 (www.fluidigm.com/software).

### Analysis of Immune Cells in Mice Following Zika Virus-Infected Mosquito Bites or Intradermal Injection.

For the analysis of immune cells following Zika virus-infected mosquito bites, AG129 mice were allowed to be fed by Zika virus-infected A. aegypti mosquitoes on the ear. For the analysis of immune cells following AgBR1 injection, AG129 mice were intradermally injected with AgBR1 in the ear as described above. After 24 hours, mice were sacrificed and both the bitten or injected and naive ears were cut off at the base and split into dorsal and ventral halves. Ears were incubated for 1.5 hours in 2 mg/ml of Dispase II (Sigma) in DMEM media with 10% FBS, and then cut into small pieces. Small pieces were then digested for 1.5 hours in 5 mg/ml of collagenase (Gibco) in media. Digested samples were then individually passed through 70 μM filters to obtain single-cell suspensions.

After washing once with PBS containing 2% FBS (FACS buffer), cells were stained using the LIVE/DEAD™ Fixable Violet Dead Cell Stain Kit (Thermo Fisher) and then incubated with fluorochrome-conjugated monoclonal antibodies against CD45 (PerCP—BD Pharmingen; Clone 30-F11), MHCII (APC-Cy7—Biolegend; Clone M4/114.15.2), CD11b (PE—Biolegend; Clone M1/70), CD11c (PE-Cy7—BD Pharmingen; Clone HL3), and Ly6G (FITC—Tonbo; Clone RB6-8C5) for 30 minutes at room temperature, washed twice with FACS buffer. Samples were run on a BD LSRII flow cytometer and analyzed using FlowJo software.

### Statistical Analysis

GraphPad Prism software was used to analyze all the data. Animals were randomly allocated into different groups. No statistical methods were used to predetermine sample size. Rp49 and mouse β actin normalized viral RNA levels were analyzed using the two-sided Wilcoxon-Mann-Whitney test. Host responses in vitro and in vivo were performed using a two-way ANOVA for multiple comparisons or using the two-sided Wilcoxon-Mann-Whitney or the two-sided Wilcoxon matched-pairs signed rank test for two sample comparisons, as indicated in the figure legends. Survival was assessed by a Gehan-Wilcoxon test. A p value of <0.05 was considered statistically significant.

### Example 1: Identification and Characterization of Potential Target Salivary Gland Proteins

To breed mosquitos with Zika virus, eggs were hatched in a shallow dish with distilled water with 2 parts brewer's yeast (Bioserv #1710) and 3 parts desiccated liver powder (Bioserv #1320). After pupae emerged, mosquitoes were collected and placed in a small crystal dish with distilled water inside a 12″×12″×12″ metal mesh cage (BioQuip #1450B). Adult mosquitoes were maintained on 10% sucrose feeders in walk-in incubator at 28° C. and ˜80% humidity. Egg masses were generated via blood meal on naïve mice. For Zika virus injection experiments, mosquitoes were knocked-down on ice before transfer to a cold plate under a dissecting microscope. A pulled microcapillary needle was filled with ZIKV using a Nanoject II auto-nanoliter injector (Drummond). The Zika virus-filled needle was carefully inserted into the thorax of each mosquito and 69 nl of virus is injected. Infected mosquitoes were placed back in paper cups with mesh lids and maintained in triple containment for 10 days. All mosquitoes, clean and infected were housed in a warm chamber.

A murine model of the Zika virus was created as follows. Ae. aegypti mosquitoes intrathoracically injected with ZIKV were allowed to feed on AG129 mice which lack interferon α/β and γ receptors, so as to easily and efficiently transmit ZIKV to these mice (33). Ae. aegypti mosquitoes feed and engorge on AG129 mice.

Passive immunizations were performed by IP injections of antigen-specific IgG (total volume not exceeding 150 μl) in PBS one day prior to mosquito feeding. For active immunization of mice, 10 μg of antigen were administered subcutaneously in Freund's adjuvant at 2-3 sites in a total volume not greater than 0.2 ml. A post-immunization sample (test bleed) was similarly drawn by retro-orbital bleeding 7 days after the final immunization to confirm reactivity to recombinant antigens by western blotting or ELISA.

AG129 mice are susceptible to very small amounts of Zika virus and develop viremia, leading to the death at 1-5 weeks (34). Therefore, this model is useful to access vaccine efficacy in the natural context of mosquito-bite infection to test vaccine efficacy on several strains of Zika virus, including pre-epidemic and post-epidemic strains.

Mosquito feeding generally resulted in minimal discomfort and injury to mice. Not more than 5 Zika virus-infected mosquitoes were placed on mice. Animals to be infected or immunized were restrained in an appropriate apparatus designed to limit the chances of injury and reduce the amount of distress of the animal during these procedures. Mice were observed daily for symptoms of Zika virus after mosquito feeding. If any animal showed 20% weight loss, scruffy fur or paralysis, it was euthanized.

Mice were anesthetized during the process of mosquito feeding with IP injection of Ketamine/Xylazine (100 mg/10 mg per kg body weight). Anesthetized mice were laid on top of mesh lids to allow mosquitoes to feed. The mice did not experience undue stress during the process of mosquito feeding. The application of feeding can be classified as minimal distress and did not affect the general health of the animals. Mosquitoes were fed for approximately 10-30 min. Mosquito carcasses after dissection were placed in 10% chlorine bleach solution for 20 minutes prior to autoclaving.

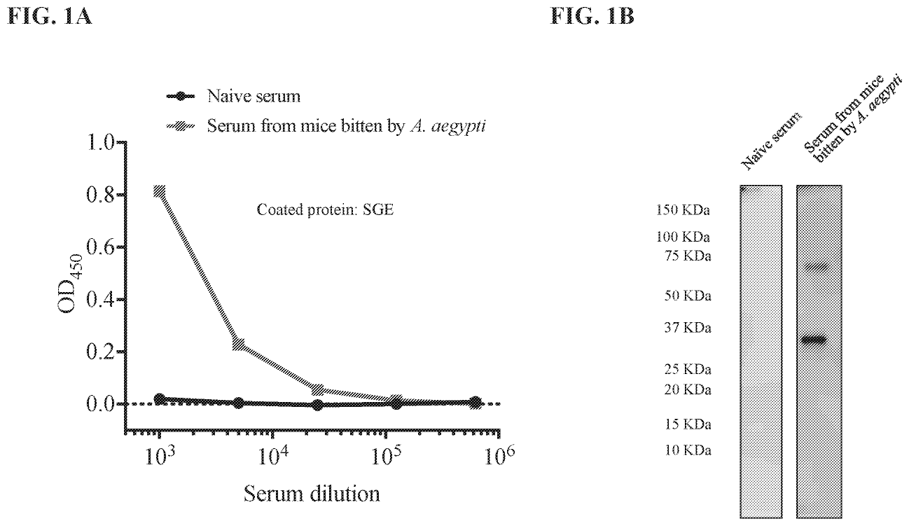

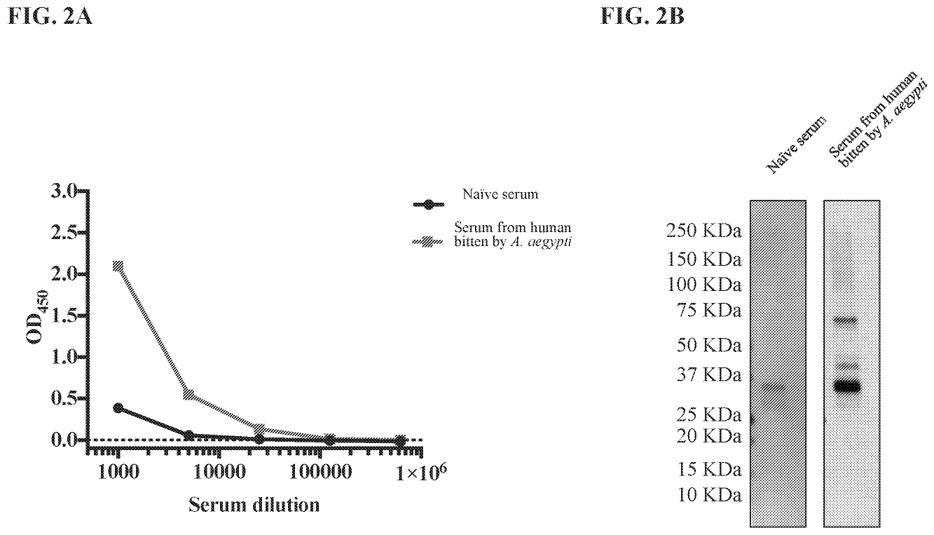

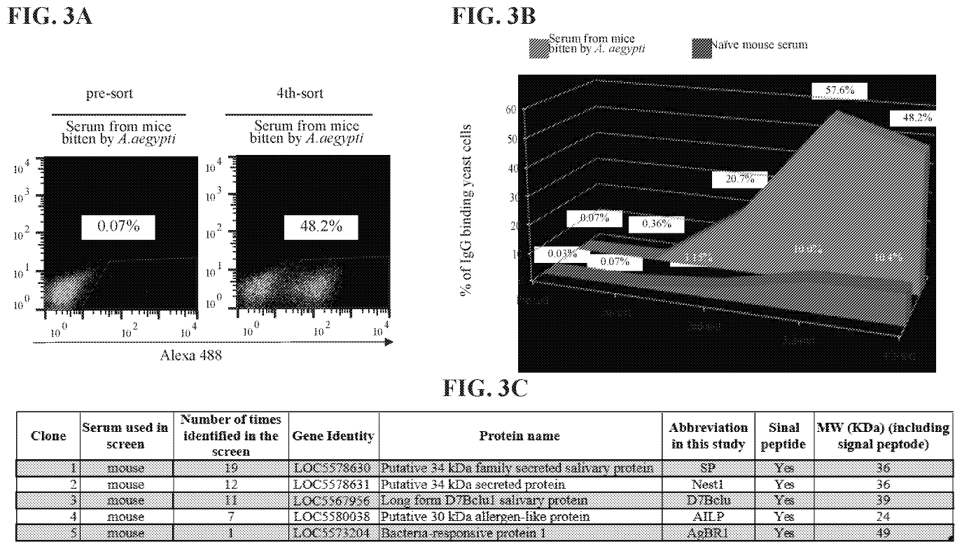

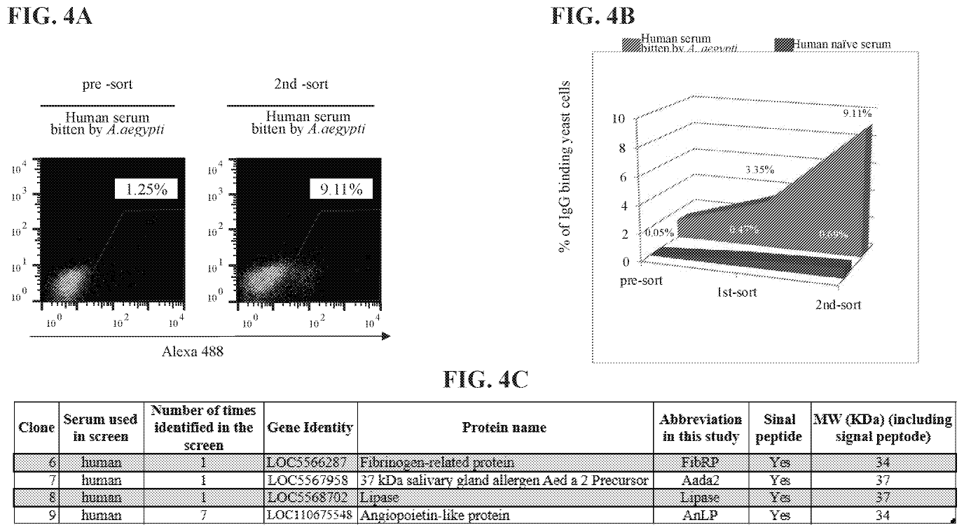

Antigenic proteins in mice that were bitten by Ae. aegypti were identified using a screen overcoming disadvantages inherent in SDS-PAGE and proteomics for detection of proteins of low-abundance and low-antigenicity. Yeast surface display screening was used because the method can identify uncommon proteins by iterative rounds of magnetic-activated cell sorting (50). An Ae. aegypti salivary gland yeast surface display library was generated by isolating RNA from salivary glands, reverse transcribing these transcripts to cDNA and cloning into the pYD1 vector before transformation into yeast cells. The protocol for enrichment of yeast clones expressing salivary gland proteins is adapted from a recent publication (12). An A. aegypti salivary gland yeast surface display library was generated and probed with IgG from mice repeatedly bitten by A. aegypti (FIGS. 1A and 1B) or humans bitten by Ae. aegypti. These mouse and human sera were highly reactive with an Ae. aegypti salivary gland extract, as demonstrated by ELISA and immunoblot (FIGS. 1A, 1B, 2A and 2B). Magnetic-activated cell sorting was used to enrich for yeast cells expressing salivary proteins recognized by mouse antibodies (FIGS. 3A and 3B) and human antibodies (FIGS. 4A and 4B) and after four rounds of sorting individual yeast clones were isolated, and the recombinant plasmids were recovered. Five clones, encoding unique mosquito genes were antigenic in mice (LOC5578630, LOC5578631 (NeSt1), LOC5567956, LOC5580038, LOC5573204 (AgBR1)) (FIG. 3C) and four unique mosquito genes were antigenic in humans (LOC5566287, LOC5567958, LOC5568702, and LOC110675548) (FIG. 4C) were identified using this method.

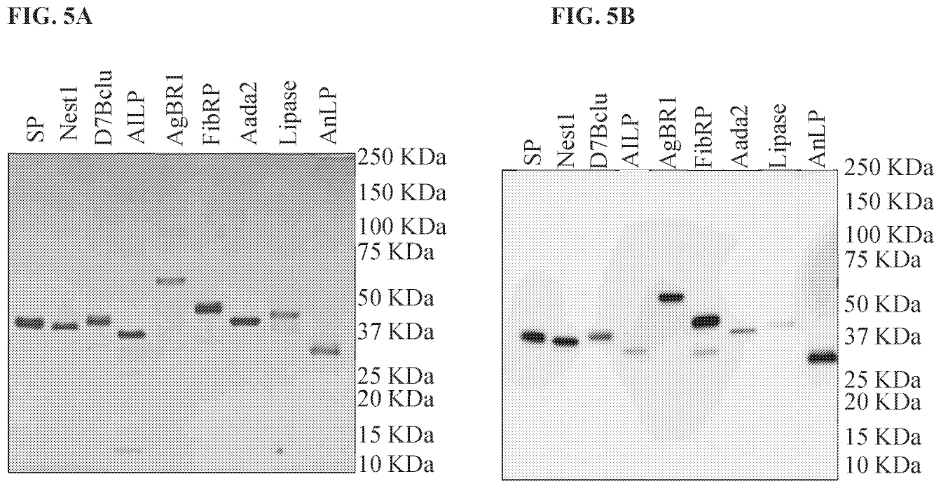

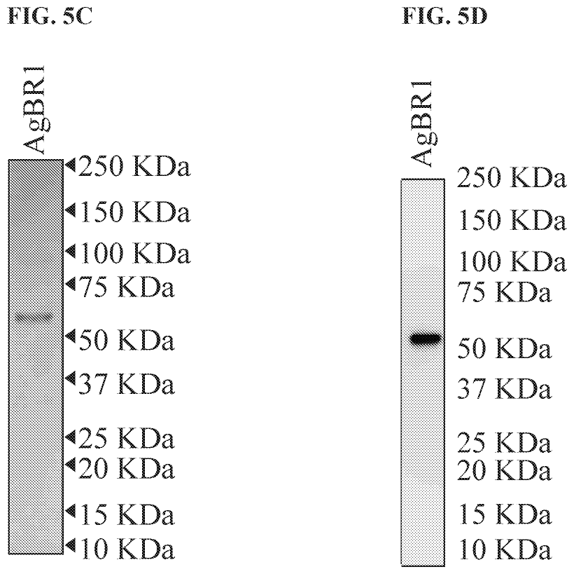

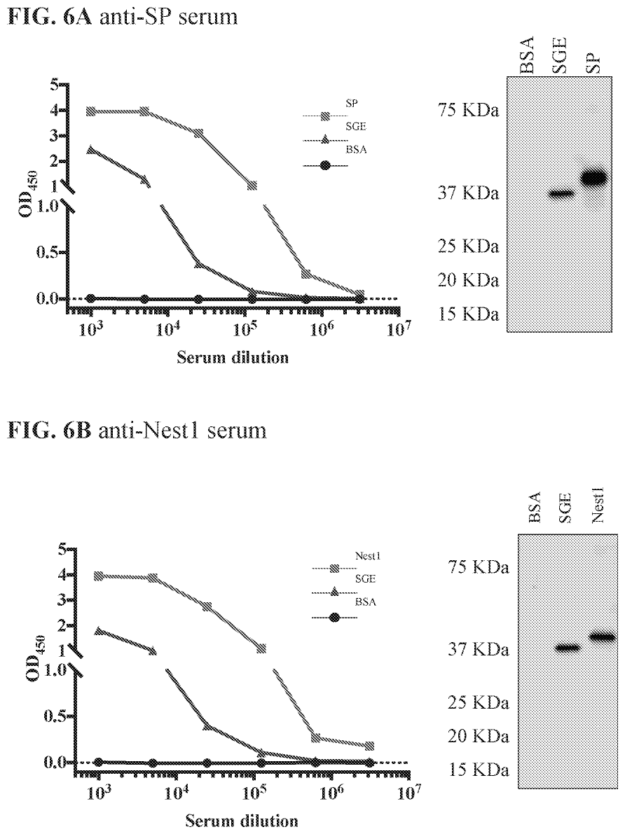

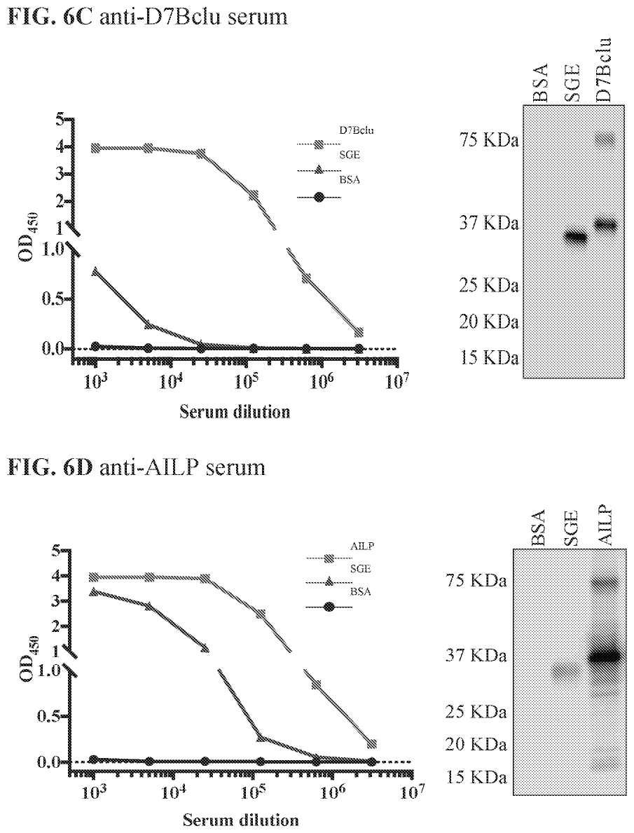

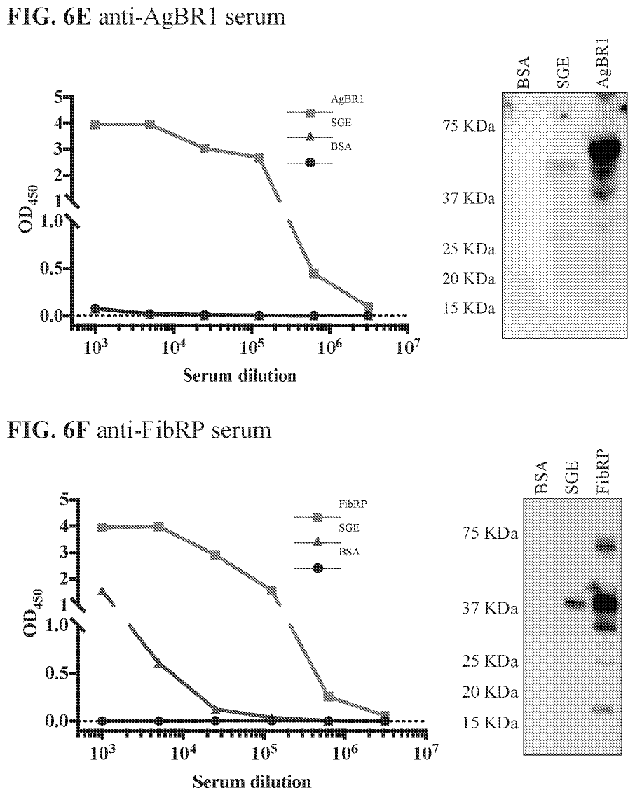

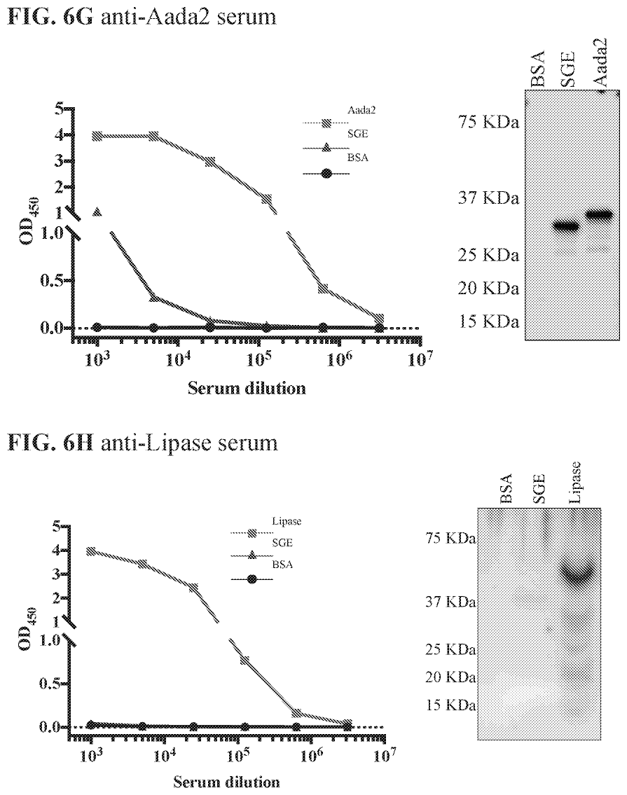

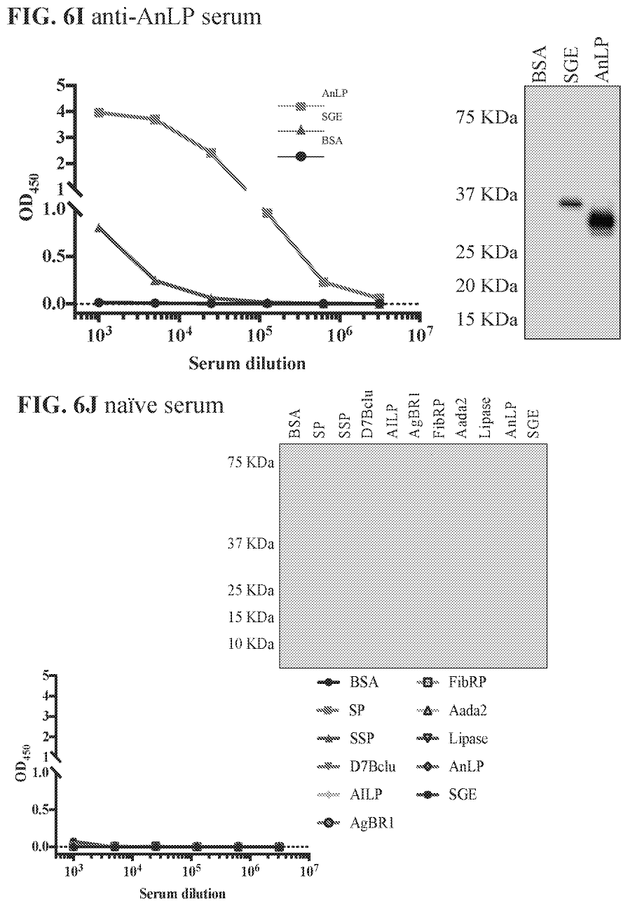

To examine the effect of these nine proteins on Zika infection, nucleic acid sequences encoding each of these proteins were cloned into the pMT Drosophila vector, expressed the recombinant proteins in Drosophila S2 cells, and purified using nickel affinity chromatography. The nine proteins were run on an SDS-PAGE gel, then stained with Coomassie Brilliant Blue (FIG. 5A) and analyzed by Western blot with anti-His antibody (FIG. 5B). To generate antibodies to use in passive immunization experiments and for the further analysis of these proteins, rabbit serum was generated against all identified antigenic proteins and validated by immunoblot using the recombinant proteins and mosquito salivary gland extract (FIG. 6).

### Example 2: Mixture of Antiserum Against Antigenic Mosquito Salivary Gland Proteins Protects Against Zika Virus Infection

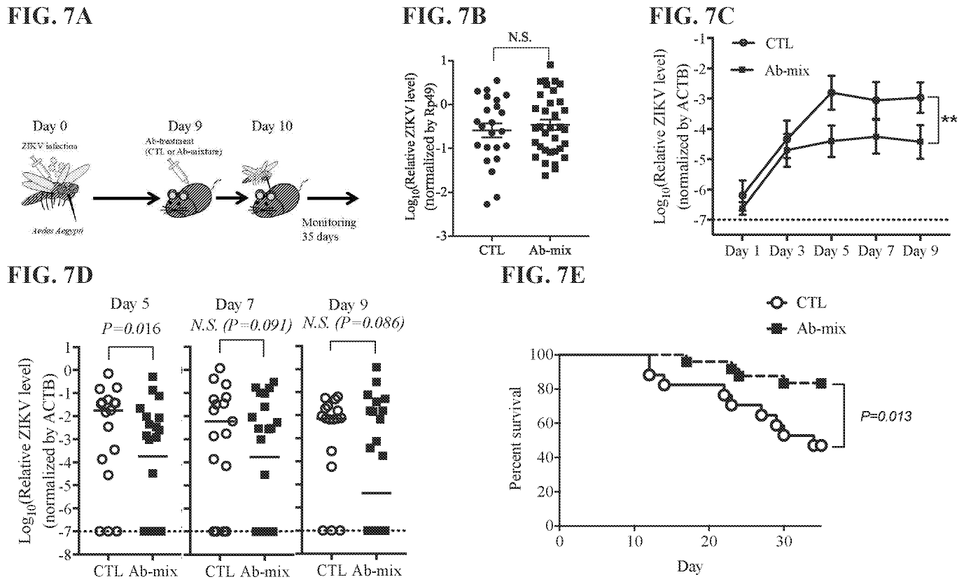

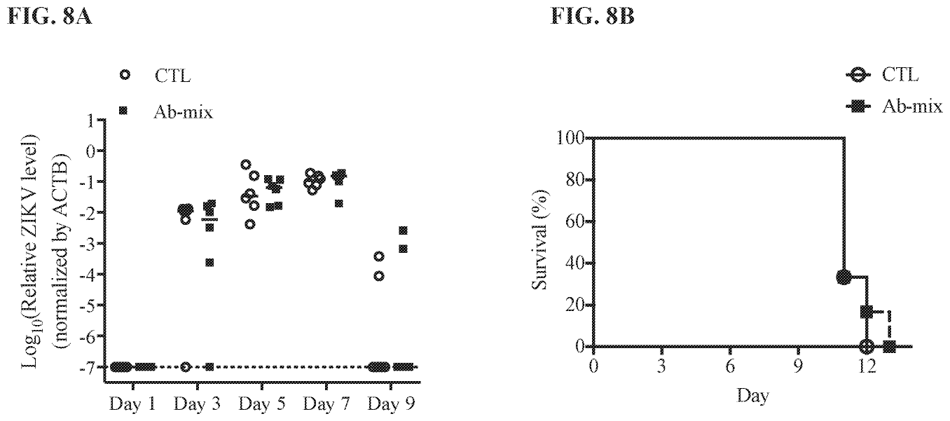

Mice were passively immunized intraperitoneally with 150 μl of a mixture of all specific antisera, or control antiserum. One day later, the passively immunized mice were fed upon by Zika virus-infected mosquitoes with similar levels of virus in their salivary glands (FIG. 7B). Blood was collected every other day for 9 days from mice fed on by Zika virus-infected mosquitoes and analyzed for Zika virus infection by qRT-PCR using primers described previously (40). The tested pooled antisera significantly reduced Zika virus levels over the course of infection (FIG. 7C) and in mice on day 5 (FIG. 7D), and conferred significant protection against pathogenesis, measured by survival, compared with the control group (FIG. 7D). This mixture of antibodies against these antigenic proteins does not affect Zika virus replication (FIG. 8A) or pathogenesis (FIG. 8B) when mice are passively immunized and then infected using subcutaneous needle infection.

### Example 3: Effect of AgBR1 Protein in Zika Virus Infection

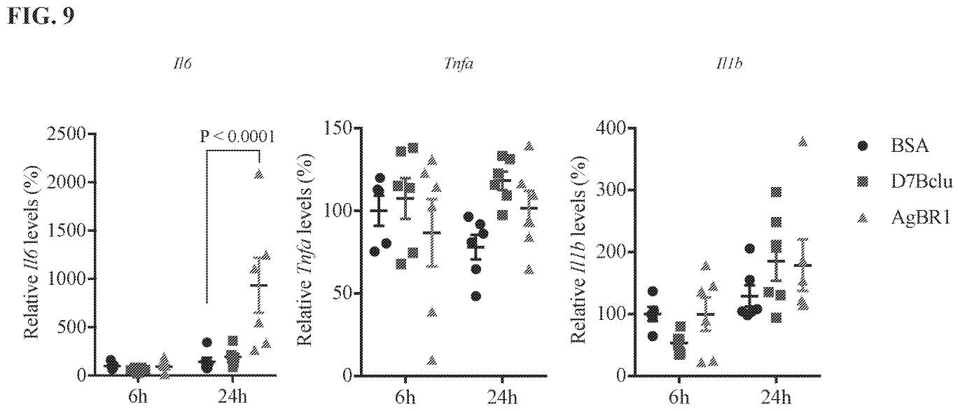

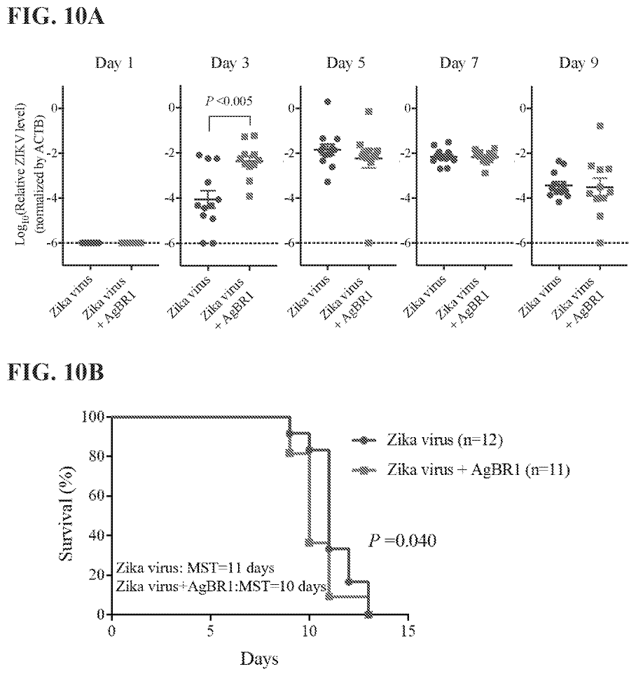

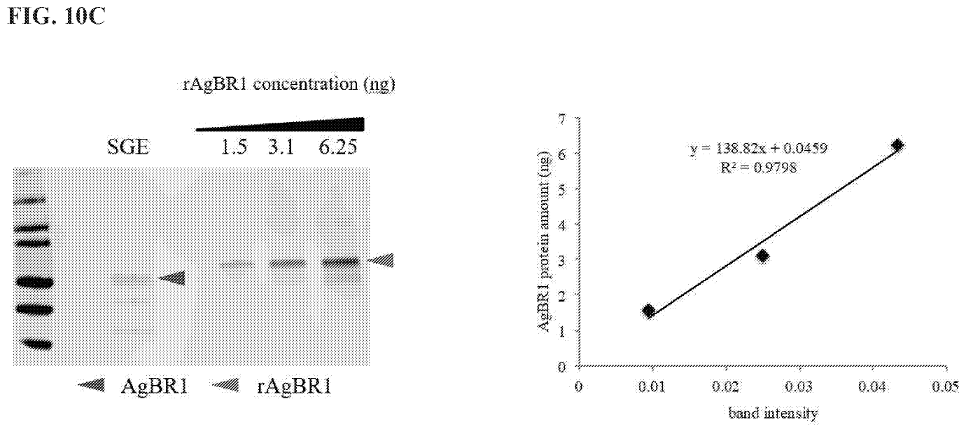

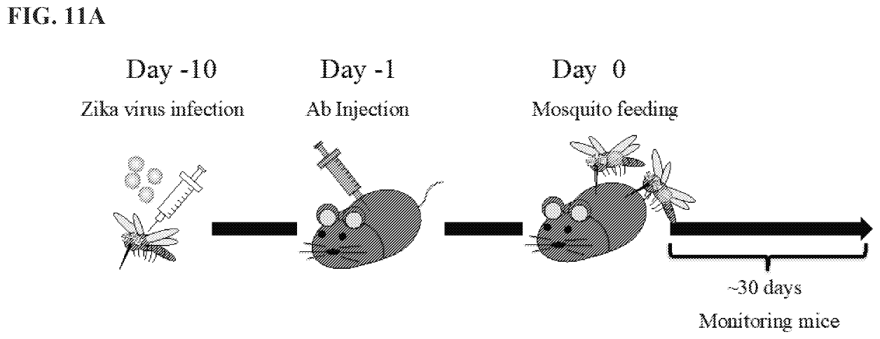

To identify if any of these proteins is capable of inducing an immune response in cells, splenocytes were treated with antigenic mosquito salivary gland proteins. One protein, called AgBR1 is a Bacteria-responsive protein 1 which belongs to group V chitinase-like proteins (41). Next, it was examined whether AgBR1 stimulates inflammatory responses in vitro. Murine splenocytes stimulated with recombinant AgBR1 (see FIGS. 5C and 5D) produced in S2 cells (5 μg/ml) demonstrated significantly higher levels of Il6 expression compared with controls, with the data shown in FIG. 9. As increased vascular permeability contributes to flavivirus pathogenicity (42) and IL-6 is associated with these processes (43), it was next examined whether AgBR1 influences Zika virus infection in vivo. Given previous studies demonstrating that approximately half of the protein in the salivary glands is discharged during a blood meal (44,45), the concentration of AgBR1 in mosquito saliva can be estimated to be between 1.6-8.2 μM (FIG. 10C, Table 1). Therefore, AG129 mice were injected with Zika virus and AgBR1 (5.1 μM, 10 μg of AgBR1 in total volume 40 μl).

At day 3 post infection, significantly higher viremia levels were observed in the group of mice inoculated with Zika virus in conjunction with AgBR1, compared to those of mice challenged with Zika virus alone. When AgBR1 is co-inoculated along with Zika virus, higher levels of virus are detected in the blood by qRT-PCR at day 3 after subcutaneous injection (FIG. 10A). In addition, AgBR1 protein significantly impaired the survival of Zika virus-infected mice. Mice injected with a mixture of virus and AgBR1 also experienced significantly more pathogenesis as measured by survival (FIG. 10B). AgBR1 protein significantly impaired the survival of Zika virus-infected mice (FIG. 10B). These results demonstrate that AgBR1 can exacerbate Zika virus infection and disease in vivo.

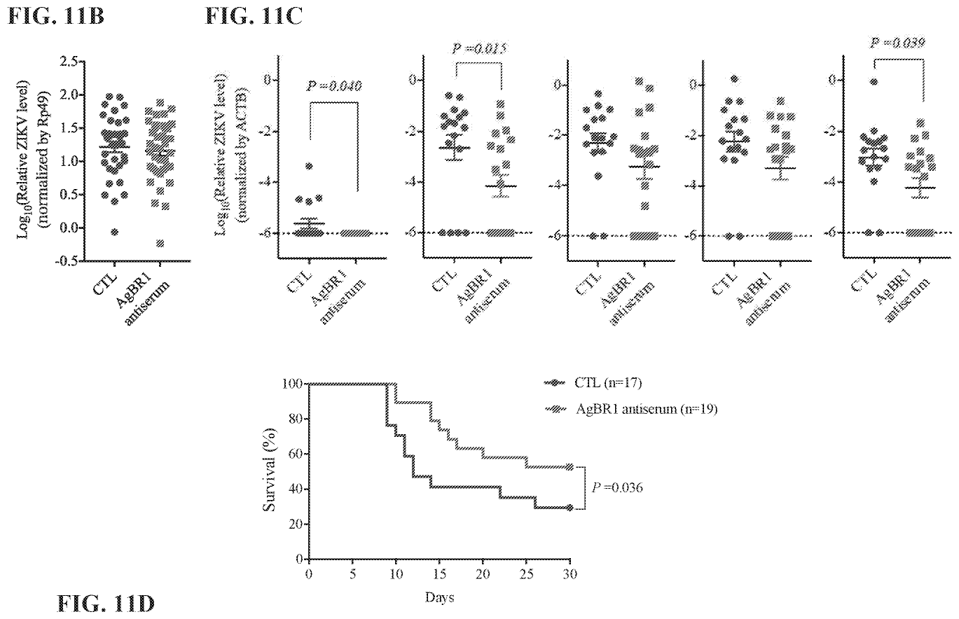

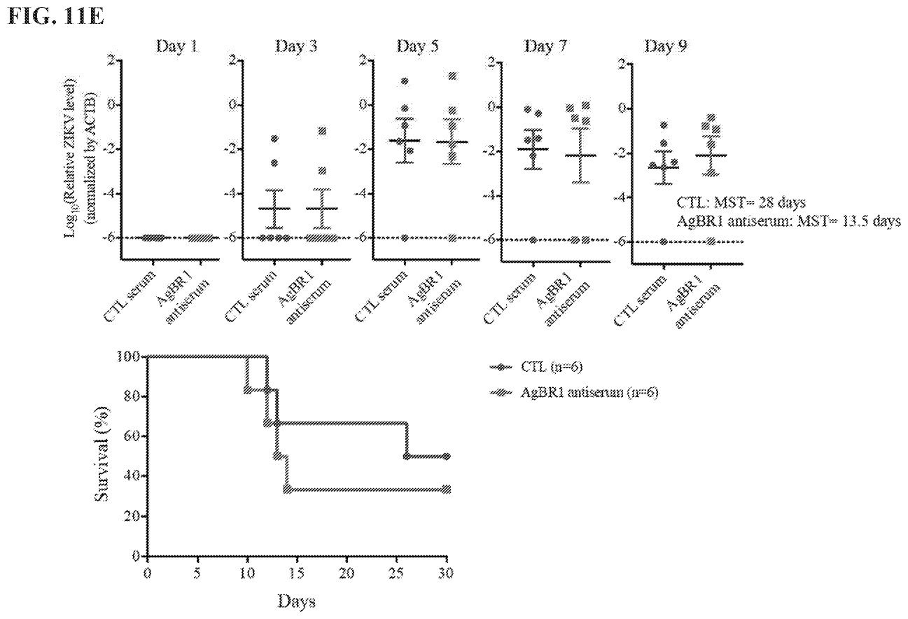

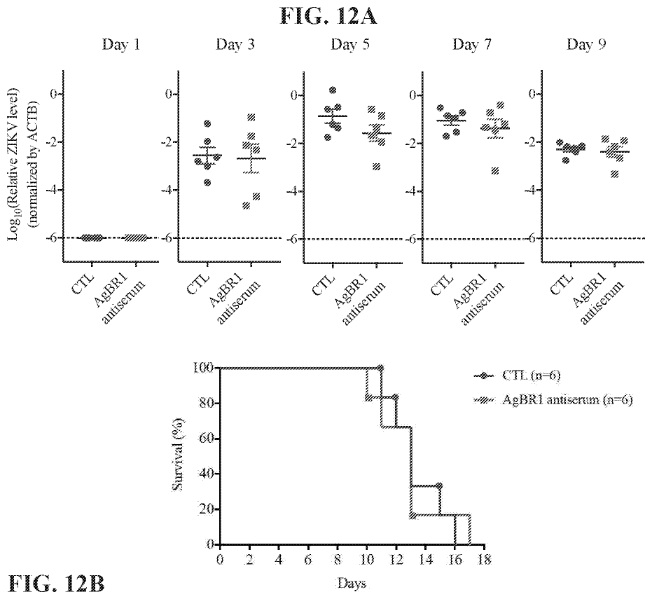

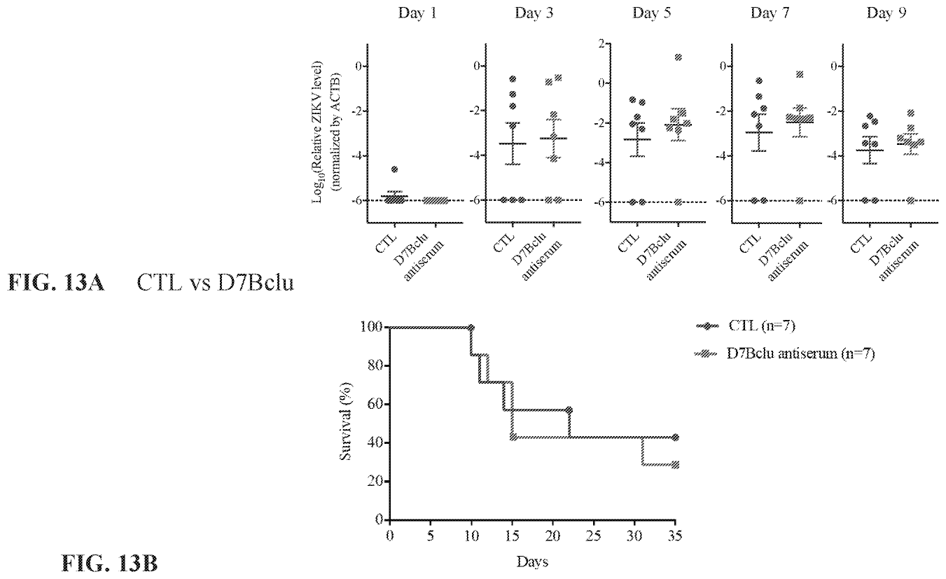

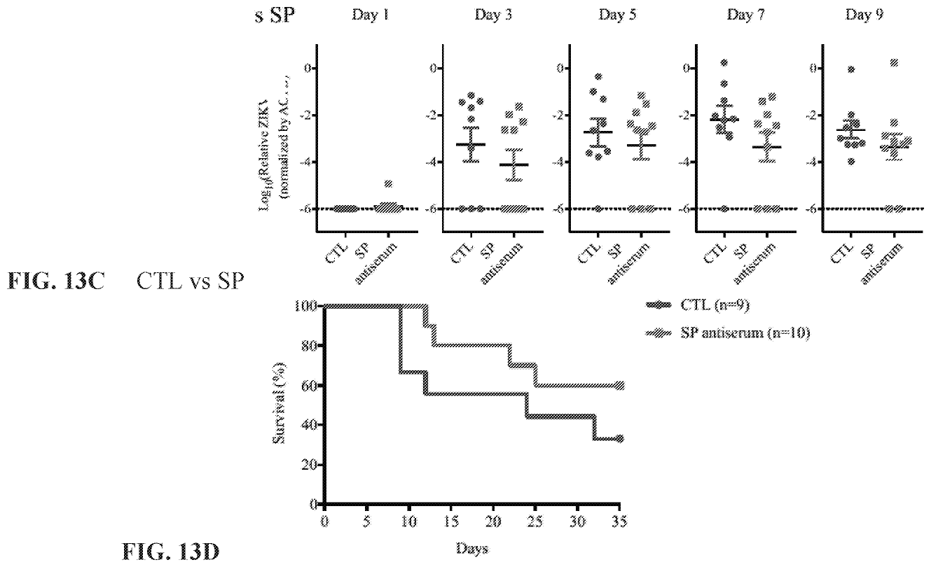

To determine if passive immunization can protect mice from Zika virus infection, AG129 mice were injected with 150 uL of rabbit serum against AgBR1 before infection via mosquito bite. Mosquitoes with similar levels of viral infection (FIG. 11A) were allowed to feed on mice, and then blood was collected from mice for 9 days. AgBR1 antiserum significantly reduced Zika virus levels on days 1, 3 and 9 after infection (FIG. 11B). This also significantly protected mice from pathogenesis as measured by survival over 30 days (FIG. 11C). This protection is not from a non-specific interaction as this antiserum failed to protect from needle infection with Zika virus (FIG. 12). Antiserum against two other abundant antigenic salivary gland proteins is not protective against Zika virus infection (FIG. 13).

Here, AG129 mice were used that lack both Type I and II IFN receptors but can elicit B-cell and T-cell responses (76,77). As type I interferon signaling can contribute to optimal antibody responses (78), it is possible that further efforts using alternative adjuvants, protein concentration or different animal models could enhance the effect of active immunization. Overall, these results indicate that immunization with AgBR1 partially influenced mosquito-transmitted Zika virus infection.

To determine whether the effects observed with AgBR1 extended to other proteins identified in the screen, two additional proteins were selected: D7Bclu and SP, whose expression has been identified as upregulated salivary gland proteins during flavivirus infection (67). D7Bclu and SP antisera were generated in a similar fashion to the AgBR1 antiserum and performed passive immunization experiments. Neither the D7Bclu nor SP antisera altered the viremia or protected mice from lethal mosquito-borne Zika virus infection, as shown in FIGS. 13A-D.

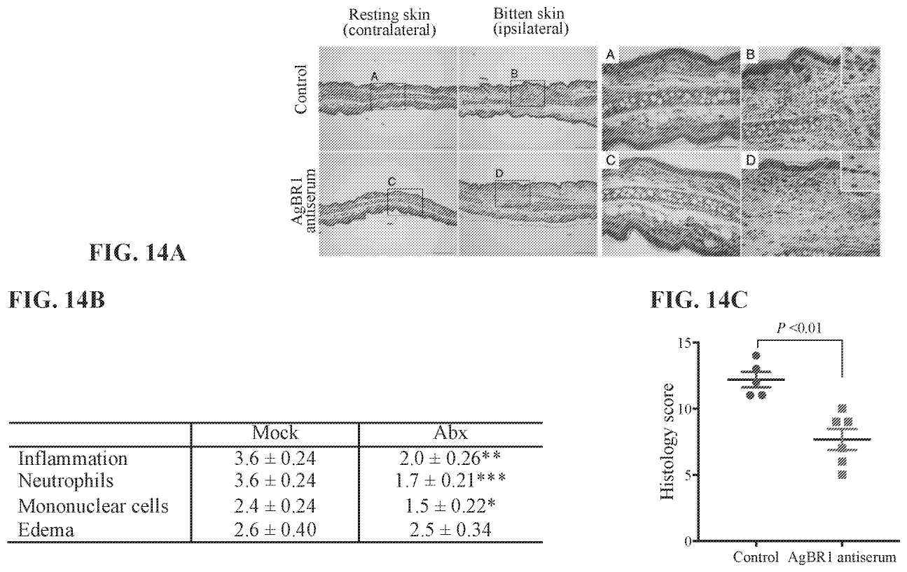

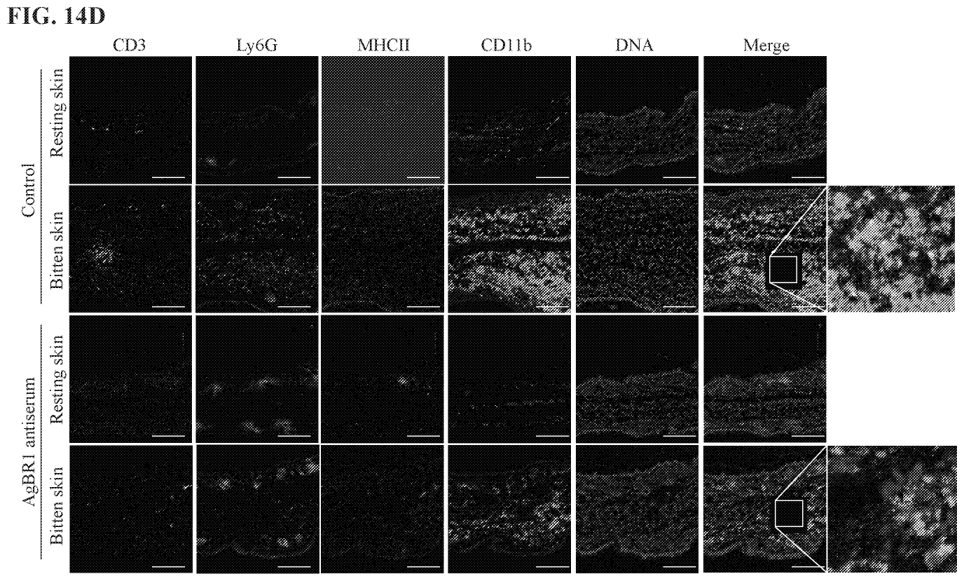

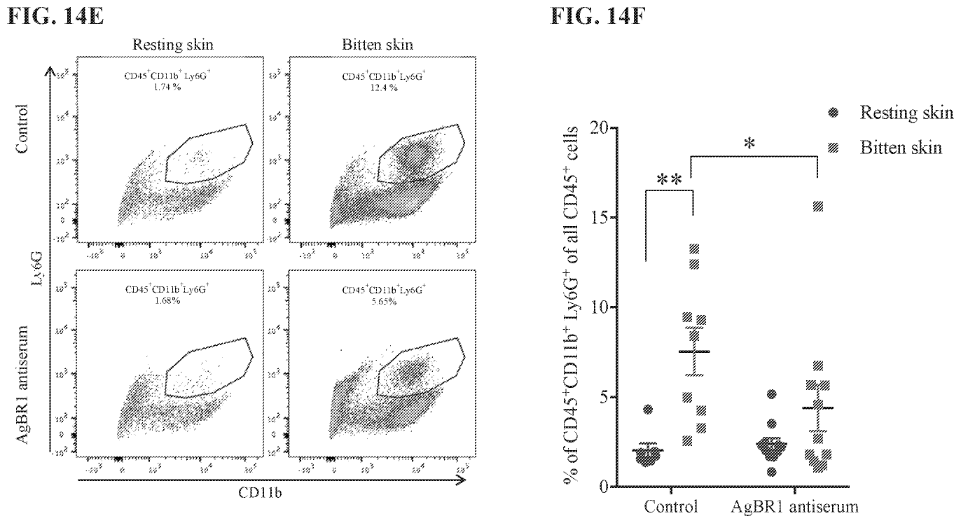

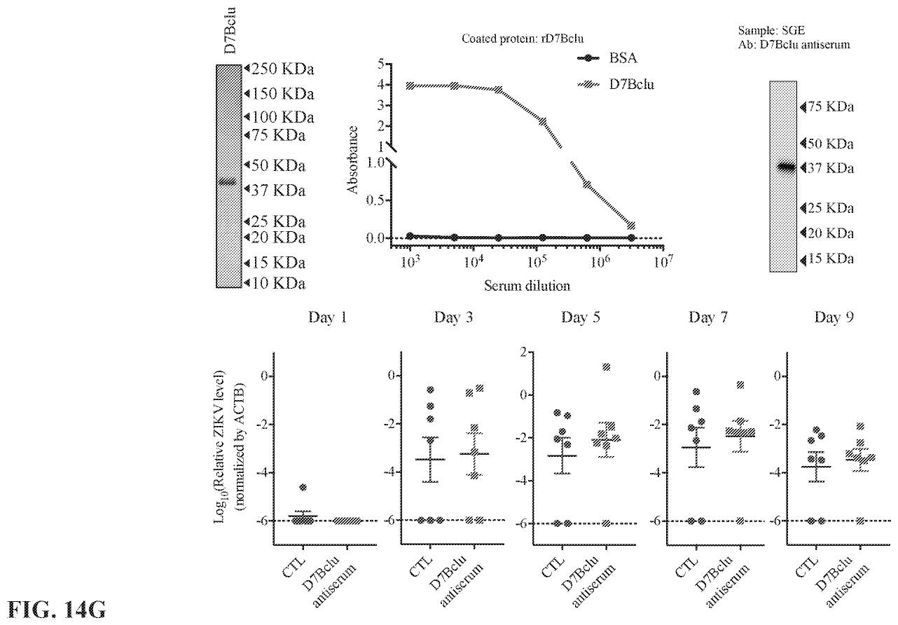

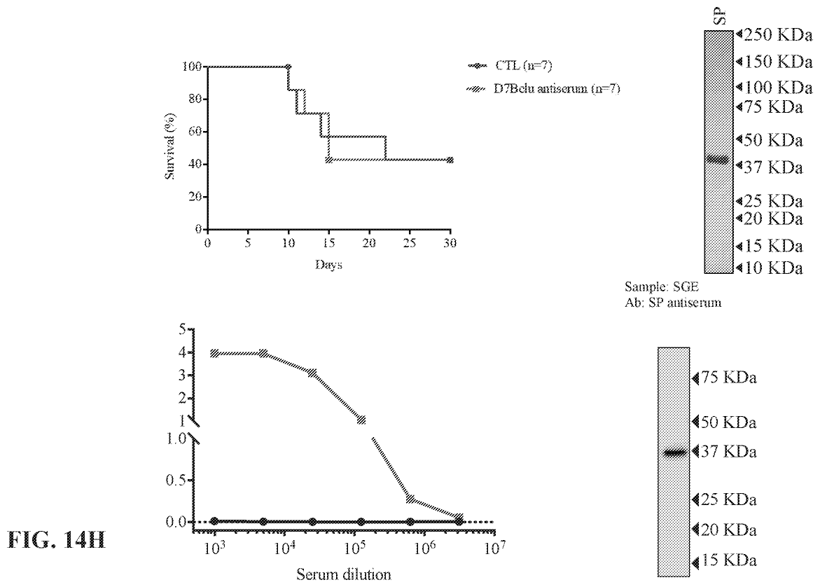

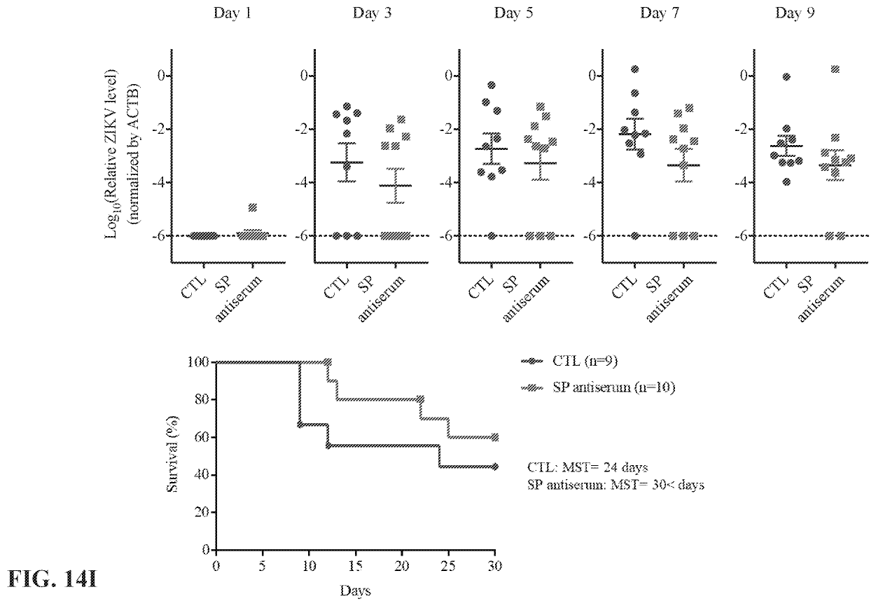

To more fully understand the underlying mechanism of protection of immunization with AgBR1, assays were performed to determine whether AgBR1 antiserum influenced the early innate immune response at the bite site after exposure of mice to Zika virus-infected mosquitoes. A histological analysis of the bite site 24 hours post-feeding by Zika virus-infected mosquitoes showed prominent inflammatory cell infiltration mainly composed of neutrophils in the dermis of mice administered naïve serum (control), which was less apparent in mice administered AgBR1 antiserum. The data is shown in FIGS. 14A and 14B. Consistent with these findings, histology scores were significantly lower in mice administered AgBR1 antiserum compared with mice administered naïve serum, as shown in FIG. 14C. Furthermore, imaging mass cytometry showed that infiltrating cells at the bite site of Zika virus-infected mosquito bites were mainly Ly6G⁺ and CD11b⁺ cells, supporting the observation made with hematoxylin and eosin staining that the infiltrating cells are predominantly composed of neutrophils, monocytes, and macrophages, with some minor populations of T cells or other immune cells (FIG. 14D). In addition, the infiltration of Ly6G⁺ cells and CD11b⁺ cells is reduced in mice administered AgBR1 antiserum, in contrast to control animals (FIG. 14D). The alteration of infiltrating cell populations in the skin of bitten mice administered AgBR1 antiserum indicates that the AgBR1 antiserum influenced the number of CD45⁺CD11b⁺Ly6 G⁺ neutrophils at the bite site. See FIGS. 14E and 14F. These results suggest that AgBR1 antiserum suppressed acute inflammation, and particularly the neutrophilic response, at the mosquito bite site.

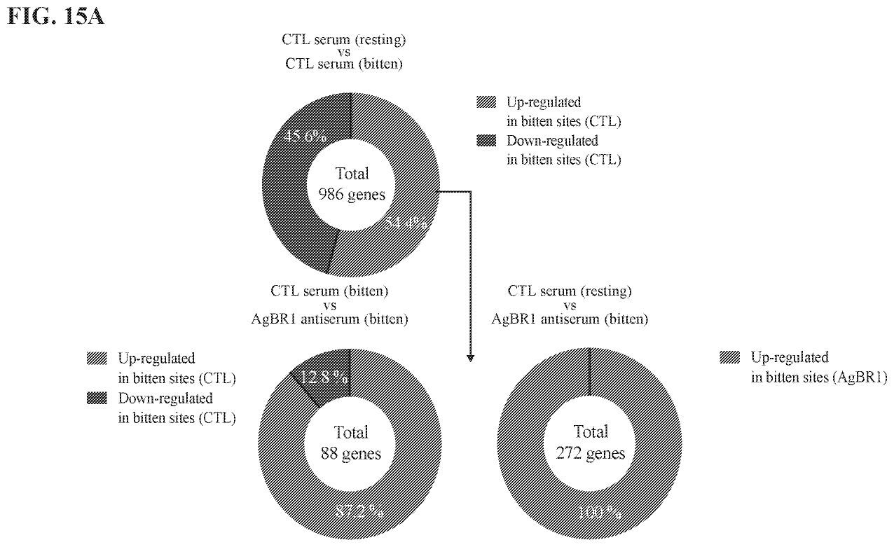

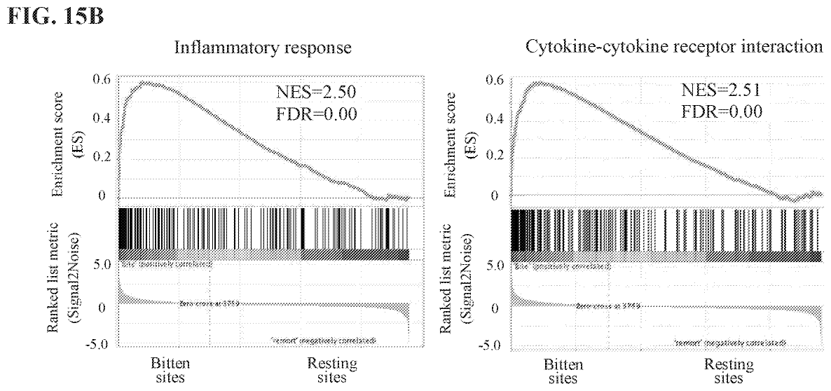

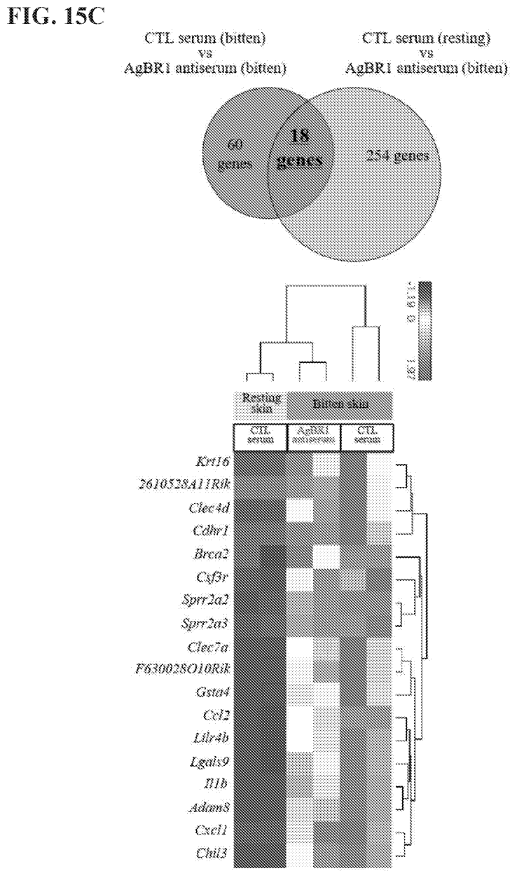

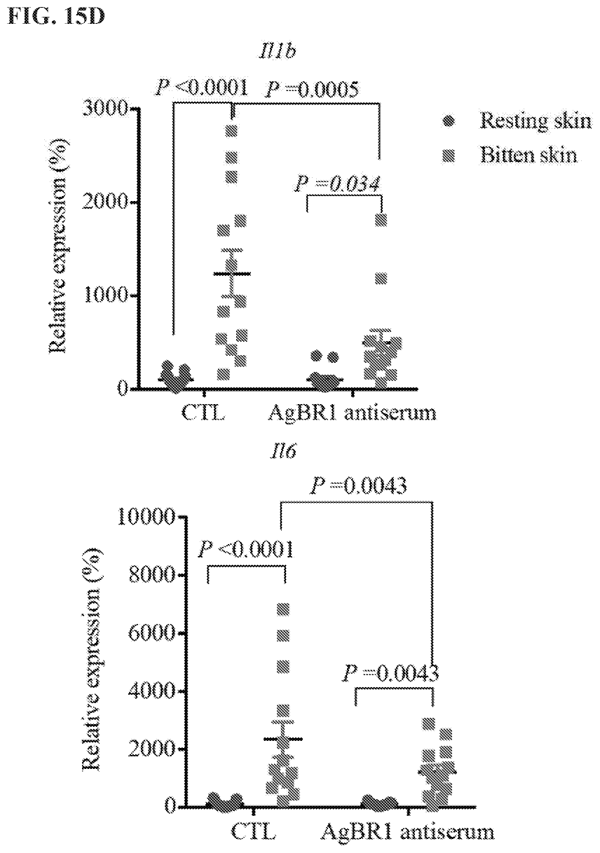

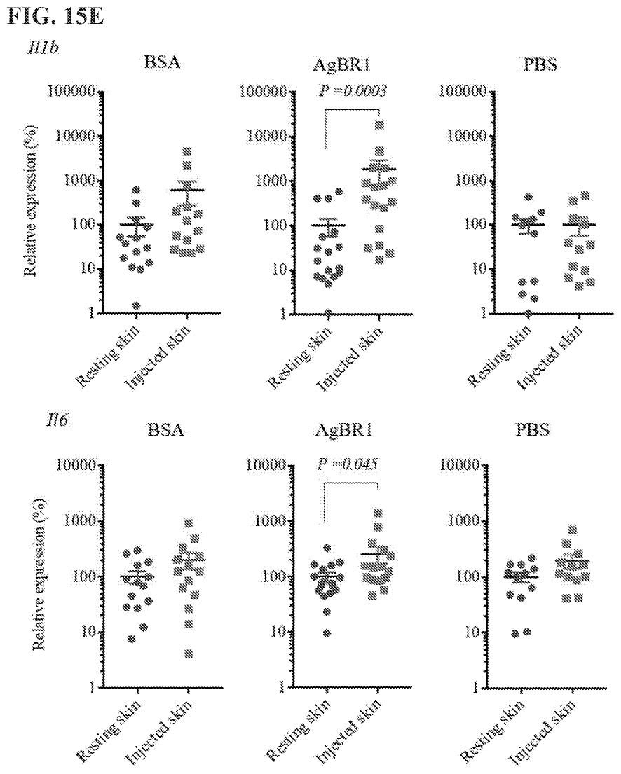

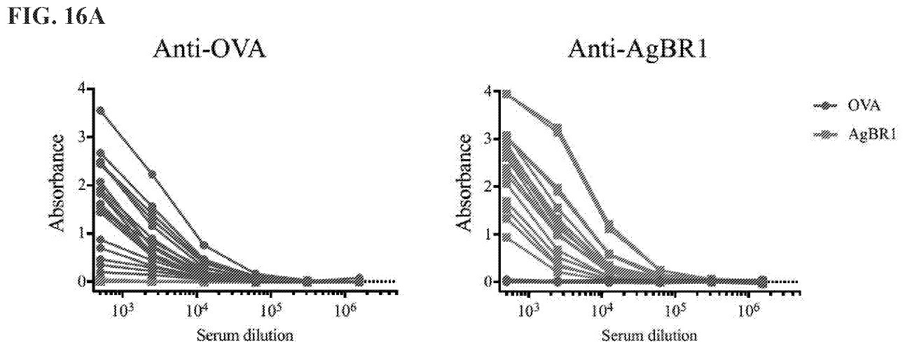

Analysis by RNAseq also indicates an increase in inflammatory and cytokine response in the control serum group as compared to AgBR1 antiserum (FIG. 15).

To further understand how AgBR1 may influence mosquito-borne Zika infection, RNA sequencing was performed on tissue collected at the bite site of mice 24 hours after Zika virus-infected mosquito feeding. 536 upregulated genes were found out of 986 differentially expressed genes between the bite site and resting site in control mice following Zika virus-infected mosquito feedings. See FIG. 15A and Table 2. A variety of cytokine and chemokine genes, including neutrophil-attracting chemokines Cxcl1, proinflammatory cytokine Il1b, monocytic chemoattractive chemokines Ccl2 and Ccl6, were significantly upregulated at the bite site compared to the resting site. This result was consistent with a previous report describing a detrimental role for inflammatory neutrophils that express IL-1β in the induction of cutaneous inflammatory responses at the bite site (31,49). GSEA analysis also revealed that inflammatory responses and cytokine signaling, which are mediated by host immune cells, were highly enriched in bitten skin, supporting these histological findings (FIG. 15B, Tables 4-7).

The impact of AgBR1 antiserum on inflammatory responses induced by Zika virus-infected mosquito bites was evaluated. By focusing on genes that were upregulated in the bite site, 18 genes were identified, including Il1b, Cxcl1 and Ccl2, which were attenuated in mice inoculated with AgBR1 antiserum (FIGS. 15A and 15C). The reduction of Il1b was also confirmed by qPCR (FIG. 15D). In addition, Il6 expression levels were examined in each group because AgBR1 stimulates Il6 expression in vitro. Though Il6 was initially not included in the differential gene expression analysis due to the low expression in control skin, Il6 expression levels were nonetheless significantly suppressed in AgBR1 antiserum-treated mice compared with control mice, consistent with the in vitro data (FIG. 15D). It was also found that the direct inoculation of AgBR1 into the skin significantly induces IL1b and Il6 expression (FIG. 15E).

Arboviral infection triggers the recruitment of peripheral neutrophils and monocytes to the site of infection (31,46) and, previous studies showed that neutrophils are important targets of flaviviruses in vivo and that infiltration of neutrophils contribute to the initial flavivirus infection and dissemination (31,46-48). Here, it is shown that AgBR1 induces neutrophil recruitment at the bite site and blocking this effect suppresses the early host response. These data suggest that targeting AgBR1 blocks the early host responses caused by the bite of Zika virus-infected mosquitoes, leading to the suppression of viral dissemination and protection against lethal Zika virus infection.

### Example 4: Examine the Efficacy of AgBR1 as a Vaccine Candidate

Standard immunization protocols approved by the Yale Animal Care and Use Committee are followed to immunize mice with rAgBR1. Briefly, four to six-week-old AG129 mice are actively immunized by subcutaneous injection of 10 μg of purified recombinant protein in Freund's adjuvant. Either one or two booster immunizations with 10 μg of recombinant protein in adjuvant are provided once every two weeks. Control animals are immunized with ovalbumin (InvivoGen, CA).

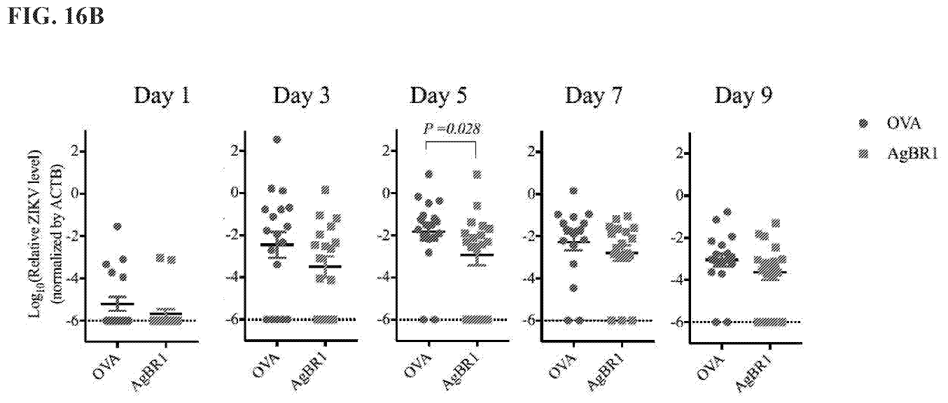

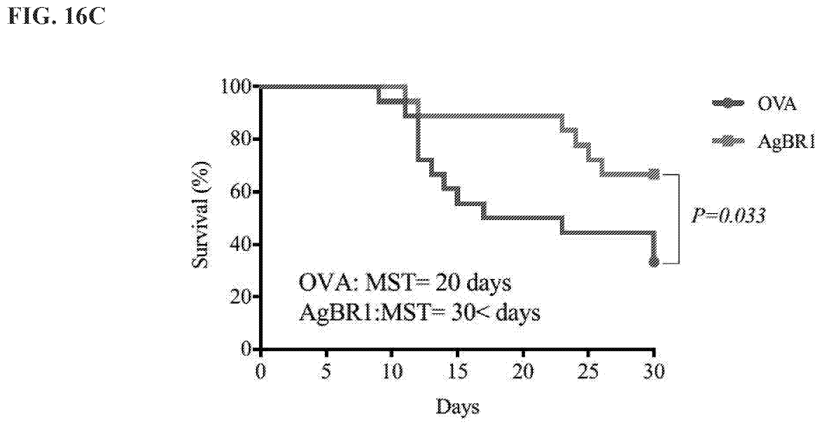

Five-10 μl samples of blood are collected by retro-orbital bleed two weeks after every booster immunization. Reactivity against both rAgBR1 and the native mosquito antigens in a salivary gland extract are assessed by ELISA (FIG. 16A). Using data obtained from active immunization testing, a determination is made whether active immunization with rAgBR1 is likely to elicit sufficient concentrations of anti-AgBR1 specific antibodies for protection against Zika virus transmission. From the data regarding reactivity to native and recombinant protein, an assessment is made as to which immunization protocol is optimal for effective antibody production in mice. Immunization with recombinant proteins often fails to result in constant, high concentrations of antibodies. Mosquito bites may boost the existing immune response and therefore help maintain a high concentration of antibodies.

Wild-type mice are not susceptible to infection with Zika virus, and AG129 mice, which lack IFN receptor α and γ subunits, must be used in challenge experiments. However, AG129 mice are often immunocompromised and may fail to mount a sufficiently robust antibody response. In such case, wild-type mice are immunized and their interferon receptors are inactivated by passive immunization of IFNAR1-blocking mAb (MAR1-5A3) as described in Lazear et al. (39).

The protective effect of active immunization with AgBR1 antigen in preventing mosquito-borne Zika virus infection was assessed using a murine model. Two weeks after the final immunization with the optimal conditions, mice are fed on by either two Zika virus infected mosquitoes. Every other day until nine days after mosquito feeding (representing the time point when ZIKV is expected to have fully disseminated throughout the mice), transmission is assessed by taking 10-15 μl of blood from control and experimental animals by retro-orbital bleed and virus burden is assessed by RNA isolation and qRT-PCR as described previously (41). The cDNA template in each reaction is normalized to mouse β-actin. The significance of difference in virus level in mice and survival between the groups is assessed by a Gehan-Wilcoxon test, respectively. Mice immunized with AgBR1 protein have less virus at day 5 (FIG. 16B) and are protected from Zika virus pathogenesis, as shown in FIG. 16C.

Animals can be immunized with heat inactivated antigens or truncated recombinant antigens. Salivary antigens secreted into the host skin upon future Ae. aegypti mosquito bites (both infected and uninfected), have the potential advantage of boosting the anamnestic response in the host and are preferred vaccine candidates.

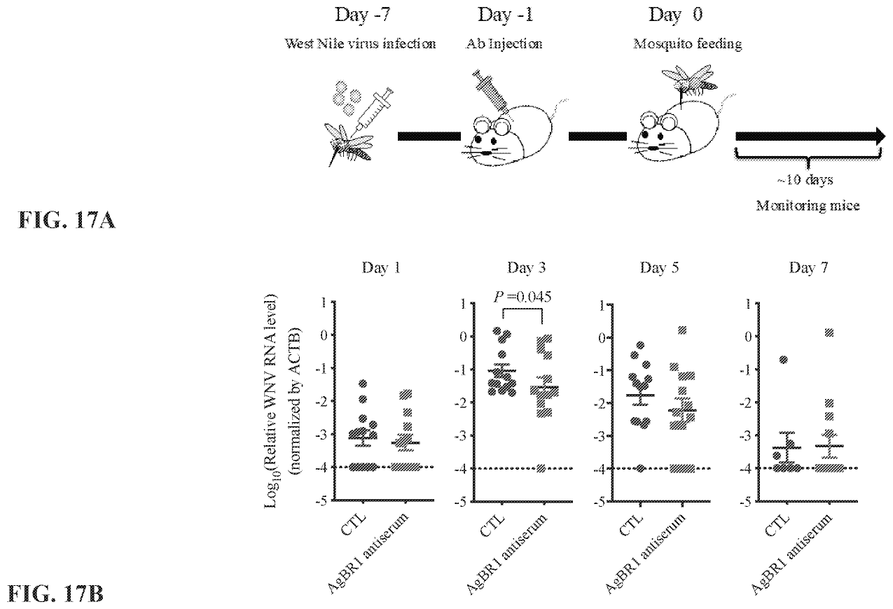

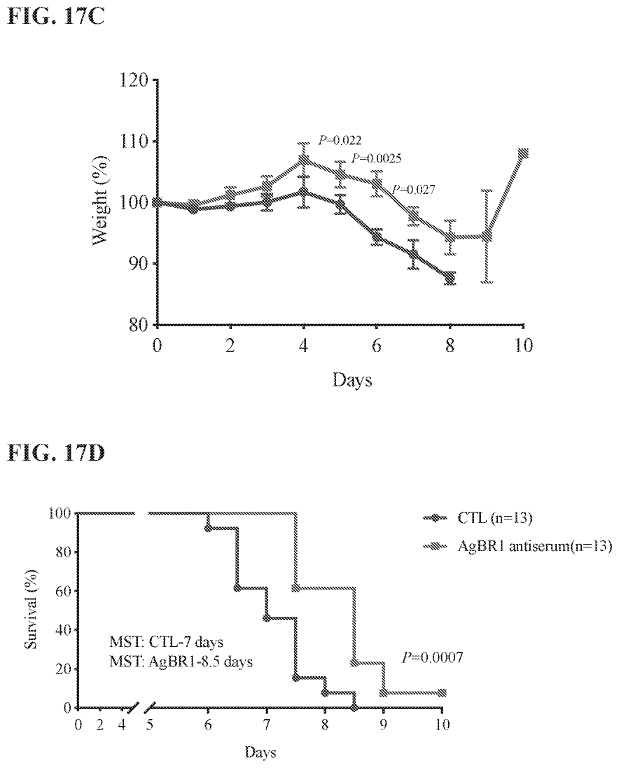

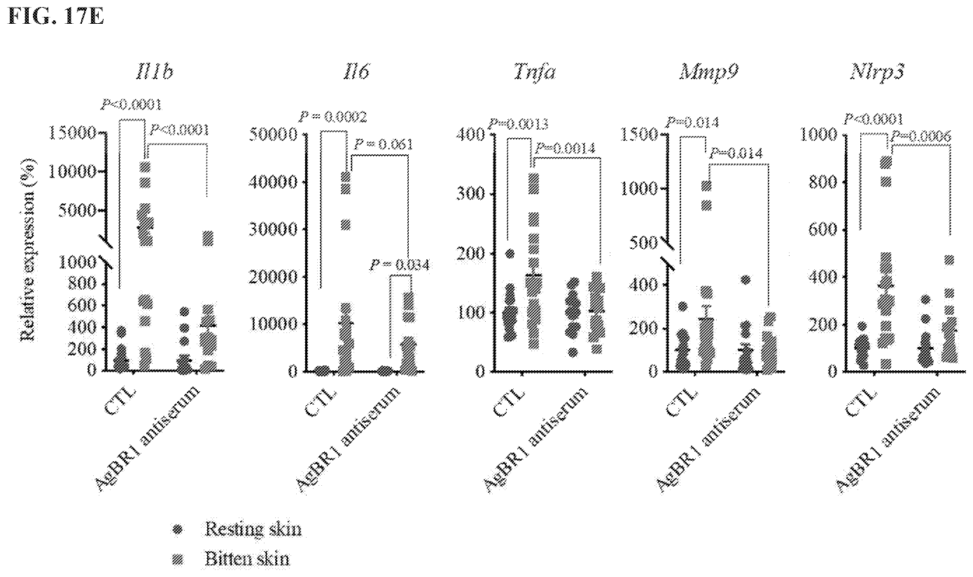

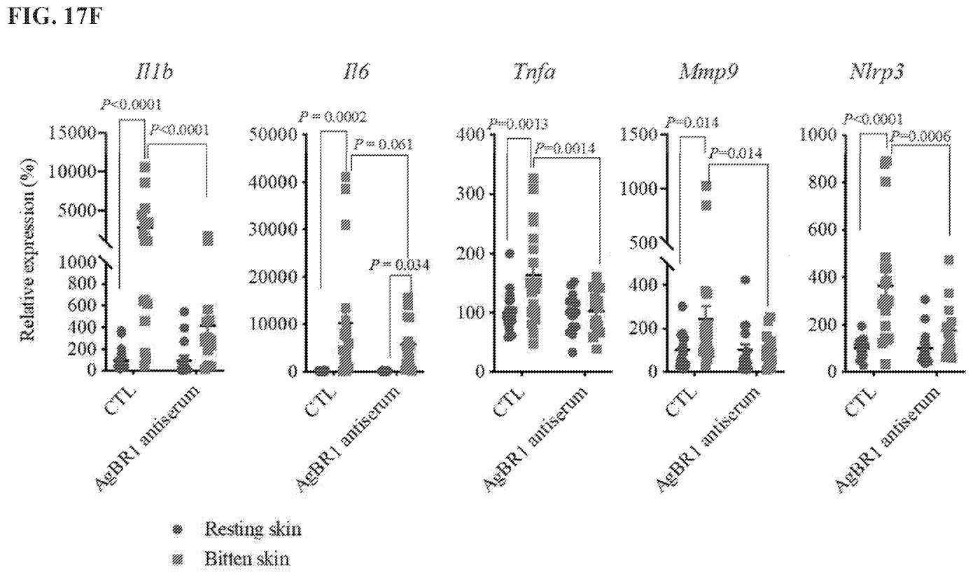

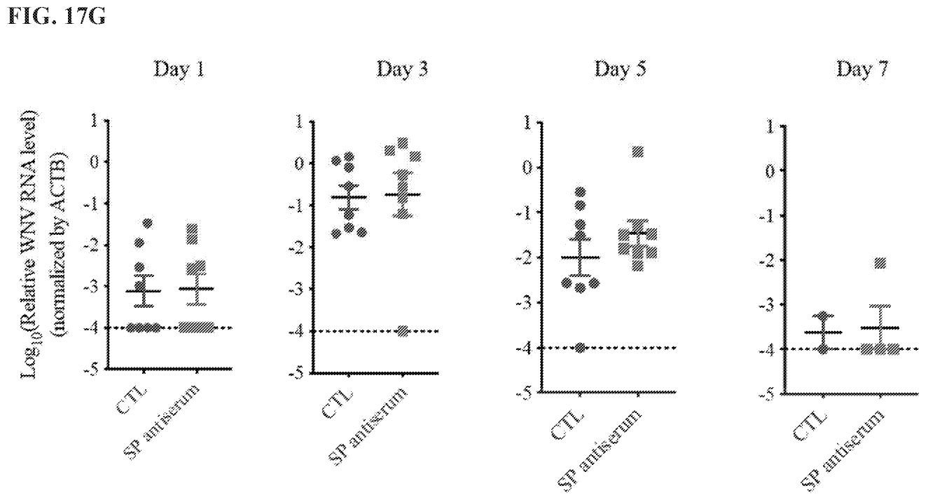

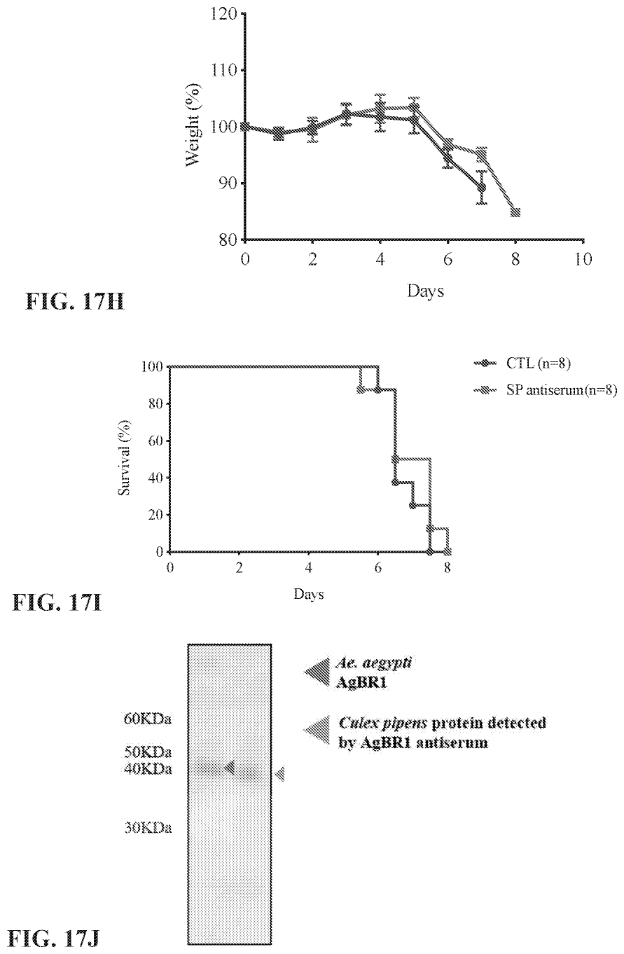

The passive immunization experiments were repeated using the same techniques in West Nile virus (WNV) infection and it was demonstrated that blocking AgBR1 can also enhance survival after WNV infection with mosquito bite, as shown in FIG. 17.

### Example 5: Effect of AgBR1 on Neutrophil Recruitment in the Skin

To examine whether suppression of AgBR1 gene and AgBR1 protein expression in the salivary glands alters the levels of CD45⁺CD11b⁺Ly6G⁺ cells infiltration after mosquito bites. dsRNA against AgBR1 was injected intrathoracically into Aedes aegypti mosquitoes. Expression level of AgBR1 RNA after knockdown was measured using qRT-PCR (FIG. 18A) and protein levels were measured using Western Blot using AgBR1 rabbit antiserum (FIG. 18B). Mosquitoes were infected intrathoracically with Zika virus and after 10 days mosquitoes were collected, and RNA was isolated. Using Zika virus specific primers, viral levels in mosquito were measured, and the levels of Zika virus replication was not altered in the AgBR1 knockdown group (FIG. 18C). The levels of CD45⁺CD11b⁺Ly6G⁺ cells were significantly increased in mice bitten by control mosquitoes, but not in mice bitten by AgBR1 dsRNA-treated mosquitoes (FIG. 18D). These results further demonstrate that AgBR1 plays a role in recruiting CD45⁺CD11b⁺Ly6G⁺ cells to the Zika virus infected-mosquito bite site.

### Example 6: Role of Antigenic Salivary Gland Proteins in Stimulation of Neutrophils

Mosquito saliva can stimulate local immune cells to express IL1ß, CCL2 and CXCL2 at the bite site to change the local immune environment, which leads to an increase of flavivirus-susceptible myeloid-lineage cells. To assess whether any of the antigenic proteins are capable of stimulating immune cells to express these molecules, each of the antigenic proteins were used to treat primary naïve neutrophils harvested from uninfected WT mice and RAW 264.7 macrophage cells. Most SG proteins showed no effect on primary neutrophils ex vivo, but one SG protein, LOC5578631, induced the expression of IL1ß (FIG. 19), CXCL2 (FIG. 20) and CCL2 (FIG. 21). This protein fails to induce IL1ß (FIG. 22) or CXCL2 (FIG. 23) in the RAW macrophage cell line. These data suggest that the LOC5578631 protein is capable of activating neutrophils and thus LOC5578631 protein is called Neutrophil Stimulating Factor 1 (NeSt1).

### Example 7: NeSt1 Enhances Zika Virus Infection In Vivo

To determine if passive immunization against NeSt1 can protect mice from Zika virus infection, AG129 mice were injected with 150 uL of rabbit serum against NeSt1 before infection via mosquito bite. Mosquitoes with similar levels of viral infection (FIG. 24) were allowed to feed on mice, and then blood was collected from mice for 9 days. NeSt1 antiserum significantly reduced Zika virus levels on day 1 after infection (FIG. 25), with no virus detected in 5 out of 12 animals in NeSt1 antiserum group. Passive immunization against NeSt1 also significantly protected mice from pathogenesis as measured by survival over 30 days, as shown in FIG. 26.

In order to examine whether blocking the NeSt1 protein can change the immune microenvironment at the bite site, serum generated from rabbits inoculated against NeSt1 and pre-immune sera from the same animals were used to passive immunize WT mice before feeding naïve mosquitoes on the ear of these animals. Three hours elapsed to allow for infiltration or expansion of immune cells at the local bite site before harvesting the ears and examining the immune response by flow cytometry (FIG. 27). No difference was detected in langerhans cell percentage in the ears of naïve and bitten mice or between NeSt1 antiserum treated compared to naïve sera treated group (FIG. 28). More neutrophils were seen after mosquito bite in both the naïve and NeSt1 antisera, but no differences were detected through the two groups (FIG. 29). The percentage of macrophages in the bitten ear was increased in the control group (FIG. 30) and the percentage of dendritic cells was decreased after mosquito bite in the control group (FIG. 31). Mice treated with NeSt1 antisera did not experience this change in macrophage and dendritic cell percentage (FIGS. 30 and 31) indicating that blocking NeSt1 was capable of preventing the infiltration of macrophages, which are susceptible to Zika virus infection, into the bite site.

To determine whether blocking NeSt1 at the bite site can affect the induction of pro-IL-1β, CXCL2, and/or CCL2 at the bite site, mice were first passively immunized against NeSt1. Mosquitoes were then allowed to feed on one of the ears (bitten), while leaving the other ear unbitten (naive). After 3 h, ear tissue at the bite site was removed and assayed for pro-IL-1β, CXCL2, and CCL2. Mice that had been passively immunized against NeSt1 were shown to express significantly lower levels of pro-IL-1β (FIG. 32A) and CXCL2 (FIG. 32B) after a mosquito bite. No significant differences in CCL2 expression levels were observed between the two groups (FIG. 32C). These data suggest that NeSt1 is capable of inducing pro-IL-1β and CXCL2, two molecules that are capable of increasing the number of ZIKV-susceptible cells at the bite site.

### Example 8: AgBR1 Antibodies Delays Lethal Aedes aegypti-Borne West Nile Virus Infection in Mice

To determine whether targeting AgBR1 altered pathogenesis during mosquito-borne WNV infection, immunized mice were passively immunized with AgBR1 antiserum before challenging them with WNV by mosquito bite. Ae. aegypti mosquitoes were used as a vector model, since the well-annotated whole genome sequence and easy maintenance make this species ideal for laboratory viral transmission studies (63,71).

### Mosquitoes and Animals

Ae. aegypti (Orlando strain, collected from Orlando, Fla. in 1952) and Cx. pipiens mosquitoes were maintained on 10% sucrose feeders inside a 12″×12″×12″ metal mesh cage (BioQuip #1450B) at 28° C. and ˜80% humidity. Eggs were generated via blood meal feeding on an artificial membrane feeder with defibrinated sheep's blood (Hemostat Laboratories). All mosquitoes were housed in a warm chamber in a space approved for BSL3 and ACL3 research. Four-week-old male mice (Swiss Webster mice from Charles River) were used in the WNV infection studies. All mice were kept in a pathogen-free facility at the Connecticut Agricultural Experiment Station.

### Mosquito Injection and Dissections

For WNV injection, WNV-filled needles were carefully inserted into the thorax of each mosquito and 69 nl of virus (3.4×10³ PFU) was injected using a Nanoject II auto-nanoliter injector (Drummond). Infected mosquitoes were placed back in paper cups with mesh lids and maintained in triple containment for 7 days in a warm chamber. After feeding infected mosquitoes on naïve mice, they were knocked-down on ice and salivary glands were dissected to examine the virus levels.

### Passive Immunization Studies

Mice were injected intraperitoneally with 150 μl per animal of AgBR1 or SP antiserum or naive rabbit serum one day before challenge. On the following day, mice were anesthetized with ketamine-xylazine and fed on by a single WNV-infected mosquito per mouse. The blood of fed mice was collected at 1, 3, 5 and 7 days post infection. Survivals and weights were monitored every day. Mice exhibiting weight loss of >20% of initial body weight or neurologic disease were euthanized. Viremia levels were examined at 1, 3, 5 and 7 days post infection by quantitative real time-PCR.

Analysis of Local Immune Responses after Bites of West Nile Virus Infected Mosquitoes

Mice were passively immunized with either AgBR1 or naive antiserum 24 hours prior to allowing infected Ae. aegypti mosquitoes to feed on the left ear. After 6 or 24 hours post feedings, mice were euthanized, and the locations bitten by mosquitoes and naïve locations on the opposite ear were punched using a Disposable Biopsy Punch. Total RNA was extracted by the RNeasy Fibrous Tissue Mini Kit (QIAGEN) according to the manufacturer's instructions. For quantitative RT-PCR, the cDNA was generated with iScript cDNA Synthesis Kit (Bio-rad) according to manufacturer's protocol. Gene expression was examined by qRT-PCR using IQ™ SYBR Green Supermix. Target gene mRNA levels were normalized to mouse β actin RNA levels according to the 2^{-ΔΔCt} calculations. The qRT-PCR primer sequences are available upon request.

### Immunoblot

Three sets of salivary glands from Ae. aegypti and Cx. pipiens were placed in 20 μl Novex 2× Tris-Glycine SDS Sample Buffer, heated to 85° C. for 5 min, diluted 1:1 with water and the whole sample was loaded on a 16% Tris-glycine gel. AgBR1 and homologous proteins were examined with AgBR1 antiserum (1:1000 dilution), followed by incubation with HRP-conjugated secondary antibodies.

### Statistical Analysis

GraphPad Prism software was used to analyze all the data. Mouse β actin-normalized viral RNA levels and body weights were analyzed using the Wilcoxon-Mann-Whitney test. Host responses in vivo was performed using a two-way ANOVA for multiple comparisons. Survival was assessed by a Gehan-Wilcoxon test. A p value of <0.05 was considered statistically significant.

To determine whether targeting AgBR1 altered pathogenesis during mosquito-borne WNV infection, mice were passively immunized with AgBR1 antiserum before challenging them with WNV by mosquito bite. Ae. aegypti mosquitoes were used as a vector model, since the well-annotated whole genome sequence and easy maintenance make this species ideal for laboratory viral transmission studies (63,71). Wild type Swiss Webster mice were administered AgBR1 or control antiserum and 24 hours later were bitten by WNV-infected Ae. aegypti mosquitoes (FIG. 17A). Passive immunization with AgBR1 antiserum significantly reduced WNV RNA levels in the murine bloodstream at an early stage (3 days) of infection (FIG. 17B). Components of mosquito saliva can modulate local host responses and recruit several immune cells which can be target of virus replication (31,69,72), which may lead to virus dissemination at an earlier, rather than later, time point. Although a significant difference as not detected at Day 1, it may be due to the complex interplay of recruited immune cells at the bite site, which leads to shifting populations of WNV-susceptible cells over the first hours and days of infection. In addition, pretreatment with AgBR1 antiserum delayed virally-induced weight loss (FIG. 17C) and prolonged median survival time of mice by 20% (FIG. 17D). As the mosquito-borne WNV infection model used in this study (survival rate: 0%, median survival time: 7 days in control) is much more virulent than a mosquito-borne Zika infection model (survival rate: 30-45%, mean survival time: 12-25 days in control) (69), the 1.5-day delay of fatal outcome is noteworthy. Overall, these results indicate that blocking AgBR1 suppresses virus replication and/or dissemination at early time points and alters mosquito-borne WNV infection.

An experiment was then performed to determine whether the effects observed with AgBR1 were specific to this protein or if any antigenic Ae. aegypti salivary gland protein was capable of affecting WNV infection. An additional protein was selected: the putative 34 kDa family secreted salivary protein (SP). Sera from mice bitten by mosquitoes showed strong reactivity to the SP protein (69). SP has also been reported as a salivary gland protein that was upregulated during flavivirus infection (67).

Identical passive immunization experiments were performed using SP antiserum. SP antiserum did not alter viremia, weight loss or survival time after lethal mosquito-borne WNV infection. The results are shown in FIGS. 17G-17I. In FIG. 17G, the virus levels in blood of mice treated with SP antiserum, fed by an infected mosquito. Blood was collected every other day for 7 days from mice fed on by WNV-infected mosquitoes and analyzed by qRT-PCR. WNV RNA levels were normalized to mouse β actin RNA levels. Mice immunized with naïve serum served as controls. Error bars represent mean±SEM. Each data point represents one mouse. Normalized viral RNA levels were analyzed using one-tailed Wilcoxon-Mann-Whitney test. In FIG. 17H, the weight of mice fed by an infected mosquito. Mice were monitored daily after WNV infection. Error bars represent mean±SEM. Weight at each time point were compared using one-tailed Wilcoxon-Mann-Whitney test. FIG. 17I shows the results of survival assessment by a Gehan-Wilcoxon test (n=8/each group biologically independent samples pooled from two separate experiments).

An experiment was performed to determine whether AgBR1 antibodies alter the early host responses after feeding by mosquitoes infected with WNV. Proinflammatory genes including Il1b, Il6 and Tnfα, were significantly suppressed at the bite site in mice treated with AgBR1 antiserum at 6 hours post feeding (FIG. 17E). In addition to these genes, the expression levels of Mmp9, which is previously reported to play an important role in WNV entry into the brain (73), and those of Nlrp3, which is a key molecule of NLRP3 inflammasome that drives IL-1β signaling and is involved in WNV control in the central nervous system (CNS) (74) were examined. As shown in FIG. 17H, Mmp9 and Nlrp3 genes were significantly suppressed in AgBR1 antiserum-treated mice at 6 hours post feedings. Interestingly, no differences in the expression level of any of these genes was seen 24 hours after bites with WNV-infected mosquitoes (FIG. 17F). The present study of WNV took advantage of immunocompetent wild type mice, as contrasted with the immuno-incompetent Ifnar^{-/-} Ifngr^{-/-} mice, such that it is possible that the difference of mouse models could cause the time lag of early host responses. Overall, these results demonstrated that AgBR1 antiserum suppresses the early local host responses after WNV-infected mosquito feedings, suggesting that the suppression of host responses by AgBR1 antibodies leads to the delay of viral dissemination and fatal outcome.

AgBR1 antiserum also specifically recognizes a protein in Culex pipiens salivary glands (FIG. 17J). Since Ae. aegypti AgBR1 has high homology (amino acid identities=80%) with Culex spp. chitotriosidase-1 protein, which is predicted in silico to be secreted from the salivary gland, it is hypothesized that this Culex protein recognized by AgBR1 antiserum may have similar function during WNV infection.

In conclusion, this example demonstrates that passive immunization with AgBR1 antiserum delays lethal Ae. aegypti-borne WNV infection in mice, similar to that shown for Zika virus infection transmitted by the same mosquitoes. A strategy of targeting individual arthropod salivary factors such as AgBR1 might be broadly applicable to other mosquito-borne pathogens.

LIST OF SEQUENCES:
LOC5573204 (SEQ ID NO: 1; Genbank Accession
No. ABF18180.1)
MWFFKVGALLFLAALVSANNATTGPKVLCYYDGQMSLREGLGKITVTDIE
LALPFCTHLLYGFAGVNPETYRLKALDESLELDSGKGQYRLATTLKRRYP
NLKVLLSVGGYKDLTEEKPFEKYLTLLESAGSRTAFVNSVYSTLKTYDFD
GLDLAWQFPQTKPKRIRGWTGKVWHGFKKLFTGDSVLDPKADEHREEFTA
LVRDLKNALVADNFILGLTVLPHVNESIFMDVPLLKDNLDYVNLASFDQQ
TPERNPKEGDYTAPIYEPSERVEGNNVDAEASYWQGTPAGKIVIGIPTYG
RGWKLVEKSGITGVPPIPADGPSIPGPHSGINGFYSWAEVCAKLPNPGNA
NLQGADQPLRKIGDPTRRFGAYAFRIPDENEEHGIWLSYEDPDTAGNKAA
YVKAKGLGGISIFDLGNDDVRGACAGDKFPILRAAKYRL
LOC5578630 (SEQ ID NO: 2; Uniprot Accession
No. Q1HRF7-1)
MSPSKKILVLLFFPILLVSSHPIPAEDPAKQCNLSEDDLTKLKAAISSAS
SAKAANEDILPSTTLAACPMLKNFTEMLKTVATDMEVLKTQGVSNMEVQL
LRESFEEKLNDLAKNKDIFERQANQDTSKAEGEMVEKINKLQLEMAKLQE
EIEEQTKQMYVDMIEYIFERLKMNDTEAIDSYAQIVMKTKMHELIMKLKT
DRLVLWEMVKYVEGKKNKWVGRKVLNTILDQVNKLKLYKPEEVEIGKNSL
VVVWCWKFNSETVYGTTDEDQKSFHLAKLFFPKEKGCKECADVKSRTMCN
NDYPKVMVKAFG
LOC5578631 (SEQ ID NO: 3; Uniprot Accession
No. 5578631)
METSLPITVVFLIVLITGAQTKPTQGSCTLTDEDISDIKSAVQKASKAAV
NDIVLDPTLIDKCPMLEKITASLKSVATEIVQMRDSATSTDQVDQLKQNF
EDQVNQIVKSRDIFEKQSGTQATKEHGEMLERMTALQVKVTELEQQIAKQ
TASMYEDMAELIFQRLQMNSTESVRSYTKHMMEEKLEELMNKLETNYRIY
LGALRFLNHMNDQELIGKVFDGILKRLGDMKADSDDVKENGRNLLVNLLC
WTVNNDFLGKKYKERQVDLYRMALKFYPKTYEKAANEADVRSRQFCEENF
PANLITWFAVSWNDRG
LOC5567956 (SEQ ID NO: 4; Uniprot Accession
No. 5567956)
MFPPRKFLLSSFILAALHVTAAPLWDAKDPEQLRFITSRCMEDWYPKAKN
PKAALQNWLGWKLEPSDDQATQCYTKCVLEKIGFYEPGEKRFKGVRVMQQ
WETFHKYLNADREKVHDLTSTFDFIPPLKSSSCSEVFEAFKKVNGKHSET
IRAILFGKGESSKKYYQEKGVKIKQKEQSLFMHCEALNYPKGSPQRKDLC
GIRKYQMGSGIVFERHMECIFKGLRYMTSKNELDVDEIARDFIVVKKKPD
AMKAMMKTCKANLKEKNPGKIAVHYYKCLMNDSKVTNDFKEAFDYREVRS
KDYFAALTGKLKPYSRSDVRKQVDDIDKIQCS
LOC5580038 (SEQ ID NO: 5; Uniprot Accession
No. Q8T9T8-1)
MKYLLTFLMALSLVNLMLTRPTPEDDGGTSEEPQTQETTGSDEKNGASEE
PNADDASKPDDVEEKGDDDTAKKEDDGESKDGEGSEKSDKEKGEPKNDPR
ETYNKVIEQLDQIKVDNVEDGHERSELAADIQRYLRNPIVDVIGSAGDFS
KIAKCFKSMVGDAKKAIEEDVKGFKECTAKKDSNAYQCSQDRSTVQDKIA
KMSSKIASCVASNRS
LOC5573204 Antigenic Peptide
(SEQ ID NO: 6)
NLKVLLSVGGY
LOC5573204 Antigenic Peptide
(SEQ ID NO: 7)
FKVGALLFLAALVSA
LOC5573204 Antigenic Peptide
(SEQ ID NO: 8)
TALVRDLKNALVADNFILGLTVLPHVNES
LOC5573204 Antigenic Peptide
(SEQ ID NO: 9)
KITVTDIELALPFCTHLLYGFAGV
LOC5573204 Antigenic Peptide
(SEQ ID NO: 10)
TAFVNSVYSTLKTY
LOC5573204 Antigenic Peptide
(SEQ ID NO: 11)
FMDVPLLKDNLDYVNLASF
LOC5573204 Antigenic Peptide
(SEQ ID NO: 12)
ITGVPPIPADGP
LOC5573204 Antigenic Peptide
(SEQ ID NO: 13)
RGACAGDKFPILRAAK
LOC5578630 Antigenic Peptide
(SEQ ID NO: 14)
NSLVVVWCWK
LOC5578630 Antigenic Peptide
(SEQ ID NO: 15)
KKILVLLFFPILLVSSHPIPAE
LOC5578630 Antigenic Peptide
(SEQ ID NO: 16)
LPSTTLAACPML
LOC5578630 Antigenic Peptide
(SEQ ID NO: 17)
TDRLVLWEMVKYVE
LOC5578630 Antigenic Peptide
(SEQ ID NO: 18)
SFHLAKLFFP
LOC5578630 Antigenic Peptide
(SEQ ID NO: 19)
LNTILDQVNKLKLYKPEEV
LOC5578630 Antigenic Peptide
(SEQ ID NO: 20)
TKLKAAISSASSA
LOC5578630 Antigenic Peptide
(SEQ ID NO: 21)
QMYVDMIEYIFE
LOC5578631 Antigenic Peptide
(SEQ ID NO: 22)
SLPITVVFLIVLITG
LOC5578631 Antigenic Peptide
(SEQ ID NO: 23)
NLLVNLLCWTV
LOC5578631 Antigenic Peptide
(SEQ ID NO: 24)
ALQVKVTELEQQIAKQ
LOC5578631 Antigenic Peptide
(SEQ ID NO: 25)
IKSAVQKASKAAVNDIVLDPTLIDKCPML
LOC5578631 Antigenic Peptide
(SEQ ID NO: 26)
ITASLKSVATEIV
LOC5578631 Antigenic Peptide
(SEQ ID NO: 27)
ANLITWFAVS
LOC5578631 Antigenic Peptide
(SEQ ID NO: 28)
IGKVFDGILKR
LOC5578631 Antigenic Peptide
(SEQ ID NO: 29)
ERQVDLYRMALKFYPKT
LOC5567956 Antigenic Peptide
(SEQ ID NO: 30)
ATQCYTKCVLEKI
LOC5567956 Antigenic Peptide
(SEQ ID NO: 31)
KIAVHYYKCLMND
LOC5567956 Antigenic Peptide
(SEQ ID NO: 32)
RKFLLSSFILAALHVTAAPLW
LOC5567956 Antigenic Peptide
(SEQ ID NO: 33)
RDFIVVKKK
LOC5567956 Antigenic Peptide
(SEQ ID NO: 34)
TFDFIPPLKSSSCSEVFEAFKK
LOC5567956 Antigenic Peptide
(SEQ ID NO: 35)
LFMHCEALNYP
LOC5567956 Antigenic Peptide
(SEQ ID NO: 36)
YFAALTGKLKPY
LOC5580038 Antigenic Peptide
(SEQ ID NO: 37)
KMSSKIASCVAS
LOC5580038 Antigenic Peptide
(SEQ ID NO: 38)
LLTFLMALSLVNLMLT
LOC5580038 Antigenic Peptide
(SEQ ID NO: 39)
SELAADIQRYLRNPIVDVIGSA
LOC5580038 Antigenic Peptide
(SEQ ID NO: 40)
NKVIEQLDQIKVDNV
LOC5580038 Antigenic Peptide
(SEQ ID NO: 41)
FSKIAKCFKSMVG
LOC5580038 Antigenic Peptide
(SEQ ID NO: 42)
AYQCSQDRSTVQDK
LOC5566287 (SEQ ID NO: 43; Uniprot Accession
No. Q17NC0)
MNRQLWIIIFAILCVAQAEEDNPTTEKMEELGIATINNFTREFYSYVEAV
SQVLADLELTTTASITQIKHRIKHLLQEKCNLCSAKAEGPALDQGYVTTS
NGSVIPVSYEQTRFGGGWIVLMQRYDGTVRFNRSWAEYRDGFGMVGHEFW
LGLERIHQMTKDAEYELMIEMQDFEGNYKYAGYDAFAVGPEEERYPLAKV
GKFNKTAYVDSFGKHRGYGFSTYDNDDNGCSNQYGRGGWWYYRKSCFGAS
LTGIWQNKQDWKSISWVWFSTEKKQVPLKFARMMMRLKTAE
LOC5567958 (SEQ ID NO: 44; Uniprot Accession
No. P18153)
MKLPLLLAIVTTFSVVASTGPFDPEEMLFTFTRCMEDNLEDGPNRLPMLA
KWKEWINEPVDSPATQCFGKCVLVRTGLYDPVAQKFDASVIQEQFKAYPS
LGEKSKVEAYANAVQQLPSTNNDCAAVFKAYDPVHKAHKDTSKNLFHGNK
ELTKGLYEKLGKDIRQKKQSYFEFCENKYYPAGSDKRQQLCKIRQYTVLD
DALFKEHTDCVMKGIRYITKNNELDAEEVKRDFMQVNKDTKALEKVLNDC
KSKEPSNAGEKSWHYYKCLVESSVKDDFKEAFDYREVRSQIYAFNLPKKQ
VYSKPAVQSQVMEIDGKQCPQ
LOC5568702 (SEQ ID NO: 45; Uniprot Accession
No. Q173Q2)
MVQFPVLLITLSLAFEVHSSYAENRRLQLVRDIDGTQQLVNPNPYRVLNA
HLERSFNAQSDIIFRLYTRKNPEKHQILKPNDTSSILNSNFNADLPTRFL
IHGWNQNGESDILIELRRSYLSVEDFNVIGVDWGEGALTINYVMARKRVE
SVGLVTSQLIDTLVDASGVILDSIYVIGHSLGAHVAGIVGKHQRGQLNTI
VGLDPAGPLFSLNSSDILNQNHAQYVEMVSTGARLLGTYEPLGDANFYPN
GGLEQAGCGLDLFGICAHARSWIYFAETVTNGKGFRGIKCAMIEDLEGET
CNLSGLPNVWMGGEPSNHERGVKGIFMVHTNSEAPFAKD
LOC110675548 (SEQ ID NO: 46; Uniprot Accession
No. Q17NB9)
MILQFWVVTFSVLFAARADENHSILIKLNDLDHRFTQMFSQQFYRHTQQV
TDRVSALKISIDTNLLELDQQIQQALDGIQSNESSSSASATKPPGLTTIP
IGSEPRVPALYERERYGGDWLVVMHRYDGSVKFDRTWAEYRDGFGMVGQE
FWYGLERLHQLTKEKSYELMVEMEDFNGSLKYAWYDKFVVGPEEQRYALV
ELGTFNGTTDGDSLKPHKGSGFSTYDNDDFGCSNKYAKGGWWYYSGKCYG
SSLTGIWKNELAYSSIVWMKFSDVSNTPLKLVRMMIRPKN
LOC5566287 Antigenic Peptide
(SEQ ID NO: 47)
LWIIIFAILCVAQA
LOC5566287 Antigenic Peptide
(SEQ ID NO: 48)
FYSYVEAVSQVLADLE
LOC5566287 Antigenic Peptide
(SEQ ID NO: 49)
GSVIPVSYE
LOC5566287 Antigenic Peptide
(SEQ ID NO: 50)
ITQIKHRIKHLLQEKCNLCSAK
LOC5566287 Antigenic Peptide
(SEQ ID NO: 51)
KKQVPLKF
LOC5566287 Antigenic Peptide
(SEQ ID NO: 52)
YPLAKVG
LOC5566287 Antigenic Peptide
(SEQ ID NO: 53)
RKSCFGASLTG
LOC5566287 Antigenic Peptide
(SEQ ID NO: 54)
GWIVLMQ
LOC5566287 Antigenic Peptide
(SEQ ID NO: 55)
LDQGYVTT
LOC5566287 Antigenic Peptide
(SEQ ID NO: 56)
YAGYDAFAVG
LOC5566287 Antigenic Peptide
(SEQ ID NO: 57)
TAYVDSF
LOC5566287 Antigenic Peptide
(SEQ ID NO: 58)
ISWVWFS
LOC5566287 Antigenic Peptide
(SEQ ID NO: 59)
WLGLERI
LOC5567958 Antigenic Peptide
(SEQ ID NO: 60)
PLLLAIVTTFSVVAST
LOC5567958 Antigenic Peptide
(SEQ ID NO: 61)
WHYYKCLVESS
LOC5567958 Antigenic Peptide
(SEQ ID NO: 62)
PVDSPATQCFGKCVLVRTG
LOC5567958 Antigenic Peptide
(SEQ ID NO: 63)
CAAVFKAYDPVHKA
LOC5567958 Antigenic Peptide
(SEQ ID NO: 64)
YREVRSQIYAFNLPKKQVYSKPAVQSQVM
LOC5567958 Antigenic Peptide
(SEQ ID NO: 65)
KVEAYANAVQQLP
LOC5567958 Antigenic Peptide
(SEQ ID NO: 66)
YDPVAQKFDASVIQEQFKAYPSL
LOC5567958 Antigenic Peptide
(SEQ ID NO: 67)
QQLCKIRQYTVLDDA
LOC5567958 Antigenic Peptide
(SEQ ID NO: 68)
LEKVLNDC
LOC5567958 Antigenic Peptide
(SEQ ID NO: 69)
YFEFCENKYYPA
LOC5567958 Antigenic Peptide
(SEQ ID NO: 70)
FKEHTDCVMKG
LOC5568702 Antigenic Peptide
(SEQ ID NO: 71)
FPVLLITLSLAFEVHSS
LOC5568702 Antigenic Peptide
(SEQ ID NO: 72)
VESVGLVTSQLIDTLVDASGVILDSIYVIGHSLGAHVAGIVGKH
LOC5568702 Antigenic Peptide
(SEQ ID NO: 73)
QAGCGLDLFGICAHARSWIYFAE
LOC5568702 Antigenic Peptide
(SEQ ID NO: 74)
LQLVRD
LOC5568702 Antigenic Peptide
(SEQ ID NO: 75)
TQQLVNPNPYRVLNAH
LOC5568702 Antigenic Peptide
(SEQ ID NO: 76)
TINYVMAR
LOC5568702 Antigenic Peptide
(SEQ ID NO: 77)
AQYVEMV
LOC5568702 Antigenic Peptide
(SEQ ID NO: 78)
NTIVGLDPAGPLFSLNSS
LOC5568702 Antigenic Peptide
(SEQ ID NO: 79)
KGIFMVH
LOC5568702 Antigenic Peptide
(SEQ ID NO: 80)
CNLSGLPNV
LOC5568702 Antigenic Peptide
(SEQ ID NO: 81)
DILIELRRSYLSVEDF
LOC5568702 Antigenic Peptide
(SEQ ID NO: 82)
PTRFLIHG
LOC5568702 Antigenic Peptide
(SEQ ID NO: 83)
SDIIFRLY
LOC5568702 Antigenic Peptide
(SEQ ID NO: 84)
GIKCAMI
LOC5568702 Antigenic Peptide
(SEQ ID NO: 85)
GARLLGTY
LOC110675548 Antigenic Peptide
(SEQ ID NO: 86)
QFWVVTFSVLFAA
LOC110675548 Antigenic Peptide
(SEQ ID NO: 87)
LAYSSIVWMKFSDVSNTPLKLVRM
LOC110675548 Antigenic Peptide
(SEQ ID NO: 88)
DWLVVMHR
LOC110675548 Antigenic Peptide
(SEQ ID NO: 89)
EPRVPALY
LOC110675548 Antigenic Peptide
(SEQ ID NO: 90)
HSILIKLND
LOC110675548 Antigenic Peptide
(SEQ ID NO: 91)
RYALVELG
LOC110675548 Antigenic Peptide
(SEQ ID NO: 92)
TDRVSALKIS
LOC110675548 Antigenic Peptide
(SEQ ID NO: 93)
SGKCYGSSLTG
LOC110675548 Antigenic Peptide
(SEQ ID NO: 94)
SLKYAWYDKFVVGP
LOC110675548 Antigenic Peptide
(SEQ ID NO: 95)
LERLHQL
LOC110675548 Antigenic Peptide
(SEQ ID NO: 96)
DTNLLELDQQIQQALDG
LOC110675548 Antigenic Peptide
(SEQ ID NO: 97)
SQQFYRHTQQ
LOC110675548 Antigenic Peptide
(SEQ ID NO: 98)
DGSVKF
LOC110675548 Antigenic Peptide
(SEQ ID NO: 99)
PPGLTTIPIG
(Aedes aegypti Polypeptide)
SEQ ID NO: 100
MAKAPAVGIDLGTTYSCVGVFQHGKVEIIANDQGNRTTPSYVAFTDTERL
IGDAAKNQVAMNPTNTIFDAKRLIGRKFDDPAIQADMKHWPFDVISVEGK
PKIQVEYKGETKNFFPEEISSMVLTKMKETAEAYLGKTVSNAVVTVPAYF
NDSQRQATKDAGTISGLNVLRIINEPTAAAIAYGLDKKTAGERNVLIFDL
GGGTFDVSILSIDDGIFEVKSTAGDTHLGGEDFDNRLVNHFAQEFKRKHK
KDLSTNKRALRRLRTACERAKRTLSSSTQASIEIDSLFEGTDFYTSITRA
RFEELNADLFRSTMEPVEKAIRDAKMDKASIHDIVLVGGSTRIPKVQKLL
QDFFNGKELNKSINPDEAVAYGAAVQAAILHGDKSEEVQDLLLLDVTPLS
LGIETAGGVMSVLIKRNTTIPTKQTQTFTTYSDNQPGVLIQVFEGERAMT
KDNNLLGKFELSGIPPAPRGVPQIEVTFDIDANGILNVTALEKSTNKENK
ITITNDKGRLSKEDIERMVNEAEKYRSEDEKQKETISAKNALESYCFNMK
ATMEDDKLKDKITDSDKTLIMDKCNDTIKWLDANQLAEKEEYEHRQKELE
SVCNPIITKLYQSAGGAPGGMPGFPGGAPGAGAGAAPGAGSGSGPTIEEV
D
(Aedes aegypti Polypeptide)
SEQ ID NO: 101
MSVNRTISAHQAAKEHVLAVSRDFISQPRLTYKTVSGVNGPLVILDEVKF
PKFAEIVQLRLNDGTVRSGQVLEVSGSKAVVQVFEGTSGIDAKNTVCEFT
GDILRTPVSEDMLGRVFNGSGKPIDKGPPILAEDFLDIQGQPINPWSRIY
PEEMIQTGISAIDVMNSIARGQKIPIFSAAGLPHNEIAAQICRQAGLVKH
TGKSVLDEHEDNFAIVFAAMGVNMETARFFKQDFEENGSMENVCLFLNLA
NDPTIERIITPRLALTAAEFLAYQCEKHVLVILTDMSSYAEALREVSAAR
EEVPGRRGFPGYMYTDLATIYERAGRVEGRNGSITQIPILTMPNDDITHP
IPDLTGYITEGQIYVDRQLHNRQIYPPVNVLPSLSRLMKSAIGEGMTRKD
HSDVSNQLYACYAIGKDVQAMKAVVGEEALTPDDLLYLEFLTKFEKNFIS
QGNYENRTVFESLDIGWQLLRIFPKEMLKRIPASILAEFYPRDSRH
(Aedes aegypti Polypeptide)
SEQ ID NO: 102
MPANIIMKILITSILILKLAIHVVPQHLISSGASAVESKPVSARPTYEDY
KRQRENFLQAEEYHFLGANVTLNENEQLVNKFLMRLKLEEMVKGFNDSYN
FIPARHIFEVLDRFGQSKVFKVIQRLPKGGVLHAHDMALGSTDLIVNATY
RENLWQKGNFGVSHGPQFKFSKEKPGKEWSLVSEIRQWMTDKVYDAKVGE
IFSLYNADPLNAYKSLDDVWSKFQNLFGSLAPLITFAPVWRQYYHDSLKQ
FYDDHVQYLEFRGVLPDVYDLDGKIYSAEEIVQMYYEETEEFKSSHPEFI
GAKFIYAPGRFATDDEFLKIIDTAKRLHKKFPTFLAGFDLVGQEDPGRSL
LEFAPALLKLPASINFFFHAGETNWYGMKTDQNLIDAVLLGSKRIGHGFA
VLKHPKVLKEIKRRQICIEINPISNQVLKLVQDQRNHPAALLFSDNYPVV
VSSDDPSFWRSTPLSHDFYVAFTGIASAKQDLRLLKQLALNSIEYSAMNS
EEKTSAKEKWSQAWHDQISALATDIVAGSV
(Aedes aegypti polypeptide)
SEQ ID NO: 103
MAGRPGYSEVIFLYVVSVAVIARATDNMPVNKDVSKLFPLTLIHINDLHA
RFEETNMKSNVCTQKDQCIAGIARVYQKIKDLLKEYESKNPIYLNAGDNF
QGTLWYNLLRWNVTADFIKKLKPAAMTLGNHEFDHTPKGLAPYLAELNKE
GIPTIVANLVMNNDPDLKSSKIPKSIKLTVGKRKIGIIGVLYDKTHEIAQ
TGKVTLSNAVEAVRREAAALKKDNIDIIVVLSHCSYEEDKKIAAEAGDDI
DVIVGAHSHSFLYSPDSKQPHDPKDKVEGPYPTLVESKNKRKIPIVQAKS
FGKYVGRLTLYFDEEGEVKNWEGYPVFIDHKVQQDPQILKDLVPWRAKVE
AIGSTVVGETMIELDRDSCRDQECTLGVLYADGFADQYTNDTFRPFAIIQ
AGNFRNPIKVGKITNGDIIEAAPFGSTADLIRLKGADIWDVAEHSFALDD
EGRTNCLQVSGLRIVIDISKPVRSRVKKIEVMDYTNPKSDKLKPLDKEAE
YYIVVPSYLADGKDGFSAMKRATARRTGPLDSDVFKNYVEKIKKVDNLKL
GRVIVCKGSKCT
(Aedes aegypti polypeptide)
SEQ ID NO: 104
MIDQCACSHQLSAALSTEDMLRTSSIVFLTCCLTFLIEGSSFKLKIIHFN
DIHARFDEVTNSSSPCSGNGETCVAGIARLVTTIEKLRKQNENHLVLNAG
DVFQGTIWYTLLKWNVSQQFMNMVKADAMTLGNHEFDDSFPVLIPFLENT
KNVTPVVVSNLVFPKQLSRDVTKFRSLIKEDPLVLTVGGQSIGIIGVIFD
ETDKIGNSDPLKFKSSIETVRIAAKQLKSKGVNIIIVLSHCGVFDDKKIA
EQAGEDIDIIVGGHTHTLLYNGDPPSKHAALDKYPIVVETGNNHKVLIVQ
AFCHGHYVGNIDLTFDDEGEITAFEGQPIYQENRIEKNALVEARVRELRK
DVEVKSLVKVGESKLELSNDCRLKDCTFGSVLADAYVWHFRSRSNAPMIA
MIHPGNFRISLAAGAITRGQILTALPFNSNANRVTVLGSTIKKAIEFGTS
INPRRCSFNALQTAGIKIDVDYGKPVGNRTVILLKTGGKYKRLVESKKYD
ILVNSYVFKGGDGFDMFKHLAVKGRAPFDAELLEQYIVARKGIQKSGLLQ
SRMNVSHVEKALSEVKSCKQSR
(Aedes aegypti polypeptide)
SEQ ID NO: 105
MCSTGFCLVFFLAQVVFQMNYSEQQTTVVMENGAISESEINVDIVMEQYI
LKFYTKRFVEGQNLVVAPLLTFRVFMSLYKAMDASAKFDLHSLLGIQQDT
SVEKMSEIEAFANKHTLPVDEKQISVETRLYYDKSIGNARSVLTAKSLKP
IGTSFSDKRAFCEKVNSWIRNAPIKGTDNLVRDYDLNNETQAFVAGALSI
YWNTQLKSSTDQKGFQGENVKFLEGSISAGYAKLDNLKVEVVELISDKVD
GVKLWLIMPDRASSIKDFNDQLSVESIRQIENGLTAQKVDVSLALPMVTI
EYNSQEDAYVTEVFEVFSSLFTKPSVKLVDGKDDLYVIKNFLMKCILRFV
ESDASADSKAQSTGMLVKFDRPFVMMMLSKEGNVPILLANYFSPTDKLRA
LEAKERRLKAEANEHLDL
(Aedes aegypti polypeptide)
SEQ ID NO: 106
MNLWIIGFCSIYFACSVRSQFTSVPVSYDAQNDHNEFSWNAFKKVFTDYK
ENFVMSPYSLRRLFSCFQGVKLLTSASGTNLQQELSNVLKIVPNQQPSGQ
DHRPYVEQWVRYSSAKYLNRTAMAVAIGSEKVSTVYESIINNCVIYTGHL
QPSNAQRMGQVINDALKNITNNAVQSYLTDTDINPNWKFFAIDSWQFEGL
WKFKFQEEFSATCYFYASREKKGLTKFLYLEEMLKYGNFPEWNVQAVELP
YHDQSPLSCLLMMPLDGNYESLIHSMNQSRFKEVLSKLNEIKTTVRIPQF
GLQTTVPGRQLLESMGMKVPFNQGVFKVFEQGQDVALGEIVQKMEMSIAA
DGEKQAQSFVDKRQDKQFTAHQPFLFVVYDRNELVPILVGFYLKTPPEAA
MGLEDKQKCDDPPVGYQ
(Aedes aegypti polypeptide)
SEQ ID NO: 107
MNRQLWIIIFAILCVAQAEEDNPTTEKMEELGIATINNFTREFYSYVEAV
SQVLADLELTTTASITQIKHRIKHLLQEKCNLCSAKAEGPALDQGYVTTS
NGSVIPVSYEQTRFGGGWIVLMQRYDGTVRFNRSWAEYRDGFGMVGHEFW
LGLERIHQMTKDAEYELMIEMQDFEGNYKYAGYDAFAVGPEEERYPLAKV
GKFNKTAYVDSFGKHRGYGFSTYDNDDNGCSNQYGRGGWWYYRKSCFGAS
LTGIWQNKQDWKSISWVWFSTEKKQVPLKFARMMMRLKTAE
(Aedes aegypti polypeptide)
SEQ ID NO: 108
MILQFWFVTFSVLFAARADENHSILIKLNDLDHRFTQMFSQQFYRHTREV
TDRVSALKASIDTNLLELDQQIQQALDGIQSNESSSSTSATKSSGLTTIP
IGSEPRVPALYERERYGGDWLVVMHRYDGSVKFDRTWAEYRDGFGMVGQE
FWYGLERLHQLTKEKSYELMVEMEDFNGNLKYAWYDKFVVGPEEQRYALV
ELGTFNGTTDGDSLKPHKGSGFSTYDNDDFGCSNKYAKGGWWYYSGKCYG
SSLTGIWKNELAYSSIVWVKFSDVSNTPLKLVRMMIRPKN
(Aedes aegypti polypeptide)
SEQ ID NO: 109
MSPSNKILVLLLFPILLVSSHPIPAEDPAKQCNLSEDDLTKLKAAISGAS
SAKAANEDILPNTTLAACPMLKNFTEMLKTVATDMEVLKTQGVSNMEVQL
LRESFEEKLNDLAKNKDIFERQANQDTSKAEGEMVEKINKLQLEMAKLQE
EIEEQTKQMYVDMIEYIFERLKMNDTEAIDSYAQIVMKTKMHELIMKLKT
DRLVLWEMVKYVEGKKNKWVGRKVLNTILDQVNKLKLYKPEEVEIGKNSL
VVVWCWKFNSETVYGTTDEDQKSFHLAKLFFPKEKGCKECANVKSRTMCN
NDYPKVMVKAFG
(Aedes aegypti polypeptide)
SEQ ID NO: 110
MKLPLLLAIVTTFSVVASTGPFDPEEMLFTFTRCMEDNLEDGPNRLPMLA
KWKEWINEPVDSPATQCFGKCVLVRTGLYDPVAQKFDASVIQEQFKAYPS
LGEKSKVEAYANAVQQLPSTNNDCAAVFKAYDPVHKAHKDTSKNLFHGNK
ELTKGLYEKLGKDIRQKKQSYFEFCENKYYPAGSDKRQQLCKIRQYTVLD
DALFKEHTDCVMKGIRYITKNNELDAEEVKRDFMQVNKDTKALEKVLNDC
KSKEPSNAGEKSWHYYKCLVESSVKDDFKEAFDYREVRSQIYAFNLPKKQ
VYSKPAVQSQVMEIDGKQCPQ
(Aedes aegypti polypeptide)
SEQ ID NO: 111
MHSPKSFLLLAVVFVALRVTAAPLWNAKNPEQLQYIAARCMEEWSPKAKD
PKAALKNWMEWKLQPSNEEATQCYTKCMLENIGYYEPGEKRLKGVRVMQQ
WETFNRYQSADRNKVHDLTDTFDFIKPLKSSSCSDVFNAYKDVHAKHLET
IKAILFCDGKSAEKYYKDKGKNVKQKGESIFVHCEEIHYPVGSPQRNELC
KVRKYELGTGKPFENLMECIFKGVRYFNDKNELNIDEIARDFTQVGKKPD
AVKAAMENCKSKTKETDPGKKAVEYYKCLLADSKVKKDFMEAFDYREIRS
KDYYAQITGKLKPYSASDVRKEVNDIDSNKCV
(Aedes aegypti polypeptide)
SEQ ID NO: 112
MKLKVYICQVIFSFLAVSVFCEENCNIPESELSKIDHVLRHMEKPIYSEE
QFASDNEECTNLLNGIHAQLRRLTQRYKLMNKGYVKVEEYQRMADDYEKQ
LKTLNDELVELQQHTSEKASATIAKLKEDIKKLDEEVGTLHEKLKGIKQD
FEKVKRDLCVTYLNSNQMSKAKAKLKEMASTYLIEIVQQQLNKSNANIMP
MLEFSAAIPDLDDMGEAYKEIYKFLEEQKRLEGEDSVLLEATVLKMNASL
KEGSNITDERRTQIEGLLKDLATKSTIVFSTWTKELKKINDAVVIKNALD
HMFVSQMKVFGALVGDTSDFGSIRNFVKLTVVCNNYYKVAAYKELIDRKI
GNALGTIMFDLLTLEVNEMKFDPHVPDEIPKLFEATLSSLPNSLTELRTC
LGKVQIYNKKTNKCVVATGNDFDVHKDKLGDFYRVVVADYGCTSFRLEAS
GDKASVRIVTPSGNPMSNVNLHLEGNSLHNYVATPKSNKPDRTPSSSDEW
ILDANYNNDTIKIESQFSDYKTKKTEVDHLLVRDINHLPHVLVARYGFMG
LKNSDAKDTIEWNLKCGS
(Aedes aegypti polypeptide)
SEQ ID NO: 113
METSLPITVVFLIVLITGAQTKPTQGSCTLTDEDISDIKSAVQKASKAAV
NDIVLDPTLIDKCPMLEKITASLKSVATEIVQMRDSAISTDQVDQLKQNF
EDQVNQIVKSRDIFEKQSGTQATKEHGEMLERMTALQVKVTELEQQIAKQ
TASMYEDMAELIFQRLQMNSTESVRSYTKHMMEEKLEELMNKLETNYRIY
LGALRFLNHMNDQELIGKVFDGILKRLGDMKADSDDVKENGRNLLVNLLC
WTVNNDFLGKKYKERQVDLYRMALKFYPKTYEKAANEADVRSRQFCEENF
PANLITWFAVSWNDRG
(Aedes aegypti polypeptide)
SEQ ID NO: 114
MKYLLTFLMALSLVNLMLTRPTPEDDGGTSEEPQTQETTGSDEKNGASEE
PNADDASKPDDVEEKGDDDTAKKEDDGESKDGEGSEKSDKEKGEPKNDPR
ETYNKVIEQLDQIKVDNVEDGHERSELAADIQRYLRNPIVDVIGSAGDFS
KIAKCFKSMVGDAKKAIEEDVKGFKECTAKKDSNAYQCSQDRSTVQDKIA
KMSSKIASCVASNRS
(Aedes albopictus polypeptide)
SEQ ID NO: 115
MHSLKSSPLLAAVFLALHVTGAPFWNAKNPDELQSIAARCMDEWSPKAKD
PKAALKNWKEWRLQPSNDEATKCYTKCMLENIGFYEPAEKRLKGVRIMQQ
WETFSRYQSADREKVHDLTDTFNFIRPLKSSSCTDVFNAYKDVHARHLET
IKAILFCDGKSAEKYYKDKGKTSKQKKVLCTGS
(Aedes albopictus polypeptide)
SEQ ID NO: 116
MKTSLPIVVLLTAVISGVHPNPTPKSCTVSEEDLTTIRNAIQKASRASLD
DVNLDEDLIAKCPLLKTITASLKSVASEIATLKDTGISEEQVDELKQSYE
QQVNEIVKSRDIFEKQSGGDVMKEQGAMINRMTELQVQVAQLQQQIGEQT
SRMYDDMAELIFQRLAMNSTDSIRNYTAHMMEQKLHTLMTKLETNYRIFL
GALRYLDHLGDQPLIDKVFDGILKRLDEMSLETNKERENGKYVLVNLLCW
TVNNRFLTEKYRKKQLELFRIALKFYPKTGNKEANEADIRGRQFCDANFP
VNVITWFAVSRAAEGWGLRGTL
(Aedes aegypti polypeptide)
SEQ ID NO: 117
MSTLKKISDEDRESKFGYVFAVSGPVVTAERMSGSAMYELVRVGYYELVG
EIIRLEGDMATIQVYEETSGVTVGDPVLRTGKPLSVELGPGIMGSIFDGI
QRPLKDINELTSSIYIPKGVNIPCLSRTQSWGFNPLNVKVGSHITGGDLY
GLVHENTLVKHKLLVPPRAKGTVRYIAPPGNYTVDDIILETEFDGEINKW
SMLQVWPVRQPRPVTEKLPANHPLLTGQRVLDSLFPCVQGGTTAIPGAFG
CGKTVISQALSKYSNSDVIIYVGCGERGNEMSEVLRDFPELSVEIDGVTE
SIMKRTALVANTSNMPVAAREASIYTGITLSEYFRDMGYNVSMMADSTSR
WAEALREISGRLAEMPADSGYPAYLGARLASFYERAGRVKCLGNPEREGS
VSIVGAVSPPGGDFSDPVTSATLGIVQVFWGLDKKLAQRKHFPSINWLIS
YSKYMRALDDFYDKNFQEFVPLRTKVKEILQEEEDLSEIVQLVGKASLAE
TDKITLEVAKLLKDDFLQQNSYSAYDRFCPFYKTVGMLRNMIGFYDMARH
AVETTAQSENKITWNVIRDSMGNILYQLSSMKFKDPVKDGEAKIKADFDQ
LYEDLQQAFRNLED
(Aedes albopictus polypeptide)
SEQ ID NO: 118
MAGKPGIQLFVIFILLSSFAAVVWTTDNMPADKDVSKLFPLTLIHINDLH
ARFDETNMKSNACTAKDQCIAGIARVYQKIQDLLKEYKSKNAIYLNAGDN
FQGTLWYNLLRWQVTADFITKLKPTAMTLGNHEFDHTPKGLAPYLAELDK
AGIPTLVANLVMNDDPDLKSSKIQKSIKVTVGGKTIGIIGVLYDKTHEIA
QTGKVTLSNAVETVKREAAALKKDKVDIIVVLSHCSYDEDKKIAKEAGQD
IDVIVGAHSHSFLYSKESNKPYDQKDKIEGPYPTIVESNNKRKIPIVQAK
SFGKYVGRLTLYFDNEGEVKHWEGYPEFIDNKVKQDPKILEALIPWRKKV
QEIGSTKVGETTIELDRDSCRDKECTLGVLYADAFADHYTNSSFRPFAII
QAGNFRNPIKVGKITNGDIIEAAPFGSTADLIRLKGDSLWAVAEHSFALD
DENRTNCLQVSGLRIVIDPSKKIGSRVVKIDVMDNRNPKSEDLKPLDKNA
EYFIALPSYLADGKDGFSAMKKATARWTGPLDSDVFKSYVEKIKKVDKLK
WGRVIVCKAGSPCT
(Aedes albopictus polypeptide)
SEQ ID NO: 119
MKWSVYIALLVFAFLTSPVFSEENCNIPESELSKIDDVLRHMEKPIYSED
HYTSNNEECTNLLNGIHAQLRRLTQRYKLMNKGYVKVEEYKRMAEDYENQ
LKTLNAELLELQEHTSDKANAAIAKLKEDIKKLDEDVDTLHNKLKGIKQD
FEKVKRDLCLTYLNSNQMSNAKAKVKEMASTYLIEIIQQRLNTKYANIIP
MLDFSTAIPDLDDRGEAYKEIYKFIETHERLDGEDAVLLEASLLKMNATL
KEGSNITDERRTEIEKMLKELAEKSAVVFKTWSTELKGIEDTIIKYALDH
LFVNQMKVFGGIVGDTFEFAPIRHLLKLLVVCNNYYKVAAYKELIDRKIG
NVLGTIMFDLTTLEANEMSFDLHVPDEIPKLFNATLGSLPNSLTQLLPCL
NKVHVYNAKTNMCIVAPEDRFDVQQEKLTDFHRVVLAKYGCTAFRLESSP
NKASVKFVKPSGNALSSINLQLENDQWHSHVGTPTANKPDRKPSSSDEWI
LDANYVNDTVKIQSEFNEYKASQAEVDHLLVMDVKYLPHVVVGRYGVRGL
KRSSAKDTIEWYLKCAS
(Aedes albopictus polypeptide)
SEQ ID NO: 120
MAFNGIALLITATIFIGSCYANYCDSSLCRQGPHVACNAPQQFGPACGNN
RKFVPMDSKLKTIILNTHNKLRAEIANGMHGFPQAARMPTLVWDDELAHI
ASFNARKCIFAHDKCRNTRQFKFSGQNLAITTFYGFNFQAGDRAENFTQE
WFNEHKDCPKSYVDAYPSSHRGPQIGHFTQLVNDRTWKVGCSMMHYITNG
KMINYYLVCNYTMTNMIGEPIYTKGRTGSKCETGQNPQFKGLCSPREKVK
SESYNG
(Aedes albopictus polypeptide)
SEQ ID NO: 121
MCSTGLCLVFFIAQAVFLMNYSEQQTTVVMENGAISEKETNVDEVMTQFI
MKFYTKRFVEGQNLVVAPLLIFRVFMSMYGEMDASAKFDLHSLVGIPQEA
SAEKMSEFEAFANKYALPVGVQRNLVETRLYYDKSIGKIRSSLEAKSLKP
FPTNFADKQTFCNEVNTWIRNTPINGTDDLVHDYYLNNETAAFVAGALSI
DWNMQLKTSSDVKAFEGENVKFLEGSISTRYAKLDNLKVEVVEMVTDNLS
GVKLWLIMPDEASSIKKFNDQLSIASIRQIEKGLTALQKEDVALTVPMVT
IEYNSQEDAYVTEVFEVFSSLFSKPAVKPWFRVSVKDDLYAVKNFLMKCI
LRFVGSDAPADSKGQSTEKAVSFNRPFVMMILSKESNVPILLANYFSPKD
KLRALEAKERHLRMKAKEHLDL
(Aedes albopictus polypeptide)
SEQ ID NO: 122
MKILLAVVFVLNLTNLAVPQHLITSSPSLPESKPVGRRPTYEEYKQQRES
FLQTEDHHLLGANVTLTENEQLVNKFIMQMKLDEMEKGFNDSYNFIPARH
IFEVLDRFGQSKVFNVIRRLPKGGVLHAHDMALGSTDLIVNATYLENLWQ
KGNFGLNHGPEFKFSRERPGKEWSLVSEIRQWMTNEVYDAKVAEVFSLYN
ADPLNAYKSLDNVWSKFQNLFACLAPLITFAPVWRQYYHDSLKQFYDDHV
QYLEFRGVLPEVYDLDGKVYSAEEIVQLYYEETEQFKAKYPDFIGVKFIY
APGRYASDEEFQKLLDTTNRLHKKFPNFLAGFDLVGQEDPGRSLFEFAPA
LLKLPASINFFFHAGETNWYGMKTDQNLVDAVLLGTKRIGHGFAVLKHPK
VLKEIKRRQICIEINPISNQVLKLVQDQRNHPAALLFSDNYPVVVSSDDP
SFGRSTPLSHDFYVAFTGIASAKQDWRWLKQLALNSIEYSAMNSEEKTVA
KEKWNQAWDHQFSRLAVDFVAGKILENWIMKIV
(Aedes aegypti Polypeptide)
SEQ ID NO: 123
MQPRILHLTVLATIIGVALTANVPSTPGRKLNIPAFSNAGKTKGIEIWRI
ENFQPVAVPKAEYGKFYTGDSYLVLNTNEDKNKKKSYDVHFWLGLKTTQD
EAGSAAILTVQLDDLLGGGPVQHREVEGSESDLFLSYFKGGIRYLEGGVA
SGFKHVQTNAAHPKRLFHVKGAKNIRLRQVELAVSAMNKGDCFILDSDRD
VFVWVGPKANRVEKLKAINVANDIRDRDHNGRATVHIVDEFSTLSDQESF
FKSLGSGSPSTVPDQSTAKEDAAFEKADAARVELYKVTDSKAGKLAVEPI
TQKPLKQEMLKPDDAFILDTGSGLYVWIGKSATQQEKTQSLVKAQEFIKN
KKYPAWTPVERIVQNAETAPFKHFFQTWRDAGSTGSRLV
(Polypeptide)
SEQ ID NO: 124
MKSIVSITITVLAIICEGQATNYCDPSLCARGTPHIACNGLSTLSRTCGA
GSFEVALNRADQQLIVDLHNKLRSKVAMGQQKNSAGQRFQQACRMATLQW
DPELAHIAATNARRCVYGHDTCRNTASMKFAGQNIAIKYYYGMTFTNEQL
LTGFINSWFSEFKDATPQQIARYPANYRGPAIGHFTQIVSDRTSRIGCSM
VSYNKNGFINKLFVCNYGLTNIINQPVYVAGNVCSGCTTGCNKVFPGLCN
TAERVSNNP
(Polypeptide)
SEQ ID NO: 125
MKVYICQVIFSFLAVSVFCEENCNIPESELSKIDHVLRHMEKPIYSEEQF
ASDNEECTNLLNGIHAQLRRLTQRYKLMNKGYVKVEEYQRMADNYEKQLK
TLNDELVELQQHTSEKASATIAKLKEDIKKLDEEVGTLHEKLKGIKQDFE
KVKRDLCVTYLNSNQMSKAKAKLKEMASTYLIEIVQQQLNKSNANIMPML
EFSAAIPDLDDMGEAYKEIYKFLEEQKRLEGEDSVLLEATVLKMNASLKE
GSNITDERRTQIEGLLKDLATKSTIVFSTWTKELKKINDAVVIKNALDHM
FVSQMKVFGALVGDTSDFGSIRNFVKLTIVCNNYYKVAAYKELIDRKIGN
ALGTIMFDLLTLEVNEMKFDPHVPDEIPKLFEATLSSLPNSLTELRTCLG
KVQIYNKKTNKCVVATGNDFDVHKDKLGDFYRVVVADYGCTSFRLEASGD
KASVRIVTPSGNPMSNVNLHLEGNSLHNYVATPKSNKPDRTPSSSDEWIL
DANYNNDTIKIESQFSDYKTKKTEVDHLLVRDINHLPHVLVARYGFMGLK
NSDAKDTIEWNLKCGS
(Oligonucleotide)
SEQ ID NO: 126
TAATACGACTCACTATAGGGGATGGACAGATGTCTCTTCGTG
(Oligonucleotide)
SEQ ID NO: 127
TAATACGACTCACTATAGGGCCAAATCCAATCCATCGAAA
(Oligonucleotide)
SEQ ID NO: 128
TAATACGACTCACTATAGGGGTGAGCAAGGGCGAGGAG
(Oligonucleotide)
SEQ ID NO: 129
TAATACGACTCACTATAGGGCATGATATAGACGTTGTGGCTGTT

TABLE 1
Endogenous AgBR1 concentration of an A. aegypti salivary gland.

| Salivary gland | Relative Band | Estimated | Concentration |
|---|---|---|---|
| extract (SGE, μg) | Intensity | AgBR1 (ng) | (μM) |
| 4 | 0.0111 | 1.6 | 1.6-8.2 |

TABLE 2
List of upregulated genes at the bite site of mice
treated with control (naïve) serum.

| | | P-value | FDR step up | Ratio |
|---|---|---|---|---|
| | | (Naive- | (Naive- | (Naive) |
| | | control* | control* | control* |
| | | Bites | Bites | Bites |
| | | vs. | vs. | vs. |
| | | Naive- | Naive- | Naive- |
| | | control* | control* | control* |
| | Total | No | No | No |
| Gene ID | counts | bites) | bites) | bites) |
| Serpine1 | 9.20E+01 | 5.30E−06 | 2.67E−02 | 5.59E+00 |
| Chil3 | 5.58E+01 | 1.79E−06 | 2.67E−02 | 2.01E+02 |
| Ly6c1 | 5.53E+02 | 5.80E−04 | 9.70E−02 | 2.03E+00 |
| Ccl9 | 1.82E+02 | 3.19E−05 | 7.01E−02 | 8.69E+00 |
| Il1b | 2.91E+01 | 3.58E−06 | 2.67E−02 | 2.41E+01 |
| Msr1 | 5.87E+01 | 3.48E−05 | 7.01E−02 | 1.15E+01 |
| Ccl2 | 8.25E+01 | 1.76E−05 | 5.31E−02 | 3.01E+01 |
| Ly6a | 1.45E+03 | 1.63E−04 | 9.70E−02 | 2.49E+00 |
| Ifitm3 | 1.01E+03 | 4.38E−05 | 7.01E−02 | 3.15E+00 |
| Hsd11b1 | 1.23E+02 | 4.85E−03 | 1.67E−01 | 1.77E+00 |
| Cd53 | 1.35E+02 | 3.85E−05 | 7.01E−02 | 3.29E+00 |
| Ccl7 | 1.04E+02 | 4.63E−05 | 7.01E−02 | 2.07E+01 |
| Ccl8 | 3.03E+02 | 1.93E−04 | 9.70E−02 | 3.02E+00 |
| Hp | 1.59E+03 | 3.42E−02 | 3.01E−01 | 2.92E+00 |
| Lilrb4a | 7.02E+01 | 5.78E−05 | 7.28E−02 | 7.41E+00 |
| Ms4a6d | 8.59E+01 | 9.10E−05 | 8.61E−02 | 6.10E+00 |
| Ccl6 | 3.16E+02 | 1.03E−02 | 2.09E−01 | 1.67E+00 |
| Adm | 8.62E+01 | 9.03E−03 | 2.03E−01 | 3.22E+00 |
| Lyve1 | 2.09E+02 | 2.66E−03 | 1.46E−01 | 4.01E+00 |
| Slfn2 | 7.26E+01 | 2.94E−04 | 9.70E−02 | 4.01E+00 |
| Wfdc17 | 8.84E+01 | 2.27E−04 | 9.70E−02 | 4.79E+00 |
| Akr1b8 | 9.98E+01 | 3.92E−04 | 9.70E−02 | 2.32E+00 |
| Lrg1 | 1.82E+02 | 4.84E−02 | 3.35E−01 | 2.23E+00 |
| Icam1 | 1.41E+02 | 8.49E−05 | 8.56E−02 | 2.78E+00 |
| Cxcl14 | 3.03E+02 | 9.29E−04 | 1.13E−01 | 2.10E+00 |
| Ifitm2 | 1.94E+03 | 6.61E−04 | 1.04E−01 | 1.87E+00 |
| Ctsb | 3.28E+03 | 5.24E−03 | 1.70E−01 | 1.58E+00 |
| Upp1 | 1.37E+01 | 8.52E−04 | 1.09E−01 | 4.35E+00 |
| Bst1 | 2.99E+01 | 5.09E−04 | 9.70E−02 | 3.15E+00 |
| Timp1 | 1.49E+02 | 4.23E−04 | 9.70E−02 | 1.32E+01 |
| Ccnd3 | 4.42E+02 | 1.81E−03 | 1.30E−01 | 1.77E+00 |
| Csf1 | 2.94E+02 | 3.00E−03 | 1.48E−01 | 2.09E+00 |
| Li1r4b | 5.34E+01 | 6.39E−05 | 7.29E−02 | 5.52E+00 |
| Ifi204 | 2.09E+01 | 2.16E−02 | 2.62E−01 | 2.96E+00 |
| Ctla2a | 1.39E+02 | 2.31E−03 | 1.40E−01 | 3.49E+00 |
| Capn2 | 4.59E+02 | 1.98E−03 | 1.31E−01 | 1.72E+00 |
| Tmsb10 | 3.60E+02 | 6.31E−04 | 1.03E−01 | 2.56E+00 |
| C5ar1 | 1.41E+01 | 1.25E−03 | 1.20E−01 | 5.38E+00 |
| Serpina3m | 3.18E+01 | 6.71E−04 | 1.05E−01 | 6.61E+00 |
| Ccr5 | 1.90E+01 | 4.71E−04 | 9.70E−02 | 1.09E+01 |
| Gsta4 | 5.53E+01 | 1.03E−04 | 9.12E−02 | 4.54E+00 |
| Ddr2 | 4.69E+02 | 9.25E−04 | 1.13E−01 | 2.70E+00 |
| Ifi207 | 2.69E+01 | 1.74E−02 | 2.47E−01 | 2.14E+00 |
| Cfb | 1.58E+02 | 4.37E−02 | 3.26E−01 | 2.05E+00 |
| AC160962.1 | 2.06E+01 | 3.06E−03 | 1.48E−01 | 4.65E+00 |
| Gm6560 | 6.28E+02 | 2.07E−04 | 9.70E−02 | 1.92E+00 |
| Fcgr2b | 1.62E+02 | 1.59E−03 | 1.30E−01 | 2.79E+00 |
| Ccr1 | 4.16E+01 | 1.68E−04 | 9.70E−02 | 7.47E+00 |
| AB124611 | 1.28E+01 | 9.80E−04 | 1.14E−01 | 4.62E+00 |
| Spp1 | 1.26E+02 | 8.04E−04 | 1.08E−01 | 6.08E+00 |
| Hspa5 | 2.59E+03 | 4.64E−04 | 9.70E−02 | 1.79E+00 |
| Trim46 | 1.01E+01 | 3.43E−02 | 3.01E−01 | 1.92E+00 |
| Clec7a | 2.60E+01 | 1.48E−04 | 9.70E−02 | 8.27E+00 |
| Dok2 | 5.82E+01 | 4.70E−03 | 1.66E−01 | 3.96E+00 |
| Srgn | 1.02E+02 | 5.09E−04 | 9.70E−02 | 4.53E+00 |
| Sprr1b | 1.09E+02 | 2.63E−04 | 9.70E−02 | 2.37E+01 |
| Calr | 2.92E+03 | 3.18E−04 | 9.70E−02 | 2.12E+00 |
| Clec5a | 1.64E+01 | 1.58E−03 | 1.30E−01 | 3.67E+00 |
| Ms4a4a | 3.02E+01 | 7.70E−04 | 1.08E−01 | 5.15E+00 |
| Pxdc1 | 9.46E+01 | 6.38E−03 | 1.82E−01 | 1.51E+00 |
| Pdpn | 7.96E+01 | 9.06E−03 | 2.03E−01 | 2.62E+00 |
| Serpina3n | 1.93E+03 | 4.84E−04 | 9.70E−02 | 6.74E+00 |
| Ptafr | 2.48E+01 | 4.85E−03 | 1.67E−01 | 2.74E+00 |
| Mrc1 | 3.18E+02 | 3.42E−03 | 1.52E−01 | 3.22E+00 |
| Tmem8 | 3.89E+01 | 1.07E−03 | 1.15E−01 | 4.00E+00 |
| Plaur | 4.55E+01 | 1.81E−03 | 1.30E−01 | 5.19E+00 |
| Gm15922 | 2.45E+01 | 3.32E−03 | 1.50E−01 | 4.81E+00 |
| Cd163 | 1.66E+02 | 6.23E−03 | 1.81E−01 | 2.30E+00 |
| Samhd1 | 2.75E+02 | 2.34E−04 | 9.70E−02 | 2.42E+00 |
| Lcp2 | 2.05E+01 | 4.09E−03 | 1.62E−01 | 2.68E+00 |
| Ifnar2 | 1.29E+02 | 5.96E−03 | 1.80E−01 | 1.67E+00 |
| Mfsd10 | 1.95E+02 | 3.35E−04 | 9.70E−02 | 1.64E+00 |
| Ccr2 | 1.38E+02 | 1.95E−03 | 1.31E−01 | 6.36E+00 |
| Tnfaip2 | 9.92E+01 | 6.50E−03 | 1.82E−01 | 1.56E+00 |
| Cd68 | 1.53E+02 | 1.50E−02 | 2.34E−01 | 2.29E+00 |
| Lbp | 2.77E+02 | 9.22E−03 | 2.04E−01 | 2.45E+00 |
| Lrrc25 | 2.93E+01 | 1.42E−03 | 1.29E−01 | 3.85E+00 |
| Anxa3 | 4.44E+01 | 3.56E−02 | 3.05E−01 | 2.61E+00 |
| Hdc | 3.97E+01 | 1.79E−03 | 1.30E−01 | 3.52E+00 |
| Lyz2 | 1.25E+03 | 1.18E−02 | 2.19E−01 | 2.37E+00 |
| Cebpd | 1.35E+02 | 1.29E−02 | 2.24E−01 | 1.96E+00 |
| Clec4d | 2.23E+01 | 1.87E−04 | 9.70E−02 | 2.17E+01 |
| Tnfrsf1b | 6.68E+01 | 2.67E−03 | 1.46E−01 | 3.77E+00 |
| Manf | 4.85E+02 | 1.55E−04 | 9.70E−02 | 1.86E+00 |
| Hk3 | 1.48E+01 | 5.47E−03 | 1.72E−01 | 5.51E+00 |
| Cyp7b1 | 3.90E+01 | 5.84E−04 | 9.70E−02 | 3.99E+00 |
| Parm1 | 3.93E+01 | 2.22E−02 | 2.63E−01 | 1.96E+00 |
| Msn | 8.65E+02 | 4.35E−03 | 1.64E−01 | 1.90E+00 |
| Plek | 1.10E+02 | 7.40E−04 | 1.06E−01 | 3.14E+00 |
| Tlr13 | 3.26E+01 | 6.50E−04 | 1.04E−01 | 4.59E+00 |
| Sema3a | 6.32E+01 | 2.90E−02 | 2.84E−01 | 2.07E+00 |
| Slc27a3 | 3.50E+02 | 3.33E−04 | 9.70E−02 | 3.11E+00 |
| Csf3r | 2.63E+01 | 1.43E−04 | 9.70E−02 | 1.39E+01 |
| Nek6 | 1.13E+02 | 1.20E−02 | 2.20E−01 | 1.54E+00 |
| Tubb6 | 2.04E+02 | 9.10E−03 | 2.03E−01 | 2.76E+00 |
| Capns1 | 1.15E+03 | 5.29E−03 | 1.71E−01 | 1.65E+00 |
| Lyn | 7.33E+01 | 3.21E−03 | 1.48E−01 | 2.74E+00 |
| Krt16 | 8.15E+02 | 2.27E−04 | 9.70E−02 | 4.32E+01 |
| Apbb1ip | 7.21E+01 | 1.65E−02 | 2.41E−01 | 2.50E+00 |
| Sirpb1c | 2.98E+01 | 5.22E−04 | 9.70E−02 | 1.59E+01 |
| Gm27029 | 1.25E+01 | 1.16E−02 | 2.18E−01 | 2.33E+00 |
| Fcer1g | 1.53E+02 | 2.65E−03 | 1.46E−01 | 3.38E+00 |
| Ptx3 | 2.16E+01 | 1.81E−03 | 1.30E−01 | 5.03E+00 |
| Cd14 | 6.62E+01 | 1.72E−03 | 1.30E−01 | 3.77E+00 |
| Cd7 | 1.69E+01 | 4.83E−02 | 3.35E−01 | 2.41E+00 |
| Mpeg1 | 1.56E+02 | 2.34E−03 | 1.40E−01 | 2.62E+00 |
| Cpne2 | 1.51E+02 | 7.32E−04 | 1.05E−01 | 1.92E+00 |
| Pdia4 | 4.10E+02 | 5.88E−03 | 1.79E−01 | 1.69E+00 |
| Creld2 | 2.13E+02 | 2.69E−04 | 9.70E−02 | 1.86E+00 |
| Atrip | 1.59E+01 | 3.82E−02 | 3.15E−01 | 1.65E+00 |
| C3ar1 | 5.14E+01 | 6.02E−03 | 1.80E−01 | 3.00E+00 |
| Svep1 | 2.91E+02 | 4.85E−02 | 3.35E−01 | 2.98E+00 |
| Gm3788 | 1.07E+01 | 1.85E−03 | 1.30E−01 | 3.38E+00 |
| Rnasel | 8.01E+01 | 3.56E−02 | 3.05E−01 | 1.70E+00 |
| Thbs1 | 1.83E+03 | 4.43E−02 | 3.28E−01 | 1.59E+00 |
| C1qb | 3.71E+02 | 3.90E−02 | 3.15E−01 | 2.05E+00 |
| Ftl1 | 2.68E+03 | 1.58E−02 | 2.38E−01 | 1.63E+00 |
| Ptprc | 1.11E+02 | 1.36E−02 | 2.27E−01 | 1.89E+00 |
| Angptl4 | 7.60E+01 | 2.04E−02 | 2.58E−01 | 2.15E+00 |
| Relt | 2.52E+01 | 5.73E−03 | 1.77E−01 | 3.00E+00 |
| Pdia6 | 9.19E+02 | 2.85E−03 | 1.48E−01 | 1.73E+00 |
| Serpina3e-ps | 1.36E+01 | 1.51E−03 | 1.30E−01 | 4.57E+00 |
| Clec4n | 4.65E+01 | 5.79E−04 | 9.70E−02 | 4.75E+00 |
| Wisp2 | 2.20E+02 | 7.80E−03 | 1.91E−01 | 3.16E+00 |
| Fgr | 2.53E+01 | 2.47E−03 | 1.42E−01 | 3.83E+00 |
| Layn | 1.39E+01 | 1.86E−02 | 2.52E−01 | 2.54E+00 |
| Pcdhb22 | 3.76E+01 | 1.25E−02 | 2.20E−01 | 2.10E+00 |
| Adam8 | 1.38E+02 | 5.23E−05 | 7.20E−02 | 2.74E+00 |
| Lgmn | 8.96E+02 | 8.53E−03 | 1.98E−01 | 2.26E+00 |
| March1 | 2.46E+01 | 4.18E−02 | 3.22E−01 | 1.53E+00 |
| Clec4a2 | 5.48E+01 | 2.46E−03 | 1.42E−01 | 5.46E+00 |
| Dab2 | 4.96E+02 | 1.77E−02 | 2.49E−01 | 2.56E+00 |
| Lcp1 | 3.02E+02 | 2.04E−02 | 2.58E−01 | 2.15E+00 |
| Cd300lb | 1.58E+01 | 7.28E−03 | 1.85E−01 | 4.52E+00 |
| Slco2a1 | 4.88E+02 | 3.11E−03 | 1.48E−01 | 1.54E+00 |
| Slc11a1 | 1.75E+01 | 2.38E−02 | 2.67E−01 | 3.01E+00 |
| Ceacam1 | 2.84E+01 | 3.88E−02 | 3.15E−01 | 2.64E+00 |
| Emilin2 | 1.63E+02 | 2.20E−02 | 2.63E−01 | 3.08E+00 |
| Dnajb11 | 4.71E+02 | 3.48E−04 | 9.70E−02 | 1.56E+00 |
| Rnf141 | 3.86E+02 | 1.41E−03 | 1.29E−01 | 1.52E+00 |
| N1rp3 | 9.65E+00 | 1.05E−03 | 1.15E−01 | 4.64E+00 |
| Itgb2 | 9.96E+01 | 1.89E−02 | 2.53E−01 | 3.05E+00 |
| Mcemp1 | 1.05E+01 | 1.40E−03 | 1.29E−01 | 5.17E+00 |
| Tmem150a | 4.66E+01 | 1.87E−02 | 2.53E−01 | 1.73E+00 |
| Cyba | 1.65E+02 | 2.91E−02 | 2.84E−01 | 2.16E+00 |
| Itgal | 1.54E+01 | 3.59E−03 | 1.55E−01 | 2.65E+00 |
| Gm9844 | 8.95E+01 | 4.47E−03 | 1.64E−01 | 2.40E+00 |
| Slc15a3 | 5.39E+01 | 1.67E−03 | 1.30E−01 | 1.84E+00 |
| Pfn1 | 1.74E+03 | 5.76E−04 | 9.70E−02 | 1.63E+00 |
| Fam49b | 1.71E+02 | 3.62E−03 | 1.55E−01 | 1.86E+00 |
| Ssbp4 | 1.49E+02 | 1.48E−02 | 2.34E−01 | 1.50E+00 |
| Cyth4 | 7.95E+01 | 1.40E−02 | 2.28E−01 | 3.25E+00 |
| Gda | 2.89E+02 | 6.60E−03 | 1.82E−01 | 2.17E+00 |
| Syk | 8.97E+01 | 1.21E−02 | 2.20E−01 | 1.88E+00 |
| Samsn1 | 1.35E+01 | 8.64E−03 | 1.99E−01 | 3.22E+00 |
| Nqo1 | 1.33E+02 | 6.75E−05 | 7.29E−02 | 2.30E+00 |
| Pik3r5 | 3.23E+01 | 1.47E−02 | 2.33E−01 | 2.30E+00 |
| Tpsab1 | 2.10E+02 | 5.58E−03 | 1.74E−01 | 2.82E+00 |
| Hsp90b1 | 2.00E+03 | 9.00E−03 | 2.03E−01 | 1.56E+00 |
| Mgp | 1.14E+03 | 3.24E−02 | 2.95E−01 | 2.61E+00 |
| F630028O10Rik | 1.25E+01 | 5.57E−04 | 9.70E−02 | 5.60E+00 |
| AI662270 | 2.54E+01 | 2.21E−02 | 2.63E−01 | 2.76E+00 |
| Cd200r1 | 1.63E+01 | 1.63E−02 | 2.41E−01 | 3.25E+00 |
| Pf4 | 1.79E+02 | 1.43E−02 | 2.29E−01 | 3.06E+00 |
| Fth1 | 6.18E+03 | 1.47E−02 | 2.33E−01 | 1.60E+00 |
| Smim3 | 7.18E+01 | 3.50E−02 | 3.03E−01 | 1.68E+00 |
| Sash3 | 2.15E+01 | 2.41E−02 | 2.67E−01 | 2.86E+00 |
| Cd48 | 3.84E+01 | 4.03E−02 | 3.17E−01 | 1.93E+00 |
| Mcoln2 | 2.57E+01 | 1.52E−02 | 2.35E−01 | 2.57E+00 |
| Adgrg3 | 1.46E+01 | 2.66E−02 | 2.78E−01 | 2.84E+00 |
| Coro1a | 2.53E+02 | 2.16E−02 | 2.62E−01 | 2.72E+00 |
| Pkm | 4.24E+03 | 4.04E−03 | 1.62E−01 | 1.79E+00 |
| Pgk1-rs7 | 5.00E+02 | 1.32E−02 | 2.25E−01 | 1.58E+00 |
| Tyrobp | 1.88E+02 | 9.84E−03 | 2.09E−01 | 2.62E+00 |
| Gm5537 | 5.58E+02 | 1.17E−02 | 2.19E−01 | 1.59E+00 |
| Slc7a8 | 4.67E+01 | 1.65E−02 | 2.41E−01 | 2.89E+00 |
| Armc6 | 6.56E+01 | 3.68E−02 | 3.10E−01 | 1.54E+00 |
| Fam69a | 1.81E+02 | 3.29E−04 | 9.70E−02 | 1.66E+00 |
| Gcnt1 | 3.90E+01 | 6.25E−03 | 1.81E−01 | 2.81E+00 |
| Ccl22 | 2.26E+01 | 2.84E−02 | 2.83E−01 | 3.27E+00 |
| Sprr2a2 | 1.35E+03 | 6.12E−04 | 1.01E−01 | 2.83E+01 |
| Pirb | 7.96E+01 | 2.93E−02 | 2.85E−01 | 2.92E+00 |
| Myo1g | 5.57E+01 | 4.37E−02 | 3.26E−01 | 2.24E+00 |
| Ddah1 | 2.20E+01 | 2.39E−02 | 2.67E−01 | 4.12E+00 |
| Ms4a6c | 4.63E+01 | 1.62E−02 | 2.40E−01 | 3.36E+00 |
| Neto2 | 1.22E+01 | 7.31E−03 | 1.85E−01 | 2.24E+00 |
| Il1rl1 | 1.93E+01 | 2.39E−02 | 2.67E−01 | 3.21E+00 |
| Ptprj | 1.25E+02 | 1.13E−02 | 2.16E−01 | 1.51E+00 |
| Sdf2l1 | 8.91E+01 | 1.90E−02 | 2.53E−01 | 1.89E+00 |
| Sprr2a3 | 7.74E+01 | 5.39E−04 | 9.70E−02 | 3.08E+01 |
| Pira2 | 1.72E+01 | 4.88E−03 | 1.67E−01 | 5.17E+00 |
| Serpinb1a | 4.03E+02 | 4.35E−02 | 3.26E−01 | 1.77E+00 |
| Gm9025 | 2.82E+01 | 3.00E−02 | 2.86E−01 | 2.17E+00 |
| Flot2 | 3.35E+02 | 2.20E−02 | 2.63E−01 | 1.82E+00 |
| Il2rg | 2.43E+01 | 3.86E−02 | 3.15E−01 | 3.01E+00 |
| Nfkbie | 3.26E+01 | 1.59E−02 | 2.38E−01 | 1.74E+00 |
| Thy1 | 8.20E+02 | 1.03E−02 | 2.09E−01 | 2.42E+00 |
| Tmprss11g | 4.99E+01 | 6.90E−03 | 1.84E−01 | 2.67E+00 |
| Clec4a1 | 5.67E+01 | 3.25E−02 | 2.95E−01 | 2.57E+00 |
| Zyx | 4.88E+02 | 3.28E−02 | 2.96E−01 | 1.52E+00 |
| Ecscr | 7.33E+01 | 4.97E−02 | 3.38E−01 | 2.39E+00 |
| Pdia3 | 2.30E+03 | 2.49E−03 | 1.42E−01 | 1.56E+00 |
| 2610528A11Rik | 3.03E+02 | 5.30E−04 | 9.70E−02 | 1.95E+02 |
| Tlr2 | 5.09E+01 | 4.91E−02 | 3.37E−01 | 1.56E+00 |
| Impdh2 | 2.79E+02 | 2.43E−04 | 9.70E−02 | 1.70E+00 |
| Krt6b | 9.71E+02 | 1.63E−03 | 1.30E−01 | 2.84E+01 |
| Fscn1 | 9.94E+01 | 5.80E−03 | 1.77E−01 | 3.12E+00 |
| Tmem173 | 5.77E+01 | 1.69E−02 | 2.44E−01 | 2.91E+00 |
| Cdhr1 | 5.32E+01 | 1.08E−04 | 9.12E−02 | 4.82E+01 |
| Ifitm1 | 6.70E+01 | 2.95E−03 | 1.48E−01 | 3.26E+00 |
| Cytip | 5.53E+01 | 1.17E−02 | 2.19E−01 | 1.72E+00 |
| Psmb10 | 1.52E+02 | 2.89E−02 | 2.84E−01 | 1.57E+00 |
| Marcks | 3.94E+02 | 2.22E−02 | 2.63E−01 | 1.52E+00 |
| Pcbp3 | 2.45E+01 | 5.12E−03 | 1.69E−01 | 2.23E+00 |
| Cfl1 | 2.01E+03 | 1.55E−03 | 1.30E−01 | 1.70E+00 |
| Hck | 1.58E+01 | 4.45E−03 | 1.64E−01 | 3.18E+00 |
| Tnfrsf13b | 1.79E+01 | 3.38E−02 | 3.00E−01 | 2.71E+00 |
| Traf1 | 1.33E+01 | 2.52E−02 | 2.71E−01 | 2.54E+00 |
| Ap1m1 | 3.61E+02 | 1.70E−03 | 1.30E−01 | 1.53E+00 |
| Jam1 | 2.09E+01 | 3.51E−02 | 3.03E−01 | 1.63E+00 |
| Vav1 | 2.92E+01 | 1.33E−02 | 2.25E−01 | 2.43E+00 |
| Csf2rb | 1.09E+02 | 1.10E−02 | 2.14E−01 | 2.27E+00 |
| Impdh1 | 1.57E+02 | 2.19E−03 | 1.37E−01 | 1.62E+00 |
| Irf8 | 3.21E+01 | 2.87E−02 | 2.84E−01 | 2.17E+00 |
| Slc7a11 | 7.83E+01 | 1.87E−03 | 1.30E−01 | 2.47E+00 |
| Igsf6 | 1.94E+01 | 1.37E−02 | 2.27E−01 | 3.35E+00 |
| Cd300a | 2.59E+01 | 2.02E−02 | 2.58E−01 | 2.81E+00 |
| Dynlt1b | 2.52E+02 | 9.72E−03 | 2.07E−01 | 1.62E+00 |
| Laptm5 | 2.92E+02 | 2.87E−02 | 2.84E−01 | 1.96E+00 |
| Ccl12 | 2.35E+01 | 6.88E−03 | 1.84E−01 | 9.89E+00 |
| Ldha | 3.49E+03 | 2.87E−02 | 2.84E−01 | 1.51E+00 |
| Tagln2 | 1.10E+03 | 3.16E−03 | 1.48E−01 | 1.60E+00 |
| Irak4 | 6.75E+01 | 1.09E−02 | 2.13E−01 | 1.54E+00 |
| Fyb | 3.08E+01 | 9.37E−03 | 2.05E−01 | 2.20E+00 |
| Cxcl1 | 2.67E+01 | 5.21E−04 | 9.70E−02 | 1.31E+01 |
| Nme1 | 4.78E+02 | 2.33E−03 | 1.40E−01 | 1.85E+00 |
| Ripor2 | 1.85E+01 | 1.83E−02 | 2.51E−01 | 2.27E+00 |
| Ncf4 | 4.40E+01 | 2.91E−02 | 2.84E−01 | 2.05E+00 |
| Impdh2-ps | 7.61E+02 | 5.07E−04 | 9.70E−02 | 1.71E+00 |
| Alg8 | 2.43E+01 | 5.03E−03 | 1.67E−01 | 1.92E+00 |
| Mthfd2 | 4.07E+01 | 4.38E−02 | 3.26E−01 | 1.90E+00 |
| Flvcr1 | 8.70E+01 | 1.35E−02 | 2.26E−01 | 1.54E+00 |
| Ncf2 | 3.28E+01 | 3.34E−02 | 2.98E−01 | 1.93E+00 |
| Dusp6 | 1.79E+02 | 3.18E−02 | 2.93E−01 | 1.78E+00 |
| Flna | 1.39E+03 | 3.94E−02 | 3.15E−01 | 1.68E+00 |
| Etv4 | 3.57E+01 | 1.29E−02 | 2.24E−01 | 1.78E+00 |
| Rab31 | 2.88E+02 | 4.64E−03 | 1.65E−01 | 2.07E+00 |
| Bak1 | 1.33E+02 | 1.05E−02 | 2.11E−01 | 1.58E+00 |
| Lgals9 | 4.93E+02 | 1.10E−03 | 1.16E−01 | 2.58E+00 |
| Gm3839 | 1.15E+01 | 4.20E−02 | 3.22E−01 | 2.53E+00 |
| Prss12 | 1.39E+02 | 8.73E−03 | 1.99E−01 | 1.90E+00 |
| Hyou1 | 4.35E+02 | 4.51E−03 | 1.64E−01 | 1.57E+00 |
| 1810055G02Rik | 1.34E+02 | 4.12E−02 | 3.20E−01 | 1.51E+00 |
| Gm5837 | 1.14E+02 | 6.56E−03 | 1.82E−01 | 1.51E+00 |
| Ripk3 | 4.69E+01 | 9.61E−03 | 2.07E−01 | 3.15E+00 |
| Mmp9 | 6.44E+01 | 2.34E−02 | 2.67E−01 | 1.64E+00 |
| Ran | 1.04E+03 | 1.17E−03 | 1.19E−01 | 1.64E+00 |
| Gsdmc | 1.25E+02 | 2.91E−03 | 1.48E−01 | 4.77E+00 |
| Me2 | 7.01E+01 | 3.19E−02 | 2.93E−01 | 2.03E+00 |
| Ccl24 | 1.77E+01 | 2.03E−02 | 2.58E−01 | 5.13E+00 |
| Lrrc59 | 5.81E+02 | 4.04E−04 | 9.70E−02 | 1.61E+00 |
| Gmfg | 2.50E+01 | 3.57E−02 | 3.06E−01 | 1.77E+00 |
| Ddx39 | 3.46E+02 | 8.84E−04 | 1.11E−01 | 1.72E+00 |
| Tubb5 | 2.95E+03 | 1.10E−02 | 2.14E−01 | 2.01E+00 |
| Gmfg-ps | 3.56E+01 | 4.32E−02 | 3.24E−01 | 1.71E+00 |
| Dhfr | 1.31E+02 | 7.84E−03 | 1.92E−01 | 1.56E+00 |
| Gm4737 | 5.23E+02 | 1.84E−02 | 2.51E−01 | 1.68E+00 |
| Gm11451 | 2.09E+01 | 1.95E−02 | 2.55E−01 | 2.16E+00 |
| Csf2ra | 4.93E+01 | 4.77E−02 | 3.34E−01 | 1.74E+00 |
| Cdkn2d | 1.24E+02 | 4.99E−03 | 1.67E−01 | 1.56E+00 |
| Lrp8 | 4.18E+01 | 3.10E−04 | 9.70E−02 | 2.56E+00 |
| Evi2b | 2.81E+01 | 4.52E−02 | 3.28E−01 | 2.70E+00 |
| Pgk1 | 5.29E+02 | 2.82E−02 | 2.83E−01 | 1.69E+00 |
| Spi1 | 8.95E+01 | 3.31E−02 | 2.97E−01 | 2.16E+00 |
| Sorcs2 | 4.03E+01 | 4.56E−02 | 3.29E−01 | 2.15E+00 |
| Crispld2 | 1.11E+03 | 3.30E−03 | 1.50E−01 | 2.16E+00 |
| AC154572.2 | 5.38E+01 | 4.30E−02 | 3.23E−01 | 1.55E+00 |
| Ier5l | 3.26E+01 | 1.88E−02 | 2.53E−01 | 2.09E+00 |
| Ruvbl2 | 1.69E+02 | 1.54E−03 | 1.30E−01 | 1.63E+00 |
| Parp12 | 8.76E+01 | 6.96E−03 | 1.84E−01 | 1.54E+00 |
| AC123061.1 | 3.70E+01 | 4.53E−02 | 3.29E−01 | 1.67E+00 |
| Osmr | 2.05E+02 | 4.29E−03 | 1.63E−01 | 1.83E+00 |
| Dtymk | 1.43E+02 | 1.84E−02 | 2.51E−01 | 1.75E+00 |
| Gm6180 | 9.49E+01 | 5.39E−03 | 1.72E−01 | 1.74E+00 |
| Gm5851 | 9.09E+00 | 1.07E−02 | 2.12E−01 | 2.58E+00 |
| Gm2564 | 2.21E+01 | 1.21E−02 | 2.20E−01 | 3.89E+00 |
| Apol8 | 2.75E+01 | 2.33E−03 | 1.40E−01 | 2.46E+00 |
| Lcn2 | 2.92E+02 | 9.67E−03 | 2.07E−01 | 8.26E+00 |
| Hnrnpa3 | 9.53E+02 | 1.27E−02 | 2.23E−01 | 1.50E+00 |
| Eno1 | 5.06E+02 | 1.19E−03 | 1.20E−01 | 2.04E+00 |
| Csf2rb2 | 7.09E+01 | 3.42E−02 | 3.01E−01 | 1.76E+00 |
| Bcl3 | 5.31E+01 | 4.51E−03 | 1.64E−01 | 5.59E+00 |
| Ngf | 1.84E+01 | 7.12E−03 | 1.85E−01 | 3.69E+00 |
| Hpgd | 2.17E+02 | 2.34E−02 | 2.67E−01 | 1.53E+00 |
| Cks1b | 1.47E+02 | 3.47E−03 | 1.54E−01 | 1.62E+00 |
| Lsm4 | 1.98E+02 | 2.21E−02 | 2.63E−01 | 1.55E+00 |
| H13 | 6.32E+02 | 1.23E−03 | 1.20E−01 | 1.50E+00 |
| Fam162a | 1.70E+02 | 1.17E−02 | 2.19E−01 | 1.81E+00 |
| Eno1b | 1.75E+03 | 7.07E−04 | 1.05E−01 | 1.88E+00 |
| Txn1 | 8.38E+02 | 5.09E−04 | 9.70E−02 | 1.55E+00 |
| Marcksl1 | 1.50E+02 | 4.96E−04 | 9.70E−02 | 1.92E+00 |
| Vcan | 1.54E+02 | 3.92E−02 | 3.15E−01 | 2.86E+00 |
| Hmox1 | 9.55E+02 | 1.90E−03 | 1.31E−01 | 1.91E+00 |
| Il4ra | 3.07E+02 | 6.55E−03 | 1.82E−01 | 1.95E+00 |
| Crlf2 | 3.39E+01 | 1.69E−02 | 2.44E−01 | 1.92E+00 |
| Gm15725 | 3.82E+01 | 2.85E−02 | 2.84E−01 | 2.00E+00 |
| Enkd1 | 2.62E+01 | 1.04E−02 | 2.10E−01 | 1.80E+00 |
| Snrpa | 2.82E+02 | 6.65E−03 | 1.82E−01 | 1.51E+00 |
| Gins1 | 4.26E+01 | 1.92E−02 | 2.55E−01 | 1.88E+00 |
| Ifi35 | 1.96E+02 | 2.39E−02 | 2.67E−01 | 1.53E+00 |
| Gm15590 | 1.42E+01 | 4.46E−02 | 3.28E−01 | 1.99E+00 |
| Gjb1 | 1.03E+01 | 4.49E−02 | 3.28E−01 | 1.96E+00 |
| Ranbp1 | 3.45E+02 | 3.64E−03 | 1.55E−01 | 1.57E+00 |
| Brca2 | 9.93E+01 | 9.35E−04 | 1.13E−01 | 2.56E+00 |
| Slc39a14 | 9.67E+01 | 2.39E−03 | 1.41E−01 | 1.95E+00 |
| Arf2 | 3.56E+02 | 2.93E−02 | 2.85E−01 | 1.57E+00 |
| Gpr132 | 1.04E+01 | 4.78E−02 | 3.34E−01 | 1.77E+00 |
| Cad | 2.46E+02 | 3.64E−03 | 1.55E−01 | 1.67E+00 |
| Serpinc1 | 1.40E+01 | 1.74E−02 | 2.47E−01 | 1.98E+00 |
| Tslp | 1.96E+01 | 2.74E−02 | 2.81E−01 | 1.62E+00 |
| Snrpf | 1.18E+02 | 5.22E−03 | 1.70E−01 | 1.57E+00 |
| Hsp90aa1 | 8.11E+02 | 4.44E−03 | 1.64E−01 | 1.51E+00 |
| Gm6992 | 1.30E+01 | 4.43E−03 | 1.64E−01 | 2.58E+00 |
| 1110038B12Rik | 6.80E+01 | 1.47E−02 | 2.33E−01 | 1.50E+00 |
| Gm12758 | 2.09E+01 | 2.15E−02 | 2.62E−01 | 2.14E+00 |
| Gm11847 | 5.18E+01 | 2.49E−02 | 2.70E−01 | 1.77E+00 |
| Dusp2 | 1.09E+01 | 1.41E−02 | 2.28E−01 | 2.25E+00 |
| Cenps | 7.19E+01 | 1.64E−02 | 2.41E−01 | 1.64E+00 |
| Gm17383 | 3.70E+01 | 2.56E−02 | 2.73E−01 | 2.60E+00 |
| 2610203C20Rik | 1.13E+02 | 3.94E−02 | 3.15E−01 | 1.57E+00 |
| Alg3 | 5.95E+01 | 3.22E−03 | 1.48E−01 | 1.62E+00 |
| Gm5844 | 1.03E+02 | 1.31E−02 | 2.25E−01 | 1.57E+00 |
| Cenpt | 7.41E+01 | 5.15E−03 | 1.69E−01 | 1.71E+00 |
| Pglyrp1 | 1.35E+01 | 1.80E−02 | 2.50E−01 | 2.60E+00 |
| Pold2 | 2.44E+02 | 1.99E−02 | 2.57E−01 | 1.57E+00 |
| Slc16a14 | 1.38E+02 | 2.32E−02 | 2.66E−01 | 1.50E+00 |
| Nup43 | 8.94E+01 | 3.14E−03 | 1.48E−01 | 1.70E+00 |
| Acy1 | 6.13E+01 | 1.39E−02 | 2.28E−01 | 1.57E+00 |
| Fhl2 | 2.24E+01 | 9.21E−03 | 2.04E−01 | 1.98E+00 |
| Mcm3 | 2.79E+02 | 2.99E−02 | 2.86E−01 | 1.79E+00 |
| Gm20390 | 3.17E+02 | 4.84E−03 | 1.67E−01 | 1.83E+00 |
| Pfkl | 2.14E+02 | 2.96E−02 | 2.86E−01 | 1.54E+00 |
| Gm26809 | 8.67E+01 | 1.36E−04 | 9.70E−02 | 2.14E+00 |
| Mmp3 | 2.61E+02 | 8.43E−03 | 1.97E−01 | 4.04E+00 |
| Ints7 | 1.06E+02 | 3.89E−02 | 3.15E−01 | 1.53E+00 |
| Orc1 | 3.19E+01 | 1.25E−02 | 2.20E−01 | 2.08E+00 |
| Slc19a1 | 9.69E+01 | 1.94E−02 | 2.55E−01 | 1.53E+00 |
| Rbm14 | 2.04E+02 | 3.62E−03 | 1.55E−01 | 1.58E+00 |
| Gemin6 | 5.88E+01 | 1.78E−02 | 2.49E−01 | 1.52E+00 |
| Aldh18a1 | 1.28E+02 | 2.26E−02 | 2.65E−01 | 1.62E+00 |
| Dhrs13 | 2.46E+01 | 6.82E−03 | 1.84E−01 | 2.39E+00 |
| Mcm4 | 3.38E+02 | 2.13E−02 | 2.62E−01 | 1.51E+00 |
| Lsm2 | 8.84E+01 | 2.68E−03 | 1.46E−01 | 1.66E+00 |
| Pop1 | 5.15E+01 | 3.84E−03 | 1.58E−01 | 1.79E+00 |
| Chil1 | 2.16E+03 | 4.32E−03 | 1.63E−01 | 2.93E+00 |
| Nup93 | 2.32E+02 | 4.05E−03 | 1.62E−01 | 1.56E+00 |
| Slc29a2 | 4.36E+01 | 6.81E−03 | 1.84E−01 | 2.38E+00 |
| Vstm2a | 9.31E+00 | 2.41E−02 | 2.67E−01 | 1.99E+00 |
| Cdt1 | 1.24E+02 | 3.34E−02 | 2.98E−01 | 1.61E+00 |
| Rad51 | 7.52E+01 | 3.31E−02 | 2.97E−01 | 1.90E+00 |
| Mcm6 | 4.00E+02 | 3.61E−02 | 3.07E−01 | 1.84E+00 |
| Ercc6l | 4.30E+01 | 4.75E−02 | 3.34E−01 | 1.87E+00 |
| Ccne1 | 3.17E+01 | 2.26E−02 | 2.65E−01 | 2.01E+00 |
| Casp6 | 8.71E+01 | 2.83E−02 | 2.83E−01 | 1.51E+00 |
| Abcg2 | 2.24E+02 | 1.62E−03 | 1.30E−01 | 1.66E+00 |
| Gm5855 | 1.17E+01 | 6.87E−03 | 1.84E−01 | 2.34E+00 |
| Gm6977 | 5.53E+01 | 4.47E−02 | 3.28E−01 | 1.69E+00 |
| Tbl3 | 2.19E+02 | 1.60E−03 | 1.30E−01 | 1.58E+00 |
| BC100530 | 7.99E+01 | 2.73E−02 | 2.81E−01 | 1.93E+01 |
| Rpp40 | 4.22E+01 | 1.54E−02 | 2.37E−01 | 1.55E+00 |
| Lyar | 1.62E+02 | 4.42E−03 | 1.64E−01 | 1.53E+00 |
| Clec2l | 2.17E+01 | 3.35E−03 | 1.51E−01 | 3.76E+00 |
| Sprr2h | 4.07E+01 | 1.29E−02 | 2.24E−01 | 7.43E+00 |
| Ppia | 3.97E+03 | 3.64E−03 | 1.55E−01 | 1.50E+00 |
| Ndc1 | 1.34E+02 | 9.31E−03 | 2.05E−01 | 1.54E+00 |
| Bfsp1 | 1.48E+01 | 2.35E−02 | 2.67E−01 | 1.90E+00 |
| Skap2 | 4.01E+02 | 1.47E−03 | 1.29E−01 | 1.50E+00 |
| Smyd5 | 1.15E+02 | 2.39E−03 | 1.41E−01 | 1.60E+00 |
| Car12 | 2.72E+02 | 2.84E−03 | 1.48E−01 | 2.26E+00 |
| Casp3 | 1.40E+02 | 4.46E−02 | 3.28E−01 | 1.52E+00 |
| Fbxo10 | 9.27E+01 | 2.90E−02 | 2.84E−01 | 1.60E+00 |
| Lsm5 | 3.60E+01 | 3.32E−02 | 2.97E−01 | 1.61E+00 |
| Gstcd | 6.05E+01 | 1.74E−02 | 2.47E−01 | 1.64E+00 |
| Stom | 5.92E+02 | 6.92E−03 | 1.84E−01 | 1.66E+00 |
| Gm8203 | 3.02E+02 | 5.83E−04 | 9.70E−02 | 1.59E+00 |
| Gm13461 | 2.84E+02 | 6.90E−03 | 1.84E−01 | 1.64E+00 |
| Pgm3 | 6.75E+01 | 2.46E−02 | 2.69E−01 | 1.51E+00 |
| Erh | 2.54E+02 | 7.60E−03 | 1.88E−01 | 1.54E+00 |
| Fam171a2 | 1.20E+02 | 3.53E−04 | 9.70E−02 | 2.36E+00 |
| Xdh | 3.95E+02 | 4.86E−03 | 1.67E−01 | 1.55E+00 |
| Nfyb | 2.65E+02 | 4.29E−03 | 1.63E−01 | 1.61E+00 |
| Hbegf | 6.68E+01 | 1.45E−02 | 2.31E−01 | 1.78E+00 |
| Myof | 4.58E+02 | 2.93E−02 | 2.85E−01 | 1.57E+00 |
| Prss27 | 5.47E+01 | 1.71E−02 | 2.46E−01 | 2.14E+00 |
| Slc14a1 | 2.12E+01 | 8.28E−03 | 1.96E−01 | 3.46E+00 |
| Entpd7 | 3.80E+01 | 5.03E−03 | 1.67E−01 | 1.74E+00 |
| Gm7899 | 1.79E+01 | 4.70E−02 | 3.34E−01 | 2.05E+00 |
| Gm12988 | 1.34E+01 | 2.38E−02 | 2.67E−01 | 1.84E+00 |
| Tpcn2 | 9.41E+01 | 6.44E−03 | 1.82E−01 | 1.61E+00 |
| Srm | 3.12E+02 | 2.90E−03 | 1.48E−01 | 1.60E+00 |
| Has3 | 2.28E+01 | 1.64E−02 | 2.41E−01 | 4.85E+00 |
| Wdhd1 | 7.23E+01 | 4.28E−02 | 3.23E−01 | 1.76E+00 |
| Gm13092 | 1.22E+02 | 3.27E−02 | 2.95E−01 | 1.59E+00 |
| Socs3 | 3.30E+02 | 2.97E−03 | 1.48E−01 | 1.98E+00 |
| Anapc15-ps | 8.79E+00 | 3.30E−02 | 2.97E−01 | 1.73E+00 |
| Fosl1 | 1.73E+01 | 4.43E−02 | 3.28E−01 | 1.72E+00 |
| Nfkbid | 1.15E+01 | 3.99E−02 | 3.16E−01 | 1.64E+00 |
| Pim2 | 3.55E+01 | 1.39E−02 | 2.28E−01 | 1.60E+00 |
| Ada | 4.45E+01 | 1.62E−02 | 2.40E−01 | 1.76E+00 |
| Gm5791 | 1.93E+01 | 2.07E−02 | 2.60E−01 | 2.18E+00 |
| Gm11675 | 6.42E+01 | 4.76E−02 | 3.34E−01 | 1.55E+00 |
| Sema4c | 1.95E+02 | 2.51E−03 | 1.42E−01 | 1.52E+00 |
| Tes | 1.05E+02 | 6.69E−03 | 1.82E−01 | 1.54E+00 |
| Lrp11 | 2.50E+01 | 3.62E−02 | 3.07E−01 | 1.54E+00 |
| Tyms | 2.24E+02 | 4.88E−02 | 3.36E−01 | 1.53E+00 |
| Mtcl1 | 4.53E+01 | 2.00E−02 | 2.57E−01 | 2.78E+00 |
| Rfng | 3.04E+02 | 8.59E−04 | 1.09E−01 | 1.53E+00 |
| Snhg4 | 1.31E+02 | 1.59E−02 | 2.38E−01 | 1.61E+00 |
| Ndnf | 9.79E+01 | 4.44E−02 | 3.28E−01 | 3.12E+00 |
| Gm3379 | 3.54E+01 | 2.22E−02 | 2.63E−01 | 1.93E+00 |
| Trip13 | 4.31E+01 | 1.12E−02 | 2.16E−01 | 1.86E+00 |
| Sprr2g | 1.50E+01 | 3.00E−02 | 2.86E−01 | 6.51E+00 |
| Gpr176 | 1.05E+01 | 2.27E−02 | 2.66E−01 | 2.59E+00 |
| Panx1 | 1.73E+02 | 1.97E−03 | 1.31E−01 | 1.64E+00 |
| Stat3 | 1.34E+03 | 4.24E−03 | 1.63E−01 | 1.64E+00 |
| Gemin4 | 4.19E+01 | 4.68E−02 | 3.33E−01 | 2.10E+00 |
| Slc9a1 | 7.23E+02 | 7.90E−04 | 1.08E−01 | 1.58E+00 |
| Gm6210 | 5.01E+01 | 4.00E−02 | 3.16E−01 | 1.87E+00 |
| Eeflakmt4 | 5.38E+01 | 1.44E−02 | 2.30E−01 | 1.51E+00 |
| Gm5620 | 1.74E+01 | 3.96E−02 | 3.15E−01 | 1.67E+00 |
| Dnph1 | 1.53E+02 | 9.13E−03 | 2.03E−01 | 1.52E+00 |
| Gm12666 | 1.17E+01 | 4.36E−02 | 3.26E−01 | 1.63E+00 |
| S100a9 | 3.12E+02 | 2.90E−02 | 2.84E−01 | 1.76E+01 |
| Mrto4 | 1.67E+02 | 6.54E−03 | 1.82E−01 | 1.54E+00 |
| Stfa3 | 1.83E+01 | 7.01E−03 | 1.84E−01 | 5.45E+00 |
| Nkain1 | 1.22E+01 | 4.62E−03 | 1.65E−01 | 2.87E+00 |
| Psmg4 | 7.14E+01 | 5.49E−03 | 1.72E−01 | 1.50E+00 |
| S100a8 | 1.94E+02 | 1.37E−02 | 2.27E−01 | 1.22E+01 |
| Ampd3 | 1.73E+02 | 1.74E−02 | 2.47E−01 | 1.70E+00 |
| Duxbl2 | 1.76E+01 | 2.24E−02 | 2.63E−01 | 1.75E+00 |
| Rab15 | 3.41E+02 | 2.48E−02 | 2.70E−01 | 1.62E+00 |
| Gm27219 | 3.66E+01 | 2.15E−02 | 2.62E−01 | 1.71E+00 |
| Hmgb1-ps3 | 1.22E+01 | 3.76E−02 | 3.13E−01 | 1.96E+00 |
| Noc4l | 1.88E+02 | 1.99E−03 | 1.31E−01 | 1.78E+00 |
| Gm12722 | 1.10E+01 | 4.77E−02 | 3.34E−01 | 1.63E+00 |
| Dsg1c | 7.44E+01 | 2.07E−03 | 1.32E−01 | 2.04E+00 |
| 1700052K11Rik | 6.55E+01 | 4.96E−02 | 3.38E−01 | 1.55E+00 |
| Krt6a | 1.25E+03 | 2.14E−03 | 1.36E−01 | 3.99E+00 |
| Hirip3 | 1.12E+02 | 2.12E−02 | 2.62E−01 | 1.64E+00 |
| Pgam1 | 9.91E+02 | 2.48E−03 | 1.42E−01 | 1.63E+00 |
| B4galnt4 | 1.07E+01 | 4.00E−02 | 3.16E−01 | 1.83E+00 |
| Rpl10-ps5 | 8.71E+01 | 1.61E−02 | 2.40E−01 | 1.68E+00 |
| Hrh2 | 1.09E+01 | 2.93E−03 | 1.48E−01 | 2.72E+00 |
| AC131178.1 | 2.54E+01 | 1.76E−02 | 2.48E−01 | 1.99E+00 |
| Gadl1 | 7.43E+02 | 1.32E−02 | 2.25E−01 | 1.71E+00 |
| BC055324 | 3.24E+01 | 3.50E−02 | 3.03E−01 | 1.56E+00 |
| Gm6063 | 1.46E+01 | 4.49E−02 | 3.28E−01 | 1.93E+00 |
| Trex1 | 1.69E+02 | 2.64E−02 | 2.78E−01 | 1.54E+00 |
| Galnt6 | 8.87E+01 | 5.31E−03 | 1.71E−01 | 2.60E+00 |
| Gm3448 | 3.70E+01 | 3.41E−02 | 3.01E−01 | 1.89E+00 |
| Nat8l | 5.68E+02 | 2.80E−02 | 2.83E−01 | 1.66E+00 |
| AC022775.2 | 1.95E+01 | 2.54E−02 | 2.73E−01 | 1.59E+00 |
| Gm6793 | 2.30E+01 | 3.60E−02 | 3.06E−01 | 1.97E+00 |
| Mgat5b | 1.84E+01 | 9.60E−04 | 1.14E−01 | 3.43E+00 |
| Rpl29 | 4.19E+01 | 2.29E−02 | 2.66E−01 | 1.51E+00 |
| Gm11805 | 9.02E+00 | 3.77E−02 | 3.13E−01 | 1.72E+00 |
| Pla2g7 | 1.06E+02 | 3.05E−02 | 2.90E−01 | 1.58E+00 |
| AC154176.1 | 1.50E+02 | 3.09E−02 | 2.90E−01 | 1.62E+00 |
| Top2a | 3.70E+02 | 2.35E−02 | 2.67E−01 | 1.96E+00 |
| H2afx | 1.38E+02 | 1.79E−03 | 1.30E−01 | 1.90E+00 |
| Gtse1 | 4.83E+01 | 1.03E−03 | 1.14E−01 | 2.30E+00 |
| Gm12933 | 9.99E+00 | 2.85E−02 | 2.84E−01 | 2.18E+00 |
| Enah | 3.35E+02 | 1.80E−02 | 2.50E−01 | 1.51E+00 |
| Sbno2 | 6.21E+02 | 1.80E−02 | 2.50E−01 | 1.53E+00 |
| Tmem266 | 1.99E+01 | 7.37E−03 | 1.85E−01 | 2.19E+00 |
| Sprr2e | 1.87E+01 | 4.12E−02 | 3.20E−01 | 7.70E+00 |
| Slpi | 6.30E+01 | 1.39E−02 | 2.28E−01 | 5.25E+00 |
| Aldh1a3 | 2.09E+01 | 8.71E−03 | 1.99E−01 | 2.52E+00 |
| Kcnh1 | 1.81E+02 | 2.89E−02 | 2.84E−01 | 2.08E+00 |
| Cxcr2 | 5.99E+01 | 2.04E−02 | 2.58E−01 | 3.05E+00 |
| Rpl32-ps | 2.85E+01 | 4.88E−02 | 3.36E−01 | 1.52E+00 |
| Prnd | 1.11E+01 | 3.30E−02 | 2.97E−01 | 1.80E+00 |
| Cdh3 | 3.06E+02 | 4.83E−02 | 3.35E−01 | 1.65E+00 |
| Gpx2 | 7.00E+01 | 1.62E−02 | 2.40E−01 | 2.34E+00 |
| Grwd1 | 1.86E+02 | 1.62E−02 | 2.40E−01 | 1.56E+00 |
| Cenpw | 3.70E+01 | 1.98E−02 | 2.56E−01 | 1.72E+00 |
| Nop56 | 3.64E+02 | 7.01E−03 | 1.84E−01 | 1.65E+00 |
| Trp53i11 | 4.41E+02 | 3.23E−02 | 2.95E−01 | 1.57E+00 |
| Tctn3 | 4.25E+01 | 1.81E−02 | 2.50E−01 | 1.54E+00 |
| Gls2 | 1.11E+01 | 4.39E−02 | 3.26E−01 | 1.70E+00 |
| Gpcpd1 | 5.26E+02 | 5.81E−03 | 1.77E−01 | 1.58E+00 |
| Gpatch4 | 2.11E+02 | 1.83E−02 | 2.51E−01 | 1.61E+00 |
| Shank1 | 1.21E+01 | 3.30E−02 | 2.97E−01 | 1.65E+00 |
| Atp1a1 | 3.80E+03 | 3.26E−04 | 9.70E−02 | 1.95E+00 |
| Dctd | 5.11E+01 | 1.33E−02 | 2.25E−01 | 1.58E+00 |
| Ly6k | 2.85E+01 | 6.90E−04 | 1.05E−01 | 3.05E+00 |
| Rrp12 | 1.71E+02 | 1.86E−02 | 2.52E−01 | 1.54E+00 |
| Gch1 | 3.89E+01 | 2.92E−02 | 2.84E−01 | 1.58E+00 |
| Il13ra2 | 2.07E+01 | 2.45E−02 | 2.69E−01 | 2.24E+00 |
| Ccnf | 7.57E+01 | 1.80E−02 | 2.50E−01 | 1.56E+00 |
| Rp17a-ps10 | 1.33E+01 | 3.03E−02 | 2.88E−01 | 1.68E+00 |
| Plbd1 | 9.06E+02 | 1.25E−02 | 2.20E−01 | 1.57E+00 |
| Lrrc4 | 2.92E+01 | 2.30E−02 | 2.66E−01 | 3.11E+00 |
| Tdg-ps | 7.95E+01 | 4.43E−03 | 1.64E−01 | 1.57E+00 |
| Gm8666 | 4.81E+01 | 4.42E−02 | 3.28E−01 | 1.52E+00 |
| Gpr35 | 3.53E+01 | 4.74E−02 | 3.34E−01 | 1.67E+00 |
| Itpk1 | 1.47E+02 | 3.03E−02 | 2.88E−01 | 1.54E+00 |
| Nolc1 | 3.74E+02 | 6.02E−03 | 1.80E−01 | 1.52E+00 |
| Incenp | 1.22E+02 | 3.98E−02 | 3.16E−01 | 1.63E+00 |
| Oplah | 5.00E+02 | 1.14E−02 | 2.17E−01 | 1.50E+00 |
| Nt5dc2 | 3.10E+02 | 6.29E−03 | 1.82E−01 | 1.65E+00 |
| Tubb3 | 3.04E+01 | 2.63E−03 | 1.46E−01 | 2.40E+00 |
| Txn-ps1 | 1.37E+01 | 4.48E−02 | 3.28E−01 | 1.82E+00 |
| AI506816 | 2.81E+01 | 5.48E−03 | 1.72E−01 | 2.05E+00 |
| Gm15452 | 1.42E+01 | 4.10E−02 | 3.19E−01 | 1.67E+00 |
| Depdc1b | 1.71E+01 | 3.68E−02 | 3.10E−01 | 1.87E+00 |
| Epb4113 | 1.75E+02 | 1.49E−03 | 1.29E−01 | 1.52E+00 |
| Btbd19 | 9.47E+01 | 1.24E−02 | 2.20E−01 | 1.51E+00 |
| Igsf3 | 1.77E+02 | 2.30E−03 | 1.40E−01 | 1.71E+00 |
| Dusp9 | 1.28E+01 | 2.46E−02 | 2.69E−01 | 2.04E+00 |
| Cd44 | 7.00E+02 | 3.82E−04 | 9.70E−02 | 1.76E+00 |
| Gm45902 | 3.26E+01 | 2.80E−02 | 2.83E−01 | 1.74E+00 |
| Wfdc12 | 1.09E+02 | 1.08E−02 | 2.13E−01 | 4.99E+00 |
| Tdh | 8.38E+01 | 1.64E−02 | 2.41E−01 | 3.18E+00 |
| Rps13-ps2 | 5.42E+01 | 2.04E−02 | 2.58E−01 | 1.83E+00 |
| Poc1a | 4.29E+01 | 1.47E−02 | 2.33E−01 | 1.68E+00 |
| Gja1 | 9.24E+02 | 3.97E−02 | 3.16E−01 | 1.62E+00 |
| 9330102E08Rik | 2.34E+01 | 1.14E−02 | 2.17E−01 | 1.74E+00 |
| 2700038G22Rik | 1.48E+01 | 2.27E−02 | 2.66E−01 | 1.89E+00 |
| Rpl27 | 1.05E+02 | 3.76E−03 | 1.57E−01 | 1.70E+00 |
| Cdca8 | 1.05E+02 | 4.48E−02 | 3.28E−01 | 1.64E+00 |
| Stx11 | 3.97E+01 | 2.07E−02 | 2.60E−01 | 1.60E+00 |
| Fam167a | 5.88E+01 | 6.34E−03 | 1.82E−01 | 3.53E+00 |
| Pinx1 | 6.70E+01 | 1.88E−02 | 2.53E−01 | 1.54E+00 |
| Cks2 | 5.38E+01 | 2.41E−02 | 2.67E−01 | 1.56E+00 |
| Gm10182 | 1.40E+02 | 4.39E−02 | 3.26E−01 | 1.57E+00 |

| | | LSMean | LSMean |
|---|---|---|---|
| | | (Naive- | (Naive- |
| | Fold change | control*Bites) | control*No |
| | (Naive- | (Naive- | bites) (Naive- |
| | control*Bites | control*Bites | control*Bites |
| | vs. Naive- | vs. Naive- | vs. Naive- |
| | control*No | control*No | control*No |
| Gene ID | bites) | bites) | bites) |
| Serpine1 | 5.59E+00 | 1.56E+01 | 2.80E+00 |
| Chil3 | 2.01E+02 | 2.03E+01 | 1.01E−01 |
| Ly6c1 | 2.03E+00 | 6.95E+01 | 3.42E+01 |
| Cc19 | 8.69E+00 | 3.82E+01 | 4.39E+00 |
| Il1b | 2.41E+01 | 1.01E+01 | 4.18E−01 |
| Msr1 | 1.15E+01 | 1.45E+01 | 1.26E+00 |
| Ccl2 | 3.01E+01 | 2.64E+01 | 8.78E−01 |
| Ly6a | 2.49E+00 | 2.32E+02 | 9.31E+01 |
| Ifitm3 | 3.15E+00 | 1.93E+02 | 6.13E+01 |
| Hsd11b1 | 1.77E+00 | 1.35E+01 | 7.66E+00 |
| Cd53 | 3.29E+00 | 2.63E+01 | 7.98E+00 |
| Ccl7 | 2.07E+01 | 3.17E+01 | 1.53E+00 |
| Ccl8 | 3.02E+00 | 5.51E+01 | 1.82E+01 |
| Hp | 2.92E+00 | 9.19E+01 | 3.15E+01 |
| Lilrb4a | 7.41E+00 | 1.75E+01 | 2.37E+00 |
| Ms4a6d | 6.10E+00 | 1.98E+01 | 3.25E+00 |
| Ccl6 | 1.67E+00 | 4.04E+01 | 2.41E+01 |
| Adm | 3.22E+00 | 9.74E+00 | 3.03E+00 |
| Lyve1 | 4.01E+00 | 3.01E+01 | 7.50E+00 |
| Slfn2 | 4.01E+00 | 1.37E+01 | 3.41E+00 |
| Wfdc17 | 4.79E+00 | 1.90E+01 | 3.96E+00 |
| Akr1b8 | 2.32E+00 | 1.67E+01 | 7.23E+00 |
| Lrg1 | 2.23E+00 | 1.24E+01 | 5.55E+00 |
| Icam1 | 2.78E+00 | 2.64E+01 | 9.52E+00 |
| Cxcl14 | 2.10E+00 | 4.97E+01 | 2.37E+01 |
| Ifitm2 | 1.87E+00 | 2.97E+02 | 1.58E+02 |
| Ctsb | 1.58E+00 | 4.42E+02 | 2.79E+02 |
| Upp1 | 4.35E+00 | 2.60E+00 | 5.98E−01 |
| Bst1 | 3.15E+00 | 5.58E+00 | 1.77E+00 |
| Timp1 | 1.32E+01 | 3.57E+01 | 2.70E+00 |
| Ccnd3 | 1.77E+00 | 6.52E+01 | 3.68E+01 |
| Csf1 | 2.09E+00 | 4.33E+01 | 2.07E+01 |
| Li1r4b | 5.52E+00 | 1.34E+01 | 2.43E+00 |
| Ifi204 | 2.96E+00 | 1.85E+00 | 6.25E−01 |
| Ctla2a | 3.49E+00 | 2.27E+01 | 6.51E+00 |
| Capn2 | 1.72E+00 | 6.65E+01 | 3.87E+01 |
| Tmsb10 | 2.56E+00 | 6.42E+01 | 2.51E+01 |
| C5ar1 | 5.38E+00 | 2.54E+00 | 4.72E−01 |
| Serpina3m | 6.61E+00 | 6.98E+00 | 1.06E+00 |
| Ccr5 | 1.09E+01 | 4.69E+00 | 4.30E−01 |
| Gsta4 | 4.54E+00 | 1.28E+01 | 2.82E+00 |
| Ddr2 | 2.70E+00 | 7.94E+01 | 2.94E+01 |
| Ifi207 | 2.14E+00 | 2.99E+00 | 1.40E+00 |
| Cfb | 2.05E+00 | 1.64E+01 | 8.00E+00 |
| AC160962.1 | 4.65E+00 | 3.39E+00 | 7.28E−01 |
| Gm6560 | 1.92E+00 | 1.05E+02 | 5.48E+01 |
| Fcgr2b | 2.79E+00 | 2.66E+01 | 9.51E+00 |
| Ccrl | 7.47E+00 | 1.10E+01 | 1.47E+00 |
| AB124611 | 4.62E+00 | 2.61E+00 | 5.66E−01 |
| Spp1 | 6.08E+00 | 2.74E+01 | 4.52E+00 |
| Hspa5 | 1.79E+00 | 4.19E+02 | 2.34E+02 |
| Trim46 | 1.92E+00 | 1.03E+00 | 5.37E−01 |
| Clec7a | 8.27E+00 | 6.73E+00 | 8.13E−01 |
| Dok2 | 3.96E+00 | 9.02E+00 | 2.28E+00 |
| Srgn | 4.53E+00 | 2.15E+01 | 4.76E+00 |
| Sprr1b | 2.37E+01 | 3.02E+01 | 1.27E+00 |
| Calr | 2.12E+00 | 5.11E+02 | 2.41E+02 |
| Clec5a | 3.67E+00 | 2.96E+00 | 8.07E−01 |
| Ms4a4a | 5.15E+00 | 6.36E+00 | 1.24E+00 |
| Pxdc1 | 1.51E+00 | 1.27E+01 | 8.39E+00 |
| Pdpn | 2.62E+00 | 1.13E+01 | 4.30E+00 |
| Serpina3n | 6.74E+00 | 4.71E+02 | 6.98E+01 |
| Ptafr | 2.74E+00 | 3.49E+00 | 1.27E+00 |
| Mrc1 | 3.22E+00 | 5.31E+01 | 1.65E+01 |
| Tmem8 | 4.00E+00 | 7.84E+00 | 1.96E+00 |
| Plaur | 5.19E+00 | 9.07E+00 | 1.75E+00 |
| Gm15922 | 4.81E+00 | 4.46E+00 | 9.28E−01 |
| Cd163 | 2.30E+00 | 2.34E+01 | 1.01E+01 |
| Samhd1 | 2.42E+00 | 5.01E+01 | 2.07E+01 |
| Lcp2 | 2.68E+00 | 3.08E+00 | 1.15E+00 |
| Ifnar2 | 1.67E+00 | 1.84E+01 | 1.10E+01 |
| Mfsd10 | 1.64E+00 | 3.05E+01 | 1.87E+01 |
| Ccr2 | 6.36E+00 | 2.89E+01 | 4.55E+00 |
| Tnfaip2 | 1.56E+00 | 1.32E+01 | 8.48E+00 |
| Cd68 | 2.29E+00 | 2.15E+01 | 9.41E+00 |
| Lbp | 2.45E+00 | 4.15E+01 | 1.70E+01 |
| Lrrc25 | 3.85E+00 | 5.87E+00 | 1.52E+00 |
| Anxa3 | 2.61E+00 | 5.16E+00 | 1.97E+00 |
| Hdc | 3.52E+00 | 7.59E+00 | 2.16E+00 |
| Lyz2 | 2.37E+00 | 1.75E+02 | 7.42E+01 |
| Cebpd | 1.96E+00 | 1.87E+01 | 9.54E+00 |
| C1ec4d | 2.17E+01 | 7.92E+00 | 3.65E−01 |
| Tnfrsf1b | 3.77E+00 | 1.21E+01 | 3.21E+00 |
| Manf | 1.86E+00 | 8.35E+01 | 4.49E+01 |
| Hk3 | 5.51E+00 | 2.57E+00 | 4.66E−01 |
| Cyp7b1 | 3.99E+00 | 8.66E+00 | 2.17E+00 |
| Parm1 | 1.96E+00 | 5.05E+00 | 2.58E+00 |
| Msn | 1.90E+00 | 1.31E+02 | 6.90E+01 |
| Plek | 3.14E+00 | 2.08E+01 | 6.63E+00 |
| Tlr13 | 4.59E+00 | 7.40E+00 | 1.61E+00 |
| Sema3a | 2.07E+00 | 8.41E+00 | 4.07E+00 |
| Slc27a3 | 3.11E+00 | 7.11E+01 | 2.29E+01 |
| Csf3r | 1.39E+01 | 8.48E+00 | 6.08E−01 |
| Nek6 | 1.54E+00 | 1.59E+01 | 1.03E+01 |
| Tubb6 | 2.76E+00 | 3.15E+01 | 1.14E+01 |
| Capns1 | 1.65E+00 | 1.68E+02 | 1.02E+02 |
| Lyn | 2.74E+00 | 1.26E+01 | 4.60E+00 |
| Krt16 | 4.32E+01 | 3.07E+02 | 7.09E+00 |
| Apbb1ip | 2.50E+00 | 1.03E+01 | 4.13E+00 |
| Sirpb1c | 1.59E+01 | 9.34E+00 | 5.86E−01 |
| Gm27029 | 2.33E+00 | 1.85E+00 | 7.93E−01 |
| Fcer1g | 3.38E+00 | 2.86E+01 | 8.45E+00 |
| Ptx3 | 5.03E+00 | 4.54E+00 | 9.03E−01 |
| Cd14 | 3.77E+00 | 1.30E+01 | 3.44E+00 |
| Cd7 | 2.41E+00 | 1.94E+00 | 8.05E−01 |
| Mpeg1 | 2.62E+00 | 2.64E+01 | 1.01E+01 |
| Cpne2 | 1.92E+00 | 2.54E+01 | 1.32E+01 |
| Pdia4 | 1.69E+00 | 6.09E+01 | 3.60E+01 |
| Creld2 | 1.86E+00 | 3.65E+01 | 1.97E+01 |
| Atrip | 1.65E+00 | 1.83E+00 | 1.11E+00 |
| C3ar1 | 3.00E+00 | 8.24E+00 | 2.75E+00 |
| Svep1 | 2.98E+00 | 3.87E+01 | 1.30E+01 |
| Gm3788 | 3.38E+00 | 2.10E+00 | 6.20E−01 |
| Rnasel | 1.70E+00 | 9.91E+00 | 5.82E+00 |
| Thbs1 | 1.59E+00 | 2.04E+02 | 1.28E+02 |
| C1qb | 2.05E+00 | 4.70E+01 | 2.29E+01 |
| Ftl1 | 1.63E+00 | 3.83E+02 | 2.35E+02 |
| Ptprc | 1.89E+00 | 1.59E+01 | 8.43E+00 |
| Angptl4 | 2.15E+00 | 1.07E+01 | 5.01E+00 |
| Relt | 3.00E+00 | 4.45E+00 | 1.48E+00 |
| Pdia6 | 1.73E+00 | 1.44E+02 | 8.35E+01 |
| Serpina3e-ps | 4.57E+00 | 3.06E+00 | 6.68E−01 |
| Clec4n | 4.75E+00 | 1.13E+01 | 2.37E+00 |
| Wisp2 | 3.16E+00 | 3.67E+01 | 1.16E+01 |
| Fgr | 3.83E+00 | 5.17E+00 | 1.35E+00 |
| Layn | 2.54E+00 | 2.00E+00 | 7.87E−01 |
| Pcdhb22 | 2.10E+00 | 5.66E+00 | 2.70E+00 |
| Adam8 | 2.74E+00 | 3.00E+01 | 1.09E+01 |
| Lgmn | 2.26E+00 | 1.46E+02 | 6.44E+01 |
| March1 | 1.53E+00 | 2.99E+00 | 1.95E+00 |
| Clec4a2 | 5.46E+00 | 1.22E+01 | 2.23E+00 |
| Dab2 | 2.56E+00 | 7.26E+01 | 2.83E+01 |
| Lcp1 | 2.15E+00 | 4.34E+01 | 2.02E+01 |
| Cd300lb | 4.52E+00 | 3.01E+00 | 6.67E−01 |
| Slco2a1 | 1.54E+00 | 7.37E+01 | 4.78E+01 |
| Slc11a1 | 3.01E+00 | 2.36E+00 | 7.86E−01 |
| Ceacam1 | 2.64E+00 | 3.62E+00 | 1.37E+00 |
| Emilin2 | 3.08E+00 | 2.39E+01 | 7.76E+00 |
| Dnajb11 | 1.56E+00 | 7.40E+01 | 4.75E+01 |
| Rnf141 | 1.52E+00 | 5.76E+01 | 3.80E+01 |
| Nlrp3 | 4.64E+00 | 2.18E+00 | 4.70E−01 |
| Itgb2 | 3.05E+00 | 1.58E+01 | 5.18E+00 |
| Mcemp1 | 5.17E+00 | 2.41E+00 | 4.66E−01 |
| Tmem150a | 1.73E+00 | 6.77E+00 | 3.92E+00 |
| Cyba | 2.16E+00 | 2.35E+01 | 1.09E+01 |
| Itgal | 2.65E+00 | 2.65E+00 | 1.00E+00 |
| Gm9844 | 2.40E+00 | 1.58E+01 | 6.59E+00 |
| Slc15a3 | 1.84E+00 | 8.85E+00 | 4.80E+00 |
| Pfn1 | 1.63E+00 | 2.80E+02 | 1.72E+02 |
| Fam49b | 1.86E+00 | 2.77E+01 | 1.49E+01 |
| Ssbp4 | 1.50E+00 | 2.09E+01 | 1.39E+01 |
| Cyth4 | 3.25E+00 | 1.31E+01 | 4.03E+00 |
| Gda | 2.17E+00 | 4.60E+01 | 2.12E+01 |
| Syk | 1.88E+00 | 1.32E+01 | 7.00E+00 |
| Samsn1 | 3.22E+00 | 2.15E+00 | 6.68E−01 |
| Nqo1 | 2.30E+00 | 2.66E+01 | 1.16E+01 |
| Pik3r5 | 2.30E+00 | 4.93E+00 | 2.14E+00 |
| Tpsab1 | 2.82E+00 | 3.19E+01 | 1.13E+01 |
| Hsp90b1 | 1.56E+00 | 2.94E+02 | 1.89E+02 |
| Mgp | 2.61E+00 | 1.57E+02 | 6.01E+01 |
| F630028O10Rik | 5.60E+00 | 3.34E+00 | 5.97E−01 |
| AI662270 | 2.76E+00 | 3.93E+00 | 1.42E+00 |
| Cd200r1 | 3.25E+00 | 2.68E+00 | 8.25E−01 |
| Pf4 | 3.06E+00 | 2.90E+01 | 9.49E+00 |
| Fth1 | 1.60E+00 | 8.98E+02 | 5.60E+02 |
| Smim3 | 1.68E+00 | 9.30E+00 | 5.53E+00 |
| Sash3 | 2.86E+00 | 3.44E+00 | 1.20E+00 |
| Cd48 | 1.93E+00 | 5.05E+00 | 2.62E+00 |
| Mcoln2 | 2.57E+00 | 4.01E+00 | 1.56E+00 |
| Adgrg3 | 2.84E+00 | 2.36E+00 | 8.32E−01 |
| Coro1a | 2.72E+00 | 4.10E+01 | 1.51E+01 |
| Pkm | 1.79E+00 | 6.66E+02 | 3.71E+02 |
| Pgk1-rs7 | 1.58E+00 | 7.17E+01 | 4.53E+01 |
| Tyrobp | 2.62E+00 | 3.24E+01 | 1.24E+01 |
| Gm5537 | 1.59E+00 | 8.07E+01 | 5.07E+01 |
| Slc7a8 | 2.89E+00 | 7.92E+00 | 2.74E+00 |
| Armc6 | 1.54E+00 | 8.89E+00 | 5.77E+00 |
| Fam69a | 1.66E+00 | 3.03E+01 | 1.83E+01 |
| Gcnt1 | 2.81E+00 | 6.91E+00 | 2.45E+00 |
| Ccl22 | 3.27E+00 | 2.99E+00 | 9.14E−01 |
| Sprr2a2 | 2.83E+01 | 4.92E+02 | 1.74E+01 |
| Pirb | 2.92E+00 | 1.24E+01 | 4.25E+00 |
| Myo1g | 2.24E+00 | 8.08E+00 | 3.61E+00 |
| Ddah1 | 4.12E+00 | 3.60E+00 | 8.74E−01 |
| Ms4a6c | 3.36E+00 | 8.35E+00 | 2.49E+00 |
| Neto2 | 2.24E+00 | 1.93E+00 | 8.63E−01 |
| Il1rl1 | 3.21E+00 | 2.81E+00 | 8.75E−01 |
| Ptprj | 1.51E+00 | 1.79E+01 | 1.19E+01 |
| Sdf2l1 | 1.89E+00 | 1.35E+01 | 7.16E+00 |
| Sprr2a3 | 3.08E+01 | 2.89E+01 | 9.40E−01 |
| Pira2 | 5.17E+00 | 3.76E+00 | 7.26E−01 |
| Serpinb1a | 1.77E+00 | 5.57E+01 | 3.15E+01 |
| Gm9025 | 2.17E+00 | 4.03E+00 | 1.85E+00 |
| Flot2 | 1.82E+00 | 4.82E+01 | 2.65E+01 |
| Il2rg | 3.01E+00 | 3.64E+00 | 1.21E+00 |
| Nfkbie | 1.74E+00 | 4.85E+00 | 2.79E+00 |
| Thy1 | 2.42E+00 | 1.41E+02 | 5.83E+01 |
| Tmprss11g | 2.67E+00 | 8.58E+00 | 3.21E+00 |
| Clec4a1 | 2.57E+00 | 8.87E+00 | 3.46E+00 |
| Zyx | 1.52E+00 | 6.58E+01 | 4.32E+01 |
| Ecscr | 2.39E+00 | 1.03E+01 | 4.30E+00 |
| Pdia3 | 1.56E+00 | 3.57E+02 | 2.28E+02 |
| 2610528A11Rik | 1.95E+02 | 1.39E+02 | 7.12E−01 |
| Tlr2 | 1.56E+00 | 6.58E+00 | 4.23E+00 |
| Impdh2 | 1.70E+00 | 4.59E+01 | 2.70E+01 |
| Krt6b | 2.84E+01 | 3.27E+02 | 1.15E+01 |
| Fscn1 | 3.12E+00 | 1.84E+01 | 5.90E+00 |
| Tmem173 | 2.91E+00 | 9.67E+00 | 3.33E+00 |
| Cdhr1 | 4.82E+01 | 2.33E+01 | 4.84E−01 |
| Ifitm1 | 3.26E+00 | 1.36E+01 | 4.17E+00 |
| Cytip | 1.72E+00 | 8.14E+00 | 4.73E+00 |
| Psmb10 | 1.57E+00 | 2.14E+01 | 1.37E+01 |
| Marcks | 1.52E+00 | 5.74E+01 | 3.77E+01 |
| Pcbp3 | 2.23E+00 | 4.12E+00 | 1.85E+00 |
| Cfl1 | 1.70E+00 | 3.31E+02 | 1.94E+02 |
| Hck | 3.18E+00 | 3.04E+00 | 9.57E−01 |
| Tnfrsfl3b | 2.71E+00 | 2.70E+00 | 9.96E−01 |
| Traf1 | 2.54E+00 | 1.96E+00 | 7.73E−01 |
| Ap1m1 | 1.53E+00 | 5.52E+01 | 3.61E+01 |
| Jaml | 1.63E+00 | 2.86E+00 | 1.76E+00 |
| Vav1 | 2.43E+00 | 4.93E+00 | 2.03E+00 |
| Csf2rb | 2.27E+00 | 1.80E+01 | 7.93E+00 |
| Impdh1 | 1.62E+00 | 2.50E+01 | 1.54E+01 |
| Irf8 | 2.17E+00 | 5.10E+00 | 2.36E+00 |
| Slc7a11 | 2.47E+00 | 1.52E+01 | 6.15E+00 |
| Igsf6 | 3.35E+00 | 3.56E+00 | 1.06E+00 |
| Cd300a | 2.81E+00 | 4.31E+00 | 1.53E+00 |
| Dynlt1b | 1.62E+00 | 3.86E+01 | 2.39E+01 |
| Laptm5 | 1.96E+00 | 4.40E+01 | 2.24E+01 |
| Ccl12 | 9.89E+00 | 6.08E+00 | 6.15E−01 |
| Ldha | 1.51E+00 | 4.92E+02 | 3.27E+02 |
| Tagln2 | 1.60E+00 | 1.74E+02 | 1.09E+02 |
| Irak4 | 1.54E+00 | 9.84E+00 | 6.38E+00 |
| Fyb | 2.20E+00 | 5.19E+00 | 2.36E+00 |
| Cxcl1 | 1.31E+01 | 9.04E+00 | 6.89E−01 |
| Nme1 | 1.85E+00 | 8.14E+01 | 4.39E+01 |
| Ripor2 | 2.27E+00 | 2.92E+00 | 1.29E+00 |
| Ncf4 | 2.05E+00 | 6.86E+00 | 3.35E+00 |
| Impdh2-ps | 1.71E+00 | 1.27E+02 | 7.42E+01 |
| Alg8 | 1.92E+00 | 4.05E+00 | 2.11E+00 |
| Mthfd2 | 1.90E+00 | 6.00E+00 | 3.15E+00 |
| Flvcrl | 1.54E+00 | 1.29E+01 | 8.39E+00 |
| Ncf2 | 1.93E+00 | 4.90E+00 | 2.54E+00 |
| Dusp6 | 1.78E+00 | 2.62E+01 | 1.48E+01 |
| Flna | 1.68E+00 | 1.99E+02 | 1.18E+02 |
| Etv4 | 1.78E+00 | 5.37E+00 | 3.02E+00 |
| Rab31 | 2.07E+00 | 4.88E+01 | 2.36E+01 |
| Bak1 | 1.58E+00 | 2.02E+01 | 1.27E+01 |
| Lgals9 | 2.58E+00 | 9.86E+01 | 3.82E+01 |
| Gm3839 | 2.53E+00 | 1.70E+00 | 6.69E−01 |
| Prss12 | 1.90E+00 | 2.25E+01 | 1.18E+01 |
| Hyou1 | 1.57E+00 | 6.65E+01 | 4.25E+01 |
| 1810055G02Rik | 1.51E+00 | 1.85E+01 | 1.23E+01 |
| Gm5837 | 1.51E+00 | 1.73E+01 | 1.15E+01 |
| Ripk3 | 3.15E+00 | 9.30E+00 | 2.95E+00 |
| Mmp9 | 1.64E+00 | 9.20E+00 | 5.62E+00 |
| Ran | 1.64E+00 | 1.71E+02 | 1.04E+02 |
| Gsdmc | 4.77E+00 | 2.90E+01 | 6.08E+00 |
| Me2 | 2.03E+00 | 1.10E+01 | 5.40E+00 |
| Ccl24 | 5.13E+00 | 3.24E+00 | 6.31E−01 |
| Lrrc59 | 1.61E+00 | 9.52E+01 | 5.92E+01 |
| Gmfg | 1.77E+00 | 3.57E+00 | 2.02E+00 |
| Ddx39 | 1.72E+00 | 5.81E+01 | 3.37E+01 |
| Tubb5 | 2.01E+00 | 4.95E+02 | 2.46E+02 |
| Gmfg-ps | 1.71E+00 | 5.06E+00 | 2.96E+00 |
| Dhfr | 1.56E+00 | 1.95E+01 | 1.25E+01 |
| Gm4737 | 1.68E+00 | 8.02E+01 | 4.77E+01 |
| Gm11451 | 2.16E+00 | 3.35E+00 | 1.55E+00 |
| Csf2ra | 1.74E+00 | 7.54E+00 | 4.34E+00 |
| Cdkn2d | 1.56E+00 | 1.92E+01 | 1.23E+01 |
| Lrp8 | 2.56E+00 | 8.36E+00 | 3.26E+00 |
| Evi2b | 2.70E+00 | 4.61E+00 | 1.71E+00 |
| Pgk1 | 1.69E+00 | 7.93E+01 | 4.70E+01 |
| Spi1 | 2.16E+00 | 1.47E+01 | 6.78E+00 |
| Sorcs2 | 2.15E+00 | 6.15E+00 | 2.86E+00 |
| Crispld2 | 2.16E+00 | 1.94E+02 | 8.97E+01 |
| AC154572.2 | 1.55E+00 | 7.70E+00 | 4.98E+00 |
| Ier5l | 2.09E+00 | 5.47E+00 | 2.62E+00 |
| Ruvbl2 | 1.63E+00 | 2.72E+01 | 1.67E+01 |
| Parp12 | 1.54E+00 | 1.32E+01 | 8.63E+00 |
| AC123061.1 | 1.67E+00 | 5.30E+00 | 3.17E+00 |
| Osmr | 1.83E+00 | 3.47E+01 | 1.90E+01 |
| Dtymk | 1.75E+00 | 2.24E+01 | 1.27E+01 |
| Gm6180 | 1.74E+00 | 1.58E+01 | 9.09E+00 |
| Gm5851 | 2.58E+00 | 1.81E+00 | 6.99E−01 |
| Gm2564 | 3.89E+00 | 4.66E+00 | 1.20E+00 |
| Apol8 | 2.46E+00 | 5.24E+00 | 2.13E+00 |
| Lcn2 | 8.26E+00 | 8.19E+01 | 9.91E+00 |
| Hnrnpa3 | 1.50E+00 | 1.45E+02 | 9.67E+01 |
| Eno1 | 2.04E+00 | 9.34E+01 | 4.59E+01 |
| Csf2rb2 | 1.76E+00 | 1.07E+01 | 6.05E+00 |
| Bcl3 | 5.59E+00 | 1.37E+01 | 2.46E+00 |
| Ngf | 3.69E+00 | 3.70E+00 | 1.00E+00 |
| Hpgd | 1.53E+00 | 3.26E+01 | 2.13E+01 |
| Cks1b | 1.62E+00 | 2.40E+01 | 1.48E+01 |
| Lsm4 | 1.55E+00 | 3.05E+01 | 1.97E+01 |
| H13 | 1.50E+00 | 9.97E+01 | 6.63E+01 |
| Fam162a | 1.81E+00 | 2.80E+01 | 1.55E+01 |
| Eno1b | 1.88E+00 | 3.12E+02 | 1.66E+02 |
| Txn1 | 1.55E+00 | 1.38E+02 | 8.91E+01 |
| Marcksl1 | 1.92E+00 | 2.71E+01 | 1.42E+01 |
| Vcan | 2.86E+00 | 2.83E+01 | 9.88E+00 |
| Hmox1 | 1.91E+00 | 1.71E+02 | 8.95E+01 |
| Il4ra | 1.95E+00 | 5.34E+01 | 2.74E+01 |
| Crlf2 | 1.92E+00 | 5.52E+00 | 2.87E+00 |
| Gm15725 | 2.00E+00 | 5.95E+00 | 2.98E+00 |
| Enkd1 | 1.80E+00 | 4.37E+00 | 2.43E+00 |
| Snrpa | 1.51E+00 | 4.47E+01 | 2.95E+01 |
| Gins1 | 1.88E+00 | 6.83E+00 | 3.62E+00 |
| Ifi35 | 1.53E+00 | 2.93E+01 | 1.92E+01 |
| Gm15590 | 1.99E+00 | 2.19E+00 | 1.10E+00 |
| Gjb1 | 1.96E+00 | 1.56E+00 | 7.98E−01 |
| Ranbp1 | 1.57E+00 | 5.49E+01 | 3.49E+01 |
| Brca2 | 2.56E+00 | 1.93E+01 | 7.55E+00 |
| Slc39a14 | 1.95E+00 | 1.76E+01 | 9.01E+00 |
| Arf2 | 1.57E+00 | 5.40E+01 | 3.44E+01 |
| Gpr132 | 1.77E+00 | 1.59E+00 | 8.96E−01 |
| Cad | 1.67E+00 | 3.92E+01 | 2.35E+01 |
| Serpinc1 | 1.98E+00 | 2.28E+00 | 1.15E+00 |
| Tslp | 1.62E+00 | 2.73E+00 | 1.69E+00 |
| Snrpf | 1.57E+00 | 1.87E+01 | 1.20E+01 |
| Hsp90aa1 | 1.51E+00 | 1.27E+02 | 8.46E+01 |
| Gm6992 | 2.58E+00 | 2.61E+00 | 1.01E+00 |
| 1110038B12Rik | 1.50E+00 | 1.06E+01 | 7.05E+00 |
| Gm12758 | 2.14E+00 | 3.60E+00 | 1.68E+00 |
| Gm11847 | 1.77E+00 | 8.10E+00 | 4.56E+00 |
| Dusp2 | 2.25E+00 | 2.13E+00 | 9.45E−01 |
| Cenps | 1.64E+00 | 1.10E+01 | 6.71E+00 |
| Gm17383 | 2.60E+00 | 6.52E+00 | 2.51E+00 |
| 2610203C20Rik | 1.57E+00 | 1.72E+01 | 1.09E+01 |
| Alg3 | 1.62E+00 | 9.64E+00 | 5.96E+00 |
| Gm5844 | 1.57E+00 | 1.66E+01 | 1.06E+01 |
| Cenpt | 1.71E+00 | 1.23E+01 | 7.15E+00 |
| Pglyrp1 | 2.60E+00 | 2.66E+00 | 1.02E+00 |
| Pold2 | 1.57E+00 | 3.72E+01 | 2.38E+01 |
| Slc16a14 | 1.50E+00 | 2.41E+01 | 1.61E+01 |
| Nup43 | 1.70E+00 | 1.47E+01 | 8.66E+00 |
| Acy1 | 1.57E+00 | 9.59E+00 | 6.09E+00 |
| Fhl2 | 1.98E+00 | 4.08E+00 | 2.06E+00 |
| Mcm3 | 1.79E+00 | 4.47E+01 | 2.50E+01 |
| Gm20390 | 1.83E+00 | 5.62E+01 | 3.07E+01 |
| Pfkl | 1.54E+00 | 3.22E+01 | 2.09E+01 |
| Gm26809 | 2.14E+00 | 1.72E+01 | 8.06E+00 |
| Mmp3 | 4.04E+00 | 6.56E+01 | 1.62E+01 |
| Ints7 | 1.53E+00 | 1.59E+01 | 1.04E+01 |
| Orc1 | 2.08E+00 | 5.60E+00 | 2.69E+00 |
| Slc19a1 | 1.53E+00 | 1.46E+01 | 9.57E+00 |
| Rbm14 | 1.58E+00 | 3.26E+01 | 2.07E+01 |
| Gemin6 | 1.52E+00 | 8.96E+00 | 5.88E+00 |
| Aldh18a1 | 1.62E+00 | 2.02E+01 | 1.25E+01 |
| Dhrs13 | 2.39E+00 | 5.01E+00 | 2.10E+00 |
| Mcm4 | 1.51E+00 | 5.11E+01 | 3.39E+01 |
| Lsm2 | 1.66E+00 | 1.49E+01 | 9.00E+00 |
| Pop1 | 1.79E+00 | 8.74E+00 | 4.88E+00 |
| Chil1 | 2.93E+00 | 4.85E+02 | 1.66E+02 |
| Nup93 | 1.56E+00 | 3.72E+01 | 2.39E+01 |
| Slc29a2 | 2.38E+00 | 8.24E+00 | 3.47E+00 |
| Vstm2a | 1.99E+00 | 1.57E+00 | 7.86E−01 |
| Cdt1 | 1.61E+00 | 1.93E+01 | 1.20E+01 |
| Rad51 | 1.90E+00 | 1.24E+01 | 6.55E+00 |
| Mcm6 | 1.84E+00 | 6.43E+01 | 3.49E+01 |
| Ercc6l | 1.87E+00 | 7.10E+00 | 3.80E+00 |
| Ccne1 | 2.01E+00 | 5.56E+00 | 2.77E+00 |
| Casp6 | 1.51E+00 | 1.30E+01 | 8.58E+00 |
| Abcg2 | 1.66E+00 | 3.83E+01 | 2.30E+01 |
| Gm5855 | 2.34E+00 | 2.21E+00 | 9.44E−01 |
| Gm6977 | 1.69E+00 | 8.99E+00 | 5.33E+00 |
| Tbl3 | 1.58E+00 | 3.58E+01 | 2.27E+01 |
| BC100530 | 1.93E+01 | 2.68E+01 | 1.39E+00 |
| Rpp40 | 1.55E+00 | 6.65E+00 | 4.28E+00 |
| Lyar | 1.53E+00 | 2.59E+01 | 1.70E+01 |
| Clec2l | 3.76E+00 | 5.58E+00 | 1.48E+00 |
| Sprr2h | 7.43E+00 | 1.22E+01 | 1.64E+00 |
| Ppia | 1.50E+00 | 6.35E+02 | 4.22E+02 |
| Ndc1 | 1.54E+00 | 2.12E+01 | 1.38E+01 |
| Bfsp1 | 1.90E+00 | 2.57E+00 | 1.36E+00 |
| Skap2 | 1.50E+00 | 7.17E+01 | 4.78E+01 |
| Smyd5 | 1.60E+00 | 1.92E+01 | 1.20E+01 |
| Car12 | 2.26E+00 | 6.44E+01 | 2.85E+01 |
| Casp3 | 1.52E+00 | 2.10E+01 | 1.39E+01 |
| Fbxo10 | 1.60E+00 | 1.40E+01 | 8.71E+00 |
| Lsm5 | 1.61E+00 | 5.77E+00 | 3.58E+00 |
| Gstcd | 1.64E+00 | 9.84E+00 | 6.00E+00 |
| Stom | 1.66E+00 | 9.74E+01 | 5.88E+01 |
| Gm8203 | 1.59E+00 | 5.21E+01 | 3.28E+01 |
| Gm13461 | 1.64E+00 | 4.69E+01 | 2.87E+01 |
| Pgm3 | 1.51E+00 | 1.03E+01 | 6.78E+00 |
| Erh | 1.54E+00 | 4.10E+01 | 2.65E+01 |
| Fam171a2 | 2.36E+00 | 2.41E+01 | 1.02E+01 |
| Xdh | 1.55E+00 | 6.32E+01 | 4.08E+01 |
| Nfyb | 1.61E+00 | 4.35E+01 | 2.71E+01 |
| Hbegf | 1.78E+00 | 1.10E+01 | 6.19E+00 |
| Myof | 1.57E+00 | 7.10E+01 | 4.53E+01 |
| Prss27 | 2.14E+00 | 1.01E+01 | 4.73E+00 |
| Slc14a1 | 3.46E+00 | 4.84E+00 | 1.40E+00 |
| Entpd7 | 1.74E+00 | 6.59E+00 | 3.79E+00 |
| Gm7899 | 2.05E+00 | 3.22E+00 | 1.57E+00 |
| Gm12988 | 1.84E+00 | 2.26E+00 | 1.23E+00 |
| Tpcn2 | 1.61E+00 | 1.55E+01 | 9.64E+00 |
| Srm | 1.60E+00 | 5.26E+01 | 3.30E+01 |
| Has3 | 4.85E+00 | 5.99E+00 | 1.23E+00 |
| Wdhd1 | 1.76E+00 | 1.15E+01 | 6.56E+00 |
| Gm13092 | 1.59E+00 | 1.99E+01 | 1.25E+01 |
| Socs3 | 1.98E+00 | 6.09E+01 | 3.08E+01 |
| Anapc15-ps | 1.73E+00 | 1.48E+00 | 8.54E−01 |
| Fosl1 | 1.72E+00 | 2.62E+00 | 1.52E+00 |
| Nfkbid | 1.64E+00 | 1.84E+00 | 1.12E+00 |
| Pim2 | 1.60E+00 | 5.79E+00 | 3.63E+00 |
| Ada | 1.76E+00 | 7.47E+00 | 4.25E+00 |
| Gm5791 | 2.18E+00 | 3.62E+00 | 1.66E+00 |
| Gm11675 | 1.55E+00 | 1.03E+01 | 6.67E+00 |
| Sema4c | 1.52E+00 | 3.19E+01 | 2.10E+01 |
| Tes | 1.54E+00 | 1.70E+01 | 1.10E+01 |
| Lrp11 | 1.54E+00 | 3.74E+00 | 2.44E+00 |
| Tyms | 1.53E+00 | 3.44E+01 | 2.25E+01 |
| Mtcl1 | 2.78E+00 | 9.61E+00 | 3.45E+00 |
| Rfng | 1.53E+00 | 4.98E+01 | 3.26E+01 |
| Snhg4 | 1.61E+00 | 2.49E+01 | 1.54E+01 |
| Ndnf | 3.12E+00 | 2.30E+01 | 7.37E+00 |
| Gm3379 | 1.93E+00 | 6.41E+00 | 3.32E+00 |
| Trip13 | 1.86E+00 | 7.69E+00 | 4.13E+00 |
| Sprr2g | 6.51E+00 | 4.66E+00 | 7.15E−01 |
| Gpr176 | 2.59E+00 | 2.19E+00 | 8.47E−01 |
| Panx1 | 1.64E+00 | 2.94E+01 | 1.79E+01 |
| Stat3 | 1.64E+00 | 2.28E+02 | 1.39E+02 |
| Gemin4 | 2.10E+00 | 7.39E+00 | 3.52E+00 |
| Slc9a1 | 1.58E+00 | 1.20E+02 | 7.58E+01 |
| Gm6210 | 1.87E+00 | 8.75E+00 | 4.67E+00 |
| Eef1akmt4 | 1.51E+00 | 8.52E+00 | 5.63E+00 |
| Gm5620 | 1.67E+00 | 2.78E+00 | 1.66E+00 |
| Dnph1 | 1.52E+00 | 2.48E+01 | 1.64E+01 |
| Gm12666 | 1.63E+00 | 1.96E+00 | 1.20E+00 |
| S100a9 | 1.76E+01 | 1.19E+02 | 6.74E+00 |
| Mrto4 | 1.54E+00 | 2.72E+01 | 1.76E+01 |
| Stfa3 | 5.45E+00 | 5.02E+00 | 9.20E−01 |
| Nkain1 | 2.87E+00 | 2.51E+00 | 8.77E−01 |
| Psmg4 | 1.50E+00 | 1.16E+01 | 7.70E+00 |
| S100a8 | 1.22E+01 | 6.75E+01 | 5.51E+00 |
| Ampd3 | 1.70E+00 | 2.89E+01 | 1.70E+01 |
| Duxbl2 | 1.75E+00 | 2.82E+00 | 1.61E+00 |
| Rab15 | 1.62E+00 | 6.56E+01 | 4.04E+01 |
| Gm27219 | 1.71E+00 | 6.41E+00 | 3.74E+00 |
| Hmgb1-ps3 | 1.96E+00 | 2.20E+00 | 1.12E+00 |
| Noc4l | 1.78E+00 | 3.35E+01 | 1.88E+01 |
| Gm12722 | 1.63E+00 | 1.81E+00 | 1.11E+00 |
| Dsg1c | 2.04E+00 | 1.31E+01 | 6.39E+00 |
| 1700052K11Rik | 1.55E+00 | 1.03E+01 | 6.69E+00 |
| Krt6a | 3.99E+00 | 3.43E+02 | 8.58E+01 |
| Hirip3 | 1.64E+00 | 1.82E+01 | 1.11E+01 |
| Pgam1 | 1.63E+00 | 1.70E+02 | 1.04E+02 |
| B4galnt4 | 1.83E+00 | 1.81E+00 | 9.87E−01 |
| Rpl10-ps5 | 1.68E+00 | 1.54E+01 | 9.15E+00 |
| Hrh2 | 2.72E+00 | 2.19E+00 | 8.06E−01 |
| AC131178.1 | 1.99E+00 | 4.69E+00 | 2.35E+00 |
| Gadl1 | 1.71E+00 | 1.33E+02 | 7.81E+01 |
| BC055324 | 1.56E+00 | 5.06E+00 | 3.25E+00 |
| Gm6063 | 1.93E+00 | 2.60E+00 | 1.34E+00 |
| Trex1 | 1.54E+00 | 2.68E+01 | 1.73E+01 |
| Galnt6 | 2.60E+00 | 2.01E+01 | 7.71E+00 |
| Gm3448 | 1.89E+00 | 7.58E+00 | 4.01E+00 |
| Nat8l | 1.66E+00 | 1.07E+02 | 6.49E+01 |
| AC022775.2 | 1.59E+00 | 3.33E+00 | 2.09E+00 |
| Gm6793 | 1.97E+00 | 4.34E+00 | 2.21E+00 |
| Mgat5b | 3.43E+00 | 4.53E+00 | 1.32E+00 |
| Rpl29 | 1.51E+00 | 6.90E+00 | 4.56E+00 |
| Gm11805 | 1.72E+00 | 1.63E+00 | 9.46E−01 |
| Pla2g7 | 1.58E+00 | 1.72E+01 | 1.09E+01 |
| AC154176.1 | 1.62E+00 | 2.60E+01 | 1.60E+01 |
| Top2a | 1.96E+00 | 7.01E+01 | 3.58E+01 |
| H2afx | 1.90E+00 | 2.64E+01 | 1.39E+01 |
| Gtse1 | 2.30E+00 | 1.01E+01 | 4.37E+00 |
| Gm12933 | 2.18E+00 | 1.96E+00 | 8.98E−01 |
| Enah | 1.51E+00 | 5.55E+01 | 3.68E+01 |
| Sbno2 | 1.53E+00 | 9.96E+01 | 6.50E+01 |
| Tmem266 | 2.19E+00 | 4.45E+00 | 2.03E+00 |
| Sprr2e | 7.70E+00 | 6.61E+00 | 8.59E−01 |
| Slpi | 5.25E+00 | 1.97E+01 | 3.75E+00 |
| Aldh1a3 | 2.52E+00 | 4.26E+00 | 1.69E+00 |
| Kcnh1 | 2.08E+00 | 4.04E+01 | 1.94E+01 |
| Cxcr2 | 3.05E+00 | 1.48E+01 | 4.83E+00 |
| Rpl32-ps | 1.52E+00 | 5.03E+00 | 3.31E+00 |
| Prnd | 1.80E+00 | 1.97E+00 | 1.09E+00 |
| Cdh3 | 1.65E+00 | 5.78E+01 | 3.50E+01 |
| Gpx2 | 2.34E+00 | 1.73E+01 | 7.39E+00 |
| Grwd1 | 1.56E+00 | 3.09E+01 | 1.98E+01 |
| Cenpw | 1.72E+00 | 6.62E+00 | 3.86E+00 |
| Nop56 | 1.65E+00 | 6.31E+01 | 3.82E+01 |
| Trp53i11 | 1.57E+00 | 7.86E+01 | 5.00E+01 |
| Tctn3 | 1.54E+00 | 6.83E+00 | 4.45E+00 |
| Gls2 | 1.70E+00 | 1.86E+00 | 1.10E+00 |
| Gpcpd1 | 1.58E+00 | 8.83E+01 | 5.58E+01 |
| Gpatch4 | 1.61E+00 | 3.57E+01 | 2.22E+01 |
| Shank1 | 1.65E+00 | 2.02E+00 | 1.22E+00 |
| Atp1a1 | 1.95E+00 | 7.45E+02 | 3.81E+02 |
| Dctd | 1.58E+00 | 8.57E+00 | 5.44E+00 |
| Ly6k | 3.05E+00 | 7.01E+00 | 2.30E+00 |
| Rrp12 | 1.54E+00 | 2.91E+01 | 1.88E+01 |
| Gch1 | 1.58E+00 | 6.69E+00 | 4.25E+00 |
| Il13ra2 | 2.24E+00 | 4.34E+00 | 1.93E+00 |
| Ccnf | 1.56E+00 | 1.28E+01 | 8.21E+00 |
| Rpl7a-ps10 | 1.68E+00 | 2.46E+00 | 1.46E+00 |
| Plbd1 | 1.57E+00 | 1.60E+02 | 1.02E+02 |
| Lrrc4 | 3.11E+00 | 8.15E+00 | 2.62E+00 |
| Tdg-ps | 1.57E+00 | 1.39E+01 | 8.82E+00 |
| Gm8666 | 1.52E+00 | 7.81E+00 | 5.14E+00 |
| Gpr35 | 1.67E+00 | 6.13E+00 | 3.67E+00 |
| Itpk1 | 1.54E+00 | 2.41E+01 | 1.56E+01 |
| Nolc1 | 1.52E+00 | 6.28E+01 | 4.12E+01 |
| Incenp | 1.63E+00 | 2.14E+01 | 1.31E+01 |
| Oplah | 1.50E+00 | 8.39E+01 | 5.58E+01 |
| Nt5dc2 | 1.65E+00 | 5.77E+01 | 3.49E+01 |
| Tubb3 | 2.40E+00 | 6.40E+00 | 2.67E+00 |
| Txn-ps1 | 1.82E+00 | 2.55E+00 | 1.40E+00 |
| AI506816 | 2.05E+00 | 6.04E+00 | 2.95E+00 |
| Gm15452 | 1.67E+00 | 2.66E+00 | 1.59E+00 |
| Depdc1b | 1.87E+00 | 3.33E+00 | 1.78E+00 |
| Epb4113 | 1.52E+00 | 2.95E+01 | 1.94E+01 |
| Btbd19 | 1.51E+00 | 1.59E+01 | 1.06E+01 |
| Igsf3 | 1.71E+00 | 3.18E+01 | 1.86E+01 |
| Dusp9 | 2.04E+00 | 2.48E+00 | 1.21E+00 |
| Cd44 | 1.76E+00 | 1.30E+02 | 7.39E+01 |
| Gm45902 | 1.74E+00 | 5.89E+00 | 3.38E+00 |
| Wfdc12 | 4.99E+00 | 3.58E+01 | 7.17E+00 |
| Tdh | 3.18E+00 | 2.23E+01 | 7.01E+00 |
| Rps13-ps2 | 1.83E+00 | 1.00E+01 | 5.49E+00 |
| Poc1a | 1.68E+00 | 7.80E+00 | 4.63E+00 |
| Gja1 | 1.62E+00 | 1.63E+02 | 1.00E+02 |
| 9330102E08Rik | 1.74E+00 | 4.30E+00 | 2.48E+00 |
| 2700038G22Rik | 1.89E+00 | 2.88E+00 | 1.52E+00 |
| Rpl27 | 1.70E+00 | 1.91E+01 | 1.13E+01 |
| Cdca8 | 1.64E+00 | 1.92E+01 | 1.17E+01 |
| Stx11 | 1.60E+00 | 6.82E+00 | 4.28E+00 |
| Fam167a | 3.53E+00 | 1.59E+01 | 4.51E+00 |
| Pinx1 | 1.54E+00 | 1.14E+01 | 7.40E+00 |
| Cks2 | 1.56E+00 | 9.48E+00 | 6.08E+00 |
| Gm10182 | 1.57E+00 | 2.43E+01 | 1.55E+01 |

Control-resting skin: n = 2, Control-bitten skin: n = 2, AgBR1 antiserum-bitten skin: n = 2 biologically independent samples.
Normalized read counts were statistically modeled using Partek Flow's Gene Specific Analysis (GSA) approach.

TABLE 3
Oligonucleotide primers used in the experiments.
Oligonucleotide primers for qRT-PCR

| | |
|---|---|
| Zika | F: TTGGTCATGATACTGCTGATTGC (SEQ ID NO: 130) |
| virus | R: CCTTCCACAAAGTCCCTATTGC (SEQ ID NO: 131) |
| Mosquito | F: GCTATGACAAGCTTGCCCCCA (SEQ ID NO: 132) |
| Rp49 | R: TCATCAGCACCTCCAGCT (SEQ ID NO: 133) |
| Mouse | F: GATGACGATATCGCTGCGCTG (SEQ ID NO: 134) |
| βactin | R: GTACGACCAGAGGCATACAGG (SEQ ID NO: 135) |
| Mouse | F: TGGAACTGGCAGAAGAGGCACT (SEQ ID NO: 136) |
| Tnfa | R: GAGATAGCAAATCGGCTGACGG (SEQ ID NO: 137) |
| Mouse | F: GCTTCAGGCAGGCAGTATCAC (SEQ ID NO: 138) |
| Il1b | R: CGACAGCACGAGGCTTTTT (SEQ ID NO: 139) |
| Mouse | F: ATGAAGTTCCTCTCTGCAAGAGACT (SEQ ID NO: 140) |
| Il6 | R: CACTAGGTTTGCCGAGTAGATCTC (SEQ ID NO: 141) |
| Mosquito | F: CGTCAACTTGGCTTCGTTCG (SEQ ID NO: 142) |
| AgBR1 | R: GATGCCGGATTTCTCCACCA (SEQ ID NO: 143) |

Oligonucleotide primers for cloning into
the expression vector

| | |
|---|---|
| AgBR1 | F: CTCGCTCGGGAGATCTAACAATGCCACTACCGGCCCAAAGGTCCTC |
| | (SEQ ID NO: 144) |
| | R: GCCCTCTAGACTCGAGCAGCCTATACTTAGCAGCCCTCAG |
| | (SEQ ID NO: 145) |
| SP | F:CTCGCTCGGGAGATCTCACCCAATTCCAGCCGAAGATCCCGCCAAGC |
| | (SEQ ID NO: 146) |
| | R:CCCTCTAGACTCGAGACCAAAAGCCTTCACCATGACCTTCGGATAG |
| | (SEQ ID NO: 147) |
| D7Bclu | F: CTCGCTCGGGAGATCTGCACCTTTATGGGATGCAAAGGATCCAGAGC |
| | (SEQ ID NO: 148) |
| | R: GCCCTCTAGACTCGAGGCTACACTGGATCTTGTCGATATCG |
| | (SEQ ID NO: 149) |

Oligonucleotide primers for dsRNA preparation

| | |
|---|---|
| dsAgBR1 | F: TAATACGACTCACTATAGGGGATGGACAGATGTCTCTTCGTG |
| RNA | (SEQ ID NO: 150) |
| | R: TAATACGACTCACTATAGGGCCAAATCCAATCCATCGAAA |
| | (SEQ ID NO: 151) |
| dsGFP | F: TAATACGACTCACTATAGGGGTGAGCAAGGGCGAGGAG |
| RNA | (SEQ ID NO: 152) |
| | R: TAATACGACTCACTATAGGGCATGATATAGACGTTGTGGCTGTT |
| | (SEQ ID NO: 153) |

TABLE 4
List of GSEA enriched pathway at bite sites in mice
treated with control serum using hallmark gene sets

| | GS follow link to MSigDB | NES | FDR q-val |
|---|---|---|---|
| 1 | HALLMARK_INFLAMMATORY_ | 2.5049036 | 0 |
| | RESPONSE | | |
| 2 | HALLMARK_ALLOGRAFT_ | 2.3367434 | 0 |
| | REJECTION | | |
| 3 | HALLMARK_IL6_JAK_STAT3_ | 2.016875 | 8.00E−04 |
| | SIGNALING | | |
| 4 | HALLMARK_TNFA_SIGNALING_ | 1.8812603 | 0.001689706 |
| | VIA_NFKB | | |
| 5 | HALLMARK_IL2_STAT5_ | 1.5615453 | 0.028370652 |
| | SIGNALING | | |

Control-resting skin: n = 2, Control-bitten skin: n = 2, AgBR1 antiserum-bitten skin: n = 2 biologically independent samples.
FDR statistics were performed based on study described in Subramanian, A. et al. Gene set enrichment analysis: a knowledge-based approach for interpreting genome-wide expression profiles. Proc. Natl Acad. Sci. USA 102, 15545-15550 (2005).

TABLE 5
List of GSEA enriched pathway at bite sites in
mice treated with control serum using KEGG gene sets

| | | | | LEADING |
|---|---|---|---|---|
| | GS follow link to MSigDB | NES | FDR q-val | EDGE |
| 1 | KEGG_CYTOKINE_CYTOKINE_ | 2.5147192 | 0 | tags = 34%, |
| | RECEPTOR_INTERACTION | | | list = 10%, |
| | | | | signal = 37% |
| 2 | KEGG_HEMATOPOIETIC_CELL_ | 2.1477058 | 3.80E−04 | tags = 34%, |
| | LINEAGE | | | list = 12%, |
| | | | | signal = 38% |
| 3 | KEGG_RIBOSOME | 2.0992713 | 0.00114546 | tags = 64%, |
| | | | | list = 21%, |
| | | | | signal = 80% |
| 4 | KEGG_NOD_LIKE_RECEPTOR_ | 2.0794337 | 0.001266238 | tags = 18%, |
| | SIGNALING_PATHWAY | | | list = 3%, |
| | | | | signal = 18% |
| 5 | KEGG_TYPE_I_DIABETES_MELLITUS | 2.047159 | 0.001789215 | tags = 47%, |
| | | | | list = 9%, |
| | | | | signal = 52% |

Control-resting skin: n = 2,
Control-bitten skin: n = 2,
AgBR1 antiserum-bitten skin: n = 2 biologically independent samples.
FDR statistics were performed based on study described in Subramanian, A. et al. Gene set enrichment analysis: a knowledge-based approach for interpreting genome-wide expression profiles. Proc. Natl Acad. Sci. USA 102, 15545-15550 (2005).

TABLE 6
Gene List in gene sets of enriched pathway in Table 4.

| | | | |
|---|---|---|---|
| | | RANK | CORE |
| GENE | | METRIC | ENRICH- |
| SYMBOL | GENE_TITLE | SCORE | MENT |

HALLMARK_INFLAMMATORY_RESPONSE

| | | | |
|---|---|---|---|
| SELL | selectin L (lymphocyte adhesion molecule 1) | 4.533662319 | Yes |
| IL1B | interleukin 1, beta | 3.462865114 | Yes |
| IL6 | interleukin 6 (interferon, beta 2) | 3.456357479 | Yes |
| IL18RAP | interleukin 18 receptor accessory protein | 3.251766205 | Yes |
| CSF3R | colony stimulating factor 3 receptor | 3.238983631 | Yes |
| | (granulocyte) | | |
| OSM | oncostatin M | 2.948629141 | Yes |
| MSR1 | macrophage scavenger receptor 1 | 2.937763929 | Yes |
| C5AR1 | complement component 5a receptor 1 | 2.387494087 | Yes |
| MEFV | Mediterranean fever | 2.353091955 | Yes |
| MARCO | macrophage receptor with collagenous | 2.29198432 | Yes |
| | structure | | |
| TNFRSF9 | tumor necrosis factor receptor superfamily, | 2.029400826 | Yes |
| | member 9 | | |
| SERPINE1 | serpin peptidase inhibitor, clade E (nexin, | 1.842594028 | Yes |
| | plasminogen activator inhibitor type 1), | | |
| | member 1 | | |
| CCL5 | chemokine (C-C motif) ligand 5 | 1.827621102 | Yes |
| CLEC5A | C-type lectin domain family 5, member A | 1.796642661 | Yes |
| BDKRB1 | bradykinin receptor B1 | 1.789603353 | Yes |
| PTAFR | platelet-activating factor receptor | 1.573449492 | Yes |
| PROK2 | prokineticin 2 | 1.485739708 | Yes |
| CCL22 | chemokine (C-C motif) ligand 22 | 1.440871716 | Yes |
| CSF3 | colony stimulating factor 3 (granulocyte) | 1.422928095 | Yes |
| TLR1 | toll-like receptor 1 | 1.34019506 | Yes |
| PLAUR | plasminogen activator, urokinase receptor | 1.308592319 | Yes |
| FPR1 | formyl peptide receptor 1 | 1.262866974 | Yes |
| TNFRSF1B | tumor necrosis factor receptor superfamily, | 1.259392858 | Yes |
| | member 1B | | |
| SLAMF1 | signaling lymphocytic activation molecule | 1.241883397 | Yes |
| | family member 1 | | |
| CCL24 | chemokine (C-C motif) ligand 24 | 1.233121157 | Yes |
| GPR132 | G protein-coStupled receptor 132 | 1.176985025 | Yes |
| LYN | v-yes-1 Yamaguchi sarcoma viral related | 1.097683549 | Yes |
| | oncogene homolog | | |
| LCP2 | lymphocyte cytosolic protein 2 (SH2 | 1.090578794 | Yes |
| | domain containing leukocyte protein of 76kDa) | | |
| PIK3R5 | phosphoinositide-3-kinase, regulatory | 1.057828546 | Yes |
| | subunit 5, p101 | | |
| C3AR1 | complement component 3a receptor 1 | 1.036981106 | Yes |
| RGS1 | regulator of G-protein signalling 1 | 0.960995197 | Yes |
| CCL2 | chemokine (C-C motif) ligand 2 | 0.932862163 | Yes |
| CD14 | CD14 molecule | 0.848022759 | Yes |
| CCRL2 | chemokine (C-C motif) receptor-like 2 | 0.822621703 | Yes |
| STAB1 | stabilin 1 | 0.800862968 | Yes |
| IL10 | interleukin 10 | 0.777381659 | Yes |
| TNFSF9 | tumor necrosis factor (ligand) superfamily, | 0.771818101 | Yes |
| | member 9 | | |
| ADM | adrenomedullin | 0.746417522 | Yes |
| ICAM1 | intercellular adhesion molecule 1 (CD54), | 0.746375382 | Yes |
| | human rhinovirus receptor | | |
| RTP4 | receptor transporter protein 4 | 0.680959046 | Yes |
| TLR2 | toll-like receptor 2 | 0.671268702 | Yes |
| CCL17 | chemokine (C-C motif) ligand 17 | 0.664499223 | Yes |
| BST2 | bone marrow stromal cell antigen 2 | 0.6320346 | Yes |
| CCL20 | chemokine (C-C motif) ligand 20 | 0.625092983 | Yes |
| CCR7 | chemokine (C-C motif) receptor 7 | 0.594285488 | Yes |
| CD82 | CD82 molecule | 0.569351971 | Yes |
| LIF | leukemia inhibitory factor (cholinergic | 0.547570944 | Yes |
| | differentiation factor) | | |
| TNFAIP6 | tumor necrosis factor, alpha-induced protein 6 | 0.543578744 | Yes |
| HBEGF | heparin-binding EGF-like growth factor | 0.541148901 | Yes |
| CD48 | CD48 molecule | 0.540921032 | Yes |
| RGS16 | regulator of G-protein signalling 16 | 0.526294231 | Yes |
| SELE | selectin E (endothelial adhesion molecule 1) | 0.494481832 | Yes |
| CD40 | CD40 molecule, TNF receptor superfamily | 0.492939293 | Yes |
| | member 5 | | |
| LTA | lymphotoxin alpha (TNF superfamily, | 0.44320941 | No |
| | member 1) | | |
| HAS2 | hyaluronan synthase 2 | 0.441198289 | No |
| ITGA5 | integrin, alpha 5 (fibronectin receptor, alpha | 0.429049671 | No |
| | polypeptide) | | |
| SLC4A4 | solute carrier family 4, sodium bicarbonate | 0.396574855 | No |
| | cotransporter, member 4 | | |
| CXCL9 | chemokine (C-X-C motif) ligand 9 | 0.391491205 | No |
| IL10RA | interleukin 10 receptor, alpha | 0.343504012 | No |
| GCH1 | GTP cyclohydrolase 1 (dopa-responsive | 0.327480644 | No |
| | dystonia) | | |
| LCK | lymphocyte-specific protein tyrosine kinase | 0.318335414 | No |
| IFITM1 | interferon induced transmembrane protein 1 | 0.315781265 | No |
| | (9-27) | | |
| PDPN | podoplanin | 0.294152737 | No |
| PTGIR | prostaglandin I2 (prostacyclin) receptor (IP) | 0.285321265 | No |
| OLR1 | oxidised low density lipoprotein (lectin-like) | 0.284122884 | No |
| | receptor 1 | | |
| INHBA | inhibin, beta A (activin A, activin AB alpha | 0.280317694 | No |
| | polypeptide) | | |
| ITGB3 | integrin, beta 3 (platelet glycoprotein IIIa, | 0.256395549 | No |
| | antigen CD61) | | |
| CCL7 | chemokine (C-C motif) ligand 7 | 0.251811057 | No |
| CXCL10 | chemokine (C-X-C motif) ligand 10 | 0.226761773 | No |
| IRF1 | interferon regulatory factor 1 | 0.214157283 | No |
| OSMR | oncostatin M receptor | 0.207781732 | No |
| LAMP3 | lysosomal-associated membrane protein 3 | 0.205396608 | No |
| SLC28A2 | solute carrier family 28 (sodium-coupled | 0.192293972 | No |
| | nucleoside transporter), member 2 | | |
| EBI3 | Epstein-Barr virus induced gene 3 | 0.188898429 | No |
| SLC7A2 | solute carrier family 7 (cationic amino acid | 0.175343886 | No |
| | transporter, y+ system), member 2 | | |
| ICAM4 | intercellular adhesion molecule 4 | 0.169187918 | No |
| | (Landsteiner-Wiener blood group) | | |
| CD69 | CD69 molecule | 0.166904241 | No |
| ADRM1 | adhesion regulating molecule 1 | 0.161846325 | No |
| PTGER4 | prostaglandin E receptor 4 (subtype EP4) | 0.143603638 | No |
| NDP | Norrie disease (pseudoglioma) | 0.107874334 | No |
| SCARF1 | scavenger receptor class F, member 1 | 0.093449399 | No |
| IL15RA | interleukin 15 receptor, alpha | 0.084517181 | No |
| CXCL11 | chemokine (C-X-C motif) ligand 11 | 0.079039596 | No |
| CYBB | cytochrome b-245, beta polypeptide | 0.067997418 | No |
| | (chronic granulomatous disease) | | |
| IFNAR1 | interferon (alpha, beta and omega) receptor 1 | 0.059620846 | No |
| IFNGR2 | interferon gamma receptor 2 (interferon | 0.046017136 | No |
| | gamma transducer 1) | | |
| TPBG | trophoblast glycoprotein | 0.045252148 | No |
| CSF1 | colony stimulating factor 1 (macrophage) | 0.042810541 | No |
| CXCR6 | chemokine (C-X-C motif) receptor 6 | 0.031615365 | No |
| GPC3 | glypican 3 | 0.026109839 | No |
| EIF2AK2 | eukaryotic translation initiation factor 2- | 0.021446833 | No |
| | alpha kinase 2 | | |
| CHST2 | carbohydrate (N-acetylglucosamine-6-0) | 0.006915596 | No |
| | sulfotransferase 2 | | |
| EDN1 | endothelin 1 | 0.002488923 | No |
| P2RX4 | purinergic receptor P2X, ligand-gated ion | 0.002422775 | No |
| | channel, 4 | | |
| PTPRE | protein tyrosine phosphatase, receptor type, E | −0.01096273 | No |
| PTGER2 | prostaglandin E receptor 2 (subtype EP2), 53kDa | −0.01937426 | No |
| TACR3 | tachykinin receptor 3 | −0.02168826 | No |
| NPFFR2 | neuropeptide FF receptor 2 | −0.03000635 | No |
| NFKBIA | nuclear factor of kappa light polypeptide | −0.03587324 | No |
| | gene enhancer in B-cells inhibitor, alpha | | |
| DCBLD2 | discoidin, CUB and LCCL domain | −0.04324392 | No |
| | containing 2 | | |
| IL15 | interleukin 15 | −0.04502484 | No |
| IL1R1 | interleukin 1 receptor, type I | −0.05148519 | No |
| FZD5 | frizzled homolog 5 (Drosophila) | −0.05248347 | No |
| ADORA2B | adenosine A2b receptor | −0.07098219 | No |
| CMKLR1 | chemokine-like receptor 1 | −0.07226631 | No |
| TLR3 | toll-like receptor 3 | −0.0735488 | No |
| CDKN1A | cyclin-dependent kinase inhibitor 1A p21, Cip1) | −0.08443426 | No |
| KCNA3 | potassium voltage-gated channel, shaker- | −0.09990013 | No |
| | related subfamily, member 3 | | |
| MMP14 | matrix metallopeptidase 14 (membrane-inserted) | −0.10503554 | No |
| HRH1 | histamine receptor H1 | −0.11474121 | No |
| IRAK2 | interleukin-1 receptor-associated kinase 2 | −0.13551585 | No |
| ATP2B1 | ATPase, Ca++ transporting, plasma membrane 1 | −0.14145075 | No |
| SCN1B | sodium channel, voltage-gated, type I, beta | −0.14982769 | No |
| SLC31A2 | solute carrier family 31 (copper transporters), member 2 | −0.15675473 | No |
| PVR | poliovirus receptor | −0.15986347 | No |
| SLC11A2 | solute carrier family 11 (proton-coupled | −0.16051796 | No |
| | divalent metal ion transporters), member 2 | | |
| KLF6 | Kruppel-like factor 6 | −0.16283926 | No |
| ITGB8 | integrin, beta 8 | −0.16627799 | No |
| PDE4B | phosphodiesterase 4B, cAMP-specific | −0.16894662 | No |
| | (phosphodiesterase E4 dunce homolog, Drosophila) | | |
| MXD1 | MAX dimerization protein 1 | −0.16998833 | No |
| BTG2 | BTG family, member 2 | −0.17541341 | No |
| SLC7A1 | solute carrier family 7 (cationic amino acid | −0.17681299 | No |
| | transporter, y+ system), member 1 | | |
| RIPK2 | receptor-interacting serine-threonine kinase 2 | −0.1842809 | No |
| SRI | sorcin | −0.19541027 | No |
| P2RX7 | purinergic receptor P2X, ligand-gated ion channel, 7 | −0.1960772 | No |
| P2RY2 | purinergic receptor P2Y, G-protein coupled, 2 | −0.19852374 | No |
| LDLR | low density lipoprotein receptor (familial | −0.20240238 | No |
| | hypercholesterolemia) | | |
| HIF1A | hypoxia-inducible factor 1, alpha subunit | −0.20901471 | No |
| | (basic helix-loop-helix transcription factor) | | |
| AHR | aryl hydrocarbon receptor | −0.21279255 | No |
| CX3CL1 | chemokine (C-X3-C motif) ligand 1 | −0.22508623 | No |
| MET | met proto-oncogene (hepatocyte growth | −0.2252703 | No |
| | factor receptor) | | |
| LY6E | lymphocyte antigen 6 complex, locus E | −0.22614777 | No |
| NMI | N-myc (and STAT) interactor | −0.22712676 | No |
| SEMA4D | sema domain, immunoglobulin domain (Ig), | −0.22815116 | No |
| | transmembrane domain (TM) and short | | |
| | cytoplasmic domain, (semaphorin) 4D | | |
| IL1A | interleukin 1, alpha | −0.22882077 | No |
| ABI1 | abl-interactor 1 | −0.22907911 | No |
| GNA15 | guanine nucleotide binding protein | −0.23037907 | No |
| | (G protein), alpha 15 (Gq class) | | |
| SPHK1 | sphingosine kinase 1 | −0.23109274 | No |
| NFKB1 | nuclear factor of kappa light polypeptide | −0.25809258 | No |
| | gene enhancer in B-cells 1 (p105) | | |
| ACVR2A | activin A receptor, type IIA | −0.26500779 | No |
| AQP9 | aquaporin 9 | −0.2774868 | No |
| IRF7 | interferon regulatory factor 7 | −0.28254756 | No |
| ACVR1B | activin A receptor, type IB | −0.29110909 | No |
| IL7R | interleukin 7 receptor | −0.30657914 | No |
| SLC31A1 | solute carrier family 31 (copper | −0.30956858 | No |
| | transporters), member 1 | | |
| KIF1B | kinesin family member 1B | −0.31039682 | No |
| IL2RB | interleukin 2 receptor, beta | −0.31792504 | No |
| TAPBP | TAP binding protein (tapasin) | −0.32166621 | No |
| GNAI3 | guanine nucleotide binding protein (G | −0.32649222 | No |
| | protein), alpha inhibiting activity | | |
| | polypeptide 3 | | |
| RELA | v−rel reticuloendotheliosis viral oncogene | −0.32651395 | No |
| | homolog A, nuclear factor of kappa light | | |
| | polypeptide gene enhancer in B-cells 3, p65 | | |
| | (avian) | | |
| ATP2A2 | ATPase, Ca++ transporting, cardiac muscle, | −0.32773513 | No |
| | slow twitch 2 | | |
| ABCA1 | ATP-binding cassette, sub-family A | −0.33476061 | No |
| | (ABC1), member 1 | | |
| RAF1 | v-raf-1 murine leukemia viral oncogene homolog 1 | −0.3348152 | No |
| PCDH7 | BH-protocadherin (brain-heart) | −0.34278125 | No |
| CD55 | CD55 molecule, decay accelerating factor | −0.34951958 | No |
| | for complement (Cromer blood group) | | |
| TACR1 | tachykinin receptor 1 | −0.36803696 | No |
| RASGRP1 | RAS guanyl releasing protein 1 (calcium | −0.37512192 | No |
| | and DAG-regulated) | | |
| PSEN1 | presenilin 1 (Alzheimer disease 3) | −0.38747761 | No |
| ATP2C1 | ATPase, Ca++ transporting, type 2C, member 1 | −0.39031595 | No |
| CALCRL | calcitonin receptor-like | −0.41768777 | No |
| SLC1A2 | solute carrier family 1 (glial high affinity | −0.48958847 | No |
| | glutamate transporter), member 2 | | |
| IL18 | interleukin 18 (interferon-gamma-inducing factor) | −0.5164243 | No |
| EMP3 | epithelial membrane protein 3 | −0.51683706 | No |
| GABBR1 | gamma-aminobutyric acid (GABA) B receptor, 1 | −0.54345095 | No |
| AXL | AXL receptor tyrosine kinase | −0.57041121 | No |
| MYC | v-myc myelocytomatosis viral oncogene | −0.57849312 | No |
| | homolog (avian) | | |
| GP1BA | glycoprotein Ib (platelet), alpha polypeptide | −0.59780264 | No |
| TNFSF15 | tumor necrosis factor (ligand) superfamily, member 15 | −0.65267819 | No |
| FFAR2 | free fatty acid receptor 2 | −0.66044772 | No |
| NMUR1 | neuromedin U receptor 1 | −0.66688561 | No |
| HPN | hepsin (transmembrane protease, serine 1) | −0.68898129 | No |
| KCNJ2 | potassium inwardly-rectifying channel, subfamily J, | −0.70222563 | No |
| | member 2 | | |
| TIMP1 | TIMP metallopeptidase inhibitor 1 | −0.70523745 | No |
| TNFSF10 | tumor necrosis factor (ligand) superfamily, member 10 | −0.78845328 | No |
| IL18R1 | interleukin 18 receptor 1 | −0.81675339 | No |
| RHOG | ras homolog gene family, member G (rhoG) | −0.9650324 | No |
| EREG | epiregulin | −1.24036241 | No |
| ROS1 | v-ros UR2 sarcoma virus oncogene homolog 1 (avian) | −1.97980785 | No |

HALLMARK_ALLOGRAFT_REJECTION

| | | | |
|---|---|---|---|
| IL1B | interleukin 1, beta | 3.462865114 | Yes |
| IL6 | interleukin 6 (interferon, beta 2) | 3.456357479 | Yes |
| IL18RAP | interleukin 18 receptor accessory protein | 3.251766205 | Yes |
| STAT4 | signal transducer and activator of transcription 4 | 3.148304939 | Yes |
| TNF | tumor necrosis factor (TNF superfamily, member 2) | 2.751254082 | Yes |
| GZMB | granzyme B (granzyme 2, cytotoxic T- | 2.667121887 | Yes |
| | lymphocyte-associated serine esterase 1) | | |
| CCR1 | chemokine (C-C motif) receptor 1 | 2.132987738 | Yes |
| CCR5 | chemokine (C-C motif) receptor 5 | 2.054755688 | Yes |
| CCL5 | chemokine (C-C motif) ligand 5 | 1.827621102 | Yes |
| ITGAL | integrin, alpha L (antigen CD11A (p180), lymphocyte | 1.800091743 | Yes |
| | function-associated antigen 1; alpha polypeptide) | | |
| CD7 | CD7 molecule | 1.628337383 | Yes |
| FGR | Gardner-Rasheed feline sarcoma viral | 1.552860975 | Yes |
| | (v-fgr) oncogene homolog | | |
| CCL22 | chemokine (C-C motif) ligand 22 | 1.440871716 | Yes |
| TLR1 | toll-like receptor 1 | 1.34019506 | Yes |
| IL12A | interleukin 12A (natural killer cell stimulatory factor 1, | 1.315529704 | Yes |
| | cytotoxic lymphocyte maturation factor 1, p35) | | |
| CCL4 | chemokine (C-C motif) ligand 4 | 1.303452969 | Yes |
| GPR65 | G protein-coupled receptor 65 | 1.301272631 | Yes |
| CCR2 | chemokine (C-C motif) receptor 2 | 1.17247808 | Yes |
| PRF1 | perforin 1 (pore forming protein) | 1.171788573 | Yes |
| IGSF6 | immunoglobulin superfamily, member 6 | 1.100607872 | Yes |
| LYN | v-yes-1 Yamaguchi sarcoma viral related oncogene | 1.097683549 | Yes |
| | homolog | | |
| LCP2 | lymphocyte cytosolic protein 2 (SH2 domain | 1.090578794 | Yes |
| | containing leukocyte protein of 76kDa) | | |
| IL2RG | interleukin 2 receptor, gamma (severe | 1.083326221 | Yes |
| | combined immunodeficiency) | | |
| DYRK3 | dual-specificity tyrosine-(Y)- | 1.04365015 | Yes |
| | phosphorylation regulated kinase 3 | | |
| IRF8 | interferon regulatory factor 8 | 0.984531879 | Yes |
| TLR6 | toll-like receptor 6 | 0.974328399 | Yes |
| CCL2 | chemokine (C-C motif) ligand 2 | 0.932862163 | Yes |
| SOCS1 | suppressor of cytokine signaling 1 | 0.912176847 | Yes |
| FYB | FYN binding protein (FYB-120/130) | 0.841911793 | Yes |
| STAB1 | stabilin 1 | 0.800862968 | Yes |
| CCL19 | chemokine (C-C motif) ligand 19 | 0.784207284 | Yes |
| ITGB2 | integrin, beta 2 (complement component 3 receptor 3 | 0.783176661 | Yes |
| | and 4 subunit) | | |
| IL10 | interleukin 10 | 0.777381659 | Yes |
| BCL3 | B-cell CLL/lymphoma 3 | 0.768477619 | Yes |
| ICAM1 | intercellular adhesion molecule 1 | 0.746375382 | Yes |
| | (CD54), human rhinovirus receptor | | |
| MMP9 | matrix metallopeptidase 9 (gelatinase B, 92kDa | 0.740974486 | Yes |
| | gelatinase, 92kDa type IV collagenase) | | |
| IL13 | interleukin 13 | 0.6945768 | Yes |
| IL11 | interleukin 11 | 0.684957564 | Yes |
| TLR2 | toll-like receptor 2 | 0.671268702 | Yes |
| GCNT1 | glucosaminyl (N-acetyl) transferase 1, core 2 | 0.671001792 | Yes |
| | (beta-1,6-N-acetylglucosaminyltransferase) | | |
| IL4 | interleukin 4 | 0.65638262 | Yes |
| CDKN2A | cyclin-dependent kinase inhibitor 2A | 0.650471985 | Yes |
| | (melanoma, p16, inhibits CDK4) | | |
| CD86 | CD86 molecule | 0.631752431 | Yes |
| CD80 | CD80 molecule | 0.600226164 | Yes |
| LTB | lymphotoxin beta (TNF superfamily, member 3) | 0.579620421 | Yes |
| LIF | leukemia inhibitory factor (cholinergic | 0.547570944 | Yes |
| | differentiation factor) | | |
| WAS | Wiskott-Aldrich syndrome (eczema-thrombocytopenia) | 0.525824308 | Yes |
| PTPRC | protein tyrosine phosphatase, receptor type, C | 0.522265971 | Yes |
| RPS19 | ribosomal protein S19 | 0.506382108 | Yes |
| CD40 | CD40 molecule, TNF receptor superfamily member 5 | 0.492939293 | Yes |
| NCF4 | neutrophil cytosolic factor 4, 40kDa | 0.465533584 | Yes |
| LY86 | lymphocyte antigen 86 | 0.449851602 | Yes |
| FCGR2B | Fc fragment of IgG, low affinity IIb, receptor (CD32) | 0.436047524 | Yes |
| HCLS1 | hematopoietic cell-specific Lyn substrate 1 | 0.427405208 | Yes |
| CD3D | CD3d molecule, delta (CD3-TCR complex) | 0.415081888 | Yes |
| CXCL9 | chemokine (C-X-C motif) ligand 9 | 0.391491205 | Yes |
| CXCR3 | chemokine (C-X-C motif) receptor 3 | 0.380579948 | Yes |
| BRCA1 | breast cancer 1, early onset | 0.38035053 | Yes |
| RPL9 | ribosomal protein L9 | 0.372396767 | Yes |
| CD8A | CD8a molecule | 0.337146819 | No |
| SIT1 | signaling threshold regulating transmembrane adaptor 1 | 0.334993273 | No |
| CD28 | CD28 molecule | 0.331128299 | No |
| LCK | lymphocyte-specific protein tyrosine kinase | 0.318335414 | No |
| IFNAR2 | interferon (alpha, beta and omega) receptor 2 | 0.281477422 | No |
| INHBA | inhibin, beta A (activin A, activin AB alpha polypeptide) | 0.280317694 | No |
| UBE2N | ubiquitin-conjugating enzyme E2N (UBC13 homolog, | 0.279983729 | No |
| | yeast) | | |
| CCL7 | chemokine (C-C motif) ligand 7 | 0.251811057 | No |
| CD79A | CD79a molecule, immunoglobulin-associated alpha | 0.249655664 | No |
| MAP4K1 | mitogen-activated protein kinase kinase kinase kinase 1 | 0.249342158 | No |
| ETS1 | v-ets erythroblastosis virus E26 oncogene homolog | 0.217027962 | No |
| | 1 (avian) | | |
| FAS | Fas (TNF receptor superfamily, member 6) | 0.173293099 | No |
| ST8SIA4 | ST8 alpha-N-acetyl-neuraminide alpha- | 0.147267297 | No |
| | 2,8-sialyltransferase 4 | | |
| RPL39 | ribosomal protein L39 | 0.145141497 | No |
| GLMN | glomulin, FKBP associated protein | 0.138915822 | No |
| CRTAM | cytotoxic and regulatory T cell molecule | 0.132859111 | No |
| ZAP70 | zeta-chain (TCR) associated protein kinase 70kDa | 0.127061605 | No |
| TGFB1 | transforming growth factor, beta 1 | 0.120179698 | No |
| | (Camurati-Engelmann disease) | | |
| SPI1 | spleen focus forming virus (SFFV) | 0.119634412 | No |
| | proviral integration oncogene spi1 | | |
| STAT1 | signal transducer and activator of transcription 1, 91kDa | 0.050059285 | No |
| IFNGR2 | interferon gamma receptor 2 (interferon gamma | 0.046017136 | No |
| | transducer 1) | | |
| CSF1 | colony stimulating factor 1 (macrophage) | 0.042810541 | No |
| CCL11 | chemokine (C-C motif) ligand 11 | 0.039969306 | No |
| CD3E | CD3e molecule, epsilon (CD3-TCR complex) | 0.002899456 | No |
| APBB1 | amyloid beta (A4) precursor protein-binding, family B, | −0.002237323 | No |
| | member 1 (Fe65) | | |
| GBP2 | guanylate binding protein 2, interferon-inducible | −0.031019775 | No |
| TAP1 | transporter 1, ATP-binding cassette, sub-family B | −0.033033174 | No |
| | (MDR/TAP) | | |
| CD3G | CD3g molecule, gamma (CD3-TCR complex) | −0.033679064 | No |
| SOCS5 | suppressor of cytokine signaling 5 | −0.042330034 | No |
| INHBB | inhibin, beta B (activin AB beta polypeptide) | −0.043366853 | No |
| IL15 | interleukin 15 | −0.045024838 | No |
| CXCL13 | chemokine (C-X-C motif) ligand 13 (B-cell | −0.045578849 | No |
| | chemoattractant) | | |
| IL12RB1 | interleukin 12 receptor, beta 1 | −0.050351642 | No |
| CD74 | CD74 molecule, major histocompatibility complex, | −0.073090956 | No |
| | class II invariant chain | | |
| TLR3 | toll-like receptor 3 | −0.073548801 | No |
| TAP2 | transporter 2, ATP-binding cassette, sub-family B | −0.081326358 | No |
| | (MDR/TAP) | | |
| UBE2D1 | ubiquitin-conjugating enzyme E2D 1 | −0.089536563 | No |
| | (UBC4/5 homolog, yeast) | | |
| ELF4 | E74-like factor 4 (ets domain transcription factor) | −0.119385988 | No |
| CD4 | CD4 molecule | −0.128431112 | No |
| PTPN6 | protein tyrosine phosphatase, non-receptor type 6 | −0.134158283 | No |
| IL16 | interleukin 16 (lymphocyte chemoattractant factor) | −0.139530182 | No |
| MAP3K7 | mitogen-activated protein kinase kinase kinase 7 | −0.140273616 | No |
| RPS9 | ribosomal protein S9 | −0.145742506 | No |
| EIF4G3 | eukaryotic translation initiation factor 4 gamma, 3 | −0.146932095 | No |
| PRKCG | protein kinase C, gamma | −0.148039475 | No |
| TPD52 | tumor protein D52 | −0.149961919 | No |
| MRPL3 | mitochondrial ribosomal protein L3 | −0.158919781 | No |
| EGFR | epidermal growth factor receptor (erythroblastic | −0.164786458 | No |
| | leukemia viral (v-erb-b) oncogene homolog, avian) | | |
| IKBKB | inhibitor of kappa light polypeptide gene enhancer in | −0.167352051 | No |
| | B-cells, kinase beta | | |
| JAK2 | Janus kinase 2 (a protein tyrosine kinase) | −0.167714015 | No |
| CCND2 | cyclin D2 | −0.172594517 | No |
| HDAC9 | histone deacetylase 9 | −0.17316395 | No |
| AARS | alanyl-tRNA synthetase | −0.175502419 | No |
| IL27RA | interleukin 27 receptor, alpha | −0.177759618 | No |
| IFNGR1 | interferon gamma receptor 1 | −0.181889921 | No |
| RIPK2 | receptor-interacting serine-threonine kinase 2 | −0.184280902 | No |
| MTIF2 | mitochondrial translational initiation factor 2 | −0.19249332 | No |
| HIF1A | hypoxia-inducible factor 1, alpha subunit (basic helix- | −0.209014714 | No |
| | loop-helix transcription factor) | | |
| CFP | complement factor properdin | −0.21020171 | No |
| F2R | coagulation factor II (thrombin) receptor | −0.219984412 | No |
| ABI1 | abl-interactor 1 | −0.229079112 | No |
| B2M | beta-2-microglobulin | −0.230768695 | No |
| LY75 | lymphocyte antigen 75 | −0.23181951 | No |
| TGFB2 | transforming growth factor, beta 2 | −0.233885586 | No |
| CD47 | CD47 molecule | −0.250719309 | No |
| ITK | IL2-inducible T-cell kinase | −0.254682004 | No |
| CSK | c-src tyrosine kinase | −0.260919124 | No |
| DARS | aspartyl-tRNA synthetase | −0.26485461 | No |
| ACVR2A | activin A receptor, type IIA | −0.265007794 | No |
| IRF4 | interferon regulatory factor 4 | −0.281879067 | No |
| IRF7 | interferon regulatory factor 7 | −0.282547563 | No |
| TRAF2 | TNF receptor-associated factor 2 | −0.292227983 | No |
| NCK1 | NCK adaptor protein 1 | −0.300779611 | No |
| GALNT1 | UDP-N-acetyl-alpha-D-galactosamine:polypeptide N- | −0.308693349 | No |
| | acetylgalactosaminyltransferase 1 (GalNAc-T1) | | |
| DEGS1 | degenerative spermatocyte homolog 1, lipid desaturase | −0.309142411 | No |
| | (Drosophila) | | |
| IL2RB | interleukin 2 receptor, beta | −0.317925036 | No |
| TAPBP | TAP binding protein (tapasin) | −0.321666211 | No |
| ACHE | acetylcholinesterase (Yt blood group) | −0.328164309 | No |
| CD247 | CD247 molecule | −0.337787271 | No |
| IL7 | interleukin 7 | −0.356599182 | No |
| CD2 | CD2 molecule | −0.376374722 | No |
| BCL10 | B-cell CLL/lymphoma 10 | −0.387535632 | No |
| PSMB10 | proteasome (prosome, macropain) subunit, beta type, 10 | −0.392836064 | No |
| IL2RA | interleukin 2 receptor, alpha | −0.422955573 | No |
| CCND3 | cyclin D3 | −0.436431259 | No |
| NPM1 | nucleophosmin (nucleolar phosphoprotein B23, | −0.437718987 | No |
| | numatrin) | | |
| EIF5A | eukaryotic translation initiation factor 5A | −0.440881759 | No |
| ABCE1 | ATP-binding cassette, sub-family E (OABP), member 1 | −0.462895155 | No |
| KLRD1 | killer cell lectin-like receptor subfamily D, member 1 | −0.488495886 | No |
| WARS | tryptophanyl-tRNA synthetase | −0.497032523 | No |
| NME1 | non-metastatic cells 1, protein (NM23A) expressed in | −0.507820129 | No |
| IL18 | interleukin 18 (interferon-gamma-inducing factor) | −0.516424298 | No |
| FLNA | filamin A, alpha (actin binding protein 280) | −0.591539323 | No |
| TRAT1 | T cell receptor associated transmembrane adaptor 1 | −0.612580657 | No |
| BCAT1 | branched chain aminotransferase 1, cytosolic | −0.632505953 | No |
| PF4 | platelet factor 4 (chemokine (C-X-C motif) ligand 4) | −0.685499907 | No |
| CD96 | CD96 molecule | −0.696216106 | No |
| TIMP1 | TIMP metallopeptidase inhibitor 1 | −0.705237448 | No |
| CTSS | cathepsin S | −0.790032566 | No |
| RARS | arginyl-tRNA synthetase | −0.869948447 | No |
| CAPG | capping protein (actin filament), gelsolin-like | −0.915236473 | No |
| AKT1 | v-akt murine thymoma viral oncogene homolog 1 | −0.990338564 | No |
| THY1 | Thy-1 cell surface antigen | −1.102289557 | No |
| RPL3L | ribosomal protein L3-like | −1.196903586 | No |
| EREG | epiregulin | −1.240362406 | No |
| KRT1 | keratin 1 (epidermolytic hyperkeratosis) | −1.605908155 | No |

HALLMARK_IL6_JAK_STAT3_SIGNALING

| | | | |
|---|---|---|---|
| IL1B | interleukin 1, beta | 3.462865114 | Yes |
| IL6 | interleukin 6 (interferon, beta 2) | 3.456357479 | Yes |
| CSF3R | colony stimulating factor 3 receptor (granulocyte) | 3.238983631 | Yes |
| TNF | tumor necrosis factor (TNF superfamily, member 2) | 2.751254082 | Yes |
| CXCL1 | chemokine (C-X-C motif) ligand 1 (melanoma growth | 2.667189121 | Yes |
| | stimulating activity, alpha) | | |
| CCR1 | chemokine (C-C motif) receptor 1 | 2.132987738 | Yes |
| TNFRSF1B | tumor necrosis factor receptor superfamily, member 1B | 1.259392858 | Yes |
| IL2RG | interleukin 2 receptor, gamma (severe | 1.083326221 | Yes |
| | combined immunodeficiency) | | |
| PIK3R5 | phosphoinositide-3-kinase, regulatory subunit 5, p101 | 1.057828546 | Yes |
| CXCL3 | chemokine (C-X-C motif) ligand 3 | 0.966697574 | Yes |
| SOCS1 | suppressor of cytokine signaling 1 | 0.912176847 | Yes |
| CSF2RB | colony stimulating factor 2 receptor, beta, | 0.888966024 | Yes |
| | low-affinity (granulocyte-macrophage) | | |
| CD14 | CD14 molecule | 0.848022759 | Yes |
| CSF2RA | colony stimulating factor 2 receptor, alpha, | 0.78864789 | Yes |
| | low-affinity (granulocyte-macrophage) | | |
| CRLF2 | cytokine receptor-like factor 2 | 0.754059553 | Yes |
| TLR2 | toll-like receptor 2 | 0.671268702 | Yes |
| CD38 | CD38 molecule | 0.640005708 | Yes |
| LTB | lymphotoxin beta (TNF superfamily, member 3) | 0.579620421 | Yes |
| CNTFR | ciliary neurotrophic factor receptor | 0.557358146 | Yes |
| CXCL9 | chemokine (C-X-C motif) ligand 9 | 0.391491205 | No |
| ITGB3 | integrin, beta 3 (platelet glycoprotein IIIa, antigen CD61) | 0.256395549 | No |
| CCL7 | chemokine (C-C motif) ligand 7 | 0.251811057 | No |
| CXCL10 | chemokine (C-X-C motif) ligand 10 | 0.226761773 | No |
| IRF1 | interferon regulatory factor 1 | 0.214157283 | No |
| ACVRL1 | activin A receptor type II-like 1 | 0.211801216 | No |
| OSMR | oncostatin M receptor | 0.207781732 | No |
| EBI3 | Epstein-Barr virus induced gene 3 | 0.188898429 | No |
| FAS | Fas (TNF receptor superfamily, member 6) | 0.173293099 | No |
| ITGA4 | integrin, alpha 4 (antigen CD49D, alpha 4 | 0.159151196 | No |
| | subunit of VLA-4 receptor) | | |
| TGFB1 | transforming growth factor, beta 1 | 0.120179698 | No |
| | (Camurati-Engelmann disease) | | |
| IL15RA | interleukin 15 receptor, alpha | 0.084517181 | No |
| CXCL11 | chemokine (C-X-C motif) ligand 11 | 0.079039596 | No |
| STAT2 | signal transducer and activator of transcription 2, 113kDa | 0.064280972 | No |
| IFNAR1 | interferon (alpha, beta and omega) receptor 1 | 0.059620846 | No |
| BAK1 | BCL2-antagonist/killer 1 | 0.059363768 | No |
| STAT1 | signal transducer and activator of transcription 1, 91kDa | 0.050059285 | No |
| IFNGR2 | interferon gamma receptor 2 (interferon gamma | 0.046017136 | No |
| | transducer 1) | | |
| CSF1 | colony stimulating factor 1 (macrophage) | 0.042810541 | No |
| IL17RB | interleukin 17 receptor B | 0.030075336 | No |
| CD9 | CD9 molecule | 0.026316533 | No |
| IL3RA | interleukin 3 receptor, alpha (low affinity) | 0.007537159 | No |
| HAX1 | HCLS1 associated protein X-1 | −0.02173438 | No |
| CXCL13 | chemokine (C-X-C motif) ligand 13 (B-cell | −0.04557885 | No |
| | chemoattractant) | | |
| IL12RB1 | interleukin 12 receptor, beta 1 | −0.05035164 | No |
| IL1R1 | interleukin 1 receptor, type I | −0.05148519 | No |
| IL9R | interleukin 9 receptor | −0.07257795 | No |
| TNFRSF21 | tumor necrosis factor receptor superfamily, member 21 | −0.09369773 | No |
| CBL | Cas-Br-M (murine) ecotropic retroviral | −0.10830661 | No |
| | transforming sequence | | |
| CD36 | CD36 molecule (thrombospondin receptor) | −0.11540288 | No |
| PTPN11 | protein tyrosine phosphatase, non-receptor | −0.14138806 | No |
| | type 11 (Noonan syndrome 1) | | |
| TYK2 | tyrosine kinase 2 | −0.16118781 | No |
| JUN | jun oncogene | −0.16524719 | No |
| IL17RA | interleukin 17 receptor A | −0.17849953 | No |
| IFNGR1 | interferon gamma receptor 1 | −0.18188992 | No |
| IL6ST | interleukin 6 signal transducer (gp130, | −0.20624523 | No |
| | oncostatin M receptor) | | |
| MAP3K8 | mitogen-activated protein kinase kinase | −0.2093222 | No |
| | kinase 8 | | |
| PIM1 | pim-1 oncogene | −0.21471494 | No |
| IL1R2 | interleukin 1 receptor, type II | −0.21576229 | No |
| PTPN2 | protein tyrosine phosphatase, non-receptor type 2 | −0.21662436 | No |
| PDGFC | platelet derived growth factor C | −0.21919613 | No |
| PTPN1 | protein tyrosine phosphatase, non-receptor type 1 | −0.23609251 | No |
| IL10RB | interleukin 10 receptor, beta | −0.26419479 | No |
| STAM2 | signal transducing adaptor molecule (SH3 domain and | −0.28349385 | No |
| | ITAM motif) 2 | | |
| ACVR1B | activin A receptor, type IB | −0.29110909 | No |
| IL7 | interleukin 7 | −0.35659918 | No |
| CD44 | CD44 molecule (Indian blood group) | −0.41463488 | No |
| IL13RA1 | interleukin 13 receptor, alpha 1 | −0.41913402 | No |
| IL2RA | interleukin 2 receptor, alpha | −0.42295557 | No |
| SOCS3 | suppressor of cytokine signaling 3 | −0.44126242 | No |
| LEPR | leptin receptor | −0.48937947 | No |
| TNFRSF12A | tumor necrosis factor receptor superfamily, member 12A | −0.51185614 | No |
| GRB2 | growth factor receptor-bound protein 2 | −0.56912065 | No |
| CSF2 | colony stimulating factor 2 (granulocyte-macrophage) | −0.62405962 | No |
| LTBR | lymphotoxin beta receptor (TNFR superfamily, | −0.67464828 | No |
| | member 3) | | |
| PF4 | platelet factor 4 (chemokine (C-X-C motif) ligand 4) | −0.68549991 | No |
| A2M | alpha-2-macroglobulin | −0.77927083 | No |
| MYD88 | myeloid differentiation primary response gene (88) | −0.79646552 | No |
| IL18R1 | interleukin 18 receptor 1 | −0.81675339 | No |
| STAT3 | signal transducer and activator of transcription 3 | −0.89553529 | No |
| | (acute-phase response factor) | | |
| TNFRSF1A | tumor necrosis factor receptor superfamily, member lA | −0.96931034 | No |
| HMOX1 | heme oxygenase (decycling) 1 | −1.15975845 | No |

HALLMARK_TNFA_SIGNALING_VIA_NFKB

| | | | |
|---|---|---|---|
| IL1B | interleukin 1, beta | 3.462865114 | Yes |
| IL6 | interleukin 6 (interferon, beta 2) | 3.456357479 | Yes |
| TNF | tumor necrosis factor (TNF superfamily, member 2) | 2.751254082 | Yes |
| CXCL1 | chemokine (C-X-C motif) ligand 1 (melanoma | 2.667189121 | Yes |
| | growth stimulating activity, alpha) | | |
| TNFRSF9 | tumor necrosis factor receptor superfamily, member 9 | 2.029400826 | Yes |
| CXCL2 | chemokine (C-X-C motif) ligand 2 | 1.990867496 | Yes |
| SERPINE1 | serpin peptidase inhibitor, clade E (nexin, plasminogen | 1.842594028 | Yes |
| | activator inhibitor type 1), member 1 | | |
| CCL5 | chemokine (C-C motif) ligand 5 | 1.827621102 | Yes |
| PTX3 | pentraxin-related gene, rapidly induced by IL-1 beta | 1.801822662 | Yes |
| PLAUR | plasminogen activator, urokinase receptor | 1.308592319 | Yes |
| CCL4 | chemokine (C-C motif) ligand 4 | 1.303452969 | Yes |
| TRAF1 | TNF receptor-associated factor 1 | 1.300272942 | Yes |
| PLEK | pleckstrin | 1.008932829 | Yes |
| CXCL3 | chemokine (C-X-C motif) ligand 3 | 0.966697574 | Yes |
| CCL2 | chemokine (C-C motif) ligand 2 | 0.932862163 | Yes |
| DUSP2 | dual specificity phosphatase 2 | 0.894410133 | Yes |
| IL23A | interleukin 23, alpha subunit p19 | 0.838799894 | Yes |
| CCRL2 | chemokine (C-C motif) receptor-like 2 | 0.822621703 | Yes |
| SLC2A6 | solute carrier family 2 (facilitated glucose transporter), | 0.820073187 | Yes |
| | member 6 | | |
| FOSL1 | FOS-like antigen 1 | 0.779859602 | Yes |
| TNFSF9 | tumor necrosis factor (ligand) superfamily, member 9 | 0.771818101 | Yes |
| BCL3 | B-cell CLL/lymphoma 3 | 0.768477619 | Yes |
| ICAM1 | intercellular adhesion molecule 1 (CD54), | 0.746375382 | Yes |
| | human rhinovirus receptor | | |
| NFKBIE | nuclear factor of kappa light polypeptide | 0.742087662 | Yes |
| | gene enhancer in B-cells inhibitor, epsilon | | |
| CEBPD | CCAAT/enhancer binding protein (C/EBP), delta | 0.70218122 | Yes |
| PTGS2 | prostaglandin-endoperoxide synthase 2 | 0.696858883 | Yes |
| | (prostaglandin G/H synthase and cyclooxygenase) | | |
| TLR2 | toll-like receptor 2 | 0.671268702 | Yes |
| KLF4 | Kruppel-like factor 4 (gut) | 0.639784276 | Yes |
| CCL20 | chemokine (C-C motif) ligand 20 | 0.625092983 | Yes |
| NR4A3 | nuclear receptor subfamily 4, group A, member 3 | 0.624355614 | Yes |
| CD80 | CD80 molecule | 0.600226164 | Yes |
| AREG | amphiregulin (schwannoma-derived growth factor) | 0.548579574 | Yes |
| LIF | leukemia inhibitory factor (cholinergic differentiation | 0.547570944 | Yes |
| | factor) | | |
| TNFAIP6 | tumor necrosis factor, alpha-induced protein 6 | 0.543578744 | Yes |
| PHLDA2 | pleckstrin homology-like domain, family A, member 2 | 0.543070078 | Yes |
| HBEGF | heparin-binding EGF-like growth factor | 0.541148901 | Yes |
| B4GALT5 | UDP-Gal:betaGlcNAc beta 1,4- | 0.432685524 | No |
| | galactosyltransferase, polypeptide 5 | | |
| FUT4 | fucosyltransferase 4 (alpha (1,3) | 0.386936367 | No |
| | fucosyltransferase, myeloid-specific) | | |
| TNC | tenascin C (hexabrachion) | 0.34414199 | No |
| GCH1 | GTP cyclohydrolase 1 (dopa-responsive dystonia) | 0.327480644 | No |
| OLR1 | oxidised low density lipoprotein (lectin-like) | 0.284122884 | No |
| | receptor 1 | | |
| INHBA | inhibin, beta A (activin A, activin AB alpha polypeptide) | 0.280317694 | No |
| CLCF1 | cardiotrophin-like cytokine factor 1 | 0.26978004 | No |
| TNFAIP2 | tumor necrosis factor, alpha-induced protein 2 | 0.231159508 | No |
| PHLDA1 | pleckstrin homology-like domain, family A, member 1 | 0.226918563 | No |
| CXCL10 | chemokine (C-X-C motif) ligand 10 | 0.226761773 | No |
| DUSP4 | dual specificity phosphatase 4 | 0.222908363 | No |
| IRF1 | interferon regulatory factor 1 | 0.214157283 | No |
| NFIL3 | nuclear factor, interleukin 3 regulated | 0.181677267 | No |
| NR4A2 | nuclear receptor subfamily 4, group A, member 2 | 0.1683653 | No |
| CD69 | CD69 molecule | 0.166904241 | No |
| PTGER4 | prostaglandin E receptor 4 (subtype EP4) | 0.143603638 | No |
| SERPINB2 | serpin peptidase inhibitor, clade B (ovalbumin), | 0.129979178 | No |
| | member 2 | | |
| PANX1 | pannexin 1 | 0.12138366 | No |
| FOSB | FBJ murine osteosarcoma viral oncogene homolog B | 0.113320433 | No |
| TNFAIP3 | tumor necrosis factor, alpha-induced protein 3 | 0.100990877 | No |
| BTG1 | B-cell translocation gene 1, anti-proliferative | 0.100179978 | No |
| IL15RA | interleukin 15 receptor, alpha | 0.084517181 | No |
| CXCL11 | chemokine (C-X-C motif) ligand 11 | 0.079039596 | No |
| SLC2A3 | solute carrier family 2 (facilitated glucose transporter), | 0.069677271 | No |
| | member 3 | | |
| IFIT2 | interferon-induced protein with tetratricopeptide | 0.05142466 | No |
| | repeats 2 | | |
| IFNGR2 | interferon gamma receptor 2 (interferon gamma | 0.046017136 | No |
| | transducer 1) | | |
| CSF1 | colony stimulating factor 1 (macrophage) | 0.042810541 | No |
| STAT5A | signal transducer and activator of transcription 5A | 0.015167505 | No |
| ATF3 | activating transcription factor 3 | 0.007687764 | No |
| EGR3 | early growth response 3 | 0.003858579 | No |
| EDN1 | endothelin 1 | 0.002488923 | No |
| F2RL1 | coagulation factor II (thrombin) receptor-like 1 | −0.00567373 | No |
| PTPRE | protein tyrosine phosphatase, receptor type, E | −0.01096273 | No |
| GFPT2 | glutamine -fructose -6-phosphate | −0.02297758 | No |
| | transaminase 2 | | |
| IER5 | immediate early response 5 | −0.02526059 | No |
| TANK | TRAF family member-associated NFKB activator | −0.02547635 | No |
| TAP1 | transporter 1, ATP-binding cassette, sub-family B | −0.03303317 | No |
| | (MDR/TAP) | | |
| GADD45B | growth arrest and DNA-damage-inducible, beta | −0.03472245 | No |
| NFKBIA | nuclear factor of kappa light polypeptide | −0.03587324 | No |
| | gene enhancer in B-cells inhibitor, alpha | | |
| KLF10 | Kruppel-like factor 10 | −0.03688239 | No |
| GEM | GTP binding protein overexpressed in skeletal muscle | −0.04264625 | No |
| DNAJB4 | DnaJ (Hsp40) homolog, subfamily B, member 4 | −0.05442556 | No |
| PER1 | period homolog 1 (Drosophila) | −0.05708625 | No |
| BCL6 | B-cell CLL/lymphoma 6 (zinc finger protein 51) | −0.06102759 | No |
| BIRC3 | baculoviral IAP repeat-containing 3 | −0.06623559 | No |
| LAMB3 | laminin, beta 3 | −0.07300156 | No |
| ZC3H12A | zinc finger CCCH-type containing 12A | −0.07837815 | No |
| FOS | v-fos FBJ murine osteosarcoma viral oncogene homolog | −0.08169513 | No |
| CDKN1A | cyclin-dependent kinase inhibitor 1A (p21, Cip1) | −0.08443426 | No |
| CCNL1 | cyclin Ll | −0.08534243 | No |
| MAP2K3 | mitogen-activated protein kinase kinase 3 | −0.09317061 | No |
| ZBTB10 | zinc finger and BTB domain containing 10 | −0.10083003 | No |
| TIPARP | TCDD-inducible poly(ADP-ribose) | −0.10120153 | No |
| | polymerase | | |
| TRIP10 | thyroid hormone receptor interactor 10 | −0.11554773 | No |
| SERPINB8 | serpin peptidase inhibitor, clade B | −0.1231901 | No |
| | (ovalbumin), member 8 | | |
| BMP2 | bone morphogenetic protein 2 | −0.14009446 | No |
| ATP2B1 | ATPase, Ca++ transporting, plasma membrane 1 | −0.14145075 | No |
| SOD2 | superoxide dismutase 2, mitochondrial | −0.14157586 | No |
| GADD45A | growth arrest and DNA-damage-inducible, alpha | −0.14824487 | No |
| DDX58 | DEAD (Asp-Glu-Ala-Asp) box polypeptide 58 | −0.15164469 | No |
| FJX1 | four jointed box 1 (Drosophila) | −0.15888149 | No |
| KLF6 | Kruppel-like factor 6 | −0.16283926 | No |
| DUSP1 | dual specificity phosphatase 1 | −0.16306108 | No |
| JUN | jun oncogene | −0.16524719 | No |
| REL | v-rel reticuloendotheliosis viral oncogene homolog | −0.16811736 | No |
| | (avian) | | |
| PDE4B | phosphodiesterase 4B, cAMP-specific | −0.16894662 | No |
| | (phosphodiesterase E4 dunce homolog, Drosophila) | | |
| MXD1 | MAX dimerization protein 1 | −0.16998833 | No |
| BTG2 | BTG family, member 2 | −0.17541341 | No |
| TNIP2 | TNFAIP3 interacting protein 2 | −0.18125953 | No |
| RIPK2 | receptor-interacting serine-threonine kinase 2 | −0.1842809 | No |
| NR4A1 | nuclear receptor subfamily 4, group A, member 1 | −0.19607757 | No |
| JAG 1 | jagged 1 (Alagille syndrome) | −0.19851652 | No |
| LDLR | low density lipoprotein receptor (familial | −0.20240238 | No |
| | hypercholesterolemia) | | |
| NFKB2 | nuclear factor of kappa light polypeptide | −0.20358734 | No |
| | gene enhancer in B-cells 2 (p49/p100) | | |
| IFIH1 | interferon induced with helicase C domain 1 | −0.20616461 | No |
| IL6ST | interleukin 6 signal transducer (gp130, oncostatin M | −0.20624523 | No |
| | receptor) | | |
| SLC16A6 | solute carrier family 16, member 6 | −0.20754693 | No |
| | (monocarboxylic acid transporter 7) | | |
| MAP3K8 | mitogen-activated protein kinase kinase kinase 8 | −0.2093222 | No |
| RELB | v-rel reticuloendotheliosis viral oncogene | −0.21800217 | No |
| | homolog B, nuclear factor of kappa light | | |
| | polypeptide gene enhancer in B-cells 3 (avian) | | |
| G0S2 | G0/Glswitch 2 | −0.22224158 | No |
| ETS2 | v-ets erythroblastosis virus E26 oncogene homolog 2 | −0.22630355 | No |
| | (avian) | | |
| IL1A | interleukin 1, alpha | −0.22882077 | No |
| MARCKS | myristoylated alanine-rich protein kinase C substrate | −0.22976576 | No |
| SPHK1 | sphingosine kinase 1 | −0.23109274 | No |
| FOSL2 | FOS-like antigen 2 | −0.23224656 | No |
| TRIB1 | tribbles homolog 1 (Drosophila) | −0.24160028 | No |
| EGR2 | early growth response 2 (Krox-20 homolog, | −0.2432663 | No |
| | Drosophila) | | |
| TNFAIP8 | tumor necrosis factor, alpha-induced protein8 | −0.24982262 | No |
| PDLIM5 | PDZ and LIM domain 5 | −0.25230208 | No |
| RHOB | ras homolog gene family, member B | −0.25625306 | No |
| CFLAR | CASP8 and FADD-like apoptosis regulator | −0.25688204 | No |
| NFKB1 | nuclear factor of kappa light polypeptide | −0.25809258 | No |
| | gene enhancer in B-cells 1 (p105) | | |
| NFE2L2 | nuclear factor (erythroid-derived 2)-like 2 | −0.25833997 | No |
| SMAD3 | SMAD, mothers against DPP homolog 3 (Drosophila) | −0.26333848 | No |
| PLK2 | polo-like kinase 2 (Drosophila) | −0.26362717 | No |
| TNIP1 | TNFAIP3 interacting protein 1 | −0.27545539 | No |
| SAT1 | spermidine/spermine N1-acetyltransferase 1 | −0.27614322 | No |
| EHD1 | EH-domain containing 1 | −0.28172371 | No |
| KYNU | kynureninase (L-kynurenine hydrolase) | −0.28701586 | No |
| CCND1 | cyclin D1 | −0.28974119 | No |
| SNN | stannin | −0.2964263 | No |
| IRS2 | insulin receptor substrate 2 | −0.30096406 | No |
| EIF1 | eukaryotic translation initiation factor 1 | −0.3023158 | No |
| PLAU | plasminogen activator, urokinase | −0.30320007 | No |
| IL7R | interleukin 7 receptor | −0.30657914 | No |
| MAFF | v-maf musculoaponeurotic fibrosarcoma | −0.31840307 | No |
| | oncogene homolog F (avian) | | |
| RELA | v-rel reticuloendotheliosis viral oncogene | −0.32651395 | No |
| | homolog A, nuclear factor of kappa light | | |
| | polypeptide gene enhancer in B-cells 3, p65 (avian) | | |
| ABCA1 | ATP-binding cassette, sub-family A (ABC1), | −0.33476061 | No |
| | member 1 | | |
| PNRC1 | proline-rich nuclear receptor coactivator 1 | −0.3378354 | No |
| NFAT5 | nuclear factor of activated T-cells 5, tonicity-responsive | −0.33818045 | No |
| DUSP5 | dual specificity phosphatase 5 | −0.3400479 | No |
| NINJ1 | ninjurin 1 | −0.37850687 | No |
| KLF9 | Kruppel-like factor 9 | −0.39027402 | No |
| MCL1 | myeloid cell leukemia sequence 1 (BCL2-related) | −0.40117186 | No |
| HES1 | hairy and enhancer of split 1, (Drosophila) | −0.40132076 | No |
| LITAF | lipopolysaccharide-induced TNF factor | −0.40729272 | No |
| CEBPB | CCAAT/enhancer binding protein (C/EBP), beta | −0.41097072 | No |
| CD44 | CD44 molecule (Indian blood group) | −0.41463488 | No |
| SPSB1 | splA/ryanodine receptor domain and SOCS box | −0.42047217 | No |
| | containing 1 | | |
| PFKFB3 | 6-phosphofructo-2-kinase/fructose-2,6-biphosphatase 3 | −0.43228999 | No |
| PPP1R15A | protein phosphatase 1, regulatory (inhibitor) | −0.43424553 | No |
| | subunit 15A | | |
| BIRC2 | baculoviral IAP repeat-containing 2 | −0.4354732 | No |
| TSC22D1 | TSC22 domain family, member 1 | −0.43681926 | No |
| SOCS3 | suppressor of cytokine signaling 3 | −0.44126242 | No |
| ID2 | inhibitor of DNA binding 2, dominant | −0.44699806 | No |
| | negative helix-loop-helix protein | | |
| TUBB2A | tubulin, beta 2A | −0.47803786 | No |
| EGR1 | early growth response 1 | −0.48577189 | No |
| IER3 | immediate early response 3 | −0.49368548 | No |
| EFNA1 | ephrin-A1 | −0.4986856 | No |
| CD83 | CD83 molecule | −0.50458539 | No |
| SQSTM1 | sequestosome 1 | −0.50716919 | No |
| B4GALT1 | UDP-Gal:betaGlcNAc beta 1,4- | −0.51576161 | No |
| | galactosyltransferase, polypeptide 1 | | |
| IL18 | interleukin 18 (interferon-gamma-inducing factor) | −0.5164243 | No |
| KLF2 | Kruppel-like factor 2 (lung) | −0.519408047 | No |
| CYR61 | cysteine-rich, angiogenic inducer, 61 | −0.564450562 | No |
| MYC | v-myc myelocytomatosis viral oncogene | −0.578493118 | No |
| | homolog (avian) | | |
| CSF2 | colony stimulating factor 2 (granulocyte-macrophage) | −0.624059618 | No |
| IER2 | immediate early response 2 | −0.680687428 | No |
| YRDC | yrdC domain containing (E. coli) | −0.685481429 | No |
| SDC4 | syndecan 4 (amphiglycan, ryudocan) | −0.915015876 | No |
| ZFP36 | zinc finger protein 36, C3H type, homolog (mouse) | −0.918698609 | No |
| MSC | musculin (activated B-cell factor-1) | −1.425963402 | No |
| JUNB | jun B proto-oncogene | −1.767692924 | No |

HALLMARK_IL2_STATS_SIGNALING

| | | | |
|---|---|---|---|
| SELL | selectin L (lymphocyte adhesion molecule 1) | 4.533662319 | Yes |
| MUC1 | mucin 1, cell surface associated | 2.119087458 | Yes |
| TNFRSF9 | tumor necrosis factor receptor superfamily, member 9 | 2.029400826 | Yes |
| SERPINC1 | serpin peptidase inhibitor, clade C (antithrombin), | 2.011505842 | Yes |
| | member 1 | | |
| AHCY | S-adenosylhomocysteine hydrolase | 1.540450335 | Yes |
| IL1RL1 | interleukin 1 receptor-like 1 | 1.417232871 | Yes |
| BATF | basic leucine zipper transcription factor, ATF-like | 1.339311957 | Yes |
| GPR65 | G protein-coupled receptor 65 | 1.301272631 | Yes |
| TRAF1 | TNF receptor-associated factor 1 | 1.300272942 | Yes |
| TNFRSF1B | tumor necrosis factor receptor superfamily, | 1.259392858 | Yes |
| | member 1B | | |
| ENO3 | enolase 3 (beta, muscle) | 1.107318997 | Yes |
| CCNE1 | cyclin E1 | 1.016289473 | Yes |
| IRF8 | interferon regulatory factor 8 | 0.984531879 | Yes |
| SLC29A2 | solute carrier family 29 (nucleoside | 0.945622802 | Yes |
| | transporters), member 2 | | |
| RHOH | ras homolog gene family, member H | 0.934723258 | Yes |
| SOCS1 | suppressor of cytokine signaling 1 | 0.912176847 | Yes |
| CD79B | CD79b molecule, immunoglobulin-associated beta | 0.805104673 | Yes |
| IL10 | interleukin 10 | 0.777381659 | Yes |
| CTLA4 | cytotoxic T-lymphocyte-associated protein 4 | 0.773720741 | Yes |
| PLSCR1 | phospholipid scramblase 1 | 0.761682391 | Yes |
| TLR7 | toll-like receptor 7 | 0.725582063 | Yes |
| IL13 | interleukin 13 | 0.6945768 | Yes |
| CD86 | CD86 molecule | 0.631752431 | Yes |
| LTB | lymphotoxin beta (TNF superfamily, member 3) | 0.579620421 | Yes |
| ETV4 | ets variant gene 4 (E1A enhancer binding protein, E1AF) | 0.579223096 | Yes |
| MAP6 | microtubule-associated protein 6 | 0.574204385 | Yes |
| CDC6 | CDC6 cell division cycle 6 homolog | 0.569661617 | Yes |
| | (S. cerevisiae) | | |
| LIF | leukemia inhibitory factor (cholinergic differentiation | 0.547570944 | Yes |
| | factor) | | |
| CD48 | CD48 molecule | 0.540921032 | Yes |
| APLP1 | amyloid beta (A4) precursor-like protein 1 | 0.531183481 | Yes |
| RGS16 | regulator of G-protein signalling 16 | 0.526294231 | Yes |
| ICOS | inducible T-cell co-stimulator | 0.480524778 | No |
| SHE | Src homology 2 domain containing E | 0.421151727 | No |
| AGER | advanced glycosylation end product-specific receptor | 0.402864873 | No |
| TNFSF11 | tumor necrosis factor (ligand) superfamily, member 11 | 0.372757733 | No |
| SLC39A8 | solute carrier family 39 (zinc transporter), member 8 | 0.364194393 | No |
| IL10RA | interleukin 10 receptor, alpha | 0.343504012 | No |
| GPX4 | glutathione peroxidase 4 (phospholipid hydroperoxidase) | 0.319138467 | No |
| SPP1 | secreted phosphoprotein 1 (osteopontin, | 0.311187059 | No |
| | bone sialoprotein I, early T-lymphocyte activation 1) | | |
| GALM | galactose mutarotase (aldose 1-epimerase) | 0.300109804 | No |
| PRAF2 | PRA1 domain family, member 2 | 0.275571346 | No |
| UMPS | uridine monophosphate synthetase (orotate | 0.234596103 | No |
| | phosphoribosyl transferase and orotidine-5'- | | |
| | decarboxylase) | | |
| TNFRSF8 | tumor necrosis factor receptor superfamily, member | 0.232401714 | No |
| SPRY4 | sprouty homolog 4 (Drosophila) | 0.231738269 | No |
| PHLDA1 | pleckstrin homology-like domain, family A, member 1 | 0.226918563 | No |
| CXCL10 | chemokine (C-X-C motif) ligand 10 | 0.226761773 | No |
| LRRC8C | leucine rich repeat containing 8 family, member C | 0.22627607 | No |
| CST7 | cystatin F (leukocystatin) | 0.22475262 | No |
| CASP3 | caspase 3, apoptosis-related cysteine peptidase | 0.213040709 | No |
| NFIL3 | nuclear factor, interleukin 3 regulated | 0.181677267 | No |
| AKAP2 | A kinase (PRKA) anchor protein 2 | 0.180033192 | No |
| SELP | selectin P (granule membrane protein 140kDa, | 0.175435156 | No |
| | antigen CD62) | | |
| AMACR | alpha-methylacyl-CoA racemase | 0.139369324 | No |
| ST3GAL4 | ST3 beta-galactoside alpha-2,3-sialyltransferase 4 | 0.134488985 | No |
| GBP4 | guanylate binding protein 4 | 0.095500618 | No |
| P4HA1 | procollagen-proline, 2-oxoglutarate 4- | 0.09174899 | No |
| | dioxygenase (proline 4-hydroxylase), alpha polypeptide I | | |
| SYT11 | synaptotagmin XI | 0.088011898 | No |
| XBP1 | X-box binding protein 1 | 0.087087706 | No |
| ADAM19 | ADAM metallopeptidase domain 19 (meltrin beta) | 0.073120117 | No |
| SLC2A3 | solute carrier family 2 (facilitated glucose | 0.069677271 | No |
| | transporter), member 3 | | |
| PENK | proenkephalin | 0.058247235 | No |
| CDKN1C | cyclin-dependent kinase inhibitor 1C (p57, Kip2) | 0.05285516 | No |
| SWAP70 | — | 0.047020242 | No |
| PLAGL1 | pleiomorphic adenoma gene-like 1 | 0.045933478 | No |
| PRKCH | protein kinase C, eta | 0.043467484 | No |
| CSF1 | colony stimulating factor 1 (macrophage) | 0.042810541 | No |
| SNX9 | sorting nexin 9 | 0.031658247 | No |
| F2RL2 | coagulation factor II (thrombin) receptor-like 2 | 0.016297607 | No |
| ENPP1 | ectonucleotide pyrophosphatase/phosphodiesterase 1 | 0.012228195 | No |
| IL3RA | interleukin 3 receptor, alpha (low affinity) | 0.007537159 | No |
| P2RX4 | purinergic receptor P2X, ligand-gated ion channel, 4 | 0.002422775 | No |
| TNFRSF4 | tumor necrosis factor receptor superfamily, member 4 | −0.00651959 | No |
| FGL2 | fibrinogen-like 2 | −0.00713548 | No |
| ITGA6 | integrin, alpha 6 | −0.00743946 | No |
| PTGER2 | prostaglandin E receptor 2 (subtype EP2), 53kDa | −0.01937426 | No |
| GADD45B | growth arrest and DNA-damage-inducible, beta | −0.03472245 | No |
| PTCH1 | patched homolog 1 (Drosophila) | −0.06307594 | No |
| BCL2 | B-cell CLL/lymphoma 2 | −0.06359753 | No |
| IKZF4 | IKAROS family zinc finger 4 (Eos) | −0.07420553 | No |
| TNFRSF21 | tumor necrosis factor receptor superfamily, | −0.09369773 | No |
| | member 21 | | |
| POU2F1 | POU domain, class 2, transcription factor 1 | −0.09776106 | No |
| TIAM1 | T-cell lymphoma invasion and metastasis 1 | −0.09797619 | No |
| NFKBIZ | nuclear factor of kappa light polypeptide | −0.10177696 | No |
| | gene enhancer in B-cells inhibitor, zeta | | |
| RABGAP1L | RAB GTPase activating protein 1-like | −0.1046719 | No |
| SOCS2 | suppressor of cytokine signaling 2 | −0.10708491 | No |
| FURIN | furin (paired basic amino acid cleaving enzyme) | −0.10786293 | No |
| NRP1 | neuropilin 1 | −0.10988839 | No |
| AHNAK | AHNAK nucleoprotein (desmoyokin) | −0.12126806 | No |
| IGF1R | insulin-like growth factor 1 receptor | −0.12401183 | No |
| DHRS3 | dehydrogenase/reductase (SDR family) member 3 | −0.12833633 | No |
| ALCAM | activated leukocyte cell adhesion molecule | −0.12868702 | No |
| RORA | RAR-related orphan receptor A | −0.13175237 | No |
| BMP2 | bone morphogenetic protein 2 | −0.14009446 | No |
| ARL4A | ADP-ribosylation factor-like 4A | −0.14104421 | No |
| SLC1A5 | solute carrier family 1 (neutral amino acid transporter), | −0.14482026 | No |
| | member 5 | | |
| NCOA3 | nuclear receptor coactivator 3 | −0.14673433 | No |
| HIPK2 | homeodomain interacting protein kinase 2 | −0.14735302 | No |
| CYFIP1 | cytoplasmic FMR1 interacting protein 1 | −0.15203957 | No |
| KLF6 | Kruppel-like factor 6 | −0.16283926 | No |
| NDRG1 | N-myc downstream regulated gene 1 | −0.16343482 | No |
| SH3BGRL2 | SH3 domain binding glutamic acid-rich protein like 2 | −0.16945736 | No |
| MXD1 | MAX dimerization protein 1 | −0.16998833 | No |
| NT5E | 5'-nucleotidase, ecto (CD73) | −0.17018586 | No |
| CDCP1 | CUB domain containing protein 1 | −0.17038067 | No |
| CCND2 | cyclin D2 | −0.17259452 | No |
| IFNGR1 | interferon gamma receptor 1 | −0.18188992 | No |
| TGM2 | transglutaminase 2 (C polypeptide, protein- | −0.18499345 | No |
| | glutamine-gamma-glutamyltransferase) | | |
| CDC42SE2 | CDC42 small effector 2 | −0.19735253 | No |
| BMPR2 | bone morphogenetic protein receptor, type II | −0.2040211 | No |
| | (serine/threonine kinase) | | |
| PTRH2 | peptidyl-tRNA hydrolase 2 | −0.20548932 | No |
| PDCD2L | programmed cell death 2-like | −0.20793679 | No |
| MAP3K8 | mitogen-activated protein kinase kinase kinase 8 | −0.2093222 | No |
| SPRED2 | sprouty-related, EVH1 domain containing 2 | −0.21080628 | No |
| ITGAV | integrin, alpha V (vitronectin receptor, alpha | −0.21208024 | No |
| | polypeptide, antigen CD51) | | |
| AHR | aryl hydrocarbon receptor | −0.21279255 | No |
| IGF2R | insulin-like growth factor 2 receptor | −0.21324262 | No |
| PIM1 | pim-1 oncogene | −0.21471494 | No |
| IL1R2 | interleukin 1 receptor, type II | −0.21576229 | No |
| SNX14 | sorting nexin 14 | −0.22292101 | No |
| DCPS | decapping enzyme, scavenger | −0.22333506 | No |
| COL6A1 | collagen, type VI, alpha 1 | −0.2306418 | No |
| SMPDL3A | sphingomyelin phosphodiesterase, acid-like 3A | −0.2336476 | No |
| PUS1 | pseudouridylate synthase 1 | −0.23526376 | No |
| RHOB | ras homolog gene family, member B | −0.25625306 | No |
| BCL2L1 | BCL2-like 1 | −0.26874608 | No |
| IRF6 | interferon regulatory factor 6 | −0.26956779 | No |
| MYO1E | myosin IE | −0.27304026 | No |
| IRF4 | interferon regulatory factor 4 | −0.28187907 | No |
| IKZF2 | IKAROS family zinc finger 2 (Helios) | −0.28325367 | No |
| HK2 | hexokinase 2 | −0.29201287 | No |
| TNFRSF18 | tumor necrosis factor receptor superfamily, member 18 | −0.29958081 | No |
| HUWE1 | HECT, UBA and WWE domain containing 1 | −0.30141243 | No |
| S100A1 | S100 calcium binding protein A1 | −0.30719495 | No |
| IL2RB | interleukin 2 receptor, beta | −0.31792504 | No |
| MAFF | v-maf musculoaponeurotic fibrosarcoma | −0.31840307 | No |
| | oncogene homolog F (avian) | | |
| ITGAE | integrin, alpha E (antigen CD103, human | −0.32581159 | No |
| | mucosal lymphocyte antigen 1; alpha polypeptide) | | |
| TWSG1 | twisted gastrulation homolog 1 (Drosophila) | −0.3268795 | No |
| ECM1 | extracellular matrix protein 1 | −0.32777062 | No |
| ITIH5 | inter-alpha (globulin) inhibitor H5 | −0.32929945 | No |
| MAPKAPK2 | mitogen-activated protein kinase-activated protein | −0.33747 | No |
| | kinase 2 | | |
| GATA1 | GATA binding protein 1 (globin transcription factor 1) | −0.34819636 | No |
| COCH | coagulation factor C homolog, cochlin | −0.35349667 | No |
| | (Limulus polyphemus) | | |
| FAM126B | family with sequence similarity 126, member B | −0.37080041 | No |
| GABARAPL1 | GABA(A) receptor-associated protein like 1 | −0.37952036 | No |
| UCK2 | uridine-cytidine kinase 2 | −0.39549923 | No |
| CISH | cytokine inducible SH2-containing protein | −0.40262353 | No |
| CTSZ | cathepsin Z | −0.41291174 | No |
| CD44 | CD44 molecule (Indian blood group) | −0.41463488 | No |
| LRIG1 | leucine-rich repeats and immunoglobulin-like domains 1 | −0.41774288 | No |
| IL2RA | interleukin 2 receptor, alpha | −0.42295557 | No |
| PHTF2 | putative homeodomain transcription factor 2 | −0.43087524 | No |
| FAH | fumarylacetoacetate hydrolase (fumarylacetoacetase) | −0.43588102 | No |
| CCND3 | cyclin D3 | −0.43643126 | No |
| MYO1C | myosin IC | −0.44808453 | No |
| ODC1 | ornithine decarboxylase 1 | −0.46056387 | No |
| ANXA4 | annexin A4 | −0.47317448 | No |
| CD83 | CD83 molecule | −0.50458539 | No |
| CKAP4 | cytoskeleton-associated protein 4 | −0.51768428 | No |
| MYC | v-myc myelocytomatosis viral oncogene homolog (avian) | −0.57849312 | No |
| CD81 | CD81 molecule | −0.58255219 | No |
| CSF2 | colony stimulating factor 2 (granulocyte-macrophage) | −0.62405962 | No |
| CAPN3 | calpain 3, (p94) | −0.63329792 | No |
| SYNGR2 | synaptogyrin 2 | −0.63514042 | No |
| GUCY1B3 | guanylate cyclase 1, soluble, beta 3 | −0.64079314 | No |
| CCR4 | chemokine (C-C motif) receptor 4 | −0.64653838 | No |
| PRNP | prion protein (p27-30) (Creutzfeldt-Jakob | −0.76452804 | No |
| | disease, Gerstmann-Strausler-Scheinker | | |
| | syndrome, fatal familial insomnia) | | |
| TNFSF10 | tumor necrosis factor (ligand) superfamily, member 10 | −0.78845328 | No |
| IL18R1 | interleukin 18 receptor 1 | −0.81675339 | No |
| CAPG | capping protein (actin filament), gelsolin-like | −0.91523647 | No |
| EMP1 | epithelial membrane protein 1 | −0.96930897 | No |
| IFITM3 | interferon induced transmembrane protein 3 (I-8U) | −1.08716643 | No |
| GSTO1 | glutathione S-transferase omega 1 | −1.18441701 | No |
| RNH1 | ribonuclease/angiogenin inhibitor 1 | −1.19759107 | No |
| RRAGD | Ras-related GTP binding D | −1.36585367 | No |

TABLE 7
Gene List in gene sets of enriched pathway in Table 5.

| | | | |
|---|---|---|---|
| | | RANK | CORE |
| GENE | | METRIC | ENRICH- |
| SYMBOL | GENE_TITLE | SCORE | MENT |

HALLMARK_INFLAMMATORY_RESPONSE

| | | | |
|---|---|---|---|
| PPBP | pro-platelet basic protein (chemokine | 3.837641716 | Yes |
| | (C-X-C motif) ligand 7) | | |
| IL1B | interleukin 1, beta | 3.462865114 | Yes |
| IL6 | interleukin 6 (interferon, beta 2) | 3.456357479 | Yes |
| CCL1 | chemokine (C-C motif) ligand 1 | 3.329092503 | Yes |
| IL18RAP | interleukin 18 receptor accessory protein | 3.251766205 | Yes |
| CSF3R | colony stimulating factor 3 receptor | 3.238983631 | Yes |
| | (granulocyte) | | |
| OSM | oncostatin M | 2.948629141 | Yes |
| TNF | tumor necrosis factor (TNF superfamily, | 2.751254082 | Yes |
| | member 2) | | |
| CXCL1 | chemokine (C-X-C motif) ligand 1 | 2.667189121 | Yes |
| | (melanoma growth stimulating activity, | | |
| | alpha) | | |
| CCR1 | chemokine (C-C motif) receptor 1 | 2.132987738 | Yes |
| CCR5 | chemokine (C-C motif) receptor 5 | 2.054755688 | Yes |
| TNFRSF9 | tumor necrosis factor receptor | 2.029400826 | Yes |
| | superfamily, member 9 | | |
| CXCL2 | chemokine (C-X-C motif) ligand 2 | 1.990867496 | Yes |
| CCL5 | chemokine (C-C motif) ligand 5 | 1.827621102 | Yes |
| CXCL5 | chemokine (C-X-C motif) ligand 5 | 1.754894018 | Yes |
| CCL3 | chemokine (C-C motif) ligand 3 | 1.564267635 | Yes |
| TNFSF8 | tumor necrosis factor (ligand) | 1.533434033 | Yes |
| | superfamily, member 8 | | |
| TNFSF14 | tumor necrosis factor (ligand) | 1.530670643 | Yes |
| | superfamily, member 14 | | |
| TPO | thyroid peroxidase | 1.496503949 | Yes |
| CCL22 | chemokine (C-C motif) ligand 22 | 1.440871716 | Yes |
| CSF3 | colony stimulating factor 3 | 1.422928095 | Yes |
| | (granulocyte) | | |
| IL21R | interleukin 21 receptor | 1.375825167 | Yes |
| IL12A | interleukin 12A (natural killer cell | 1.315529704 | Yes |
| | stimulatory factor 1, cytotoxic | | |
| | lymphocyte maturation factor 1, p35) | | |
| CCL4 | chemokine (C-C motif) ligand 4 | 1.303452969 | Yes |
| TNFRSF1B | tumor necrosis factor receptor | 1.259392858 | Yes |
| | superfamily, member 1B | | |
| CCL24 | chemokine (C-C motif) ligand 24 | 1.233121157 | Yes |
| CCR2 | chemokine (C-C motif) receptor 2 | 1.17247808 | Yes |
| IL2RG | interleukin 2 receptor, gamma (severe | 1.083326221 | Yes |
| | combined immunodeficiency) | | |
| IFNK | interferon, kappa | 0.981288373 | Yes |
| TNFRSF13B | tumor necrosis factor receptor | 0.980329454 | Yes |
| | superfamily, member 13B | | |
| CXCL3 | chemokine (C-X-C motif) ligand 3 | 0.966697574 | Yes |
| XCR1 | chemokine (C motif) receptor 1 | 0.960530758 | Yes |
| CCL2 | chemokine (C-C motif) ligand 2 | 0.932862163 | Yes |
| CSF2RB | colony stimulating factor 2 receptor, | 0.888966024 | Yes |
| | beta, low-affinity (granulocyte-macrophage) | | |
| IL24 | interleukin 24 | 0.877363384 | Yes |
| IL23A | interleukin 23, alpha subunit p19 | 0.838799894 | Yes |
| IL17B | interleukin 17B | 0.823239386 | Yes |
| AMH | anti-Mullerian hormone | 0.818140507 | Yes |
| CSF2RA | colony stimulating factor 2 receptor, | 0.78864789 | Yes |
| | alpha, low-affinity (granulocyte-macrophage) | | |
| CCL19 | chemokine (C-C motif) ligand 19 | 0.784207284 | Yes |
| IL10 | interleukin 10 | 0.777381659 | Yes |
| TNFSF9 | tumor necrosis factor (ligand) | 0.771818101 | Yes |
| | superfamily, member 9 | | |
| CCL25 | chemokine (C-C motif) ligand 25 | 0.763763368 | Yes |
| CRLF2 | cytokine receptor-like factor 2 | 0.754059553 | Yes |
| IL19 | interleukin 19 | 0.750038803 | Yes |
| CCR9 | chemokine (C-C motif) receptor 9 | 0.745218694 | Yes |
| IL17A | interleukin 17A | 0.707072496 | Yes |
| IL13 | interleukin 13 | 0.6945768 | Yes |
| TNFSF4 | tumor necrosis factor (ligand) superfamily, | 0.689626753 | Yes |
| | member 4 (tax-transcriptionally activated | | |
| | glycoprotein 1, 34kDa) | | |
| IL11 | interleukin 11 | 0.684957564 | Yes |
| CCL17 | chemokine (C-C motif) ligand 17 | 0.664499223 | Yes |
| IL4 | interleukin 4 | 0.65638262 | Yes |
| TNFRSF10B | tumor necrosis factor receptor | 0.651966751 | Yes |
| | superfamily, member 10b | | |
| CCL20 | chemokine (C-C motif) ligand 20 | 0.625092983 | Yes |
| VEGFC | vascular endothelial growth factor C | 0.620482028 | Yes |
| CCR7 | chemokine (C-C motif) receptor 7 | 0.594285488 | Yes |
| LTB | lymphotoxin beta (TNF superfamily, | 0.579620421 | Yes |
| | member 3) | | |
| CNTFR | ciliary neurotrophic factor receptor | 0.557358146 | Yes |
| IL20RB | interleukin 20 receptor beta | 0.556012034 | Yes |
| LIF | leukemia inhibitory factor (cholinergic | 0.547570944 | Yes |
| | differentiation factor) | | |
| CD40 | CD40 molecule, TNF receptor | 0.492939293 | Yes |
| | superfamily member 5 | | |
| CXCR4 | chemokine (C-X-C motif) receptor 4 | 0.484079331 | Yes |
| TSLP | — | 0.474735647 | Yes |
| CX3CR1 | chemokine (C-X3-C motif) receptor 1 | 0.444680154 | Yes |
| LTA | lymphotoxin alpha (TNF superfamily, | 0.44320941 | Yes |
| | member 1) | | |
| FLT4 | fms-related tyrosine kinase 4 | 0.435430467 | Yes |
| CXCL9 | chemokine (C-X-C motif) ligand 9 | 0.391491205 | No |
| CXCR3 | chemokine (C-X-C motif) receptor 3 | 0.380579948 | No |
| TNFSF11 | tumor necrosis factor (ligand) | 0.372757733 | No |
| | superfamily, member 11 | | |
| CCL28 | chemokine (C-C motif) ligand 28 | 0.358908564 | No |
| IL10RA | interleukin 10 receptor, alpha | 0.343504012 | No |
| TNFRSF25 | tumor necrosis factor receptor | 0.331647813 | No |
| | superfamily, member 25 | | |
| IFNAR2 | interferon (alpha, beta and omega) | 0.281477422 | No |
| | receptor 2 | | |
| INHBA | inhibin, beta A (activin A, activin AB | 0.280317694 | No |
| | alpha polypeptide) | | |
| CLCF1 | cardiotrophin-like cytokine factor 1 | 0.26978004 | No |
| CCL7 | chemokine (C-C motif) ligand 7 | 0.251811057 | No |
| PDGFRA | platelet-derived growth factor receptor, | 0.249525964 | No |
| | alpha polypeptide | | |
| TNFRSF8 | tumor necrosis factor receptor | 0.232401714 | No |
| | superfamily, member 8 | | |
| CXCL10 | chemokine (C-X-C motif) ligand 10 | 0.226761773 | No |
| ACVRL1 | activin A receptor type II-like 1 | 0.211801216 | No |
| OSMR | oncostatin M receptor | 0.207781732 | No |
| FAS | Fas (TNF receptor superfamily, member 6) | 0.173293099 | No |
| TNFRSF11A | tumor necrosis factor receptor | 0.15901272 | No |
| | superfamily, member 11a, NFKB activator | | |
| TNFRSF14 | tumor necrosis factor receptor | 0.15240927 | No |
| | superfamily, member 14 (herpesvirus | | |
| | entry mediator) | | |
| KDR | kinase insert domain receptor (a type III | 0.150256053 | No |
| | receptor tyrosine kinase) | | |
| TGFB1 | transforming growth factor, beta 1 | 0.120179698 | No |
| | (Camurati-Engelmann disease) | | |
| TNFSF12 | tumor necrosis factor (ligand) | 0.112915978 | No |
| | superfamily, member 12 | | |
| HGF | hepatocyte growth factor (hepapoietin | 0.098659903 | No |
| | A; scatter factor) | | |
| IL25 | interleukin 25 | 0.094349928 | No |
| IL15RA | interleukin 15 receptor, alpha | 0.084517181 | No |
| CXCL11 | chemokine (C-X-C motif) ligand 11 | 0.079039596 | No |
| EDA | ectodysplasin A | 0.07362695 | No |
| CXCL12 | chemokine (C-X-C motif) ligand 12 | 0.070668168 | No |
| | (stromal cell-derived factor 1) | | |
| IFNAR1 | interferon (alpha, beta and omega) | 0.059620846 | No |
| | receptor 1 | | |
| FLT1 | fms-related tyrosine kinase 1 (vascular | 0.050929114 | No |
| | endothelial growth factor/vascular | | |
| | permeability factor receptor) | | |
| IFNGR2 | interferon gamma receptor 2 (interferon | 0.046017136 | No |
| | gamma transducer 1) | | |
| PDGFRB | platelet-derived growth factor receptor, | 0.045934543 | No |
| | beta polypeptide | | |
| CSF1 | colony stimulating factor 1 (macrophage) | 0.042810541 | No |
| CCL11 | chemokine (C-C motif) ligand 11 | 0.039969306 | No |
| CXCR6 | chemokine (C-X-C motif) receptor 6 | 0.031615365 | No |
| IL17RB | interleukin 17 receptor B | 0.030075336 | No |
| TGFBR1 | transforming growth factor, beta | 0.027430039 | No |
| | receptor I (activin A receptor type II- | | |
| | like kinase, 53kDa) | | |
| PRLR | prolactin receptor | 0.015084264 | No |
| IL3RA | interleukin 3 receptor, alpha (low affinity) | 0.007537159 | No |
| TNFRSF17 | tumor necrosis factor receptor | 7.15E−04 | No |
| | superfamily, member 17 | | |
| TNFRSF4 | tumor necrosis factor receptor | −0.006519591 | No |
| | superfamily, member 4 | | |
| ACVR1 | activin A receptor, type I | −0.006812894 | No |
| TNFSF13B | tumor necrosis factor (ligand) | −0.012086863 | No |
| | superfamily, member 13b | | |
| CCR3 | chemokine (C-C motif) receptor 3 | −0.012453511 | No |
| IL22RA2 | interleukin 22 receptor, alpha 2 | −0.022728374 | No |
| FLT3 | fms-related tyrosine kinase 3 | −0.036438417 | No |
| INHBB | inhibin, beta B (activin AB beta | −0.043366853 | No |
| | polypeptide) | | |
| IL15 | interleukin 15 | −0.045024838 | No |
| CXCL13 | chemokine (C-X-C motif) ligand 13 | −0.045578849 | No |
| | (B-cell chemoattractant) | | |
| CCR10 | chemokine (C-C motif) receptor 10 | −0.047813334 | No |
| IL12RB1 | interleukin 12 receptor, beta 1 | −0.050351642 | No |
| IL1R1 | interleukin 1 receptor, type I | −0.051485192 | No |
| CCR8 | chemokine (C-C motif) receptor 8 | −0.062265944 | No |
| TGFB3 | transforming growth factor, beta 3 | −0.069080003 | No |
| IL9R | interleukin 9 receptor | −0.072577946 | No |
| KIT | v-kit Hardy-Zuckerman 4 feline | −0.07663402 | No |
| | sarcoma viral oncogene homolog | | |
| IL23R | interleukin 23 receptor | −0.083718598 | No |
| TNFRSF21 | tumor necrosis factor receptor | −0.093697727 | No |
| | superfamily, member 21 | | |
| NGFR | nerve growth factor receptor (TNFR | −0.105525017 | No |
| | superfamily, member 16) | | |
| IL1RAP | interleukin 1 receptor accessory protein | −0.112636082 | No |
| BMPR1B | bone morphogenetic protein receptor, | −0.112772785 | No |
| | type IB | | |
| CCR6 | chemokine (C-C motif) receptor 6 | −0.119453743 | No |
| GDF5 | growth differentiation factor 5 | −0.139077023 | No |
| | (cartilage-derived morphogenetic (protein-1) | | |
| BMP2 | bone morphogenetic protein 2 | −0.140094459 | No |
| TNFRSF19 | tumor necrosis factor receptor | −0.163563892 | No |
| | superfamily, member 19 | | |
| EGFR | epidermal growth factor receptor | −0.164786458 | No |
| | (erythroblastic leukemia viral (v-erb-b) | | |
| | oncogene homolog, avian) | | |
| BMPR1A | bone morphogenetic protein receptor, | −0.169454694 | No |
| | type IA | | |
| IL17RA | interleukin 17 receptor A | −0.178499535 | No |
| IFNGR1 | interferon gamma receptor 1 | −0.181889921 | No |
| CXCL16 | chemokine (C-X-C motif) ligand 16 | −0.20063293 | No |
| BMPR2 | bone morphogenetic protein receptor, | −0.204021096 | No |
| | type II (serine/threonine kinase) | | |
| AMHR2 | anti-Mullerian hormone receptor, type II | −0.204355285 | No |
| CSF1R | colony stimulating factor 1 receptor, | −0.204763398 | No |
| | formerly McDonough feline sarcoma | | |
| | viral (v-fms) oncogene homolog | | |
| IL6ST | interleukin 6 signal transducer (gp130, | −0.206245229 | No |
| | oncostatin M receptor) | | |
| IL1R2 | interleukin 1 receptor, type II | −0.215762287 | No |
| PDGFC | platelet derived growth factor C | −0.219196126 | No |
| CX3CL1 | chemokine (C-X3-C motif) ligand 1 | −0.225086227 | No |
| MET | met proto-oncogene (hepatocyte growth | −0.225270301 | No |
| | factor receptor) | | |
| ILIA | interleukin 1, alpha | −0.228820771 | No |
| TGFB2 | transforming growth factor, beta 2 | −0.233885586 | No |
| PDGFB | platelet-derived growth factor beta | −0.238625541 | No |
| | polypeptide (simian sarcoma viral (v-sis) | | |
| | oncogene homolog) | | |
| IL22RA1 | interleukin 22 receptor, alpha 1 | −0.240860581 | No |
| GHR | growth hormone receptor | −0.260692686 | No |
| IL10RB | interleukin 10 receptor, beta | −0.264194787 | No |
| ACVR2A | activin A receptor, type IIA | −0.265007794 | No |
| ACVR1B | activin A receptor, type IB | −0.291109085 | No |
| IL20RA | interleukin 20 receptor, alpha | −0.291834027 | No |
| PDGFA | platelet-derived growth factor alpha | −0.293814242 | No |
| | polypeptide | | |
| TNFRSF11B | tumor necrosis factor receptor | −0.294232696 | No |
| | superfamily, member 11b | | |
| | (osteoprotegerin) | | |
| TGFBR2 | transforming growth factor, beta | −0.298537552 | No |
| | receptor II (70/80kDa) | | |
| TNFRSF18 | tumor necrosis factor receptor | −0.299580812 | No |
| | superfamily, member 18 | | |
| IL7R | interleukin 7 receptor | −0.306579143 | No |
| VEGFB | vascular endothelial growth factor B | −0.315350085 | No |
| IL2RB | interleukin 2 receptor, beta | −0.317925036 | No |
| LIFR | leukemia inhibitory factor receptor alpha | −0.331205487 | No |
| EDA2R | ectodysplasin A2 receptor | −0.346847653 | No |
| EDAR | ectodysplasin A receptor | −0.348729432 | No |
| CXCL14 | chemokine (C-X-C motif) ligand 14 | −0.354058266 | No |
| CNTF | ciliary neurotrophic factor | −0.355169892 | No |
| IL7 | interleukin 7 | −0.356599182 | No |
| CTF1 | cardiotrophin 1 | −0.382776916 | No |
| EPOR | erythropoietin receptor | −0.407475233 | No |
| IL13RA1 | interleukin 13 receptor, alpha 1 | −0.419134021 | No |
| IL2RA | interleukin 2 receptor, alpha | −0.422955573 | No |
| LEPR | leptin receptor | −0.489379466 | No |
| BMP7 | bone morphogenetic protein 7 | −0.494219154 | No |
| | (osteogenic protein 1) | | |
| IL12RB2 | interleukin 12 receptor, beta 2 | −0.511588156 | No |
| TNFRSF12A | tumor necrosis factor receptor | −0.511856139 | No |
| | superfamily, member 12A | | |
| IL18 | interleukin 18 (interferon-gamma- | −0.516424298 | No |
| | inducing factor) | | |
| IL20 | interleukin 20 | −0.517831087 | No |
| MPL | myeloproliferative leukemia virus oncogene | −0.612580657 | No |
| CSF2 | colony stimulating factor 2 | −0.624059618 | No |
| | (granulocyte-macrophage) | | |
| CCR4 | chemokine (C-C motif) receptor 4 | −0.646538377 | No |
| TNFSF15 | tumor necrosis factor (ligand) | −0.652678192 | No |
| | superfamily, member 15 | | |
| LEP | leptin (obesity homolog, mouse) | −0.674438477 | No |
| LTBR | lymphotoxin beta receptor (TNFR | −0.674648285 | No |
| | superfamily, member 3) | | |
| PF4 | platelet factor 4 (chemokine (C-X-C | −0.685499907 | No |
| | motif) ligand 4) | | |
| XCL1 | chemokine (C motif) ligand 1 | −0.706809223 | No |
| TNFSF13 | tumor necrosis factor (ligand) | −0.743143797 | No |
| | superfamily, member 13 | | |
| IL5 | interleukin 5 (colony-stimulating factor, | −0.77403909 | No |
| | eosinophil) | | |
| TNFSF18 | tumor necrosis factor (ligand) | −0.779219151 | No |
| | superfamily, member 18 | | |
| TNFSF10 | tumor necrosis factor (ligand) | −0.788453281 | No |
| | superfamily, member 10 | | |
| IL18R1 | interleukin 18 receptor 1 | −0.816753387 | No |
| TNFRSF1A | tumor necrosis factor receptor | −0.969310343 | No |
| | superfamily, member 1A | | |
| EGF | epidermal growth factor (beta-urogastrone) | −1.048612952 | No |
| TNFRSF13C | tumor necrosis factor receptor | −1.065307021 | No |
| | superfamily, member 13C | | |
| ACVR2B | activin A receptor, type IIB | −1.067408323 | No |

KEGG_HEMATOPOIETIC_CELL_LINEAGE

| | | | |
|---|---|---|---|
| IL1B | interleukin 1, beta | 3.462865114 | Yes |
| IL6 | interleukin 6 (interferon, beta 2) | 3.456357479 | Yes |
| IL18RAP | interleukin 18 receptor accessory protein | 3.251766205 | Yes |
| STAT4 | signal transducer and activator of | 3.148304939 | Yes |
| | transcription 4 | | |
| TNF | tumor necrosis factor (TNF superfamily, | 2.751254082 | Yes |
| | member 2) | | |
| GZMB | granzyme B (granzyme 2, cytotoxic T- | 2.667121887 | Yes |
| | lymphocyte-associated serine esterase 1) | | |
| CCR1 | chemokine (C-C motif) receptor 1 | 2.132987738 | Yes |
| CCR5 | chemokine (C-C motif) receptor 5 | 2.054755688 | Yes |
| CCL5 | chemokine (C-C motif) ligand 5 | 1.827621102 | Yes |
| ITGAL | integrin, alpha L (antigen CD11A (p180), | 1.800091743 | Yes |
| | lymphocyte function-associated antigen 1; | | |
| | alpha polypeptide) | | |
| CD7 | CD7 molecule | 1.628337383 | Yes |
| FGR | Gardner-Rasheed feline sarcoma viral (v-fgr) | 1.552860975 | Yes |
| | oncogene homolog | | |
| CCL22 | chemokine (C-C motif) ligand 22 | 1.440871716 | Yes |
| TLR1 | toll-like receptor 1 | 1.34019506 | Yes |
| IL12A | interleukin 12A (natural killer cell | 1.315529704 | Yes |
| | stimulatory factor 1, cytotoxic lymphocyte | | |
| | maturation factor 1, p35) | | |
| CCL4 | chemokine (C-C motif) ligand 4 | 1.303452969 | Yes |
| GPR65 | G protein-coupled receptor 65 | 1.301272631 | Yes |
| CCR2 | chemokine (C-C motif) receptor 2 | 1.17247808 | Yes |
| PRF1 | perforin 1 (pore forming protein) | 1.171788573 | Yes |
| IGSF6 | immunoglobulin superfamily, member 6 | 1.100607872 | Yes |
| LYN | v-yes-1 Yamaguchi sarcoma viral related | 1.097683549 | Yes |
| | oncogene homolog | | |
| LCP2 | lymphocyte cytosolic protein 2 (SH2 | 1.090578794 | Yes |
| | domain containing leukocyte protein of | | |
| | 76kDa) | | |
| IL2RG | interleukin 2 receptor, gamma (severe | 1.083326221 | Yes |
| | combined immunodeficiency) | | |
| DYRK3 | dual-specificity tyrosine-(Y)- | 1.04365015 | Yes |
| | phosphorylation regulated kinase 3 | | |
| IRF8 | interferon regulatory factor 8 | 0.984531879 | Yes |
| TLR6 | toll-like receptor 6 | 0.974328399 | Yes |
| CCL2 | chemokine (C-C motif) ligand 2 | 0.932862163 | Yes |
| SOCS1 | suppressor of cytokine signaling 1 | 0.912176847 | Yes |
| FYB | FYN binding protein (FYB-120/130) | 0.841911793 | Yes |
| STAB1 | stabilin 1 | 0.800862968 | Yes |
| CCL19 | chemokine (C-C motif) ligand 19 | 0.784207284 | Yes |
| ITGB2 | integrin, beta 2 (complement component 3 | 0.783176661 | Yes |
| | receptor 3 and 4 subunit) | | |
| IL10 | interleukin 10 | 0.777381659 | Yes |
| BCL3 | B-cell CLL/lymphoma 3 | 0.768477619 | Yes |
| ICAM1 | intercellular adhesion molecule 1 (CD54), | 0.746375382 | Yes |
| | human rhinovirus receptor | | |
| MMP9 | matrix metallopeptidase 9 (gelatinase B, | 0.740974486 | Yes |
| | 92kDa gelatinase, 92kDa type IV | | |
| | collagenase) | | |
| IL13 | interleukin 13 | 0.6945768 | Yes |
| IL11 | interleukin 11 | 0.684957564 | Yes |
| TLR2 | toll-like receptor 2 | 0.671268702 | Yes |
| GCNT1 | glucosaminyl (N-acetyl) transferase 1, core | 0.671001792 | Yes |
| | 2 (beta-1,6-N- | | |
| | acetylglucosaminyltransferase) | | |
| IL4 | interleukin 4 | 0.65638262 | Yes |
| CDKN2A | cyclin-dependent kinase inhibitor 2A | 0.650471985 | Yes |
| | (melanoma, p16, inhibits CDK4) | | |
| CD86 | CD86 molecule | 0.631752431 | Yes |
| CD80 | CD80 molecule | 0.600226164 | Yes |
| LTB | lymphotoxin beta (TNF superfamily, | 0.579620421 | Yes |
| | member 3) | | |
| LIF | leukemia inhibitory factor (cholinergic | 0.547570944 | Yes |
| | differentiation factor) | | |
| WAS | Wiskott-Aldrich syndrome (eczema- | 0.525824308 | Yes |
| | thrombocytopenia) | | |
| PTPRC | protein tyrosine phosphatase, receptor type, C | 0.522265971 | Yes |
| RPS19 | ribosomal protein S19 | 0.506382108 | Yes |
| CD40 | CD40 molecule, TNF receptor superfamily | 0.492939293 | Yes |
| | member 5 | | |
| NCF4 | neutrophil cytosolic factor 4, 40kDa | 0.465533584 | Yes |
| LY86 | lymphocyte antigen 86 | 0.449851602 | Yes |
| FCGR2B | Fc fragment of IgG, low affinity IIb, | 0.436047524 | Yes |
| | receptor (CD32) | | |
| HCLS1 | hematopoietic cell-specific Lyn substrate 1 | 0.427405208 | Yes |
| CD3D | CD3d molecule, delta (CD3-TCR complex) | 0.415081888 | Yes |
| CXCL9 | chemokine (C-X-C motif) ligand 9 | 0.391491205 | Yes |
| CXCR3 | chemokine (C-X-C motif) receptor 3 | 0.380579948 | Yes |
| BRCA1 | breast cancer 1, early onset | 0.38035053 | Yes |
| RPL9 | ribosomal protein L9 | 0.372396767 | Yes |

KEGG_RIBOSOME

| | | | |
|---|---|---|---|
| RPL17 | ribosomal protein L17 | 1.672627926 | Yes |
| RPL29 | ribosomal protein L29 | 1.535640955 | Yes |
| RPS21 | ribosomal protein S21 | 1.305340767 | Yes |
| RPS16 | ribosomal protein S16 | 1.209875584 | Yes |
| RPL7 | ribosomal protein L7 | 1.179243684 | Yes |
| RPS6 | ribosomal protein S6 | 0.962560833 | Yes |
| RPL35 | ribosomal protein L35 | 0.879049003 | Yes |
| RPL38 | ribosomal protein L38 | 0.852828383 | Yes |
| RPL11 | ribosomal protein L11 | 0.769688666 | Yes |
| RPL4 | ribosomal protein L4 | 0.757808089 | Yes |
| RPL32 | ribosomal protein L32 | 0.755478978 | Yes |
| RP515 | ribosomal protein S15 | 0.75469929 | Yes |
| RPL14 | ribosomal protein L14 | 0.718787491 | Yes |
| RPS25 | ribosomal protein S25 | 0.702086389 | Yes |
| RPL24 | ribosomal protein L24 | 0.687731743 | Yes |
| RPS8 | ribosomal protein S8 | 0.68595165 | Yes |
| RPS24 | ribosomal protein S24 | 0.663855553 | Yes |
| RPL21 | ribosomal protein L21 | 0.640351593 | Yes |
| RPS28 | ribosomal protein S28 | 0.610872269 | Yes |
| FAU | Finkel-Biskis-Reilly murine sarcoma | 0.550771177 | Yes |
| | virus (FBR-MuSV) ubiquitously | | |
| | expressed (fox derived); ribosomal | | |
| | protein S30 | | |
| RPS5 | ribosomal protein S5 | 0.538966238 | Yes |
| RPL34 | ribosomal protein L34 | 0.533506274 | Yes |
| RPLPO | ribosomal protein, large, P0 | 0.522892892 | Yes |
| RPL6 | ribosomal protein L6 | 0.506569028 | Yes |
| RPS19 | ribosomal protein S19 | 0.506382108 | Yes |
| RPS27A | ribosomal protein S27a | 0.483083695 | Yes |
| RPL7A | ribosomal protein L7a | 0.443502933 | Yes |
| RPL13 | ribosomal protein L13 | 0.387363732 | Yes |
| RPS13 | ribosomal protein S13 | 0.383430898 | Yes |
| RPLP2 | ribosomal protein, large, P2 | 0.374124438 | Yes |
| RPL9 | ribosomal protein L9 | 0.372396767 | Yes |
| RPS18 | ribosomal protein S18 | 0.335394561 | Yes |
| RPS15A | ribosomal protein S15a | 0.329340279 | Yes |
| RPL35A | ribosomal protein L35a | 0.322530985 | Yes |
| RPL37 | ribosomal protein L37 | 0.275145084 | Yes |
| RPL22L1 | ribosomal protein L22-like 1 | 0.273913294 | Yes |
| RPL30 | ribosomal protein L30 | 0.266558886 | Yes |
| RPL23A | ribosomal protein L23a | 0.26068905 | Yes |
| RPS7 | ribosomal protein S7 | 0.257175088 | Yes |
| RPS2 | ribosomal protein S2 | 0.256049454 | Yes |
| RPL18A | ribosomal protein L18a | 0.228949845 | Yes |
| RPS20 | ribosomal protein S20 | 0.214455262 | Yes |
| RPL36A | ribosomal protein L36a | 0.185356036 | Yes |
| RPS29 | ribosomal protein S29 | 0.179608196 | Yes |
| RPL12 | ribosomal protein L12 | 0.177713573 | Yes |
| RPS3 | ribosomal protein S3 | 0.166580707 | Yes |
| RPS26 | ribosomal protein S26 | 0.166472122 | Yes |
| RPL22 | ribosomal protein L22 | 0.165959805 | Yes |
| RPS17 | ribosomal protein S17 | 0.152504608 | Yes |
| RPL37A | ribosomal protein L37a | 0.151727423 | Yes |
| RPL39 | ribosomal protein L39 | 0.145141497 | Yes |
| RPS27 | ribosomal protein S27 | 0.138581008 | Yes |
| | (metallopanstimulin 1) | | |
| RPL10 | ribosomal protein L10 | 0.114015959 | No |
| RPSA | ribosomal protein SA | 0.113453649 | No |
| RPS4X | ribosomal protein S4, X-linked | 0.107207999 | No |
| RPS12 | ribosomal protein S12 | 0.095793545 | No |
| RPL13A | ribosomal protein L13a | 0.094446741 | No |
| RPL27A | ribosomal protein L27a | 0.065862156 | No |
| RPL28 | ribosomal protein L28 | 0.041829564 | No |
| RPL5 | ribosomal protein L5 | 0.024316726 | No |
| RPL23 | ribosomal protein L23 | −0.010726028 | No |
| RPS27L | ribosomal protein S27-like | −0.061738685 | No |
| RPL31 | ribosomal protein L31 | −0.072339609 | No |
| RPL3 | ribosomal protein L3 | −0.090979747 | No |
| RPS23 | ribosomal protein S23 | −0.110026583 | No |
| RPL10A | ribosomal protein L10a | −0.118189119 | No |
| RPS9 | ribosomal protein S9 | −0.145742506 | No |
| RPL41 | ribosomal protein L41 | −0.153432697 | No |
| RPL18 | ribosomal protein L18 | −0.165799722 | No |
| RPL26 | ribosomal protein L26 | −0.168741375 | No |
| RPL36AL | ribosomal protein L36a-like | −0.17463319 | No |
| RPS10 | ribosomal protein S10 | −0.186098009 | No |
| RPL27 | ribosomal protein L27 | −0.269447058 | No |
| RPS11 | ribosomal protein S11 | −0.335371464 | No |
| UBA52 | ubiquitin A-52 residue ribosomal | −0.479723126 | No |
| | protein fusion product 1 | | |
| RPL15 | ribosomal protein L15 | −0.572703719 | No |
| MRPL13 | mitochondrial ribosomal protein L13 | −0.590085208 | No |
| RPL8 | ribosomal protein L8 | −0.731413662 | No |
| RPL36 | ribosomal protein L36 | −0.76863873 | No |
| RPL19 | ribosomal protein L19 | −1.015113831 | No |
| RPL3L | ribosomal protein L3-like | −1.196903586 | No |

KEGG_NOD_LIKE_RECEPTOR_SIGNALING_PATHWAY

| | | | |
|---|---|---|---|
| IL1B | interleukin 1, beta | 3.462865114 | Yes |
| IL6 | interleukin 6 (interferon, beta 2) | 3.456357479 | Yes |
| TNF | tumor necrosis factor (TNF superfamily, | 2.751254082 | Yes |
| | member 2) | | |
| CXCL1 | chemokine (C-X-C motif) ligand 1 | 2.667189121 | Yes |
| | (melanoma growth stimulating activity, | | |
| | alpha) | | |
| MEFV | Mediterranean fever | 2.353091955 | Yes |
| CXCL2 | chemokine (C-X-C motif) ligand 2 | 1.990867496 | Yes |
| CCL5 | chemokine (C-C motif) ligand 5 | 1.827621102 | Yes |
| CCL2 | chemokine (C-C motif) ligand 2 | 0.932862163 | Yes |
| CARD6 | caspase recruitment domain family, | 0.672573209 | No |
| | member 6 | | |
| CASP1 | caspase 1, apoptosis-related cysteine | 0.499731153 | No |
| | peptidase (interleukin 1, beta, convertase) | | |
| CCL7 | chemokine (C-C motif) ligand 7 | 0.251811057 | No |
| PSTPIP1 | proline-serine-threonine phosphatase | 0.226958573 | No |
| | interacting protein 1 | | |
| MAPK11 | mitogen-activated protein kinase 11 | 0.193452582 | No |
| TNFAIP3 | tumor necrosis factor, alpha-induced | 0.100990877 | No |
| | protein 3 | | |
| MAPK9 | mitogen-activated protein kinase 9 | 0.090808034 | No |
| CCL11 | chemokine (C-C motif) ligand 11 | 0.039969306 | No |
| MAPK8 | mitogen-activated protein kinase 8 | 0.032760639 | No |
| PYCARD | PYD and CARD domain containing | −0.03229328 | No |
| CASP8 | caspase 8, apoptosis-related cysteine | −0.03536839 | No |
| | peptidase | | |
| NFKBIA | nuclear factor of kappa light polypeptide | −0.03587324 | No |
| | gene enhancer in B-cells inhibitor, alpha | | |
| SUGT1 | SGT1, suppressor of G2 allele of SKP1 | −0.05679465 | No |
| | (S. cerevisiae) | | |
| CARD9 | caspase recruitment domain family, | −0.06568909 | No |
| | member 9 | | |
| BIRC3 | baculoviral IAP repeat-containing 3 | −0.06623559 | No |
| MAPK3 | mitogen-activated protein kinase 3 | −0.12906235 | No |
| TRIP6 | thyroid hormone receptor interactor 6 | −0.13364181 | No |
| MAPK14 | mitogen-activated protein kinase 14 | −0.13693111 | No |
| MAP3K7 | mitogen-activated protein kinase kinase | −0.14027362 | No |
| | kinase 7 | | |
| IKBKB | inhibitor of kappa light polypeptide gene | −0.16735205 | No |
| | enhancer in B-cells, kinase beta | | |
| TRAF6 | TNF receptor-associated factor 6 | −0.17263973 | No |
| MAPK1 | mitogen-activated protein kinase 1 | −0.17270541 | No |
| RIPK2 | receptor-interacting serine-threonine | −0.1842809 | No |
| | kinase 2 | | |
| IKBKG | inhibitor of kappa light polypeptide gene | −0.19543105 | No |
| | enhancer in B-cells, kinase gamma | | |
| MAPK10 | mitogen-activated protein kinase 10 | −0.25465888 | No |
| HSP90AA1 | heat shock protein 90kDa alpha | −0.25493035 | No |
| | (cytosolic), class A member 1 | | |
| NFKB1 | nuclear factor of kappa light polypeptide | −0.25809258 | No |
| | gene enhancer in B-cells 1 (p105) | | |
| CHUK | conserved helix-loop-helix ubiquitous | −0.32112595 | No |
| | kinase | | |
| RELA | v-rel reticuloendotheliosis viral | −0.32651395 | No |
| | oncogene homolog A, nuclear factor of | | |
| | kappa light polypeptide gene enhancer | | |
| | in B-cells 3, p65 (avian) | | |
| HSP90AB1 | heat shock protein 90kDa alpha | −0.35596654 | No |
| | (cytosolic), class B member 1 | | |
| NFKBIB | nuclear factor of kappa light polypeptide | −0.40253881 | No |
| | gene enhancer in B-cells inhibitor, beta | | |
| MAPK12 | mitogen-activated protein kinase 12 | −0.41511443 | No |
| BIRC2 | baculoviral IAP repeat-containing 2 | −0.4354732 | No |
| MAPK13 | mitogen-activated protein kinase 13 | −0.45562434 | No |
| HSP90B1 | heat shock protein 90kDa beta (Grp94), | −0.50010961 | No |
| | member 1 | | |
| IL18 | interleukin 18 (interferon-gamma- | −0.5164243 | No |
| | inducing factor) | | |
| CCL8 | chemokine (C-C motif) ligand 8 | −1.63008225 | No |

KEGG_TYPE_I DIABETES_MELLITUS

| | | | |
|---|---|---|---|
| IL1B | interleukin 1, beta | 3.462865114 | Yes |
| TNF | tumor necrosis factor (TNF superfamily, | 2.751254082 | Yes |
| | member 2) | | |
| GZMB | granzyme B (granzyme 2, cytotoxic T- | 2.667121887 | Yes |
| | lymphocyte-associated serine esterase 1) | | |
| IL12A | interleukin 12A (natural killer cell | 1.315529704 | Yes |
| | stimulatory factor 1, cytotoxic | | |
| | lymphocyte maturation factor 1, p35) | | |
| PRF1 | perforin 1 (pore forming protein) | 1.171788573 | Yes |
| CD86 | CD86 molecule | 0.631752431 | Yes |
| CD80 | CD80 molecule | 0.600226164 | Yes |
| LTA | lymphotoxin alpha (TNF superfamily, | 0.44320941 | Yes |
| | member 1) | | |
| CD28 | CD28 molecule | 0.331128299 | No |
| FAS | Fas (TNF receptor superfamily, member 6) | 0.173293099 | No |
| PTPRN | protein tyrosine phosphatase, receptor | 0.112962097 | No |
| | type, N | | |
| GAD1 | glutamate decarboxylase 1 (brain, | −0.03957521 | No |
| | 67kDa) | | |
| ICA1 | islet cell autoantigen 1, 69kDa | −0.0870818 | No |
| ILIA | interleukin 1, alpha | −0.22882077 | No |
| PTPRN2 | protein tyrosine phosphatase, receptor | −0.23292804 | No |
| | type, N polypeptide 2 | | |
| CPE | carboxypeptidase E | −0.5967598 | No |
| HSPD1 | heat shock 60kDa protein 1 (chaperonin) | −0.8842746 | No |

### REFERENCES

1. Dick G W, Kitchen S F, Haddow A J. Zika virus. I. Isolations and serological specificity. Transactions of the Royal Society of Tropical Medicine and Hygiene. 1952; 46(5):509-20. Epub 1952 Sep. 1. PubMed PMID: 12995440.
2. Dick G W. Zika virus. II. Pathogenicity and physical properties. Transactions of the Royal Society of Tropical Medicine and Hygiene. 1952; 46(5):521-34. Epub 1952 Sep. 1. PubMed PMID: 12995441.
3. Macnamara F N. Zika virus: a report on three cases of human infection during an epidemic of jaundice in Nigeria. Transactions of the Royal Society of Tropical Medicine and Hygiene. 1954; 48(2):139-45. Epub 1954 Mar. 1. PubMed PMID: 13157159.
4. Robin Y, Mouchet J. [Serological and entomological study on yellow fever in Sierra Leone]. Bulletin de la Societe de pathologie exotique et de ses filiales. 1975; 68(3):249-58. Epub 1975 May 1. PubMed PMID: 1243735.
5. Kirya B G, Okia N O. A yellow fever epizootic in Zika Forest, Uganda, during 1972: Part 2: Monkey serology. Transactions of the Royal Society of Tropical Medicine and Hygiene. 1977; 71(4):300-3. Epub 1977 Jan. 1. PubMed PMID: 413216.
6. Jan C, Languillat G, Renaudet J, Robin Y. [A serological survey of arboviruses in Gabon]. Bulletin de la Societe de pathologie exotique et de ses filiales. 1978; 71(2):140-6. Epub 1978 Mar. 1. PubMed PMID: 743766.
7. Fagbami A H. Zika virus infections in Nigeria: virological and seroepidemiological investigations in Oyo State. The Journal of hygiene. 1979; 83(2):213-9. Epub 1979 Oct. 1. PubMed PMID: 489960; PMCID: 2129900.
8. Saluzzo J F, Gonzalez J P, Herve J P, Georges A J. [Serological survey for the prevalence of certain arboviruses in the human population of the south-east area of Central African Republic (author's transl)]. Bulletin de la Societe de pathologie exotique et de ses filiales. 1981; 74(5):490-9. Epub 1981 Sep. 1. PubMed PMID: 6274526.
9. Olson J G, Ksiazek T G, Suhandiman, Triwibowo. Zika virus, a cause of fever in Central Java, Indonesia. Transactions of the Royal Society of Tropical Medicine and Hygiene. 1981; 75(3):389-93. Epub 1981 Jan. 1. PubMed PMID: 6275577.
10. McCrae A W, Kirya B G. Yellow fever and Zika virus epizootics and enzootics in Uganda. Transactions of the Royal Society of Tropical Medicine and Hygiene. 1982; 76(4):552-62. Epub 1982 Jan. 1. PubMed PMID: 6304948.
11. Monlun E, Zeller H, Le Guenno B, Traore-Lamizana M, Hervy J P, Adam F, Ferrara L, Fontenille D, Sylla R, Mondo M, et al. [Surveillance of the circulation of arbovirus of medical interest in the region of eastern Senegal]. Bull Soc Pathol Exot. 1993; 86(1):21-8. Epub 1993 Jan. 1. PubMed PMID: 8099299.
12. Chao G, Lau W L, Hackel B J, Sazinsky S L, Lippow S M, Wittrup K D. Isolating and engineering human antibodies using yeast surface display. Nature protocols. 2006; 1(2):755-68. doi: 10.1038/nprot.2006.94. PubMed PMID: 17406305.
13. Lanciotti R S, Kosoy O L, Laven J J, Velez J O, Lambert A J, Johnson A J, Stanfield S M, Duffy M R. Genetic and serologic properties of Zika virus associated with an epidemic, Yap State, Micronesia, 2007. Emerging infectious diseases. 2008; 14(8):1232-9. Epub 2008 Aug. 6. doi: 10.3201/eid1408.080287. PubMed PMID: 18680646; PMCID: 2600394.
14. Duffy M R, Chen T H, Hancock W T, Powers A M, Kool J L, Lanciotti R S, Pretrick M, Marfel M, Holzbauer S, Dubray C, Guillaumot L, Griggs A, Bel M, Lambert A J, Laven J, Kosoy O, Panella A, Biggerstaff B J, Fischer M, Hayes E B. Zika virus outbreak on Yap Island, Federated States of Micronesia. N Engl J Med. 2009; 360(24):2536-43. doi: 10.1056/NEJMoa0805715. PubMed PMID: 19516034.
15. Bearcroft W G. Zika virus infection experimentally induced in a human volunteer. Transactions of the Royal Society of Tropical Medicine and Hygiene. 1956; 50(5):442-8. Epub 1956 Jan. 1. PubMed PMID: 13380987.
16. Simpson D I. Zika Virus Infection in Man. Transactions of the Royal Society of Tropical Medicine and Hygiene. 1964; 58:335-8. Epub 1964 Jul. 1. PubMed PMID: 14175744.
17. Oehler E, Watrin L, Larre P, Leparc-Goffart I, Lastere S, Valour F, Baudouin L, Mallet H, Musso D, Ghawche F. Zika virus infection complicated by Guillain-Barre syndrome—case report, French Polynesia, December 2013. Euro surveillance: bulletin Europeen sur les maladies transmissibles=European communicable disease bulletin. 2014; 19(9). Epub 2014 Mar. 15. PubMed PMID: 24626205.
18. loos S, Mallet H P, Leparc Goffart I, Gauthier V, Cardoso T, Herida M. Current Zika virus epidemiology and recent epidemics. Medecine et maladies infectieuses. 2014; 44(7):302-7. doi: 10.1016/j.medmal.2014.04.008. PubMed PMID: 25001879.
19. Bogoch, I I, Brady O J, Kraemer M U G, German M, Creatore M I, Kulkarni M A, Brownstein J S, Mekaru S R, Hay S I, Groot E, Watts A, Khan K. Anticipating the international spread of Zika virus from Brazil. Lancet. 2016; 387(10016):335-6. Epub 2016 Jan. 19. doi: 10.1016/S0140-6736(16)00080-5. PubMed PMID: 26777915; PMCID: 4873159.
20. Ma W, Li S, Ma S, Jia L, Zhang F, Zhang Y, Zhang J, Wong G, Zhang S, Lu X, Liu M, Yan J, Li W, Qin C, Han D, Wang N, Li X, Gao G F. Zika Virus Causes Testis Damage and Leads to Male Infertility in Mice. Cell. 2016; 167(6):1511-24 e10. Epub 2016 Nov. 26. doi: 10.1016/j.cell.2016.11.016. PubMed PMID: 27884405.
21. Govero J, Esakky P, Scheaffer S M, Fernandez E, Drury A, Platt D J, Gorman M J, Richner J M, Caine E A, Salazar V, Moley K H, Diamond M S. Zika virus infection damages the testes in mice. Nature. 2016; 540(7633):438-42. Epub 2016 Nov. 1. doi: 10.1038/nature20556. PubMed PMID: 27798603; PMCID: 5432198.
22. Uraki R, Jurado K A, Hwang J, Szigeti-Buck K, Horvath T L, Iwasaki A, Fikrig E. Fetal Growth Restriction Caused by Sexual Transmission of Zika Virus in Mice. The Journal of infectious diseases. 2017; 215(11):1720-4. Epub 2017 May 5. doi: 10.1093/infdis/jix204. PubMed PMID: 28472297.
23. Uraki R, Hwang J, Jurado K A, Householder S, Yockey L J, Hastings A K, Homer R J, Iwasaki A, Fikrig E. Zika virus causes testicular atrophy. Science advances. 2017; 3(2):e1602899. Epub 2017 Mar. 7. doi: 10.1126/sciadv.1602899. PubMed PMID: 28261663; PMCID: 5321463.
24. Schneider B S, McGee C E, Jordan J M, Stevenson H L, Soong L, Higgs S. Prior exposure to uninfected mosquitoes enhances mortality in naturally-transmitted West Nile virus infection. PloS one. 2007; 2(11):e1171. doi: 10.1371/journal.pone.0001171. PubMed PMID: 18000543; PMCID: 2048662.
25. Conway M J, Watson A M, Colpitts T M, Dragovic S M, Li Z, Wang P, Feitosa F, Shepherd D T, Ryman K D, Klimstra W B, Anderson J F, Fikrig E. Mosquito saliva serine protease enhances dissemination of dengue virus into the mammalian host. Journal of Virology. 2014; 88(1):164-75. doi: 10.1128/JVI.02235-13. PubMed PMID: 24131723; PMCID: 3911723.
26. Jin L, Guo X, Shen C, Hao X, Sun P, Li P, Xu T, Hu C, Rose O, Zhou H, Yang M, Qin C F, Guo J, Peng H, Zhu M, Cheng G, Qi X, Lai R. Salivary factor LTRIN from Aedes aegypti facilitates the transmission of Zika virus by interfering with the lymphotoxin-beta receptor. Nature immunology. 2018. Epub 2018 Mar. 7. doi: 10.1038/s41590-018-0063-9. PubMed PMID: 29507355.
27. Machain-Williams C, Reagan K, Wang T, Zeidner N S, Blair C D. Immunization with Culex tarsalis mosquito salivary gland extract modulates West Nile virus infection and disease in mice. Viral immunology. 2013; 26(1):84-92. Epub 2013 Feb. 1. doi: 10.1089/vim.2012.0051. PubMed PMID: 23362833; PMCID: PMC3578371.
28. Rohousova I, Volf P. Sand fly saliva: effects on host immune response and Leishmania transmission. Folia parasitologica. 2006; 53(3):161-71. Epub 2006 Nov. 24. PubMed PMID: 17120496.
29. Horka H, Cerna-Kyckova K, Skallova A, Kopecky J. Tick saliva affects both proliferation and distribution of Borrelia burgdorferi spirochetes in mouse organs and increases transmission of spirochetes to ticks. International journal of medical microbiology: IJMM. 2009; 299(5):373-80. doi: 10.1016/j.ijmm.2008.10.009. PubMed PMID: 19147403.
30. Cox J, Mota J, Sukupolvi-Petty S, Diamond M S, Rico-Hesse R. Mosquito bite delivery of dengue virus enhances immunogenicity and pathogenesis in humanized mice. Journal of virology. 2012; 86(14):7637-49. doi: 10.1128/JVI.00534-12. PubMed PMID: 22573866; PMCID: 3416288.
31. Pingen M, Bryden S R, Pondeville E, Schnettler E, Kohl A, Merits A, Fazakerley J K, Graham G J, McKimmie C S. Host Inflammatory Response to Mosquito Bites Enhances the Severity of Arbovirus Infection. Immunity. 2016; 44(6):1455-69. Epub 2016 Jun. 23. doi: 10.1016/j.immuni.2016.06.002. PubMed PMID: 27332734; PMCID: 4920956.
32. Styer L M, Lim P Y, Louie K L, Albright R G, Kramer L D, Bernard K A. Mosquito saliva causes enhancement of West Nile virus infection in mice. Journal of virology. 2011; 85(4):1517-27. doi: 10.1128/JVI.01112-10. PubMed PMID: 21147918; PMCID: 3028906.
33. Uraki R, Hastings A K, Gloria-Soria A, Powell J R, Fikrig E. Altered vector competence in an experimental mosquito-mouse transmission model of Zika infection. PLoS neglected tropical diseases. 2018; 12(3):e0006350. Epub 2018 Mar. 6. doi: 10.1371/journal.pntd.0006350. PubMed PMID: 29505571.
34. Aliota M T, Caine E A, Walker E C, Larkin K E, Camacho E, Osorio J E. Characterization of Lethal Zika Virus Infection in AG129 Mice. PLoS neglected tropical diseases. 2016; 10(4):e0004682. Epub 2016 Apr. 20. doi: 10.1371/journal.pntd.0004682. PubMed PMID: 27093158; PMCID: 4836712.
35. Flach T L, Ng G, Hari A, Desrosiers M D, Zhang P, Ward S M, Seamone M E, Vilaysane A, Mucsi A D, Fong Y, Prenner E, Ling C C, Tschopp J, Muruve D A, Amrein M W, Shi Y. Alum interaction with dendritic cell membrane lipids is essential for its adjuvanticity. Nature medicine. 2011; 17(4):479-87. Epub 2011 Mar. 15. doi: 10.1038/nm.2306. PubMed PMID: 21399646.
36. Alving C R, Peachman K K, Rao M, Reed S G. Adjuvants for human vaccines. Current opinion in immunology. 2012; 24(3):310-5. Epub 2012 Apr. 24. doi: 10.1016/j.coi.2012.03.008. PubMed PMID: 22521140; PMCID: 3383374.
37. Mbow M L, De Gregorio E, Valiante N M, Rappuoli R. New adjuvants for human vaccines. Current opinion in immunology. 2010; 22(3):411-6. Epub 2010 May 15. doi: 10.1016/j.coi.2010.04.004. PubMed PMID: 20466528.
38. Coffman R L, Sher A, Seder R A. Vaccine adjuvants: putting innate immunity to work. Immunity. 2010; 33(4):492-503. Epub 2010 Oct. 30. doi: 10.1016/j.immuni.2010.10.002. PubMed PMID: 21029960; PMCID: 3420356.
39. Lazear H M et al., A Mouse Model of Zika Virus Pathogenesis. Cell Host Microbe. 2016; 19(5):720-730.
40. Uraki, R., Hastings, A. K., Gloria-Soria, A., Powell, J. R. & Fikrig, E. Altered vector competence in an experimental mosquito-mouse transmission model of Zika infection. PLoS Negl Trop Dis 12, e0006350, doi:10.1371/journal.pntd.0006350 (2018).
41. Shi L and Paskewitz, S M, Identification and molecular characterization of two immune-responsive chitinase-like proteins from Anopheles gambiae. Insect Molecular Biology. 2004, 13(4):387-398.
42. Beatty, P. R., et al. Dengue virus NS1 triggers endothelial permeability and vascular leak that is prevented by NS1 vaccination. Science Translational Medicine 7(2015).
43. Tanaka, T. & Kishimoto, T. The Biology and Medical Implications of Interleukin-6. Cancer Immunol Res 2, 288-294 (2014).
44. Calvo, E., Mans, B. J., Andersen, J. F. & Ribeiro, J. M. Function and evolution of a mosquito salivary protein family. J Biol Chem 281, 1935-1942 (2006).
45. Rathore, A. P. S. & St John, A. L. Immune responses to dengue virus in the skin. Open Biol 8(2018).
46. Shi, C. & Pamer, E. G. Monocyte recruitment during infection and inflammation. Nat Rev Immunol 11, 762-774 (2011).
47. Osuna, C. E., et al. Zika viral dynamics and shedding in rhesus and cynomolgus macaques. Nat Med 22, 1448-1455 (2016).
48. Bai, F., et al. A paradoxical role for neutrophils in the pathogenesis of West Nile virus. J Infect Dis 202, 1804-1812 (2010).
49. Fong, S. W., Kini, R. M. & Ng, L. F. P. Mosquito Saliva Reshapes Alphavirus Infection and Immunopathogenesis. J Virol 92 (2018).
50. Schuijt, T. J., et al. Identification and characterization of Ixodes scapularis antigens that elicit tick immunity using yeast surface display. PLoS One 6, e15926 (2011).
51. Nakajima, S., et al. Prostaglandin I2-IP signaling promotes Th1 differentiation in a mouse model of contact hypersensitivity. J Immunol 184, 5595-5603 (2010).
52. Chang, Q., Ornatsky, O. & Hedley, D. Staining of Frozen and Formalin-Fixed, Paraffin-Embedded Tissues with Metal-Labeled Antibodies for Imaging Mass Cytometry Analysis. Curr Protoc Cytom 82, 12 47 11-12 47 18 (2017).
53. Kramer, L. D., Styer, L. M. & Ebel, G. D. A global perspective on the epidemiology of West Nile virus. Annu Rev Entomol 53, 61-81, doi:10.1146/annurev.ento.53.103106.093258 (2008).
54. McGee, C. E., Schneider, B. S., Girard, Y. A., Vanlandingham, D. L. & Higgs, S. Nonviremic transmission of West Nile virus: evaluation of the effects of space, time, and mosquito species. Am J Trop Med Hyg 76, 424-430 (2007).
55. Reisen, W. & Brault, A. C. West Nile virus in North America: perspectives on epidemiology and intervention. Pest Manag Sci 63, 641-646, doi:10.1002/ps.1325 (2007).
56. Nikolay, B. A review of West Nile and Usutu virus co-circulation in Europe: how much do transmission cycles overlap? Trans R Soc Trop Med Hyg 109, 609-618, doi:10.1093/trstmh/trv066 (2015).
57. Petersen, L. R., Brault, A. C. & Nasci, R. S. West Nile virus: review of the literature. JAMA 310, 308-315, doi:10.1001/jama.2013.8042 (2013).
58. Vogels, C. B., Goertz, G. P., Pijlman, G. P. & Koenraadt, C. J. Vector competence of European mosquitoes for West Nile virus. Emerg Microbes Infect 6, e96, doi:10.1038/emi.2017.82 (2017).
59. Gould, E. A. & Solomon, T. Pathogenic flaviviruses. Lancet 371, 500-509, doi:10.1016/50140-6736(08)60238-X (2008).
60. Vanlandingham, D. L. et al. Relative susceptibilties of South Texas mosquitoes to infection with West Nile virus. Am J Trop Med Hyg 77, 925-928 (2007).
61. Andersen, L. K. & Davis, M. D. Climate change and the epidemiology of selected tick-borne and mosquito-borne diseases: update from the International Society of Dermatology Climate Change Task Force. Int J Dermatol 56, 252-259, doi:10.1111/ijd.13438 (2017).
62. Turell, M. J., O'Guinn, M. L., Dohm, D. J. & Jones, J. W. Vector competence of North American mosquitoes (Diptera: Culicidae) for West Nile virus. J Med Entomol 38, 130-134, doi:10.1603/0022-2585-38.2.130 (2001).
63. Matthews, B. J. et al. Improved reference genome of Aedes aegypti informs arbovirus vector control. Nature 563, 501-507, doi:10.1038/s41586-018-0692-z (2018).
64. Coutinho-Abreu, I. V., Guimaraes-Costa, A. B. & Valenzuela, J. G. Impact of Insect Salivary Proteins in Blood Feeding, Host Immunity, Disease, and in the Development of Biomarkers for Vector Exposure. Curr Opin Insect Sci 10, 98-103, doi:10.1016/j.cois.2015.04.014 (2015).
65. Fontaine, A. et al. Implication of haematophagous arthropod salivary proteins in host-vector interactions. Parasit Vectors 4, 187, doi:10.1186/1756-3305-4-187 (2011).
66. Hayashi, H. et al. Anopheline anti-platelet protein from a malaria vector mosquito has anti-thrombotic effects in vivo without compromising hemostasis. Thromb Res 129, 169-175, doi:10.1016/j.thromres.2011.09.015 (2012).
67. Conway, M. J. et al. Aedes aegypti D7 Saliva Protein Inhibits Dengue Virus Infection. PLoS Negl Trop Dis 10, e0004941, doi:10.1371/journal.pntd.0004941 (2016).
68. Jin, L. et al. Salivary factor LTRIN from Aedes aegypti facilitates the transmission of Zika virus by interfering with the lymphotoxin-beta receptor. Nat Immunol 19, 342-353, doi:10.1038/s41590-018-0063-9 (2018).
69. Uraki, R. et al. Aedes aegypti AgBR1 antibodies modulate early Zika virus infection of mice. Nat Microbiol, doi:10.1038/s41564-019-0385-x (2019).
70. Ribeiro, J. M. et al. An annotated catalogue of salivary gland transcripts in the adult female mosquito, Aedes aegypti. BMC Genomics 8, 6, doi:10.1186/1471-2164-8-6 (2007).
71. Cheng, G. et al. A C-type lectin collaborates with a CD45 phosphatase homolog to facilitate West Nile virus infection of mosquitoes. Cell 142, 714-725, doi:10.1016/j.cell.2010.07.038 (2010).
72. Hastings, A. K. et al. Aedes aegypti NeSt1 protein enhances Zika virus pathogenesis by activating neutrophils. J Virol, doi:10.1128/JVI.00395-19 (2019).
73. Wang, P. et al. Matrix metalloproteinase 9 facilitates West Nile virus entry into the brain. J Virol 82, 8978-8985, doi:10.1128/JVI.00314-08 (2008).
74. Ramos, H. J. et al. IL-1beta signaling promotes CNS-intrinsic immune control of West Nile virus infection. PLoS Pathog 8, e1003039, doi:10.1371/journal.ppat.1003039 (2012).
75. Ruckert, C. & Ebel, G. D. How Do Virus-Mosquito Interactions Lead to Viral Emergence? Trends Parasitol 34, 310-321 (2018).
76. Kieny, M. P., Excler, J. L. & Girard, M. Research and development of new vaccines against infectious diseases. Am J Public Health 94, 1931-1935 (2004).
77. Sarathy, V. V., Milligan, G. N., Bourne, N. & Barrett, A. D. Mouse models of dengue virus infection for vaccine testing. Vaccine 33, 7051-7060 (2015).
78. Zhu, J., Huang, X. & Yang, Y. Type I IFN signaling on both B and CD4 T cells is required for protective antibody response to adenovirus. J Immunol 178, 3505-3510 (2007).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A composition comprising two or more polypeptides, wherein said two or more polypeptides are selected from the group consisting of LOC5573204, LOC5578630, LOC5578631, LOC5567956, LOC5580038, LOC5566287, LOC5567958, LOC5568702, LOC110675548, and fragments, derivatives or variants thereof.

2. A composition comprising a nucleic acid molecule or two or more nucleic acid molecules encoding two or more polypeptides, wherein said two or more polypeptides are selected from the group consisting of LOC5573204, LOC5578630, LOC5578631, LOC5567956, LOC5580038, LOC5566287, LOC5567958, LOC5568702, LOC110675548, and fragments, derivatives or variants thereof.

3.-9. (canceled)

10. The composition of claim 2, wherein the two or more polypeptides comprise (i) LOC5573204 Bacteria-Responsive Protein 1 (AgBR1) or a fragment, derivative or variant thereof and (ii) LOC5578631 Neutrophil Stimulating Factor 1 (NetSt1) or a fragment, derivative or variant thereof.

11. The composition of claim 10, wherein the LOC5573204 polypeptide comprises the sequence SEQ ID NO: 1.

12.-13. (canceled)

14. The composition of claim 10, wherein the LOC5573204 polypeptide fragment comprises the sequence selected from SEQ ID NOS: 6-13.

15.-20. (canceled)

21. The composition of claim 10, wherein the LOC5578631 polypeptide comprises the sequence SEQ ID NO: 3.

22.-23. (canceled)

24. The composition of claim 10, wherein the LOC5578631 polypeptide fragment comprises the sequence selected from SEQ ID NOS: 22-29.

25.-54. (canceled)

55. The composition of claim 2, further comprising a carrier or excipient.

56. The composition of claim 2, further comprising an adjuvant.

57. (canceled)

58. A method of preventing or treating a disease in a subject in need thereof, wherein the disease is associated with a mosquito-borne infectious agent, said method comprising administering to said subject an effective amount of the composition of claim 2.

59. The method of claim 58, wherein the mosquito is Aedes aegypti.

60. The method of claim 58, wherein the mosquito-borne infectious agent is a mosquito-borne virus.

61. The method of claim 60, wherein the mosquito-borne virus is a flavivirus.

62. (canceled)

63. The method of claim 61, wherein the flavivirus is Zika virus.

64. The method of claim 61, wherein the flavivirus is West Nile virus.

65. The method of claim 60, wherein the mosquito-borne virus is an alphavirus.

66. (canceled)

67. The method of claim 58, wherein the subject is human.

68. A method of preventing or treating a disease in a subject in need thereof, wherein the disease is associated with a mosquito-borne infectious agent, said method comprising administering to said subject an effective amount of the composition of claim 1.

69. A method of preventing or treating a disease in a subject in need thereof, wherein the disease is associated with a mosquito-borne infectious agent, said method comprising administering to said subject an effective amount of two or more polypeptides, wherein said two or more polypeptides are selected from the group consisting of LOC5573204, LOC5578630, LOC5578631, LOC5567956, LOC5580038, LOC5566287, LOC5567958, LOC5568702, LOC110675548, and fragments, derivatives or variants thereof.

70. A method of preventing or treating a disease in a subject in need thereof, wherein the disease is associated with a mosquito-borne infectious agent, said method comprising administering to said subject an effective amount of two or more nucleic acid molecules encoding polypeptides selected from the group consisting of LOC5573204, LOC5578630, LOC5578631, LOC5567956, LOC5580038, LOC5566287, LOC5567958, LOC5568702, LOC110675548, and fragments, derivatives or variants thereof.
